SEQUENCE LISTING (54 sequences)

SEQ ID NO: 1 - DNA, 599 bp, Homo sapiens
gaccttgtgatctgcctgcctcggactcccaaagtgctaggattacaagtgtgagcaaccgtgcctggataatttttaaattttgtagacatggaatcttactctgttgccctggctggtctcaaactccaggttacccaaagtactgcgattacaggtgtgagccaccactctcagccttttctttttctttttttacaaaataaaatacactaagatttgaagaaatctcactcatctttaaaaagattcaatggcaatgaatcacttttctgcaaaggctggagttaggagaacccktggttgatggctttgttaattattattattattttttttaagattcaggttcacacttgtaatcccagcacattgggaggccaaggtggacagatcacctgagttcaggaattcgagaccagcctagccaacatggcaaaactccatccctactaaaaatacaaaaattagccaggtgtggtggcgcatgcctgtagtcccagctactcaggaggctgaggcaggagaatcacttaaacctgggaggggaatgttgaagtgaaccaagattgtgccactgcactccagcctgggtgaca

SEQ ID NO: 2 - DNA, 599 bp, Homo sapiens
catgtggcttatgactaagaattcccagaaatgaagggctgatcttttcccctgcagcaagatacagcatggcagcttttcctgagactcgatttagttgccaatttccttaatccaaggccagtgagttacctagaggaaacaatgttgaaggacagaaagaaaatggcctgtttggcagctctgctcatgcccaacataaaacactgctgatttatggtgagagtggagctgattctaaagaatccaatgaatcaattctacagcaagagtttatcagtgcttattcattttttaagrgattgaatgcactaaggcaaaagtgaatgcactaaggcaaaatgtaaggattggtctaatatactggttcttaaacttggttgtacactgaaatctcttggaaagtttaaaaatatatatactaatgcctgggcatcagaatttttgaaagctccccaagatctaatataaaaaacagtttgagaacaattaatctgagaaatactcttttattttgaaatttggtttttgaagctttctcatttctcatttcatttccataaccactgtatgcaccagtctaggcagagattgttctc

SEQ ID NO: 3 - DNA, 599 bp, Homo sapiens
ggaggggagaggctggaagcaggaaggctgatgagatgggagttgtgattgtctagacagtggtaggagatcactgtactctgcaggggctacggaagggttttcagcagggagagatgggaccagcatgctctccatcctgttgccccgcctcacgtctggcccctgcttctccagtcccccaccccagaccacacacgaagaagcagtcctgtcctcagcccagccctcacctcccccgacctgccatcctgcttcatgctcagggcggtgtgtggagcgcccggggctctggacccrcgctgccagataacaatgctctcgttgtctctttgctcccatctctgggggcctctgattctttctgctctacaggcacgcagcactgacagcctggatggcccaggggagggctcggtgcagcctctacccactgctggggggcccagtgtgaaggggaagcctgggaagaggtgagggtgagggaggaaagggctcagctaggagctggggagactcaaggagctgctggtgacgcatcccctgtctcccaggctctcagctcctcgaggccccttcccgcggctggctgactgcgc

SEQ ID NO: 4 - DNA, 599 bp, Homo sapiens
ttgccctgatctgttctaataggtccataagataattttcaaagaaatcggatgttattcccgaaatggacttctgcctttcctatttgtgaacagttaacacgattccacatataaattcaaaagcacatttggaaataatattcttttaattagatgttctgcttacatgtatgcaccttcctgagctattaaaacgtagaaaatccgaatctgaggcaatagcatgcaagtaaatggtagcagaatataagtgacataatatagtcaagcaatggtcatagtataatttagcctagrtatagcttgacatcatttatttgaattttctaagtaattaaaatatttatttattgctggattatttcattaagggcccatattaccacaaataacatttgtttaaagcatactctatgtaaatccttgattaaagatttagagaaaataggatggcttttgtatttcacaaaagatctgatctaaagggatcataacacatattcagaaacaagtacaaaatattaccattgccaaaataaaagatttactgaattatagcatggatttctgagatgtcattccatggagaaatatct

SEQ ID NO: 5 - DNA, 599 bp, Homo sapiens
agccagggaggtgaactgtaattaaaacgcagatggataaagcaggtactcctttaacatgcaaattgagatcctggcggccagcaagctccattttgagcctgggcggcaggctttcttagagggggctttgttagaaaggcagagggctttgttagaaaggcaccatgaaggagtggaaggaatggggattaaagaaggttaaaggacttggttaaagaggccaacaaaaccctcccaatggctgagttgcaagtttttaaattactattacctacttgagacatattttgaaaggcrtgaagaagaataaatggccccaggagcagattaagaagtaaacatccccagtctatctgaaggctcttgtgtgccccgcctcagcaatacccacctctgcctctgcagagggagcctctattctgaagtaggtgttactccagcctcatgctgtttttatacttttattacctatttggctggtttgtttattttttggagacaaagtctcgctctgtcacccaggctgaagtgcagtggcatgatcacagctcactgcagcctggacctcccaggctcaagcaatcctcctacctcag

SEQ ID NO: 6 - DNA, 599 bp, Homo sapiens
agcactcaaaatctgaagggaagtaagtgttatatgggggaagtaccaggagttaggggagcatataacagtgacaactaatccagtctagtgatcaaggaaggcttccctgaggaggtggcattaaagtcaagacctatagacaactgaaaagatgggtaggagtcattctagcagaaccactcttagatagctctataaccccaggataactgcctaatctccctggaactcattttatctttagatgtggataataattagcacaatagacagcactcaataagagttggttctccyttccttcacaggaaggtggccttccaatctaacgcaccatctatgagaaaaagagtaggtatatgtagagtctaataacacaaaccataattgttcttttattcattctgtaatatttcttgagcactggctgtcaagagcttttctagacatggatgtgcagttcctactcatcaattctaatagagaatgtatcggtcaggatggactaggttctgctgtgtaacaaacacaccaaaatcccaccagcttaacacaacaaaagtttactcctccctcatagagtctgctgcaggtct

SEQ ID NO: 7 - DNA, 599 bp, Homo sapiens
tatttacccaacacttctcccccagggagactgtttttcctccactgcaatactgtggccctcttggctcaccctgcctggtccctgacatggctgtggaagcagatggatggaatcagagaacccgcccagacagaaggaccaaggcaccgagggagaagataatataatgtggcccttcatctcctggtgtgttcctaaagtatggatgattgccattctcaaagaatttcatgatcctgatttaaagaaacttgccaacattccaggaacccagaaaacagacagcagtcctccacrgagaggcagaaaaagagacaggacgatttaaagtatcaggagcagggcctcacgggagtgagttcagattgtgcatggcaaagtcaaaacagatgatgctttagagcagtagtttccaaaccctatcacccaaagaacttgctaaagcaccgattgctggtccccaaaacagagcacctgcctcagtaactctgggtctatgtctatcaaattctcaggtgatactggtccaagagccacgccttagggggaaatggcattaatgccaactccagtaaattaggcaggataccaggaag

SEQ ID NO: 8 - DNA, 599 bp, Homo sapiens
agaggagcagctgttcctgtaatttatggtgatggtctctgtctccagcacacgtcatttctttcatgccctcccttggctttttctgcccatggctcagttagaacagcaggacagcatggccactggctgtgaggagctactttttaagtaagtttctgttctttgatcctgcgaaatggggccagggtcaagataaatgaagattttcatattcttaaagtgaagatggttccttctcttttcttttatatttctggaaggagtgaatgggaaaaaaatcaagaataacaaaaccarggtcacagatcctgggcatttgggccactgaataaaccattaaagtgtagaagagtaggcaaagatcgatgctttagataaataccactttttatatggatataaaaatgtcagaaaaggacaataatgaaattggattgtctgactcatccaagagtctatggatgcatatatgtctggtcttcctgccaggggatagcacattgtcaggtattatttcagagtttaaggaggggttttcttctccaaacaaacttcaacagatatttctggatttctcaaattgcagtgaaatgcac

SEQ ID NO: 9 - DNA, 599 bp, Homo sapiens
tatggaatgaacaagagcttcctaaactcaagggctgagtgttgaccctccagggcctgcaatgtggttgggggatgtccccttgcacaacgggcaaagaccggccaaggagcatctgctaagttcttataggagcttctgggctcaatcacccactgatcgccaagctgggtgctggcgatgcaatgggggcaggatctagccacaggcttgggggagcatatgagtcacaaataaaggcctgtgtacccccaggcttccttcccacacccatggcaggcattaccaattggtccccaygctctcttacagagctccttgtagcctcagaattgctattctgtatagcactccaggctgtcactctcaggcagagcataagacaaaacctacagcacttgatgtccctgttagtggagcaactactacattctgctgggttcagccacatctgggagagctagggcctggtcttggtctctggcctaggagagagcagccagatggtgtcaagcttagaccctcagtttgggaacttcaatggagaggcacagagggcctgggggtggggggatttggttttgctacagaatcccagc

SEQ ID NO: 10 - DNA, 599 bp, Homo sapiens
tcagcctaggtggggcttatagatttaagtgaggagactaaagaggtagttaatgtgggctttgaacaccccttggagttatagaatcattgatttagcaactttcaatgtcaatgtattctttttgccacttcaagcatgctgataaaccattagtttcctcgtgggcaggagaggaattcccccgcccagctctctctagttccagagcttatctgtggccaaaatttctggtcaaaagtaacttcatttccctggagtgataaccatgttccttagtgggtaacatctttcaacccrggtaagatttctaattaagtttagagatgttttagagtggcctatattttcttataaagaatctgaggtggctgggcactgcggctcatgcctgtagtcccagcgctttgggaggctgaggcaggaggattgcttgagtccgggagatcgagggtgcagtgagccatgattgtgccactgcactccagcttgggtgacagagtgagactttgtgtttaaaagaaaaaaaaaaggccaggcatggtggctcatgcctgtaatcctagcactctgggaggccgaggtgggtggatcacctg

SEQ ID NO: 11 - DNA, 599 bp, Homo sapiens
gagttcctacttcgattttcacgaggttacaataaaatgtccttcaggactgctgttatatgaaggcttgatgtgggctggaggacccactcaaaggtggattgttcacacagatggcaaagtgggtactggctttcgaccagttcttctctgcaatgtcatctccatggggttacttgagtgtcctggaggcatggtagctgacttcctcctgagtaagcaaatcaggaaaacaaagccaaaatgctacaacaccttttaggacctactctcagaggtaatgccttgttatttctgccrtattctattggtcacagaggtcagggctaaatttttatgggaggtcataaaaatcaagaggcaaggatcattggggaccttttagaggtctggctagcacaaatacggacgcaggctgggcacagtagctcacatctgtaatgccagcactttggaaggctgaggcaggaggatcacttgagcccagcagcttgggaccagcctgggcagcatagtgaggcccgcacttctaaaataataataataataactcatgcattgcatatgacttacagtgagaaatctagaaaaggctggca

SEQ ID NO: 12 - DNA, 599 bp, Homo sapiens
ggaggccaaggtggacagatcacctgagttcaggaattcgagaccagcctagccaacatggcaaaactccatccctactaaaaatacaaaaattagccaggtgtggtggcgcatgcctgtagtcccagctactcaggaggctgaggcaggagaatcacttaaacctgggaggggaatgttgaagtgaaccaagattgtgccactgcactccagcctgggtgacagagcaagactctgtctcaaaaaagcaaacaaacaaaaaaacaaattgcactgtttggtcaagaattcaattcacgrtaatcaagcagctcctaacacagttctctagtcacattttcagatttagcttctgtgggggaaaaaaaagtctgtatttccctgtagctctgtgagataagccttccatacctctcttgggttactatatttgaaggattcctgagatatggaatgaaagaactagcaaaaagctataatgatcactcacatttgattttaattagagaaaaaaacaaatctcttgctctcctccctcagctgggatgggattcttctccacgatgcctctatggtttctttccttgccttgagtgcaa

SEQ ID NO: 13 - DNA, 599 bp, Homo sapiens
aacactttttaaacattacattttttaaaaaataatatctttctcgatatgaaatttaaaatgtttttcaagtgagtttttttgtagaggacaaatccaaagtatagtatataattagcccatataacttcatgtgataatcagtgtctttgaagcagaccaaactgaagctgtgtctcagcgctaagggtctttttacattttactgaattggatctttagaattaatttttcaaaccctccttatcaaaagttattgttggaccctcaaatttttattttcatgtatctcatttctcytccctaaatcttgcctcccaccagattagctgccagaactttatctggttctctggcaaccctccatctgttttgtatgatttttcattcccagcagttctaattattgatttcatcaccctcttacccttataaaaggacatttttaatttggcagtcccaaggaatatagcttataaaaagaaaaaaaattaagtcactgaatgttttaggagagcactaagttaagacagactttgtacttgattaacggaattgttctgagagctttagagcttgacttttttttcttcctgtca

SEQ ID NO: 14 - DNA, 599 bp, Homo sapiens
ttgttatatgtgttttgcttaaagttacagttcccaaaaatctatcaaaaatgttaaatgaggattcattgtatttgtaaatctcattatttttctctcttttttaaagaaactaaattcaaattcattcagaaagccaggcataaagtgagactaatatgttcgttggtgtatttaagcctttcttagagttttcttttttttaagacctcagtataagaacatgccagacgtgaattgatctggaattgcagactcgggagggctttgatgctactctatctagttgccttccttgargagggattgagaagagaggtaggttatagaattaatgatttttttttgttccgtgagagtttatttcatccatagtctctaagatttagggctcaagatagccatgtgatttgagatttgcatgtagtatggcatttcttctgggagaggatgaaagagctaaagttcttgttttttggttttggtgtagtaggcagggagggttttgattctacctttaggaaagggaggtgttgaaaaattacgcaggtgtgtgctgatattacctggcacctctatgcattgtgatactgaattac

SEQ ID NO: 15 - DNA, 599 bp, Homo sapiens
gactaatatgttcgttggtgtatttaagcctttcttagagttttcttttttttaagacctcagtataagaacatgccagacgtgaattgatctggaattgcagactcgggagggctttgatgctactctatctagttgccttccttgaagagggattgagaagagaggtaggttatagaattaatgatttttttttgttccgtgagagtttatttcatccatagtctctaagatttagggctcaagatagccatgtgatttgagatttgcatgtagtatggcatttcttctgggagaggwtgaaagagctaaagttcttgttttttggttttggtgtagtaggcagggagggttttgattctacctttaggaaagggaggtgttgaaaaattacgcaggtgtgtgctgatattacctggcacctctatgcattgtgatactgaattaccagtctgccagccaaaagcgtgtaatatagtatgtctactgaggcaggtggtaaaggtagggtcctagtcactggaattctttttttttcccccttctctttgcaacatattattagtaaaggagctgaatttacttgttttccaaaatcc

SEQ ID NO: 16 - DNA, 599 bp, Homo sapiens
tccctaatagccaggacaaggactggcatgaagcgcgtgctcaataaatatttgctgaattaataagcttcacctctttaagtctcagttttatatcagaaaattaggagaagaaggtttggacaagaggtgctataatgttccttctgagttctaacatcttctgtattctctgtcttctaaaatcataggtctgtaacaagtttacaggttggtgctactcataataatttatgtattaaacattttaagtgagaaatatttcaaggtcaggtgtggtggctcatgccctataatccsagtactttgacaggctgaggtgggtggatcacacttgaggtcaggagtttgagaccagcctgaccaacatggcagaacgctgtctctactaaaaatacaaaaattagccagaagtggtggcactcgcctgtagtcccagctgtttgggaggctgaggcaggtgaattgcttgaacccaggaggtgggtgttgcagggagccaagatcataccacttccctccagcctgggcaacagagtgagactccatccccccgtaccccttcccccaccgccaccaccaaaaaaagaattatttct

SEQ ID NO: 17 - DNA, 599 bp, Homo sapiens
attagatatatcaaaactttgtggacatagctaagaagtacctagaagaaacttggtagaattaaatgtttatattagaaaaaaacgtgaaagtctaaaatgaatgatatatgattctagttcaaaaaaatagcaaactcaaccctggtttgattattgcagtcacataaaaataaaaatttaaaggtatgcagatttgaaaggaagaagtaaaactgtccctgttcatagttgacatgatcatctatgtagatcgtaaggaaggcatctacaaaaagctacaagaactagtaagtttascaaggtcataggctacatggtcattatacatgaattaattaggtataaatacatgacatagtataaaacgcattagaaacaaaaagaaattttcatttttaaaagtactgtttataacaattattttaaaataaaatactgaaggataaatctaacaaaataggtacagtatttatacactgaaaactataaaacatttctgagtaaaattaaagatactctgtttaaatggagagatatcatatgctcatggtttaaaagacaccacattgctaaggtatcagttctcccctaaaaga

SEQ ID NO: 18 - DNA, 599 bp, Homo sapiens
aaaaagaaattttcatttttaaaagtactgtttataacaattattttaaaataaaatactgaaggataaatctaacaaaataggtacagtatttatacactgaaaactataaaacatttctgagtaaaattaaagatactctgtttaaatggagagatatcatatgctcatggtttaaaagacaccacattgctaaggtatcagttctcccctaaaagaccttacatatttaacacaatctcatgcaaaaatcttaccaggcatttttatggaaattgtaaactgcatgtaaattttgtrtggaaaaatcaaaggatataaaatagccagaacaatttttaaaaagaacaaaattgcaagaatcagactacctgatttcaagacttactataaagtagattaacccaaacaatttgaaattgatatatggatagacaaatagatcaataggaaagagggaagtattcagatacagacatgaacacatatgaccaattgattttcaataaagagtccaaggcagctcaacaagggaaaatatgcaggtaatcttttcaacaaatggtgttagaacaattggatatctgttaaaaagagaa

SEQ ID NO: 19 - DNA, 599 bp, Homo sapiens
gtgtgtgggtgggggtggaggggtagggggaattagagatagataaggccataatacagagggcctcataaagtcctcaataaaccatgttaaagagcaagactgtatccttgaattcccagaagcagctggtgtaatcgtccaggctgtcttccacctagcccaaagttttttcaactctatcacttgctgcctctccctaccattaaatgagtaagcaatatactgagaaattaataaaactatgttaaatagaccacttttgagctccattttaatgcttggagaagagtcggtaarcattgtaattgattcagaaaagaaccacaatgttggaaactagaaaaaggtaaacatttgcatggctgagttaagagaacagagacataagaaactgttattacaggactaggatagactcatataccagttactaagcaggttcttgggcaagtcctttctgtacatttattttcttatttataaaatagggatgataattcaagtcaattatctgttatctacaattctgaagttccaagagctctgaaaatcagaagttggttcagttttattttgatttcttaagtttgtggcac

SEQ ID NO: 20 - DNA, 599 bp, Homo sapiens
gcaatacttttctagcattgggaaacttctaactaaaaattttgtgtgctggaatacttggaagaaaaggaaaggtcatttaaacaaatactatattttaaaatgaagaaataaagcaacattttaaatgtgtgatttttgttaagatacttgccattagtgagagttagggtgggacatatcagtaacgaatccgattgccactatggctctgcccccccaccatctttcctctgggaggatgttggggccaacttacgattgcaacctgtgtgtgtgtttatcctttgtttggatacrtgttactaggtgccccactcttggccagcaattccagtgcctgggtggtttgcctgagcaaagtgcctgagtggctaggaatgtagagatagggctttgacccagagcacagggatgataaggtttactatagtgtaaggattagaatgaaggttctcttctgcttcagaagtgggaagaggaagcaacacaggatgggatctctatctggttctaggtctcaggctttctccagcccgttttgccaacttacaagcaactaaccaccactaaacttcctctccctggagaaggaaaaa

SEQ ID NO: 21 - DNA, 599 bp, Homo sapiens
tctaactaaaaattttgtgtgctggaatacttggaagaaaaggaaaggtcatttaaacaaatactatattttaaaatgaagaaataaagcaacattttaaatgtgtgatttttgttaagatacttgccattagtgagagttagggtgggacatatcagtaacgaatccgattgccactatggctctgcccccccaccatctttcctctgggaggatgttggggccaacttacgattgcaacctgtgtgtgtgtttatcctttgtttggatacatgttactaggtgccccactcttggccrgcaattccagtgcctgggtggtttgcctgagcaaagtgcctgagtggctaggaatgtagagatagggctttgacccagagcacagggatgataaggtttactatagtgtaaggattagaatgaaggttctcttctgcttcagaagtgggaagaggaagcaacacaggatgggatctctatctggttctaggtctcaggctttctccagcccgttttgccaacttacaagcaactaaccaccactaaacttcctctccctggagaaggaaaaatttcactcacttgtgagtgagaagatc

SEQ ID NO: 22 - DNA, 599 bp, Homo sapiens
aaaatcaatggaagaagaaagagaatggatcggaatttaaaagaagaaaataaagtcaaaattctctttaaaaaactattttttacacattaatttcaagtccttgcccacatggccacattatccgtacatttatctcttgcttttcacttaacaatctgccataaagaatattgcaatatagatagacttcattatcagcctccctaacttggtatttcaccaagataacatactatttgtaagcgttttcccattttttaaacataaaaattgtttttagttttaaccataatagaygatgcagcagagaacattttgatatgcttcactttttacttttattgaatggtttttgtagactgggactataggacctaagagctgttaaaaacagctttacgggtcccagttcttggagcatcttcttttttttggagctctgctaaatatcacaattttatgaaactttactcagtctcttttttaaaattgcatcccttttcttacacccaacttgggactccttcaacacacaaacaccagaacgtttcttatctttataatcaacacaatgtttaaaagttgaggaggaggaa

SEQ ID NO: 23 - DNA, 599 bp, Homo sapiens
agcaatagtagtggatgcaagtgttaccataagtgggaaagcctaagagggatctcttagctgaagtttgaatgaacagatgatagatttgtaaatctgttgcatgaagcagagtagtgtggttgaaagtacactggtgtaacaggacccaagttctttggtttgctggcttgctgtgtggaccttcggcaactctctctctctaggcttcagttgccttacccgtacgtatgtactttggactagattatgtttaattctgagaacattttgagctctaagatgttattaatggaagtrtatattcaaatctatattgtactgttttatccacacagggaaaatattcttggagcattgctactgtttttggcaattatccccagttttcccttttcagtgatattcatgaaggtggtatagcctatttgaattacatgatgtggatcagcttacaaacgaacaggatcaaagatcacaaaatgttaaagcctgtctcacctaaggtagtgttcagtgttgcaaacattagctttaagcttccccgcaggcaagatgaagaaaaatctacaacagggttcagagtttgcccttttagg

SEQ ID NO: 24 - DNA, 599 bp, Homo sapiens
agtaagtggcgtatgtaaaattgtcattctacacaaaaaatcacgagcagagggcaaagtgaaatcgtggctgctttatcattaattttgcatgtgcagcggagagcttgtcctttgtgctctaaatccttgctacaaacggttacataaaagatctaagaaagtggagacaaaggaaggtgggtaaagttagaaggaaaaaaagagctagaaaagtgtgcaagtcacttcatacctgaattcttgacatttgactggaattgttctgattagaccatggtcctcaaggcatttcacagktttttttaagtctgcgctgccttaggggattttatccttgagacatccactggcttaactcaagtttccttcaaaatatgtagctaaatacagctgttcagctaatagctcagaggttctttggagaacaaatggaatgttatttactaatattacttgtggcatgttagcacttttgtgttctgccaagtgcttttgggtccattctcaaagccgccatggctaagctggtagtacgttggcgatggcccatatgggaagtggaagtggtagatcttcaggggactttcaaaatgctt

SEQ ID NO: 25 - DNA, 599 bp, Homo sapiens
gatctaagaaagtggagacaaaggaaggtgggtaaagttagaaggaaaaaaagagctagaaaagtgtgcaagtcacttcatacctgaattcttgacatttgactggaattgttctgattagaccatggtcctcaaggcatttcacagttttttttaagtctgcgctgccttaggggattttatccttgagacatccactggcttaactcaagtttccttcaaaatatgtagctaaatacagctgttcagctaatagctcagaggttctttggagaacaaatggaatgttatttactaatrttacttgtggcatgttagcacttttgtgttctgccaagtgcttttgggtccattctcaaagccgccatggctaagctggtagtacgttggcgatggcccatatgggaagtggaagtggtagatcttcaggggactttcaaaatgctttgaatttaactctttcttcccctttattctaattcctagtgggcaagtcctgggccgacgctgctttgaggcccggatctgtgcttgcccaggaagagacaggaaggcggatgaagatagcatcagaaagcagcaagtttcggacagtacaa

SEQ ID NO: 26 - DNA, 599 bp, Homo sapiens
tgtgttgctggtactactgtctttttaatatgtatattaaatctgattaacattaatatttaattattaagtatatttaatatgcattagtgctttagaagtgttcccaggatgaaacttgcatttttcctccaccagcgtttcgtcagaacacacatggtatccagatgacatccatcaagaaacgaagatccccagatgatgaactgttatacttaccagtaggtcttccttgggtgttcatggttgcttcattttaaccttctttgaatgggcttttacagtatgatcatcatctcrttatctgtgacaatgggaaaggaggtgtcttagtcagttgaagctgctacaacaaactaccgtagactagatggcttataaacaacagaagtttattttctagagtctggaagtctgagatcaaggtgcccctatggtcgggttctggtgagagttctcttctgagttgcaaactgccattttctccttgtattctcatatagtggagagggctagagagctctctagggtgttttttttttttttataagggcaaaatcccatttatgaaggctccactctcattacctaaattactt

SEQ ID NO: 27 - DNA, 599 bp, Homo sapiens
cagttttggactgtattactagcatgagcttctctacttacctacccacccaactaaagctaaactcctaacagaatttttcttaccctaatatttaaaattaatttgtccttcattatttgagaatgataagattatcctaggcagcatggccatagaaatttaggccgaaaatgttgtcagaaaaagttcacaaaattgataatgcagcaatgatagccttgactttgatgtcttttaggcagcactataaagaaagccaagcaaggcagaagcctgaattacagtcagccctccctmtctgcctgttccatatctgcagattcaaccaatcacagatcaaaaatagttggaaaaaaacaataaaaaaatacaccaatcaaaataatacaaatataacaacaatatagtataacaactaattgcatggcatttacattatattagatattataagtaatctagaggtaattttaaactatacagaggatatgcataggttacgtgcaaatactacaccattttatataagggacttgagcaacctaggattttagtatccatgggggccagatgggggtgggtagtcctggaatcaa

SEQ ID NO: 28 - DNA, 599 bp, Homo sapiens
tatatgcctggtccatacacactaacagtaagtacacactctatttggtagtgatgtgtatatttgaaaacatgaaatcttttctcatcccaatggattgtcttataaatctcctgggatgcacactatccacttttgggaataacactgtagaccagggatagcaaataggctttactataatataaagtgacttgtttgaatgctgtaatgagaagaattctgagacctagtgcatgataattggggaaatatctgggtgcagaaggataaggtagcatcatgttgccgtattttagmatctctgctctagattgtcttccagcagtacatttttggcataaatcaagccagatttttcaagtatattaccatatacatataacctatttatatgttaagctttgccttgaatcttggatctttcctaaagtgattttatgactgactagaatagaatagctcttatgaaaggatgagaggttgaccaatcaggaccttgtttatatttacctttctgtggttaaaaataatcacttgttagtttaattaaaatatgatatgtgtcctggaagtattgtcagagactctgtagataa

SEQ ID NO: 29 - DNA, 599 bp, Homo sapiens
ggtaggagtgcagtggcactttcttggctcactgcaacctctgcctagcgggttcaagtgattctcctgcctcagcctcccgagtagctgggattagaggcacctgccaccatgcctggcagattttttgtattttttagtagagatggggtttcaccatgttagccaggctggtcgcgaactcctgaactcaggtgatccacccacctcggcttcccagagtgctgggattacaggtgtgggccaccgtgccggccttgttattatgttttaatcaatatgaatgtatctctgatagtraagaaacactttctgagcacctgttatctacagagtggagtagacggcagggagaaaagaggcacattctctgttcaaaggactctaagctatttgagcatccaagatcctttttggcccgaagggatctcaccatttacttttagcataaaagttttgcaaattttaatggtatggttttatttgtaagcctgagcagaaagtttaatcacacagtgtattctgaaggagaattagaactcagagttgcagacacaatttccattgctaatcatatggatgttttgggatatcagatg

SEQ ID NO: 30 - DNA, 599 bp, Homo sapiens
agttttgcaaattttaatggtatggttttatttgtaagcctgagcagaaagtttaatcacacagtgtattctgaaggagaattagaactcagagttgcagacacaatttccattgctaatcatatggatgttttgggatatcagatggaaatatatactaacatatttaagtggaaaatgatttagttcttataaaaccatacaaccaggatcaaagaaacagagacctgtaacatcatttatagacccaggagggatatctacctttcctaagatatgtgtgtcatggtattaagaaayaccctcaaatttcctcctgttcactctgaaagtttgactataccaaaatgacctccaaaacatcactaagtaatatatcatgctgaactagttgctcttcagtactctttcctgtctttttttttttttttttttcagttaataagaaaaggtctcatatgtagtacaggaattggcaagctgctttttgtaaagtcctggtagtaaatatttagattttctgggtctatattgtcttttgcaactactcagctctgtggttgtagcacagaaacagccgaagataatgcataaaggaa

SEQ ID NO: 31 - DNA, 599 bp, Homo sapiens
aagaaattatttacaccacgaggatgccagaaataaacccttttattttgctgaaagttgggcaatctcatgaactgtttggaaattgtttcccagattggcttcttgaatcacatttggctatgcctttcaaaggaacaaagtcatttttatttgcttaatgctaggattcagtaaaccatgtcaccggcaagaattgatatcatcttaacagaatatgcccgaataaaaatggatggggaagcaatgtacagctaactaaagacctctaagacctaggactgagcctgattctgcaaygaggtgagagaagttacaaaagtaatttaatctatctgagtttaattaatcattgagttttttcttcattaccaattcagagataataaatctctgccctgaaaacttcataactatgaaattcaaaagtaaactgtaggctgggcgcggtggctcacacctgtaatcccagcactgtgggaggccaaggtcagggggatcacaaggtcaggggtttgagaccagcctggccaacatggtgaaaccccgtctctatggtttttcttcattaccaattcagagataataaatctctgccc

SEQ ID NO: 32 - DNA, 599 bp, Homo sapiens
tggaaattaaagattgaaagggtagactacttttcttttttttactcaaaagtttagagaatctctgtttctttccattttaaaaacatattttaagataatagcataaagactttaaaaatgttcctcccctccatcttcccacacccagtcaccagcactgtattttctgtcaccaagacaatgatttcttgttattgaggctgttgcttttgtggatgtgtgattttaattttcaataaacttttgcatcttggtttatcttgcagtttttttgtttctgtctctctcacttttttyccactaaatactagaatattctcatgtaagtgcttgaagtagcatgtgtaggtctaaggaacaagaataataatagaatcatcactttgttcctacgtacatttcttctgtcagtatttggtatttttatttaagatactctgtatcaaggtcatcaatcgtaacaaggaactaaaatgcctaaactaacttaaaaaaatactttagctctatgagttttaaagtttgtcctaggaagccagacaccatagaatcatttattgacattcctgacaaacctctagttgtatgttaattcg

SEQ ID NO: 33 - DNA, 599 bp, Homo sapiens
atactcaccaatcctttaagggctttgtgaatcaaagttattagaccaattgaaaaacaccttgcaaatataacaggagcatctggttttactaatatacttttatctcaaatataaagatttttttttctgaaatatttatctaaaatactaattttttttttttggttagaacttcttatgaagcttttaaaagcaattattcagggctcaaaacataagtaaaaaaaaaaattggacaataggttccactgttgatttctggccaaatatttgctaatgtctatgccacagaagccrtgttatttaaaacatttggatttgattgaaattcactttagtaaatttggcgaattaacatacaactagaggtttgtcaggaatgtcaataaatgattctatggtgtctggcttcctaggacaaactttaaaactcatagagctaaagtatttttttaagttagtttaggcattttagttccttgttacgattgatgaccttgatacagagtatcttaaataaaaataccaaatactgacagaagaaatgtacgtaggaacaaagtgatgattctattattattcttgttccttagacc

SEQ ID NO: 34 - DNA, 599 bp, Homo sapiens
atttgagataaaagtatattagtaaaaccagatgctcctgttatatttgcaaggtgtttttcaattggtctaataactttgattcacaaagcccttaaaggattggtgagtatgtttcgttttttgatatttgcttttctttcagaagggtttttcttttctttcagccttttttaagtaagtttttaatgtgtttatagtgacttgtatataaatgcacatatttgaaatgatggaaaatattagaaaattacagttttcccatctagagaataaatgctgcactgtctttagtagccrgataacccggctagagagaaaggtcacaggtaggcagtactgggtaaaattgagagaagatatgattctttttctgatggtccatgttttcccttttttttttttttttcctgacctttctcataacaaggatgctctgctccgccctcagtatccctttctgctcccaaattctttactgcagaaatggtttgttgaaatagatttggcctaagctatcaccaaatcctactagagggttgctttttaatgagcagtggaagattttagccaaaagaaggaatctagtggaattctag

SEQ ID NO: 35 - DNA, 599 bp, Homo sapiens
gactgcgaggctatcatcaaatcatatttttcctgaatgtgcagagtgcaaggtttagtatttcctttgagaaagagaaaattcttgcctcctcttttcccttcagttgttttctccattcccttctcctcttttcccttcaactcaaatgagtttgaaaagcagttagggaaaagaattgagcaaagacttaaaggctgacgaaaaacatggatatcccaccctcaaactgcagctaaggcattccatttgtagctttctgtatattccatttgggatatgcagaaaatgactacaaasccaatcaaactgggcatttggcagctcatcttgctgtcatgcagaggcccctacttggaaacatgcattttcaatagagatgttcatgatgtatgagagaacctactggaatgtcaaaagcatttctacacaggagaacgctgaagcccaaataactattctattgactttccaaaagtctcctgggagaaatgaaaaaaacaaagcaacagaaaaacccaatagttttccattcagcatgatcttcaatttggaaagttaccagaactcatttgtcatattatatctaacagataata

SEQ ID NO: 36 - DNA, 599 bp, Homo sapiens
ggagggcggatactgttatagtccataaaacagttggaagggaagacagcagtcctgtggttggagttcagtcactattatctctgatctgtggagtggtcaacaatgttttatcaaggaaaactactataaccagcatggaaacaagggaaagtgtctgttgataggcatattgttttcatgtctgtttccctcgccttcacttactcccttcctcctctctcacatttttacattgtcaattgcggccctcaagttggtgctcagggctgggtttccaaaagggcaataaacttgctrtctcgctccttttcctctctctctaatccttatagctctcagccaccatgggaccattattttggagttggatgtccataaaaagttttaatgttatccaaaatgacagggactttcaaaaactgatggcctttactactctgcctatgggaaatgggcacactccagaaaaggggagttgagataagcagaagaaagaaatgcatgaagcctgaaagatttgcttcccacagaggcaagagtaggttcccagatttcttgcctagtgtatatcttcttcttctgcttcatgtgcagaa

SEQ ID NO: 37 - DNA, 599 bp, Homo sapiens
caaattgagaaatgttgggaagctgtgcaattttctcctcctcacctcagcttcttcctgcgtatttctagaaagagacacccttttctcagcttttatcttagagccagtctaagttattgggcatcatcacttcctggaaaaggaatgtgacctaaatttggggaacttcattagtatccccataacatgcaaaaatgaggtaacaatacaacataataataatacaaatggttaatatttatagagtgtgttgtaatttataaaatactttcatagctgtatttacctatgagtctrtagcatcttttgccctataaagcaattgtatctcattttaccaaagaagaaactgaggctggtagagaataagtgacttgctgaagttctatagccagtcagtgaccatcaaaacctctgccacagttttctcactccaagcacaatgtgctgtttacctaccattctgccctgtgaaaatatgagctatcactgaaacaatctaaagaagagctaacgcagataaatgtaagtgatatcaccacagtcagttaaggttgaccctatttgcaatgccttctgcacccattcacagaaca

SEQ ID NO: 38 - DNA, 599 bp, Homo sapiens
cttcattagtatccccataacatgcaaaaatgaggtaacaatacaacataataataatacaaatggttaatatttatagagtgtgttgtaatttataaaatactttcatagctgtatttacctatgagtctatagcatcttttgccctataaagcaattgtatctcattttaccaaagaagaaactgaggctggtagagaataagtgacttgctgaagttctatagccagtcagtgaccatcaaaacctctgccacagttttctcactccaagcacaatgtgctgtttacctaccattcygccctgtgaaaatatgagctatcactgaaacaatctaaagaagagctaacgcagataaatgtaagtgatatcaccacagtcagttaaggttgaccctatttgcaatgccttctgcacccattcacagaacatagacacagttgctctgcattttgataccttgctttattcaaacaccaagagaccttttagataaaagtgccgtttcaggttctgaggatgccctaagtccctaattttttcacttagttatgtgactttaaacttgtcaacctctctggccttcagttgttctatct

SEQ ID NO: 39 - DNA, 599 bp, Homo sapiens
tagctacatgtaattattgagcacatggatggagctagtcaaaactgagatgtgctgtatatgcaaaatacacattagacttcagggactcaaaaatataaaataactccttgataattgtttaaattagatgttgaaaataaatattttgggttaaatatactggaattaattttacccgttcctttatatgcttactacaaaatttaaaattatgtaaatggatttcattgtagttctattgggcaattagctagaaagatgatgtaataagaagcctgttaggatgatgaaatgaakttactatttactattgtgtaattatccaggttttttggttttttactgggttgctcattagtattgcatcagttttcaacaaagtatagaataggagacaatatctttgtagttaggatttttcattttctccccataatggtaaagatattaaaactgacagagaaaagcatttcaaaattgttacatcagaatatgaatacaaaacacaatggcactctagactctggtgatggtgaaatggcttgtgtaagtttgagttttcatgtaatgatatcctagatgaattttctcaagca

SEQ ID NO: 40 - DNA, 599 bp, Homo sapiens
tgtaaatacaagaaaagcaatcagaagtaatgatgttagaacttcttatcagaggtgcatttgaaatcaatacttacaaggtagctatgtcccaaatttcatggtagttcatgagcagttaattgcattcagaaggtattttctatttcatgtacatgtgccactgaaatcattaacaaaattttagatttactatgtttatattaaaatttcttataaaattattcttcaccatatattctgttatttataaaattactaaaataaaaatataaagcattaattagacccagaagatayggataatgactactgcactgagaattattagaataatttatctaatattgttatagaatgccaaaaacctaatgttcattaaagactcttgaattatctaaaatctgcatagaatctctgagattccaagaacatatttgtgcttttgggtgcagtttctgaattatctaagaaggtaacccaaggactttccttagtatactgatttattttgtattcctagtagcaaaataaaagtatggaataaaggcctgcacataggtgggtagacattggaaccttcctagatccaggaaaga

SEQ ID NO: 41 - DNA, 599 bp, Homo sapiens
taatcttctgcatttcagtttcaacatctgccagatgagaggtctgaacccctaacttgtcagtatttcagtaatagcttttggaagaaagccgaaggatggcatgaaacaactcttccaatatagaaatggggttagcgttttcagaaaaaattcagcagtctcaaagctctaatcttcgttaaatgcagacctaaaagtgtcgggaatcttgttccttctttagaaaggtttttcatagagcactaaccatatatatttttctcagataagagtagttcactaatttaactttgtggktttacagtcatgttgggtgtgtccttgaaatttatgagatttgtaactccttacactaatcctgactctgtctatgtgccaggtgggtggcaagtactggcaggtgaacttcccttttgtgattctgattagaattctaggcctagcacactggaggaccaacgttttgcaattcaagtaaaagtctgacacaggacgaggaacaaaaattttttgcagagagagtttgacatcagaaacaccaggcagaaaataacgaatcctgtgagcgctttaactcatctgacatttgctctgta

SEQ ID NO: 42 - DNA, 599 bp, Homo sapiens
aaatatttatcaagtaccttctatgtgtcagagcctcttcgaggacaggaattacagagggtttagttaaagaccctggcctcacagagtttatattctagcaagggacataaacaataagcacacaaataagtatttaatgcaatgtcagttactgattagcacaataaaaaaaactgaagcaaggcaaaaaacaaaacacagaatgagtggtagagtgaaagatggaggctatgattagagatgaaattcaggatatcaatgattagatatgaagtccaggaattcctctctgggtaraacagcagacacctgaataagtggaaaggataagatatcttaggagtagagttccaggaaaagataacaatgaaagcaaagaccctgggagggaatatatttgtaatgttaaagaaataggaagaagccctgtatgactgcagttttgtgaggcaggccaagagtattaggagacaaaatcagggacagtggcagagaacagactagatcacatagagtcaagtaacccaaggtaaacagtggaaatttcatttaacctgactgatagaaaattattggaggattctgagtagagagga

SEQ ID NO: 43 - DNA, 599 bp, Homo sapiens
tgggaacatatagataaattactgggaaaaaatgattatgggtttaaaatgtcatccagcaacatttactttagtaattagcataaatgtatacttctcatctacctaatccattaacttggtagtcaacttagacttctttctcttgcccatctccattggtcttgttgtgcataaaccaacaccaaagagaaccctttggtgtaaaattaggtttaatggtaacattcatgcctagacaattctgcttgagaaaagcagaatgagtgaaataattcaaaagttcaaagtaggtagggwtaaaccataagggaaaagaaataatatgtgctagaagttcagttgaaaaagaaataaattcttttgtcttgtcttccttaagtttcacctccctgcctacatgttctaggtattcagtagtcatttgttgttaatcatgataatgacaatggtgtccatttgcagctcaacttgtaagtttatttttccttttaaagcagcaatatggtgcttttttaataatagagagcatattatctttctgaaagacacttatagtcattcaggttgagaagtgcacacaggcacaacattccaaa

SEQ ID NO: 44 - DNA, 599 bp, Homo sapiens
taggtttaatggtaacattcatgcctagacaattctgcttgagaaaagcagaatgagtgaaataattcaaaagttcaaagtaggtagggttaaaccataagggaaaagaaataatatgtgctagaagttcagttgaaaaagaaataaattcttttgtcttgtcttccttaagtttcacctccctgcctacatgttctaggtattcagtagtcatttgttgttaatcatgataatgacaatggtgtccatttgcagctcaacttgtaagtttatttttccttttaaagcagcaatatggtscttttttaataatagagagcatattatctttctgaaagacacttatagtcattcaggttgagaagtgcacacaggcacaacattccaaagttataacttttactgtagctatcatagccttgtatatttaggacaacaactgaagtaaagggtcttgagaaaaggacagcattttataattaacacatagctaccatatgagctagggactattctttcccctaagtatcaaagagctctctaatttggtcctcaaattttagttttaagaaccacctgagctctttatttcattcata

SEQ ID NO: 45 - DNA, 599 bp, Homo sapiens
atggtattagttatcctcctgggaagaaccaaaataagattacagaagtatatcagacctagtcctcatcttctgaaagttgacattctaacttatgaaataacacaaaagcaaatggttacagtacaaagtgatgagagctgtaactgctgcccgtccaaaatgctgaggaaataccaagaaggaagcaactaactctaccttagggattaggagagagcctcagtgaggaagtcaggattgggacttcccaggcaaagaagagagggaatgcttcccagtaaaaggaaatagcacatrcctgtttcagggaagtaaataattctgctagaaaatattaactgagtctgtaacttggggtacagaagtgtggagaagacagataaagcaaatctggtaagactggctggagtctggtaagattatggaaacttgtttgtcaggacttagaacttgattttaaatgaatatgaaggagagaaatgcaactgtgtaaagaagaaacttcagatggtgttgggaagatctgaggcactctaccagctgagtattaaaactgtctatttggcagcactgtagaggtgggttggaggaaagca

SEQ ID NO: 46 - DNA, 599 bp, Homo sapiens
agaagacagataaagcaaatctggtaagactggctggagtctggtaagattatggaaacttgtttgtcaggacttagaacttgattttaaatgaatatgaaggagagaaatgcaactgtgtaaagaagaaacttcagatggtgttgggaagatctgaggcactctaccagctgagtattaaaactgtctatttggcagcactgtagaggtgggttggaggaaagcaaaggacaagtaaagggctagtccaggtaggactaggacaaacctaacttcaggaatctcttagccatctctgaycttgcaggacatcatggaaaagctctaatcatcttgacaagagtctaattctaagactcctgttgtctccacagcaccaagcagcttgtggtaacaagtttctgtatgccacccacttctgaggtcaaaaaagaatgaaggtgtttatttacagtcccttcattattgcctctgatagaattgtgcttctttctttcagagcatttttttaaatttggaattattctttcataagtatgattatttgataaatgtctgatgaatctcactagattataatcttcaaaatattaaaagtg

SEQ ID NO: 47 - DNA, 599 bp, Homo sapiens
gaaaaatgtttagtggttttttttgaaatgataaaatattcaattcgtgatttactgtttactagataatttttttgctgtttatagccaaattttctcttcaatattgcatgtgtacactaactttgtacctattaaatttcaggtctgaaatccacttaaattgggaagtaatttgaccaaattatttaattctctggactttatatcccaagataaagtaatatcttctgtaactaaaggggacttttttaacctaaggcaaacaatttttcaatcatgactttacatttgatgagrttccattgagaaactctatgcatgtcctacttctaacaggcacacacaaaaagagacagtcaaagcacttcattaaaggcagaaagattaaggcaaaagattacacaacaaaaccacccacattcagttttgcaagtctaatagtcatcctgaatcatcaccctagagcggcctgtattccgttccaactcgtggcttcagcttaccattcaagatgcaagaaagtaaaatattaatattggtgaccaaaaaagcaggtgtgtttctctttccttagatactcttagccctggaggcag

SEQ ID NO: 48 - DNA, 599 bp, Homo sapiens
ctaaaggggacttttttaacctaaggcaaacaatttttcaatcatgactttacatttgatgagattccattgagaaactctatgcatgtcctacttctaacaggcacacacaaaaagagacagtcaaagcacttcattaaaggcagaaagattaaggcaaaagattacacaacaaaaccacccacattcagttttgcaagtctaatagtcatcctgaatcatcaccctagagcggcctgtattccgttccaactcgtggcttcagcttaccattcaagatgcaagaaagtaaaatattartattggtgaccaaaaaagcaggtgtgtttctctttccttagatactcttagccctggaggcagcaattgtttcttgatttttagttagtaaatatacatctcttgggaggtaattaaagaacaagcacatattttaaaatcaccaaagaataatacttgatcgtatttctggcaaccgaatcactgacttaagaatctcagaaactagtttttaaataagctttacccagccctggctcagccccataacaatatcctccagttattcatcctttcctttatcaggctcttctctcaaa

SEQ ID NO: 49 - DNA, 599 bp, Homo sapiens
tatgctaattttaaacaaagcacacttcagaacaaggacaattttgaaagataaagaggagtaatacataatgataaaagggttaactttctaaaaggagacaccaacccttaatgtgcatgtacccaacaacacagcataaaaatatgcaaagcaaatctgatagaactgcaaagaaaaattgacaagtccacgactatagtgggagacttcaccactcgtttaataattgatagatccaacaggtagaaatctgataaggatatagatgaccttaaaaacatcatcaatcacagtgayctaattgacatttgcagaaaactctatttaatatgatgaaatatactttttctcaagcccccatggaatattcgccaagaagaaccacattttaaatcataaaacacaccttaacaaatttaaaggaacagaagttatacaaagtatgctctcaatacacacaataaacaagaaatcagtaatataaagttgggtgaaaaatatcaaagtatctggtgattaaatagcatacatctaaataacacatgatcaaaaaagaagcctcaagataaatttaaacatgttttaactaaatgaaa

SEQ ID NO: 50 - DNA, 599 bp, Homo sapiens
gtaaaataacaagtcataaaaatacacagaagagccttagtgttgactactatgtgaaagaagccagtctgaaaagactacaaacctgtatgattttcattatgaggatattctggaaaaggtaaaactgtagataaagttaaaaagtaagtgatatccaggactcagtgagagagggagatggttaaataggtaaaattcccgtgatttttagggtattaaaactatcctgtatgatgctataatggtatgtactataatgacattatgcatttatcaaaacccattgaacttcacaayacatagtgtgaaccttaatgcatgctcattttaaaaaatcatctctgaaactggaggattttaggaagaagtgtggactgtggcaagagaatcttactgtattacagatgtatgaaaaacttcaatgaaaggtttggagggaaaagatactggctaagtaaactttggaaattacttgagtgtgtaaaacggaaagtaaaataagctacttacaagcgctatactctagttagtaaagctgtttctagcaggtgctaatactgatggtgctatactgtatactgaaattgaacaattaa

SEQ ID NO: 51 - DNA, 599 bp, Homo sapiens
aattgtgtgtgtgtgaaaaaaaattatgtgtacagatacagattacttactggcatacagatgtgtatatatacatatgtgtgtgtgtatagctctggattataaaattatagtcttgtttttaattaaagggctatacatctctgtaaacaacctaaggtgatgggaataaaaacattatttaaaaaggtaagtcacaaaatagagagaacttcttagaatctaaggaagatgggtggaatgatgccaaaagcatatggataaaaaagcatcctaattttaataggggaagtccaactkctagaacctagagcgcagcttctggttagcacttgtcaaatcaatgctctactgaattggatagataaaaactttggtcaaacttttccaagacggggcagtttcacaggatgtcgtctgccttcaaacactttgaggaacatagcttttatttatttatttatttatttatttttctgttcattgcctattagctgagaggttccttagactagtcctgtcaccaacagggttatcctttctttgctggcttaaggagaggtaacaggatagttgtatgcctttcctttctctttgtg

SEQ ID NO: 52 - DNA, 599 bp, Homo sapiens
atgctatccctctccccttcccccaagaaagctcattatttaaaaatgacaaaagtcaatttacatctttttaaaggactgaaactcagaaggttagaaacacctgcttgagatcacatggtccagcttattcattttcaaattgggaacctgaggccctgacaaggcaagcaacttgcttagagtctcatcacaaaggaaggacagatccagatcttcttatttagaatcttgaagtctttcacgaggcttcaaaaactcaacccttgggtggcataaaattatttcgagagatccaaraatgtatatacacatttggatttgtttgatttttacgtatataaagaacagcctgctatcatttgtgtgtttgcatatgacaaacctgtgtcttcttttcacaatagtcaagaactactacattatgacataatttatttcaattcaacagttgttcaatgatgcactccatgttccaggccccattggacactctgagcattataaagatgctcatgataatttttttcctacttgagattagagtctaatctgaagagagttatgttatgcatacaattaattgtaataaaggttgc

SEQ ID NO: 53 - DNA, 139374 bp, Homo sapiens
aacactttttaaacattacattttttaaaaaataatatctttctcgatatgaaatttaaaatgtttttcaagtgagtttttttgtagaggacaaatccaaagtatagtatataattagcccatataacttcatgtgataatcagtgtctttgaagcagaccaaactgaagctgtgtctcagcgctaagggtctttttacattttactgaattggatctttagaattaatttttcaaaccctccttatcaaaagttattgttggaccctcaaatttttattttcatgtatctcatttctcttccctaaatcttgcctcccaccagattagctgccagaactttatctggttctctggcaaccctccatctgttttgtatgatttttcattcccagcagttctaattattgatttcatcaccctcttacccttataaaaggacatttttaatttggcagtcccaaggaatatagcttataaaaagaaaaaaaattaagtcactgaatgttttaggagagcactaagttaagacagactttgtacttgattaacggaattgttctgagagctttagagcttgacttttttttcttcctgtcaagtattttgggttatttgagatagtacagacaacactttacctcttaagttttttgaactagaagccataaattagaagtgcacctggcatttcctgtttgtgactagtggctatcttttagataacagtgaagtcctcaatgtccttgataggttcttggtaaactgcgaccttaaacaaaatgatgtgcaggagatcttcgagtaattgtttcatacaatgttattttcttataacagtgatgagaaaaacaaattgttttgttatatgtgttttgcttaaagttacagttcccaaaaatctatcaaaaatgttaaatgaggattcattgtatttgtaaatctcattatttttctctcttttttaaagaaactaaattcaaattcattcagaaagccaggcataaagtgagactaatatgttcgttggtgtatttaagcctttcttagagttttcttttttttaagacctcagtataagaacatgccagacgtgaattgatctggaattgcagactcgggagggctttgatgctactctatctagttgccttccttgaagagggattgagaagagaggtaggttatagaattaatgatttttttttgttccgtgagagtttatttcatccatagtctctaagatttagggctcaagatagccatgtgatttgagatttgcatgtagtatggcatttcttctgggagaggatgaaagagctaaagttcttgttttttggttttggtgtagtaggcagggagggttttgattctacctttaggaaagggaggtgttgaaaaattacgcaggtgtgtgctgatattacctggcacctctatgcattgtgatactgaattaccagtctgccagccaaaagcgtgtaatatagtatgtctactgaggcaggtggtaaaggtagggtcctagtcactggaattctttttttttcccccttctctttgcaacatattattagtaaaggagctgaatttacttgttttccaaaatcccctggagttgttttatttgaacatgatatttggctttaagcagctcacccctcgatattccaaacttcctcccctccctcccaaagcaaaacaaaatttaggcaaagcttacttatccattttgacaaggatattttataaaaagaaggagtttagttcatttgttagttttcaggaacttcttttccatgtcattgttgaaatatttagttcatgtggatggccaaaactgtcatgtcagtatacaaggaaatttggttgaagatcagtacaatattaggctcatcattcttgcccatgggctggtgcttcctgggaatgaacaaactattgaatatatgtacagtatgtatacagaagaacatatattaagttctactttatatatttatacatatatgatcagaaatggccagtagggtgtgtgtgtgtgtgtgtgtgtgtgtgtgtgtgtcatcagaaagattaccaacactttgttttccacattcttttttttttttttttttttttttgagacagtctcactctattgcccaggctgtagtgcaatggtgtgatcttgactcactgcaacctctgcctcctgggttcaagcaattctcctgcctcagacttctgagtagcagagattacaggtgtgcatcaccacacctaatttttatatttttagtagagatggggtttcaccatgttgtccaggctggtctcgaactgctgaccttgtgatccacccacctcggcctcccaaaatgctgggattacaggcatgagccaccacgcttggccttccacattctttattaaagagtcactttgccagtgcaaatctggtaactgcgttctctaagagatttgcaccaaattataaaataatcaaatgaggccggacacagtgtctcacgcctataatcccagcacttctggagattgaggcgggcggatcacctgaggtcaggagtttgagaccagcctggtcaacatggtgaaaccccgtctttacccaaaatacaaaaattagctaggtgtggtggcgggcgcctgtaatcccagctactcaggaggctgaggcaggagagtcgctagaaccttggaggcagaggttgcagtgagctgagattgcaccattgcactccagcctgggtgacagagcgaagctcagtcttagaataacaaaaataatcatcatcatcatcatcattatcatcaaatggaagtaacactatacattatctattttcccctctatgaatgattagctcaagatttggggggatcatgtaagttgccagttatttctcagtgagtgtcagatatttatgacttctttgaagaggccaatcgtttttgaaaattatttcagatgtgctttatttccccccacccatcacagtattttatttctgatttcacttagatttgcataatgcaaagagccacagttgcactgggggtcctttctaaataggcaacgaggttagtgcaacccactgctgacttattgaacccaggacctgatggagtagagttgattcttaaagcaatgagtgggcattttgtgttgtttttatgttcaagctcccattgtgagaatatggttacactttagggaagaaaacctcaactcttagacaaacttctttgcttaactaaaaataagtatatatactcctgtaagaatttggtgatcatatttttatttttgttagtaagatggcctctgagctttacacttgattgtctaatcatatattttctaatcatgtgtttattcactcaaattcactctggcacgaatacaccagttccactttggcctcatactataactgttaaaactgtgtgggacgtggggaagaacactggtctgtatgtcaagatggtcccagttactgagaacttttgagcaaatctcttctcctttctggggcttagattcctcatcggcgaataaagtggctgaaccaaatgatctttaactttgaattttcctgatctaacttttccttttccttttcctccctctgcctcctcctcctcctcctccttctctctctctctctctctctctctctctctgttgcctaggctggagtgcactggtctgaggatggctcactgtaacctcaacctgctgcactcaagtgatcctctgcacctcagcctcctacagatgcccccatacctggcttttttttttttaattttgtataggcagagtcttcttgtattgcccagactggtctcaaattcttgggttcaagcaatactcctgcctagacctcccaaaatgctgggatgacaggcatgagccactgaacttggcccctgatctaatgttctaaaaaaatcttagcaaatgtgtgttaaactaaaaatgctttgaaatgggatgactcagtaatagaaacatagaaccaagcccttgctttaagatgtcaagttggatttctacagaaattctgtaattagaatataatattgtagtaaaagcattatcttcatagaactaaggaaatctgagttcacttcttcattacccaaatttagttgtgcaacctttagaaatcattttatcagggatgcactcctgaccctctgtgatatcatgaaatacgtatttggactttgtccaagtttcttggcatataactcctaaaatccttggaatttccaaaatgctgccttttttgtatgctaatgctggctcatgggttcagaatgggggtctggtcactggaaaggcaaaggcacaattagagagtcagccccaccccccagcctccaggaaggagagagaggctgaaggttaagttgatcaccacttgccaatggtttaatcaatcatggctagataatgaaaccttcataaaaacccaaaaggacaggtttcagcaaggttcaagatagtaaaacatgtggagagttttagaggttgaagcccagggagggcatggaaactctgcacctctccccccataccactccctagatgtctcttcatcttcatcctctgcaacatcctttataataaaccagtaaatgtgtttccttgaattctgtaagctgctccagcaaattaatcaaacccaaattggggtcatggaaaacccaatttgaagctggttggtcagaagttccagaggtctggatttgctcctggttgtgtgtggagtcttgggaactgagtcttcaacctgtgcgatatgacagatctccgggtagacagtgtcacagttgaattggaggacacccagcttggtgtctgctgcttggtgagtggtgaaaaactcccacacatttggccacagaagtcatcttctgtgttgatgattgttgctgtggtaatgtgagaggagaggaaaaaacacagtttgagcatttcccaaaacatcctccaagctaaatagacccccatgttttaccttctctccttttgcttttttttcaagacatgtgtcacaattttagaaattattttttttaatgtcagtacttcctttggatcaactcccttagtgtaaggtttaccgttggttccctaatagccaggacaaggactggcatgaagcgcgtgctcaataaatatttgctgaattaataagcttcacctctttaagtctcagttttatatcagaaaattaggagaagaaggtttggacaagaggtgctataatgttccttctgagttctaacatcttctgtattctctgtcttctaaaatcataggtctgtaacaagtttacaggttggtgctactcataataatttatgtattaaacattttaagtgagaaatatttcaaggtcaggtgtggtggctcatgccctataatccgagtactttgacaggctgaggtgggtggatcacacttgaggtcaggagtttgagaccagcctgaccaacatggcagaacgctgtctctactaaaaatacaaaaattagccagaagtggtggcactcgcctgtagtcccagctgtttgggaggctgaggcaggtgaattgcttgaacccaggaggtgggtgttgcagggagccaagatcataccacttccctccagcctgggcaacagagtgagactccatccccccgtaccccttcccccaccgccaccaccaaaaaaagaattatttctaacgtaaaaaaagtaagtaaattaagacatctgagtataagacaaaatgttaacatttttccatattgcttcaaatcttttccttaaataaaatataaagcaaaaagtactcctccattctatctcatttcccttctttcagccattaaaatgaataaaatatacctgtatataagaacatggataaatctgggaagcatgggaattgaaaatagattactaaggctaaatgtaggatgatgtaaattaaattttaaggacacggggagggatagcgttaggagatatacctaatgctaaatgacgagttaatgggtgcagcacaccaacatggcacatgtatacatatgtaacaaacctgcacgttgtgcacatgtaccctaaaacttaaagtataataataataaaaataaaaaaaattttaaggacacgcaaaattatattacatatatttatggatgtgtattaaaaatgtatgggagtaaataaaattctttttttataaatgaatctagtaatttgaaatggttaagaacttaatgattattaatagataaatatgaattgtgtgatttattttaaatggttaacattcaaactttagcagacttttactatgtcaacaaacactttctcaccaggttgagatacagttttcacagtgctgtcttaaaattacttaagctatgttaaacactttatgtggagaatcttagaaatatgaggcttcagtcataattgttatacatgacttttattttgatctttgcagttgagtaagaattttttaatctattttttctttcaccttaagagatgtgaactataattttttccattttatatgtgtaatggtgaagtggccaaaatgtgggttagatgttattgtatttcaaagcgagcttgggaaggggcgcaaagcagtctgcctttaaatgtgttatttccagccaggcgcggtggctcacactcgtaatcccagcactttgggaggccgaggcaggtagatcgcttgagcccaaagagtgagagaccagcctgggcaacatgggggaaaccccgtctctgctgaagaaaaagaaaaatagaaaaaaaaattaaccgggcgtagtggtgggcgcctgtaatcccagctactctggaggctgaggcaggagaatcacttgaacctgggaggcagaggttgccgtgagacaggatcgcgccactgcactccagcctgggtgacagagcgagactctgcctcaaaaataaataaataaaataaaatgtgttagttcccctttggagcagaaagaagtggacacttttataaggtaaggagggaagtgaacaagggcaggcggtccccctgctagcttggtgcctcatctacctgatagttgagttggcaccaacccgggcagaaataagttgtgaaaatggccaagcaggcagacttaagatatgcccttctgatgggtgaaagtcccaaggccaccccgtgaaggtgaaagttccatggcaggtgttctctggtttgcaaatggactatcagctctccaggagagatttgtcttggagcacacagttagaacttaccctgcagggagtatctggtgaggaggacgtgaagggttatatttgcatttctgaagggctaagtaggaaacagggaacgggaggaaaggggacaggataagagaaaataataaaaataatagtaactcattcctcttttcttagaagaaatgggagtactcagttaagactacatgaaggcagagggagggaataaaaagaggaggcagaatttaaataggattttttaactcctagtccaataaataaatgatgcccacagttgttggtgttggacaagagtctgttttctgttgcttttagatctgagaactgagccctctccttcacaacaaaattggcctattaattccagtgttacttcatgtaatttttttatttgtccagaactaaaactatcctttctctattgtcgacaattccattggctaagccaggtttcaattgtctgctgaatatcaggagctctgtgttaaaattaactcattcattctgctttgattttagaaaacagataaacaactgatagtatttttgcaagcatcttttgttctaaagcataatatgtattggtctgttttggtacttccatgtatgtatttcatgttaaaaccaagaaggctgaatttagtaaatccaaagagtttcccccatcattttcccttttcattcatttctactgccctaggttgggccttcctcatttcttgtctgaatagctcctgatgggtttccccaattttactttgcttgttcccaccacctttgtatttctgaatacaaagaactgattaggtctttcccctaccttataatcctcagtgacacctctttgcctgctcatagtagttaacggtatcttgtgacatccaaggctcctcagaatctggtaagtgcctttctagctcagcctcattgacttctgctcggctactcctcctccttcccccatcccatctatatttcatccacagttaacatccccttgcttttactgatgatattctgtcttcatggaaagcctactgttgtctcctggctttttcttcctttctttcttgggtgcacaatccaattggtgaataataacttcttttttaaggcctagtttaaatatcactacctctatgaaaattgtcttttctattttaattttctctttcttatcccagccagttcatcagctaatctggtgacttctcatcacctctactgctggtctaagccactctcatcttacacttggattattggcaggagccccctgactggtcttcctgcttccgtctttgttccacaaagtctgtccttcacataacagctacagtagtcttatagaaataaaaatcagaccacatcagttgctcaagcccctctagtggcttcctatctcacacgaaaaaagctcaagacccgtcctgctctacaaggccttgcgtgatctgggctccctattatctctgatcttggatcttaactcctaccactgcaggctttgcttacttttctgcaggtgcactggcctccatggcaagcatacccagccaggactttagcacttgctgtctctagtacttggaacactatctctccaggtatatatatggcccagctcattatttcctccagacttttttctttaatatcatcttattgaagaattctttcctaacaatcttggaaaaaataatatcctcttcacactcactcccctgactcttggctttttatactctatcccctttgaatttaccctgatttactttttcttatagcacttatcactatctgacatagtagttatttgtttattctagaattctagcatttttagggcatagactttgtctttgtttaatgctgaatttccaaaactaaaacagagccttggtaatagtatatgctcaataaaaatcaaatgtattacctagtagttaattgttctatctctgtaatcccataacacttgcagatggttcctttatacccagtgtattttggttgtttttgctgtactctgcctctagactgtgagcaacttgaggataaagatcgatttttttctttcaatttattttatatttcaagtaccatcctaggcagcatggatacataaaattctgcattccaggattttaaagtcttacttctatctgttcccagtacagaataaggacctagcattgtgcaatacatgctcagtaagagttggctagattaagtattgaatgttgtacaaatgagaagagctagctctcatttgtggtttatgatggcaactgactagcagcttttagtgtagtctctcctatgttgttgtttttatgtttgtttgtttgagatggacttttgcttttgttgtccaggccggagtgcaatggcgcaatctcagctcacggcgacccccgcctcctgagttcaagcaattctcctgcttcatcctcccgagtagctgggattacagacatgtgccaccacgcccagctaatttttttgtatttttagtagagacatggtttctccatgttggtcaggctggtctcaaactcctaacctcaggtggtccacccgtcttggcctcccaaagtgctgggatcacagatgtgagccactgcgcccggcctctcctaagtttgtattaaaatattaatattacaaattccagaaaaaattaaactaaattggacactcaagccattggcagaattttataataattttatgccctaaaagcctgataggatagaactgaaaaatgtcaaaatgaaatgaaagctgcaaacactagctgtggctatatcttgctatgttcatggaaaagtaaaaaagcgagaaagggcttatattaaaaacaaacaaaatcctatctataggggaacatcgttttaaatactcaagaaaatcaacttaggaactggaagaaatatttagaagattactttggtgcaaattaataagacagtaacactgtaaagttgggaagacatggcaaacctaaaattgcaggctgagatgtaaagaggactagtaaaaagggatgaagattaaataaggaaggacatttgaaatgcagcagaagaagtgaaagtattatttgtagtagaatagagcagaattaagtgtagaaaaccatacacatactgaagacaaatttaagaagctttcccaaaatgcaaacaataggaaaaactgcataaaatatatggcggatagaaagtggagatcctaaagtaagacttaacgtgtttctggaaactgatacgagcagaagaaacaaaaatacaattgaagaacaaaatgtattgaggtcaagcatgaaagttaaaaggacccaccaatttccaagtaacttccattttaaaaatcacatttagagacagtgccactttattttttaaaaaagataaacaaaattctacaagaaactatggaaaaataagcagcttacttaatttaaagaaataaaattcagattgaccttggacttttctacactgaaatgtacattgactcaattactctgttttaagagatctatctaaagccaatttatatactaaactgtgctatatggtgttatttttaactggagtaaaatttatacagaagtatactaaattatactattctactaaaaagttacaattaaataaataatggtatatccctttttaatactgtgacagccaccacttaaaatcatgttttaaaattagaacatgctcatgatatattaaattataaaaagaataccgtctttgtattatatgcccctagtttgtgtgtatatatgtgtgtgtgtgtgtatgtatgtatgtgtgtgtgtatgcacagaaaacggtcaacaatgttaataatggtatgtatctcttgagtaagcaagaaatcatttttttccagaattctgtgtgcacatgttttgacaatgatgaaatattaataaaaggcagattgtagaaaaagagaagttggggagacgttgacaaaagttttaaattttctgtaacggcttctcatagtgacaacactacagggcaaggtactttataccttcacatagaaggtttgaaatatttgctatccacccaaagagctgctggaaatttaatacaatgggctggcataatcaaaaaagttcttagctatagaagatattttttcaggtataaaatcaactgagtacttaatttaatgcctataagtaggagaaacctacatagagatataaggtaaccctgggaatagcaattaaaaaatctgtttagaatggtaaagaaatgtgtaaaggagaaaataagtttgagctagaaatggaacccgtgaaacaagcagaagacctagagaattaattttcttaggaatgtgagtaagtgttgtatttgatatactggttatgaggactgataaaagatttaaaagaaatgcttcacatatgactcagtattaagaattagatatatcaaaactttgtggacatagctaagaagtacctagaagaaacttggtagaattaaatgtttatattagaaaaaaacgtgaaagtctaaaatgaatgatatatgattctagttcaaaaaaatagcaaactcaaccctggtttgattattgcagtcacataaaaataaaaatttaaaggtatgcagatttgaaaggaagaagtaaaactgtccctgttcatagttgacatgatcatctatgtagatcgtaaggaaggcatctacaaaaagctacaagaactagtaagtttaccaaggtcataggctacatggtcattatacatgaattaattaggtataaatacatgacatagtataaaacgcattagaaacaaaaagaaattttcatttttaaaagtactgtttataacaattattttaaaataaaatactgaaggataaatctaacaaaataggtacagtatttatacactgaaaactataaaacatttctgagtaaaattaaagatactctgtttaaatggagagatatcatatgctcatggtttaaaagacaccacattgctaaggtatcagttctcccctaaaagaccttacatatttaacacaatctcatgcaaaaatcttaccaggcatttttatggaaattgtaaactgcatgtaaattttgtatggaaaaatcaaaggatataaaatagccagaacaatttttaaaaagaacaaaattgcaagaatcagactacctgatttcaagacttactataaagtagattaacccaaacaatttgaaattgatatatggatagacaaatagatcaataggaaagagggaagtattcagatacagacatgaacacatatgaccaattgattttcaataaagagtccaaggcagctcaacaagggaaaatatgcaggtaatcttttcaacaaatggtgttagaacaattggatatctgttaaaaagagaagaattataaacccttaactcacaccatatacaacaattgactgaaaatatagttctaaatggaagagctaagacaataaatctacttgaagaaaacatagaaaatcctcctaatgtcaggccaagatttcttaggatataaaaagcaagagctttcaagggaaaatattgataattaagtattttgggaaagcaggaaaaaagccaagctacaggctgggagaaagtatatttttaaatgtatatctgataaaagacttgtagccaaaatacataagggactcctaaaactcacattaaaaacaaaagcaacctaactgacaaattaccccaaatcgccaaaataacccaattaaaaaatggccaaaagaccggaatggatatttgtcaaaataggataagcatatgaaaaatgcccaatatcactaatcagggaaatgcaaatcaaactacaatgaaatactacttcatttagaatggctattaacaacaagataactgttagcgaggatgtggagagaagagaacctttacatactttcagtgggaatgtaaattagtacagccattatggaaaatagtatagaagttccttgaaaaattaaaaatataactaccatgtgatccagcaatctcactactaggcatttatccaaagggaataaaatcagtatgtcagagggatatcttcactcctgtttattgcagcactatttgcaatagccaagatatgcaatcaacctaagtgtccatcaaaggataaatggataagaaaatgttgtgtataggccgggcgcggtggctcacgcctgtaatcccagcactttgggaggccgaggcgggcggatcacgaggtcaggagatcgagaccatcctggctaacacggtgaaaccccgtctctactaaaaatacaaaaaattagccgggcgaggtggcgggcgcctgtagtcccagctactcgggaggctgaggcaggagaatggcgtgaaccccagggggcggagcctgcagtgagccgagattgcgccactgcactccagcctgggcgacagcgagactccgtctcaaaaaaaaaaaaaaaaaaaaaaaaaagaaaatgttgtgtatatgcacagtagaatactattcagtcagaaaaacataatgaaatcctgaggttgggcatagtggctcatgcctgtaatcccagtactttgggaggctgagacaggaggatcacttaagcctaagagtttgagacaagcttgggcaaaatagtgagaccccatctctacaaaaaacatacaccaaaattagctgggcatagtagtgcgcacctgtagccctagctactcaggaggctgaaatgggagaattgcttgagtctgggaggtcgaggatgcagtgagccgtgatggtgccactgtactgcagcctgagtgacagagcaagaacctgtctcaaaaaataaaaaaagaaatcctgatcctgtcatttgcaacaacatggataaatctgaggacattatattaagtgaaataagccaggcacagaaagatgtaaccacaagatctcactcaaatggaatctaaaaatgttaatctcatagaagttgagagtagaataggggttaccagaggctgagaaggggacacggggtgaggggaaatggggaaaagatgggtcaatgggtacaatattacagttagacagaaggaataagttctgttctattgcacagtaggatgactatagttaacagtaatgtattgcatatttcaaaatagctagaagggaggattttgaatgttcttactacaaagaaatgataaatgtttaaggcgatggatatactacatatcctaatttgatcattatacaatatagacatgtatcaaaacatcacattttacctataaatatgtacaattcttttgtatcaatcataaaataaaacctaaaaagtgaaatttctgtgttacaaagggggagaacacagtctgtattacaatttttttaactcaaaaggtacaaaatatttgaacagatgcttcaaaaaatatcaaatagttgtatagaattacataagtttgtttttatttttattatatataaatatgaatgaaataagtacatgaaaagatgttaaacattattaatcatcagaggaatgttaatttaaaccacaatgagatctgcatacataccagaatgacatcaattaaaataacagctgacaataccaaatgctgacaaggatatgaagtcctcatatattgcaagtgacaatgtaaaatggcacaaccactttgtaaaactatttgggagtttgttagtaagcatacacttacccagcaatcatattcctaggtattcaagaaaaatggaaatgtacatcctcacaaaaatttgtacatgaacgttcataatagttttatacacaatagaaaaaaatggaaataattggtgagttgacaaataatgaagtgctattcaaaaatacaatggaatgctgatacgtgcaaaaaccacagataaatctcaaaagccttatcacataagattatatactatatttttccatttacataaaattctggaaaggtaaaactagtggaggaaacaggtacatgattgttgaggccaggagctagggaggggatgaactgcaaagaagcaaaagataattggaacaggaatggaaatattctatatcttgattgtagtagtagttgcacaacggtgtacatttgtcaagccatcagcttttgcacttaaaaactggtatactttgcggaaaagtataccacataaaacaaaacccccatacaaaaaagaataaattaggccttagaatgaaaagaatctcaagtcaatgttgaggagcaatttagacgtgaattttttaaattccacagagaaagaaagatagtaaaataagccatgtgtgttcaaatttacaatttctgctgacttgaatcatcagggtattttaaaatcttgctgtaaatcagattcccatgttccaatcttgcagaagccgggaaaattggagatgatggtgctattttgtaagggatacggaagtgggcaagtctaaggacttgtttatacacatagaccagggagactgaacttagtactgggtaaaaaaaataaaggtcatcatggaacagatttgcaaacaatgaagtaaacagaatagcagagatttggctttgtaaaaagaaaaaaaagcgattgtgccagaccacttcagcgcctctagttgctgaattgcaggactggtagttgaaccaaaagcaataaacataatgcatcttggctgtggtcagatattttgatttagtcaaatatgaagccctcaccactaaaattggaaaatatggtttgcagtacagatacagtactagctgaaaagtgatatctgaattgtcctggcatgacgtggagagcagaggaaaaagagaagtgtaccacactgggaatatcacttttgttgttgtagttgttgttgcttgcttgttgcttttaatttttttaggtttcagattcagtaggtacatgttcagtattgttacatgaatatattgtataatggtgaggtttgggtttctagtgtacctgtcacccaaacagtgaacattgtacccaataggtattttttaacccgcatcccctcctaccttcctcatttttggagtcctcagtgtccattatttccatctttatgtccatgtgtatccttttggggtttttgtggttttttttcgtatgtttagtagagactggatttctccatgttggccaggcatgtcttgagctcctgcctgcctcggcctcccaaagtgctgggattacaggcatgagccactgtgcccgaccacatgtgtcttcactacttatctcccacatataagtgaggacacatgatacttgattttctttttctgagttgtttcacttaggataatggccttcagctcaatccatgttgctgcaaaggacatgatttcagggaatagcattttgaattttgggtgatgatcctatggtgtgtttgaagcattaaaacgtttgtatgggtagaacatggggtgagaaatgtgtgtgggtgggggtggaggggtagggggaattagagatagataaggccataatacagagggcctcataaagtcctcaataaaccatgttaaagagcaagactgtatccttgaattcccagaagcagctggtgtaatcgtccaggctgtcttccacctagcccaaagttttttcaactctatcacttgctgcctctccctaccattaaatgagtaagcaatatactgagaaattaataaaactatgttaaatagaccacttttgagctccattttaatgcttggagaagagtcggtaagcattgtaattgattcagaaaagaaccacaatgttggaaactagaaaaaggtaaacatttgcatggctgagttaagagaacagagacataagaaactgttattacaggactaggatagactcatataccagttactaagcaggttcttgggcaagtcctttctgtacatttattttcttatttataaaatagggatgataattcaagtcaattatctgttatctacaattctgaagttccaagagctctgaaaatcagaagttggttcagttttattttgatttcttaagtttgtggcacactcatttgatggtgaaacccaggtttcgtagtcatttatcagttagtgtgaatatttatgaattctgcagaaataatgtttgatcttatggtgctaccctaaattctgctggatacattcatatgttatgcaaactactatttttctaaaatctaaacaattctggattctgaaaattctggcccctgtggtttcagataagagacagtatgcttgtaataccttcctcttagggttgtatcaggataaaatttcttaaggcagaactcagtaaacgatgaagctattattatttatcattaattaagcttaaaaattagattcccacccccatccccgagaatgaacaagaattagcacttgacgatccttaattttattcttaatgcttttatactgtactctctcgagcttgatttatagtatatcagttattaaaatggaatgttttccttaagaaacttctacgctgaacacttaaccttcatttctgcaatctttacatacttggaaggataccttatttgcctttaggtattagttgacagacttatttttcagtcatctttcctaagggtcatcttacgaaattcatggttagattatgcgttatatacctcctaacgaaaacacgaatggatatgcttattttcttcaagttctcaataaaaggaagttcttaacatgtcataaaacaagttctgtgtaagtctttagttaaaagcactagagcaaaaatacccatctagatttcattcagtgtttcccaagtagaattttatttttgtggttgaagtttggcatgtttcctatttcttcaagctatataaaacccttttgctgcgtctaatccatggcctgcggacatggaggactgattttgcaatacttttctagcattgggaaacttctaactaaaaattttgtgtgctggaatacttggaagaaaaggaaaggtcatttaaacaaatactatattttaaaatgaagaaataaagcaacattttaaatgtgtgatttttgttaagatacttgccattagtgagagttagggtgggacatatcagtaacgaatccgattgccactatggctctgcccccccaccatctttcctctgggaggatgttggggccaacttacgattgcaacctgtgtgtgtgtttatcctttgtttggatacatgttactaggtgccccactcttggccagcaattccagtgcctgggtggtttgcctgagcaaagtgcctgagtggctaggaatgtagagatagggctttgacccagagcacagggatgataaggtttactatagtgtaaggattagaatgaaggttctcttctgcttcagaagtgggaagaggaagcaacacaggatgggatctctatctggttctaggtctcaggctttctccagcccgttttgccaacttacaagcaactaaccaccactaaacttcctctccctggagaaggaaaaatttcactcacttgtgagtgagaagatctgcatccagatcctttctgtgccatgattagctgtgctacatcaggcccggtgttttcaggagtttaggaccaaacagcccctttcagctcttgcattctcctagtctttgacatcagaaggaaagtgcaggcgttcaagggagttcttgttttacagtttcaagcgatctgacgtcaggcgaaatctcttcactctctaacagatattccatcttaaaaaaaatcccatgaaccctaattcctgtgagtgtctctaaagtatattaaagactggattaattgtaatctaggcagaaatctgtctttgtggttgttgtttaatatcactcagtacatgccaatactcttctttcacttagcttctggtaatctatgaagctactcgtggtttcattcatttaaacccctctgagcctcagcttcttcatgctaaaggaaaataacaggaaataatggcataaatgtgttttgataaacataaaaccttttgtaaacatggggttttatttatgatagcacaaaatattaatataacttcctctaataacattgacatacgtcacatctatagtagccagtagtaaacaggcagcatgcagctctaaaaagtggacagattttcattgttctgatttggaatgtaacaatatctcctgttggttctctccttcctttctccactggccccaactctaagcagtattccactgaactgaagaaactctactgccaaattgcaaagacatgccccatccagatcaaggtgatgaccccacctcctcagggagctgttatccgcgccatgcctgtctacaaaaaagctgagcacgtcacggaggtggtgaagcggtgccccaaccatgagctgagccgtgaattcaacgagggtaagcagaatttgaatctctaactgttcaacctccttgaaggtcaagattctgtgggcatttttgtttgagacccacctacctgattcagacttctgcactccgatggcagatcagtctgccttttttttttttttttttttttttggctgacagtatctaagaataatgataaataaattcatggctttcacagtggttgaactatttaatattagtaattactagatgacctgaactactggttatctttactccttcctgcttccttctttttatttttcttgcctttaaaataaaataagaggctgggtacggtggctcatgcccgtaatcccagcactttgcgaggccgaggtgggcagatcatgaggtcaggagatcgagaccatcctggctaacacagtgaaactccatctctactaaaaatacaaaaaaattagctgggcatggtggcgggcacctgtagtcccagctactcgggaggctgaggcaggagaatggtgtgaacccaggaggccaagcttgcagtgaaccaagatggcaccactgcactccagtctgggcgacagagcgagactccatctcaaaaataaaaaataaatgaaaaaaaaataagagggtgccaggagactacatggtagctattctacttggcccaggctacagtttcttcagatttagtgttttcccagaaagattctcaagtctagaccagttgagacctggaagggtaagagaaaggagcaccaaatgaaagaaataatgaaaaaaccagaagaagaaaaaggaaaaaaagatgagaaggcaagtaatgggtaaggggaataaatagaaggcctagagcatactgtggccaatcaaataaaggactgggagtggagggatgaggtatcatgaaggccacctttagacgctatatccatgaataagtcatgcctcatattctacatctcagatcctacaacaatttagcagaaaaggtcagcaatgccaagagttcccttagatctgatattatagttccatgaagggagactaaacagagagtgctgaaaagtggaaggtaagagcaggataaagcataagcaatcctcaaagagtattgagggtgccaaaggtgttgggggttaggatactttatagaaaaaatattaaaatataaggattgataaatatagagagtataattgtagagaaaatcaatggaagaagaaagagaatggatcggaatttaaaagaagaaaataaagtcaaaattctctttaaaaaactattttttacacattaatttcaagtccttgcccacatggccacattatccgtacatttatctcttgcttttcacttaacaatctgccataaagaatattgcaatatagatagacttcattatcagcctccctaacttggtatttcaccaagataacatactatttgtaagcgttttcccattttttaaacataaaaattgtttttagttttaaccataatagatgatgcagcagagaacattttgatatgcttcactttttacttttattgaatggtttttgtagactgggactataggacctaagagctgttaaaaacagctttacgggtcccagttcttggagcatcttcttttttttggagctctgctaaatatcacaattttatgaaactttactcagtctcttttttaaaattgcatcccttttcttacacccaacttgggactccttcaacacacaaacaccagaacgtttcttatctttataatcaacacaatgtttaaaagttgaggaggaggaaagaagaagaagtggaagtcatatccttgctatcttctgcttctcagtgtatttgctggtcagacttttgggcagcattttctcagttaaactgatttttttttaaattaatttgcttatttccctttcattttccaggaagctggcaaaatttcctccacgatgaggtcagagatgtagttatagagttccataaagcttgaatcattaatcttaaactccaaattataggactgttcccttaactgtagatgtgcattccagccctttctgttgctataaaggttatgacaccttcatgtatttgatatctggctatgggatctgttcgtttcttcaaggatgcacattttctttattatacagactatttcttttgccaccaacatcctgttcatgcaatttcattttattttctttatttttaaggtaaagaactaactcttttattgttttctgctctgcaggacagattgcccctcctagtcatttgattcgagtagaggggaacagccatgcccagtatgtagaagatcccatcacaggaagacagagtgtgctggtaccttatgagccaccccaggtaaaaagcaaaaaaccaaaccaaaaaacaacacctctatggactgagtagacttgagagaacatctgtttcagcaacagggatgtttctagcatctatcactgtcttagttcttgtcatcaaatatataatatttttgtatttttttctacctgttctccacatgctttcccttcagaatacctaaatatgcactgaatcaacttggtaacatggctctgtgtccatattcccatcttaagggcagataacaattgaactggcaattgggtgtatacatgagattgttttatccaactggcagttggacgggtagtggataagcaatagtagtggatgcaagtgttaccataagtgggaaagcctaagagggatctcttagctgaagtttgaatgaacagatgatagatttgtaaatctgttgcatgaagcagagtagtgtggttgaaagtacactggtgtaacaggacccaagttctttggtttgctggcttgctgtgtggaccttcggcaactctctctctctaggcttcagttgccttacccgtacgtatgtactttggactagattatgtttaattctgagaacattttgagctctaagatgttattaatggaagtatatattcaaatctatattgtactgttttatccacacagggaaaatattcttggagcattgctactgtttttggcaattatccccagttttcccttttcagtgatattcatgaaggtggtatagcctatttgaattacatgatgtggatcagcttacaaacgaacaggatcaaagatcacaaaatgttaaagcctgtctcacctaaggtagtgttcagtgttgcaaacattagctttaagcttccccgcaggcaagatgaagaaaaatctacaacagggttcagagtttgcccttttaggaggaagcgtatcacttcatcagaagtggaattccttaaatagagggaagaactgagaaggaacaacgtcagtttaaacccttgttaacacagattatttaccccttgttttcaggttggcactgaattcacgacagtcttgtacaatttcatgtgtaacagcagttgtgttggagggatgaaccgccgtccaattttaatcattgttactctggaaaccagagagtaagtggcgtatgtaaaattgtcattctacacaaaaaatcacgagcagagggcaaagtgaaatcgtggctgctttatcattaattttgcatgtgcagcggagagcttgtcctttgtgctctaaatccttgctacaaacggttacataaaagatctaagaaagtggagacaaaggaaggtgggtaaagttagaaggaaaaaaagagctagaaaagtgtgcaagtcacttcatacctgaattcttgacatttgactggaattgttctgattagaccatggtcctcaaggcatttcacagttttttttaagtctgcgctgccttaggggattttatccttgagacatccactggcttaactcaagtttccttcaaaatatgtagctaaatacagctgttcagctaatagctcagaggttctttggagaacaaatggaatgttatttactaatattacttgtggcatgttagcacttttgtgttctgccaagtgcttttgggtccattctcaaagccgccatggctaagctggtagtacgttggcgatggcccatatgggaagtggaagtggtagatcttcaggggactttcaaaatgctttgaatttaactctttcttcccctttattctaattcctagtgggcaagtcctgggccgacgctgctttgaggcccggatctgtgcttgcccaggaagagacaggaaggcggatgaagatagcatcagaaagcagcaagtttcggacagtacaaagaacggtgatggtacgaagcgccgtaagtagatgtagtggccaaatggggtagggttgaatcttctccagatgttggagaatggggtgatattggagaagctgcatgataagacctgtgaccttcagcagcaagtgggacgtcagccctcagagccagtgagaataggtatagcattgaagtggactccaggcatgtactacagctttacaaaaacaagtagtcattgtgacaatttctcagtccagggattctcaaaatgtgacttccacaccagtagcataaacatcatctattcacagacacacaccagcaagatacgggctttcctgcaacattacttaagccaaaggctatttcccagcatactgaatttcttttatgtattcttccctaccatccttccacctccttattgctaggatcacaatcaatgtaaaaacaattgtccccagttaaatggacgcttagggctagagcctctaatcttacatgtgttgctggtactactgtctttttaatatgtatattaaatctgattaacattaatatttaattattaagtatatttaatatgcattagtgctttagaagtgttcccaggatgaaacttgcatttttcctccaccagcgtttcgtcagaacacacatggtatccagatgacatccatcaagaaacgaagatccccagatgatgaactgttatacttaccagtaggtcttccttgggtgttcatggttgcttcattttaaccttctttgaatgggcttttacagtatgatcatcatctcattatctgtgacaatgggaaaggaggtgtcttagtcagttgaagctgctacaacaaactaccgtagactagatggcttataaacaacagaagtttattttctagagtctggaagtctgagatcaaggtgcccctatggtcgggttctggtgagagttctcttctgagttgcaaactgccattttctccttgtattctcatatagtggagagggctagagagctctctagggtgttttttttttttttataagggcaaaatcccatttatgaaggctccactctcattacctaaattacttcccaaaggactcaccttctaatgtcatcacaatggggttaaaatttccatctgtgaactttggaaggacagaaaccttcagtccataatggggaagaggggagacggttgagagctggtgatacaaaggagagaaaatatgtgttttttaaaattagaatattattctttctctccacagttttggactgtattactagcatgagcttctctacttacctacccacccaactaaagctaaactcctaacagaatttttcttaccctaatatttaaaattaatttgtccttcattatttgagaatgataagattatcctaggcagcatggccatagaaatttaggccgaaaatgttgtcagaaaaagttcacaaaattgataatgcagcaatgatagccttgactttgatgtcttttaggcagcactataaagaaagccaagcaaggcagaagcctgaattacagtcagccctccctatctgcctgttccatatctgcagattcaaccaatcacagatcaaaaatagttggaaaaaaacaataaaaaaatacaccaatcaaaataatacaaatataacaacaatatagtataacaactaattgcatggcatttacattatattagatattataagtaatctagaggtaattttaaactatacagaggatatgcataggttacgtgcaaatactacaccattttatataagggacttgagcaacctaggattttagtatccatgggggccagatgggggtgggtagtcctggaatcaatgctctgctgatatcaaaagacaactgtgtatgtaaatggccagaagtcttagcccaattattatgtaagaacctcattaattcctacaagggacattacttctcttgcaatcactaatgagatcactttattagattatggttgcagttagttagttgaattcttattgcattaggtctttgccatgtagggtggcaatgacgttttggtggctggcatagcccttgatgccatctaaagcgatgacatgagacacagccaagcagggccaggagatttgaccatgaactactaccctgggattgaactaacgaaagtcaacggatttaggtgtgctgcactttcttcctcatcttctttgggtcctgactctcaggggaataaattgccagtagaccaatcagacggtgagttagctgagattcccagtgtcttgtttctcaaggtatatatggaaactgacttgtgatgttaaagtaaaacattaccctgatacactaaatattccaaaagtcttaatatgttttcagctgataaaacaggaactgcttgaaaaattattttaaaatttcagtttgtactaagaaggaggtatccttcttagtgtcatgtctcaagttgagagaagacttagcctaaataatttatgggtgaggaaaaactccattaatttaatatatattttgttggtgtttggtgagtggcaaatatgctagaccttgagcatttagagttcagtaggatacaattcttactttcaactaccttgccttctattgaggacacaaacagttataatgtaataaataaatgtaatgtaataaaataaatattcctagatcaaggcctagtgctgtgtgagtacccaagaagctccaaagatgcaaaaactataaataagagtaagatcaagaaaagcttctcagagaaggggtcccacagctgggatgagtaagagttaattcaacaaaaggtggagaacatttttcatttctgaattctgactaattctgcctttcctactgggtgcagcaactggggtaaggcagttaaagtgcctagggtgcaaaatttaaatagtcactcacacccaaggcaatgcagttgtgctaagtgcctctcttgcctcactctagtcctggcccggctcatccgtttttttggtttttgtttgttttgaaacaggatctcactttgttgcccaggttggagtgtagtggcaggatcatggttcactgcagcctcagactcccagactccagcaatcctcccacctcacctccccaggtagctgggaccacaggtgtgggccaccacgcccactaatttttgcacattttgtagagaaggagtttttttcccagggtggtcttgaactactgagctcaagcaatctgttccctcccaaattgccgggattgtaggtgtgagccaccatgtctggcccctgctcataattgtaaaagcctttaagagcttttatttaaagattttaagggactctcttaaagagagaaacatattttatcccacatggaaacttttatcctatcaaacatagttacaatctattcttattttttgtagtagtttagttttatgaaggtgttgcaaaaatggtattggtggatatgaatcatctcatcgctcctaagagaaatataggattgggttcctatgcacctctggtcacatacatttcacaacagaataatatatcacttcgtttgttgtgtgtttctgttaagagacattttatttaatatataccgttgactcattaacatcagactcgtggccaattagccttataactcatgcctgaataaagtttctctaacacacacacacacacacacacacacacacacatattttctccataaggcatggaacgaccttctctcacttagggacactagatagcacttccgcactgcccttgagggtcttttaaataacaaaatcaccaatgaaaagcacaaaaatgtaaaacacgtggcgctaaatagaccatgaaaagaacgtgggtttacggtataagagctacaagatgacagagcatctccttgttcgaaccagctgggaatgcacacaggtgactcaaatttttcatcactctgcccgggtctgaaagaccacaaaagggccaagagtgttgattttcatgttacaaataaacgttagcaaataaacaaattgcaattacggaatcctcaaataatgaggattgaccacacttctaacagttctacagcttttcatgtttccttctttccttctgctcacttccataggtgaggggccgtgagacttatgaaatgctgttgaagatcaaagagtccctggaactcatgcagtaccttcctcagcacacaattgaaacgtacaggcaacagcaacagcagcagcaccagcacttacttcagaaacagtgagtgtatcaacgtgtcattttaggaggcatgagtgagggtgactttatttggatcagcaatagggtgattgatgagcaatgtggaacataatgggagatagcagattgtcatagattcagatgacctggtatggcaaccctctttcagttgcaaccttttttacgtgtcttattataaccttcccttcagaattccacttatgttctgaaattaaatacaaaccatttctggtgaattacaaagaaactcacactaacagttctcgtctctatatgcctggtccatacacactaacagtaagtacacactctatttggtagtgatgtgtatatttgaaaacatgaaatcttttctcatcccaatggattgtcttataaatctcctgggatgcacactatccacttttgggaataacactgtagaccagggatagcaaataggctttactataatataaagtgacttgtttgaatgctgtaatgagaagaattctgagacctagtgcatgataattggggaaatatctgggtgcagaaggataaggtagcatcatgttgccgtattttagaatctctgctctagattgtcttccagcagtacatttttggcataaatcaagccagatttttcaagtatattaccatatacatataacctatttatatgttaagctttgccttgaatcttggatctttcctaaagtgattttatgactgactagaatagaatagctcttatgaaaggatgagaggttgaccaatcaggaccttgtttatatttacctttctgtggttaaaaataatcacttgttagtttaattaaaatatgatatgtgtcctggaagtattgtcagagactctgtagataagtgtaaaatatatacattatgagtccacttgagtaatgtagactttcaacttcatactttaggcaaaaaagaagtcaaaaactatcttccagaggcaaagattatattttacaatagaaatgcaaatgaacaataaaaatggttattttctatgttattagctgttgtagggagtcagctgtggaaagtgtaataaagagggcttatgattattattattattattgagtctcgctctgtcacccaggtaggagtgcagtggcactttcttggctcactgcaacctctgcctagcgggttcaagtgattctcctgcctcagcctcccgagtagctgggattagaggcacctgccaccatgcctggcagattttttgtattttttagtagagatggggtttcaccatgttagccaggctggtcgcgaactcctgaactcaggtgatccacccacctcggcttcccagagtgctgggattacaggtgtgggccaccgtgccggccttgttattatgttttaatcaatatgaatgtatctctgatagtaaagaaacactttctgagcacctgttatctacagagtggagtagacggcagggagaaaagaggcacattctctgttcaaaggactctaagctatttgagcatccaagatcctttttggcccgaagggatctcaccatttacttttagcataaaagttttgcaaattttaatggtatggttttatttgtaagcctgagcagaaagtttaatcacacagtgtattctgaaggagaattagaactcagagttgcagacacaatttccattgctaatcatatggatgttttgggatatcagatggaaatatatactaacatatttaagtggaaaatgatttagttcttataaaaccatacaaccaggatcaaagaaacagagacctgtaacatcatttatagacccaggagggatatctacctttcctaagatatgtgtgtcatggtattaagaaataccctcaaatttcctcctgttcactctgaaagtttgactataccaaaatgacctccaaaacatcactaagtaatatatcatgctgaactagttgctcttcagtactctttcctgtctttttttttttttttttttcagttaataagaaaaggtctcatatgtagtacaggaattggcaagctgctttttgtaaagtcctggtagtaaatatttagattttctgggtctatattgtcttttgcaactactcagctctgtggttgtagcacagaaacagccgaagataatgcataaaggaaagagagtggctgttccataaatggaactttatggaccctaaaatttgaatttcatataattttcacatgccgtgaaatattattctccttttgattgttcctccccctattaaaaactgtaggccgggcacggtggctcacgcctgtaatcccagcactttggtaggccaaggtgggtggatcacgaggtcaggagctcgagacgagcctggtcaatatggtgaaaccccgtcgctactaaaaatacaaaaattagctgggcaaggtggcacgcacctgtagtcccagctacttgggaggctgaggcagaagaatcgcttgaacctgggaggcagaggttgcagtgagccaagattgtgccactgcactccagcctgggcaacagagagagacttcatctcaaaaagaaaaagaaaaaaaaatacatacatatatatatatatatatatatatatatatatatatatatatataaactattcttagcttgctgtagtttgatagtctgtcttatagataaatgcgtggatctcctttcattgcttcttgaggctccaattttgtttgataggtgtacttggaagttaatatgtatattccaagctagaaagttattaaaagattttaggaaatcctaaagtggtttttcaggtggcatttcagtatctaataaaaagcaacaccagccttgaacagatatcttttttgtctgagaaagtgaggcatgcccatccagatataaaatgtagttaacgacacaacctcattgtcagatgaggctggaatagttgacttgattcacgtcacatattcttattgaagatgtgctatgtgaaagcattaaggatagaggtgagaaagatgtagctcctgccttcccatggtctacaatttaatggtgaatttctaaccttaacactctgagtttgaagaaagctctggaaatttatcctcagcattgtgcaagttaataggacaattttaaacagtttccatggatcatatgagaaaaagaatagtttcatgtcattgttttagaaacttgacaagccaggaggattgacatcattcagttcaggggcatatgctaaatgtatttgtttttatggcatacatgaaaatacataaccctctttttctatacgatgagtatccagtgcttattgtctgaccggctcacattgctctaaagggaattttttattcatatttcataaaataatctgtgtccccagagaagaaaacacatgttaatattgtgttatttttaaaagcacacaagattttagggacatatttacacttttttgcatgtgtggccacagatagatagtaaagaactagtagcttcatagttccctggtgtgttcatttaactgtattatatgcatgcttttgagttggtatctcacagctgtgataacttgtgcatggaaatctaacagactgcaaaattaaatctttgcaaaatatagacatttggtctaattatgtctttgtaacattatatatacatgggtctccagtgattgatatgcatctatataaatccttcagttgaattcttcagctgagtttttttagctcttattttcatgcaaatctccaagaaagagcatgaatgactaaacaaagaaaaacaaaaatctaatttcctggcaatcatttgaaaagaataaatcagcttggaaaatacctataatagcctgtgattttctttccaaaaagatagtaccaattttatgctggtaaggaatcaattttgtatttttctttatagtagagggaaaattagtgggaagagaacgtactttctgtaaggtggcactacaatatttttactttaattacttctattaggttcccactatgtgttttcttctgcctgcttttcatttgtgcatggtttttctttttaaaaaaatttcttcgcgtttaccttcatgatcataggctttatcgaagatagactttacagagctgctgtgaatcaaattgttatagatacaagtaggctttatattacgaagaaatgtgattttagttgatttgcttacattggaataaatatagagagttatgtttctagatgccagaatctaatagaaagacaaatcggaccctatctttagacttcttgccatcctattatcaagggacatcaacgcttaagacagctttgcagaagcctcttgagacctatttagcaacgttttgacttctgtacttcagatcagtactatttcttttttcatttgttagttgttttcccttattcttggcttccaacataaaagcgaggctgacatcactttgttacagtctcgcttaggctagctcttactttcctctggaaatttatttttgagcttctacaggatgaattttatttaaatgataaaacagatctgtgacatcctgatgagcccttagtgtttccttctcttttatgattacttgacaattgtggctcattagtaacacaagtcagagattattagaacaaagcaggatctccaagtccatggttccagcccctcacaccatagccggataaactgaggctctgtgaatttatagccagtttataataaagctaagactagaatcagatttagaacatagagctctttgtaatgttgtgttctgctacattggcagttaagtaagtagttaattataaaatttaaacatctatttccatttttctgtctcttttttgtaggattgttttagagatggggtctagctattttgcccaggctggacttggacttctgggctcaagcgatcccctgcctcagcctcccgagtagctgtggaccacaggcacaccccactgtgcctggcacgtttttctgactctgttgttagttacttggtatttattcttgtaggttcactttaaagtcctgtatctttaaacttgtttggcgatggttatcatcaagaatattgatatttttttctcagattcatgtatctgagggagaagcaggcagtattttcagtattatcaatgtagaaaagagaaaggtacacggtctccaaactgatagaaaatgtcaacaccgtatttctaggttagacagagaaatcctccacgtttccagaatatctgaaattgccagctttataggttaaaaagagatgaatattgataatttcatatgattcacccttatagttattatggaactacacaagcttttacagtttcctttttttttttttttttttttgagacagagtcttgctctgtcacccaggctagagtgcagtggcatgatctcggctcactgaaacatttgcctcctgggttcgaacaactctcctgcctcagcctcctgcgtagctgggattacagatgcctgccaccatgcccggctaatttttgtattttgatatatatatataattttttttttctttttttttttttttagtagagacgggatttcaccatgttggccaggctggtctcaaacccctgacgttgtgatccccccgaccttggcctcccacagtgctgggattacaggtgtgagccaccgcgcccagcctacagtttacttttgaatttcatagttatgaagttttcagggcagagatttattatctctgaattggtaatgaagaaaaaccatagagacggggtttcaccatgttggccaggctggtctcaaacccctgaccttgtgatccccctgaccttggcctcccacagtgctgggattacaggtgtgagccaccgcgcccagcctacagtttacttttgaatttcatagttatgaagttttcagggcagagatttattatctctgaattggtaatgaagaaaaaactcaatgattaattaaactcagatagattaaattacttttgtaacttctctcacctcattgcagaatcaggctcagtcctaggtcttagaggtctttagttagctgtacattgcttccccatccatttttattcgggcatattctgttaagatgatatcaattcttgccggtgacatggtttactgaatcctagcattaagcaaataaaaatgactttgttcctttgaaaggcatagccaaatgtgattcaagaagccaatctgggaaacaatttccaaacagttcatgagattgcccaactttcagcaaaataaaagggtttatttctggcatcctcgtggtgtaaataatttcttaattatgcaaaaaattgctttcttgggtttgatatatgcatttataggccgagttttttactcaaaggattaaggtatttatccatctcaatctgggaatatccaaaatactcattaaacagttctttgtaaatgcctgatgccacaggccccattctgataatatcatatttctctatacctcacacttaggctagacaagaactgtaatgagggataataattgaccagatgacattttacaaaagattcaagtttcttcttcttcattccttttttttaattgagtccagtaaaccgtatgcgaagcactgtgttaaataccttgatctaccttatctcagtggtcctcgactgaatcgccttgtgtaacttattaaaaatatgttttcctggatgccagcttgtgttggaagggagccaggaaaatgcatttttataagtttcacaagtgattctgatgtttatttctggttaactactatagccttatcccatttcttcctcataaaaatcttgagaagtctcagtaattgtaaaagaagataaattggaagaacaaaaatagcttttcaggcactctattctgtctatactatgatcatgaaggtataaggagtgtgtttctgaattcaattgatttgaatagatgaagtcctaggccttcattttttcttttctctggttcctctctgcagtctcctttcagcctgcttcaggaatgagcttgtggagccccggagagaaactccaaaacaatctgacgtcttctttagacattccaagcccccaaaccgatcagtgtacccatagagccctatctctatattttaagtgtgtgtgttgtatttccatgtgtatatgtgagtgtgtgtgtgtgtatgtgtgtgcgtgtgtatctagccctcataaacaggacttgaagacactttggctcagagacccaactgctcaaaggcacaaagccactagtgagagaatcttttgaagggactcaaacctttacaagaaaggatgttttctgcagattttgtatccttagaccggccattggtgggtgaggaaccactgtgtttgtctgtgagctttctgttgtttcctgggagggaggggtcaggtggggaaaggggcattaagatgtttattggaacccttttctgtcttcttctgttgtttttctaaaattcacagggaagcttttgagcaggtctcaaacttaagatgtctttttaagaaaaggagaaaaaagttgttattgtctgtgcataagtaagttgtaggtgactgagagactcagtcagacccttttaatgctggtcatgtaataatattgcaagtagtaagaaacgaaggtgtcaagtgtactgctgggcagcgaggtgatcattaccaaaagtaatcaactttgtgggtggagagttctttgtgagaacttgcattatttgtgtcctcccctcatgtgtaggtagaacatttcttaatgctgtgtacctgcctctgccactgtatgttggcatctgttatgctaaagtttttcttgtacatgaaaccctggaagacctactacaaaaaaactgttgtttggcccccatagcaggtgaactcattttgtgcttttaatagaaagacaaatccaccccagtaatattgcccttacgtagttgtttaccattattcaaagctcaaaatagaatttgaagccctctcacaaaatctgtgattaatttgcttaattagagcttctatccctcaagcctacctaccataaaaccagccatattactgatactgttcagtgcatttagccaggagacttacgttttgagtaagtgagatccaagcagacgtgttaaaatcagcactcctggactggaaattaaagattgaaagggtagactacttttcttttttttactcaaaagtttagagaatctctgtttctttccattttaaaaacatattttaagataatagcataaagactttaaaaatgttcctcccctccatcttcccacacccagtcaccagcactgtattttctgtcaccaagacaatgatttcttgttattgaggctgttgcttttgtggatgtgtgattttaattttcaataaacttttgcatcttggtttatcttgcagtttttttgtttctgtctctctcactttttttccactaaatactagaatattctcatgtaagtgcttgaagtagcatgtgtaggtctaaggaacaagaataataatagaatcatcactttgttcctacgtacatttcttctgtcagtatttggtatttttatttaagatactctgtatcaaggtcatcaatcgtaacaaggaactaaaatgcctaaactaacttaaaaaaatactttagctctatgagttttaaagtttgtcctaggaagccagacaccatagaatcatttattgacattcctgacaaacctctagttgtatgttaattcgccaaatttactaaagtgaatttcaatcaaatccaaatgttttaaataacatggcttctgtggcatagacattagcaaatatttggccagaaatcaacagtggaacctattgtccaatttttttttttacttatgttttgagccctgaataattgcttttaaaagcttcataagaagttctaaccaaaaaaaaaaaattagtattttagataaatatttcagaaaaaaaaatctttatatttgagataaaagtatattagtaaaaccagatgctcctgttatatttgcaaggtgtttttcaattggtctaataactttgattcacaaagcccttaaaggattggtgagtatgtttcgttttttgatatttgcttttctttcagaagggtttttcttttctttcagccttttttaagtaagtttttaatgtgtttatagtgacttgtatataaatgcacatatttgaaatgatggaaaatattagaaaattacagttttcccatctagagaataaatgctgcactgtctttagtagccagataacccggctagagagaaaggtcacaggtaggcagtactgggtaaaattgagagaagatatgattctttttctgatggtccatgttttcccttttttttttttttttcctgacctttctcataacaaggatgctctgctccgccctcagtatccctttctgctcccaaattctttactgcagaaatggtttgttgaaatagatttggcctaagctatcaccaaatcctactagagggttgctttttaatgagcagtggaagattttagccaaaagaaggaatctagtggaattctagacatctaggaggtagatagtgtaacttgtgtctgcgcaggaattttgatattctaccacatagatagtcactagggttggaaccagaaaacctgatttgaagtcctatatgtcctaaaaccacttctcgcttttcaattttgaattttattcttcggaaactgtgcagaagcttatattgttaaagttaagaactgggacttggctccaccaggatttttcttcaaagctggaaatacgtgaaatatgattttctacatctctgttacactgaattcttagaaaaatctagtgaggctctagagaaaacaacctgaggtatacaccttacgatttttttctgcagacagctggattcttacttttttgggttttggataggagaggagagtgtaggcaagaaaaccagaatggtacaggtcctgttattcttctgaacacattttccttctctttagtttctccagaaatttttggaatatttcacttaagaactattgtattaaatttgtaattttttttaaaaaaggttgttcttcaaggctttcaaattggctgagtttattcagccttgcttcctttttcaccgtatgtagttttcagaatgcttcttctagttcagtcattttgaaattaataatagacagactaccttgcttcccttccgtgtggctttaaaactcactttcatattaaagggaaggcattccgaatttctgacctttcctgactgttaaccccattcaactgttcaccctcatttctgggtatgttcaattaagaatccaactcacctctggccacttcctcctattaattcttacatagttatcttctatgccatgggccacatatcagagtgttaaatatttgaggctggagggaaacagtgaaagaagctaagaagctgatgtttcctgagcacctacaaatcactggaccctgtccagggctgttacatgatctcattcttaatagaaatcttctagagtaggtacaatatctaggctacaggttaagaaaatgagactcaagaaacttgtacaacatcctgcgtcactaagtttgtgttagatgttttgattgacttagaatccatgttccttctttcttcatctgtggtttctgacctaccctcctcaaattagtggcttatgatgaaaaatggtaaatgaactaataaaatagtcaaaaataataaccataagctaaagaaagcaggaggttgtaacaggaattgtaagaaggaatttactctgagcgtccagaaaaaaaaaaaagcaaaacttttaaatgtatttttatttttctaattgtggctattaatgattcaagcactgctttctattagctcttgctgtacatttgactggtttgaggagggacagatagagaggtctcctcaggttacagaggaccaactgcatcaggaatatgggctacctggaatcttaggtacttttcagtgagcaaagagaattgaagaaacatcagctggcagagaatctccttactgccttgccccaagctttcctatactctgatgtggactaatggatgagcctaagtgcagccatatgtgctgactctttgaaaaggttcccccacagggacggagtggattcagaagtgcttggtagtagctggagttacctcctttacccctcttccatcttggtctcctacatttctcttactacttcatctatttcttgtccccataacctagaaaaaacaataggtgccaggagacctagttctgccctcagtgcatccatttagcaacatgtgatcttgggcatatgttttttcttagaaccttatatttacccatatgtatatttggaatagtaatttctgtccatctctgccctgctataccatgacctgctccacaaggatggtgtgacaatcattcagaaaatgtacaaagaagagttcggtaacctgaaatgttttatagttgggaggatttattgtatcaacaccagtatatatatacaatgtggatctataattgtgagtatttgataagggctaaagagaaaagtcaatgccctcctaccttaaaggcagtgattgtaaaatacacctgaattgttaatttacattaattgacatgataatttatacacattaaaatacagcatattgtttgcattcatctagctgtggctctccattgacatggaatggagccttccttattttcatgtaattatggagagaatgcagagtggcagcttcgaagaccatccctacgattgcagcacctaagtgctagggccatttttaaaatgatttttttcaaatattatgccatataggcttggaacctgggtccatttacagagcctccaaaatttgaattatacatatatatgcagataaaactcaaagaaaaaatttgtctcccctctaatgtaaatatgcctgtgacagggaaaaggtaaacaggaacaacataattcgctttgtttgagaattgttaaaggggagttttcccataacagactagacacttgcttaaagtccagtcttcattctgtggtcacagtaattcagtgtagatgctgcatagtctgtacatttcttcctcatcttctctttttcagttctcatcatagccgtataaggtaggatggattattatatccattgcctccactactctatgagtgtctcaaagatggggaccatgccttaagttttattaacttgaacatctgactcatgcagcaagtgtaaatgtatgtagtttatatagttttcttgtgagaaaacaaaggccaggatgttaagtagctcgcatgggcatctatagcatcctaacagatgacggaggaaggcttcatctctgactttgtgactctcattgtagtacacatcactagtttttatattaagataaaacatcaggaatgacagttgagttgggaatattatctgttagatataatatgacaaatgagttctggtaactttccaaattgaagatcatgctgaatggaaaactattgggtttttctgttgctttgtttttttcatttctcccaggagacttttggaaagtcaatagaatagttatttgggcttcagcgttctcctgtgtagaaatgcttttgacattccagtaggttctctcatacatcatgaacatctctattgaaaatgcatgtttccaagtaggggcctctgcatgacagcaagatgagctgccaaatgcccagtttgattgggtttgtagtcattttctgcatatcccaaatggaatatacagaaagctacaaatggaatgccttagctgcagtttgagggtgggatatccatgtttttcgtcagcctttaagtctttgctcaattcttttccctaactgcttttcaaactcatttgagttgaagggaaaagaggagaagggaatggagaaaacaactgaagggaaaagaggaggcaagaattttctctttctcaaaggaaatactaaaccttgcactctgcacattcaggaaaaatatgatttgatgatagcctcgcagtcagtttctccctattcaggaaatattctcagatggaaacaaaattaacttcttaccattaatcctagaagaatgttttcaaatgtttggtttgaggccatgttttaaacagagacctgttgaaaatcaatagtccccactcttcagtgtcccttgctcaccattatttccatgtttgtcttcctaggacctcaatacagtctccatcttcatatggtaacagctccccacctctgaacaaaatgaacagcatgaacaagctgccttctgtgagccagcttatcaaccctcagcagcgcaacgccctcactcctacaaccattcctgatggcatgggagccaacagtaagagcatctccttttagctgtggctgaaggatgaacaggctagcttaggacaagactctgtgatggggaaggcatgttcttaagctaaatgagagacacagtgggacagatcagatcaaagtggagtggcttgggtttctgactgaacctttcacttttgaggtttatcattcattgcaaaatatactactggtagagatagggacggatgtttctttagtgtagggataaggatttctgtgaaaagaacagtctacttgctgctatcctggatggagactcaccacctgcagcctttgaaatagtcaaaaggaaacaaatgcaacatgcataaagtagcatgttgcagtgaacttaaaatctagagaaataggccgggcatggtggctcacgactgcaatcctagcactttgggaggccgaggcgggaggatcacttgaggtcaggagttcgagaccaacctggccaacatggtgaaaccccgtctctattaaaaatacaaaagttagctgagcctggtggtgcatgcctgtaatcccagctactcaggaggctgaggcatgaaaatcacttgaacccgggaggcagaggttgcagtaaggtgagatcacactgctgcactccagcctgggtgacagagtgagactcagtctcaaaaaaaaaaaaaaaaatctagaaaagtagattatcgtctggactctatgaaaaaaatacctctaattacaaagaagttacttctcgaatacttaattttctatccataaaattgtcactatatatacttaacattttatgtctgttactctataatcctggccttttaaactcacctattctatcccagacacagtcagtaaggaactgatctacgaaggaacctgccttttatttcttctcacacacattcctcatatcagctaagtacaaatgaaaaaggcactgtaagaattgggcctccaagtggatgatgactggcatatagctttactataaatataattatcttctccactaggactaaatctagcaaagtggatacacaaacttgaaggaaaaaaaagtcgtggtaaaaatgcaaaagattgtataacaccctcggggatacagaagactgaacattgcatgggtatcatataatccttactacctaaaaaacagtaccctcaagcttgtcttttgaaatcctaattattttgatcaaacctctaaacgagcatttcagtgttgtctcttctgaaagaaatcaggggaggctaggattatgactttctgtggtctaatggcacacattcattgttttggttttttttttttttttttagacagagtttcgctcttgttgcccaggctggagtgcaatggcgcaatctcggctcactgtaatctctgcctcccaggttcaagtgattctcctgcctcagcctcccgaatagctgggattacaggcgtgcaccaccacgcccggctaattttttgtatttttagtagaggtgggttttcaccatgttggccagactggtcgcgaactcctgacctcaggtgatccacccacctcagcctcccaaagtgctgggattacaggcgtgagccaccgcgcctggccacacattcatattttaatgagatagaggtgtgtgtgtttgcaatggttgcctgttatggcaaacgtagactttagagtgagatcactctctcactaatgataaactcatcataaggcttaaaacctgaccccttgacaggcctactttgacataaaaagagtagaaaccatttattttcaactttattttatttcttgtgacatcttagagtcctcaagttccttcttggtatgacaaaattgtgataatagtgatgatggttcacatttgtataacttacagtttgcctactttttttcttatttgtactttacaattatttcagaagggaaacagattgagatgtgatagggtatgctctttagttagctagttagtggcaaagaccttttctaggcatactgctactcactaaagtagaaacaaattacattaatcttttagtaaattattttgttctttttgacatgaaggcaaaattcattttataaatattgatattaaactttttttctctatcagtgagtgtttacacttaattgtttagtaaaatatcagtaagtattggtggtgttcctgtatacaaccctgaaaatggaccataagtaatagaatatttaattgtatcatcatttattaggcacgtgagtccttctaaatattgaagtcagtctccctattcccaaataaatgttccaagggtcactctttcattcacagatttgtgcatttatccacttaccctaagtttactgagcacctactctgtgccaggcactgtgctagggactgtagggatctagacacctataagatctaatctctgtgcttgcacaggttagtgagggagacagaaatatcaccctcgaaggcaaatacaaggaagaatatgttaacattaagagcaattagcaattagagaattagaagcagagaaaggggaggaaagagattgttctcggacaaggtacaaaagtcaagaggtgcctaattagtttggagtctgaccgttctgtttgactagatgtaggctgtttgaaggggtgtagtgagagcgaagattaaaaaggaaggctggtagtttaggcccttgataaaatttaaccagacaagatggaccactgggatgctggtacatgatgatggcagtaaccctttttgttcctcctgcttctgttcagttcccatgatgggcacccacatgccaatggctggagacatgaatggactcagccccacccaggcactccctcccccactctccatgccatccacctcccactgcacacccccacctccgtatcccacagattgcagcattgtcaggtgagtccacagcatgtgcccctgggggcctgccctaagcatcccgggatggtggagggcggatactgttatagtccataaaacagttggaagggaagacagcagtcctgtggttggagttcagtcactattatctctgatctgtggagtggtcaacaatgttttatcaaggaaaactactataaccagcatggaaacaagggaaagtgtctgttgataggcatattgttttcatgtctgtttccctcgccttcacttactcccttcctcctctctcacatttttacattgtcaattgcggccctcaagttggtgctcagggctgggtttccaaaagggcaataaacttgctatctcgctccttttcctctctctctaatccttatagctctcagccaccatgggaccattattttggagttggatgtccataaaaagttttaatgttatccaaaatgacagggactttcaaaaactgatggcctttactactctgcctatgggaaatgggcacactccagaaaaggggagttgagataagcagaagaaagaaatgcatgaagcctgaaagatttgcttcccacagaggcaagagtaggttcccagatttcttgcctagtgtatatcttcttcttctgcttcatgtgcagaatgattgtttcatttggctactggaacgatttgtgttgattttcctgctacgtcaatcacagggatggcaaagtgcgtggccttttccacccagggtctgttctgtaatgatggtggcatcatcatcagtctcagaccagcaccaaaaggaaggggatctgtatcctgagaaggacataaatcaaaaggcatgaatgacttaatggagtagaggcctacaatttttttcttaaccacatgagccattctcacaaagcccagatacaaaacaaataaagtagagatgttctttttgaagtgtagagccaagcagcaagaaagctaggaacctgaacactggacttccctttcaccctttgcaatggttcctaggacacttacacagaaccctagaattccacagatcaccatatgataatcgctgatctgcaggaatccttatagttgattttaatttctgtagacctaaacttcatgtcaccagtaatctccagacctcagacttaaggcccacatatatattacccaatcctcatctctgatgtggggcatccaagggcaaaatatattgggttttcccttatctcgccaatgcagttggggtgaactttctttttctgtttcctccttcctcttccctcctccctctgcagtttcttagcgaggttgggctgttcatcatgtctggactatttcacgacccaggggctgaccaccatctatcagattgagcattactccatggatgtaagtaactgttagacttttctcaaattttatttcttcatttctttcctctgatgacaaccgccttgtagttcaatccctgatagtttaaaaatttgtttttgtcatgcccccaattatccattttgatagaacccataactatgttagaaagattcccaggcctcaacctatacatattagaagcttagcaatcaatcttccttagcttgtcttatcagtcaacaattgtctattgcacttacactattctcaaggtgggaaagaggagctattcccgcaatcaaaacattcataatttctaaagaattaatacaactttttgagtgtgctcattaatctgtatcagttcccagaaaggatagcagggcaccggtggtgctattctcttaaaaacatggagagatgacacagtagtcatttgccatgactatttaaaacaaaactaggaatagaacacaaacgtgtaagcttacagtctttaactttattaactggccttttctgcctttctttaaattagtaccttaagggagattcagccgttcctctcaattaaaactatatgttcctttattaattttcagtttcatcctaatagagcccagccatttctaatcattgggcattatcaatggctatggggtcaaatcccaatcaccctttttagtagcaatgagaacttggggttatcactttgcctctctgggcctcagttaataaaatattgggttcaaattactcctttctggctttcatatcctttgatttgtctttgaaacaagctgggcgagaggggactttttcttgcttcctactccactttttgaactcctgctcacacgggagctagtgttgctctggtttatggcaatctgatttgtctcttaggccgcgctcttcatgctgctctctttggggttcactagcctctcttctagcttccagagggtactttggactgcgggaagaagacagtctctgcctctgctgctacttctattgttggggactcttttttccccctccttgagcaccgatgctagagatgcccctgcagcataagaatgcccccaccatgaggtgacgtgtacatagtgtactgcccctatgcccacccctcttcaccccataaatcactcctggctgctgctccctgatagaataagttataagcaaaaactgcagggaaagctttaagagtaagttccagtattattctgataactttacagtcacttccgtccactgaagagaacagagttgccctatatttgagagagtctcctgtagtagattcctacattttggaacagagaatcttcctcttccctatatttttaaagttatactttccacttgctcacttatttccactggggaatgtgtcaggatgttcccttgatcattaggatctgcagataccctttataagttcatttctgctgatgttgactaagtcctatgccaatctgtgggccacagctttctcacctgtcagtcattagttaaaatccatccagggcatcatgacaatgaacagagtctaatttacaaagagctttgcatttactgagagcctcaaaagtataaagaatccaagaaacataaaaagctatgcttatttaggccgggcatggtggctcacacctgtaatcccagcactttgggaggccgaggcaggcggatcacgaggtcaggagaccgagaccatcctggctaacacggtgaaaccctgtctctactaaaaatacaaaaaattagccaggtgtggtggtgcacctctgtagtcccagctactcaggaggccgaggcaggaaaatggcgtgaacttgggaggcggagtttgcagtgagccgagatcacgccactgcactccagcctgggtgaaagtgcaagactccgtctcaaaaaataataaaaaaagctatgcttatttttttgtggattaccttctttataaggtattcttctgctacagtaagagattaggaatacctctaatggcctgaaaattcacaccataggaaaatatgacaccagactacaaacttccaaattgagaaatgttgggaagctgtgcaattttctcctcctcacctcagcttcttcctgcgtatttctagaaagagacacccttttctcagcttttatcttagagccagtctaagttattgggcatcatcacttcctggaaaaggaatgtgacctaaatttggggaacttcattagtatccccataacatgcaaaaatgaggtaacaatacaacataataataatacaaatggttaatatttatagagtgtgttgtaatttataaaatactttcatagctgtatttacctatgagtctatagcatcttttgccctataaagcaattgtatctcattttaccaaagaagaaactgaggctggtagagaataagtgacttgctgaagttctatagccagtcagtgaccatcaaaacctctgccacagttttctcactccaagcacaatgtgctgtttacctaccattctgccctgtgaaaatatgagctatcactgaaacaatctaaagaagagctaacgcagataaatgtaagtgatatcaccacagtcagttaaggttgaccctatttgcaatgccttctgcacccattcacagaacatagacacagttgctctgcattttgataccttgctttattcaaacaccaagagaccttttagataaaagtgccgtttcaggttctgaggatgccctaagtccctaattttttcacttagttatgtgactttaaacttgtcaacctctctggccttcagttgttctatctataaaacaggtctaataatgtttaccactaatctggggattaaatgatatcatgaatataaaataattcagggcctggcacatagaaagtactcaatcttttcttctcatctcctcaaccaagtggtggtgacattccattaatacctttcttctagtgactagccaggtaaattcaagcataagtaggtacctcatgtttctatttgggatttttgccctctcatctagctattatcccaattttaccaatgaagaaactgaggccagtagagactaagtgaagtgttctacacaggcaggaaagacacctcaaggctgtgcctttgtgcttagttccatagagttgaagactcagagaactaattttattttctaatttgtggatcaatagattcagatcaattaaaccagagcatcagggaatgataggatgctgtggactaaatgtccgtttttctccctgttttcattctccatgacaccttcccctgttgcacaggatctggcaagtctgaaaatccctgagcaatttcgacatgcgatctggaagggcatcctggaccaccggcagctccacgaattctcctccccttctcatctcctgcggaccccaagcagtgcctctacagtcagtgtgggctccagtgagacccggggtgagcgtgttattgatgctgtgcgattcaccctccgccagaccatctctttcccaccccgagatgagtggaatgacttcaactttgacatggatgctcgccgcaataagcaacagcgcatcaaagaggagggggagtgagcctcaccatgtgagctcttcctatccctctcctaactgccagccccctaaaagcactcctgcttaatcttcaaagccttctccctagctcctccccttcctcttgtctgatttcttaggggaaggagaagtaagaggctacctcttacctaacatctgacctggcatctaattctgattctggctttaagccttcaaaactatagcttgcagaactgtagctgccatggctaggtagaagtgagcaaaaaagagttgggtgtctccttaagctgcagagatttctcattgacttttataaagcatgttcacccttatagtctaagactatatatataaatgtataaatatacagtatagatttttgggtggggggcattgagtattgtttaaaatgtaatttaaatgaaagaaaattgagttgcacttattgaccattttttaatttacttgttttggatggcttgtctatactccttcccttaaggggtatcatgtatggtgataggtatctagagcttaatgctacatgtgagtgacgatgatgtacagattctttcagttctttggattctaaatacatgccacatcaaacctttgagtagatccatttccattgcttattatgtaggtaagactgtagatatgtattcttttctcagtgttggtatattttatattactgacatttcttctagtgatgatggttcacgttggggtgatttaatccagttataagaagaagttcatgtccaaacgtcctctttagtttttggttgggaatgaggaaaattcttaaaaggcccatagcagccagttcaaaaacacccgacgtcatgtatttgagcatatcagtaacccccttaaatttaataccagataccttatcttacaatattgattgggaaaacatttgctgccattacagaggtattaaaactaaatttcactactagattgactaactcaaatacacatttgctactgttgtaagaattctgattgatttgattgggatgaatgccatctatctagttctaacagtgaagttttactgtctattaatattcagggtaaataggaatcattcagaaatgttgagtctgtactaaacagtaagatatctcaatgaaccataaattcaactttgtaaaaatcttttgaagcatagataatattgtttggtaaatgtttcttttgtttggtaaatgtttcttttaaagaccctcctattctataaaactctgcatgtagaggcttgtttacctttctctctctaaggtttacaataggagtggtgatttgaaaaatataaaattatgagattggttttcctgtggcataaattgcatcactgtatcattttcttttttaaccggtaagagtttcagtttgttggaaagtaactgtgagaacccagtttcccgtccatctcccttagggactacccatagacatgaaaggtccccacagagcaagagataagtctttcatggctgctgttgcttaaaccacttaaacgaagagttcccttgaaactttgggaaaacatgttaatgacaatattccagatctttcagaaatataacacatttttttgcatgcatgcaaatgagctctgaaatcttcccatgcattctggtcaagggctgtcattgcacataagcttccattttaattttaaagtgcaaaagggccagcgtggctctaaaaggtaatgtgtggattgcctctgaaaagtgtgtatatattttgtgtgaaattgcatactttgtattttgattattttttttttcttcttgggatagtgggatttccagaaccacacttgaaacctttttttatcgtttttgtattttcatgaaaataccatttagtaagaataccacatcaaataagaaataatgctacaattttaagaggggagggaagggaaagtttttttttattatttttttaaaattttgtatgttaaagagaatgagtccttgatttcaaagttttgttgtacttaaatggtaataagcactgtaaacttctgcaacaagcatgcagctttgcaaacccattaaggggaagaatgaaagctgttccttggtcctagtaagaagacaaactgcttcccttactttgctgagggtttgaataaacctaggacttccgagctatgtcagtactattcaggtaacactagggccttggaaattcctgtactgtgtctcatggatttggcactagccaaagcgaggcacccttactggcttacctcctcatggcagcctactctccttgagtgtatgagtagccagggtaaggggtaaaaggatagtaagcatagaaaccactagaaagtgggcttaatggagttcttgtggcctcagctcaatgcagttagctgaagaattgaaaagtttttgtttggagacgtttataaacagaaatggaaagcagagttttcattaaatccttttaccttttttttttcttggtaatcccctaaaataacagtatgtgggatattgaatgttaaagggatatttttttctattatttttataattgtacaaaattaagcaaatgttaaaagttttatatgctttattaatgttttcaaaaggtattatacatgtgatacattttttaagcttcagttgcttgtcttctggtactttctgttatgggcttttggggagccagaagccaatctacaatctctttttgtttgccaggacatgcaataaaatttaaaaaataaataaaaactaattaagaaattgtgtgtgtgtctatctgtgtctgtttgcctatttgtccattcttccatcctttggccttgttgatattatcgaggaagaagagataccgagcaaaacactggagagtttcagatgttgacgtttctcatctgagaacaaataatcattcttttctcattttagccaacctacaaaataggaaagaccagaatggcattttcatttggtgaatgagacattcaaagttttaataccttatttgattgaggtcagaaagagtttgctttagaacagggactagacttcaaatattcctaatgtctaacttagtgaccttccgatgatactcagcagtgacctcatcagtactgtgggagtggaaagcaaataagctggtggtatgtgggagcacgctgcagatgatgggccctgactgggccatgttcccttttaaattcattgttaaatatcacccttcagttatgtacattgatttagccaatactatctctatcaaaatgggtgaattatttttattaaataagaatgtatattactttatgggataacattggatggcatgaatacaatataatgccttagacttataaaataaacttttcagaataacaaaaatatatactagagatatcaatttgtttttctatttatagatataatcacattataaagatatgttcatataggcaagcaggtgcagccaaaatattcaattcttagtccttgcagtctaggtgatagagtgccagaaatgaaacagttaattcaattatgctagcatgaggaatataaaagagctataaaaatgattaataattgatctgtcatttgggctctttcaattttaatttttattttttcagtagttctatttgtatgaattggtagttcatgacttttatttactcttagctgtcttctggagtaaaagaaaaacctacttttaaaaaaatgcatttgcttagaattattgggtctgttaaacccttttacaaatacaaaatgcactatggaaactcggcgattttcttctttttcatgtcttgagactttattttagccgttctagaaattgctttatttttatccatcaattctaatgagtaaaaaaagaataaaaaccaagaagaatggtgggattgcccaaaaccgtgctgtccaatatggtagtcactagctacatgtaattattgagcacatggatggagctagtcaaaactgagatgtgctgtatatgcaaaatacacattagacttcagggactcaaaaatataaaataactccttgataattgtttaaattagatgttgaaaataaatattttgggttaaatatactggaattaattttacccgttcctttatatgcttactacaaaatttaaaattatgtaaatggatttcattgtagttctattgggcaattagctagaaagatgatgtaataagaagcctgttaggatgatgaaatgaagttactatttactattgtgtaattatccaggttttttggttttttactgggttgctcattagtattgcatcagttttcaacaaagtatagaataggagacaatatctttgtagttaggatttttcattttctccccataatggtaaagatattaaaactgacagagaaaagcatttcaaaattgttacatcagaatatgaatacaaaacacaatggcactctagactctggtgatggtgaaatggcttgtgtaagtttgagttttcatgtaatgatatcctagatgaattttctcaagcacaaggctcagtggccaaggaataaagttttaaagataaaaagggtatatgcttactttaatctagatagttcttcaataggaagaattttaccatttaaggttacaacaataatctgaccatccgattttctaaaaggacatgcgatagtattattttagttctaatgattcttgtgcaccaaaatttaccaatatacttcacaagtggacaaataaaggctggtgagtatcaaaggtaatcggaagaaaaatgaggtagaaacttaactcattggattaatcattctaattggtgtttaaaaagtattttgcggttttggagtgaagaacatagtcatcctctcttcaacaaaggcatgagccatcaaggattcaaaaaaaatagttcaagtgttttatgttgatgatgtaaatacaagaaaagcaatcagaagtaatgatgttagaacttcttatcagaggtgcatttgaaatcaatacttacaaggtagctatgtcccaaatttcatggtagttcatgagcagttaattgcattcagaaggtattttctatttcatgtacatgtgccactgaaatcattaacaaaattttagatttactatgtttatattaaaatttcttataaaattattcttcaccatatattctgttatttataaaattactaaaataaaaatataaagcattaattagacccagaagatacggataatgactactgcactgagaattattagaataatttatctaatattgttatagaatgccaaaaacctaatgttcattaaagactcttgaattatctaaaatctgcatagaatctctgagattccaagaacatatttgtgcttttgggtgcagtttctgaattatctaagaaggtaacccaaggactttccttagtatactgatttattttgtattcctagtagcaaaataaaagtatggaataaaggcctgcacataggtgggtagacattggaaccttcctagatccaggaaagataactttaatcttgagtcagggcatccacagactggcagaaggcagtggttattacccgttttgccattctttctctttcatatactacagttgtcttactaggtaaactggaacataggacactttcagccttgagtatggatttcctagcttcctctttgtacccataaaaactttggtgtccattatcattcttccaaatttgatttacactatgccttttcccctggtgcttcagactcattccaaaaaaagttatgcagatgtgtagatttcacggaaatatgaagatccagagtgaaaggaacacttgactgagaggagtctggtgacttgggccagatgcttaatcttctgcatttcagtttcaacatctgccagatgagaggtctgaacccctaacttgtcagtatttcagtaatagcttttggaagaaagccgaaggatggcatgaaacaactcttccaatatagaaatggggttagcgttttcagaaaaaattcagcagtctcaaagctctaatcttcgttaaatgcagacctaaaagtgtcgggaatcttgttccttctttagaaaggtttttcatagagcactaaccatatatatttttctcagataagagtagttcactaatttaactttgtgggtttacagtcatgttgggtgtgtccttgaaatttatgagatttgtaactccttacactaatcctgactctgtctatgtgccaggtgggtggcaagtactggcaggtgaacttcccttttgtgattctgattagaattctaggcctagcacactggaggaccaacgttttgcaattcaagtaaaagtctgacacaggacgaggaacaaaaattttttgcagagagagtttgacatcagaaacaccaggcagaaaataacgaatcctgtgagcgctttaactcatctgacatttgctctgtaattggaaacgctattatttttctgtgaaagaaattatggtgactttgaatatggttcaatattccgaaattctaggactaccttctataagtaagttgattactctgcagtgcagtggcacagtcatagctcactgcagcctcgaacacctgggctcaagcaatcctcctgcctctgcctctttagtagctgagactacaggtgtgtgccaccaaaaccgctaatttattttagttttcgtagagatggggtctcgctctgtggcctaggctagtctggaactcctggcctcaagcaatcctcctgcctctgcctcccaaaatgctgggattacaggtatgagctactgcgtccagcctgctcttctgtattttagctaagcttgcatcagcggtcttcaatatttctccaacttatcaataccttcctaatacgattttgtctcactaatgattaaatgttagagaaagcacttgaaagcactcgcaatccctaaagtacagaaatggtggaattttaaaagagaatgaaaggagaaagagagaacaaaaagatcaacccattgtcagattctggactgtgaaatgaatagagaaaattctgctgtgtcatttcatggggcctcttgccaaatgcgttttggaaatgtttagaaagttcgttctaaagaattctggaattacgtggcatccaggtaccagcacatttggtatctcagaacaaatgctgacatccgtaaatatccaatggcagtcatgaaataatattctaagaaatagaacacttttgtgaaccttgcccacgcagaagaagccctaggaaatatttgcctaacaacacctggtgttcaacatggctttcaggacactgagaagtaaatatatatatatatatattttttaaaaatatatatatataaatatatatatgcatatatacaatttttgtttttactgcttggggataggaacatctcaatatcccttttccttatcttttgctttctcggggtcccactgtcaccctaagtaattggcttataagcagcacaaactctcagcaaaatgatacgaaagttgtctttggaatattccattcccatgcctgtctttcttttccctttctgctgaagacattcacattttcttttttactttctctactcaaacctgatgttcctagctaggtggtaatttccttgatggtgcatgagataccacggttatatgcattttgccaggagtttctaaccatgccttggaggtggttacctgccacttattcttaccaatccccactttaaggcccacgattctttcaagagtcacatgtaagacaggaagccatggctaagcctagtctcaccaaaggcccagcagaaggcgtaatactgtccagtggctggccagtgttcagttagcatttggcaatcaatagagttctgtcttctcagatacagcccttcttctcccctggtataatatcctattcataagaacatagtttggctggctctctcctccatctttcatctccttagcctcattatttcttgtcctggggtacaataccacagtctttgctcatcatcacagcctctgtaacactttgacctcccttggtagagtatcaggagaagtgaatgtctgcctctttcccatgattgtaatgcattctttccttccctgaggaaaagtgcttccctgtctcttggctgaaagactaattttttttttttttaaatgaactccactccaattatttccatgatattcttgtgtaataccagtctttcctcctcatgttgaatgggttgggtgccttcagtgaggagactagaggtggtaaacattgggaaaatgaaagaatctgatggagcaggtggagctcagcaaaggtctgtgtggaggctacacatgggctttggtgagggatctccttgtgcttcctgtcccaggggttactcagctgcctttgacagactctcatatgtgcaaggcactgtgggaaattcaaagttgaaaaatacacagctctatgaatcacagaagtttagacctagaaggaacttaagagccatctagtttaaactcttcattttctggagtggaaaactgaggcccaggcaggaaaaggaactatacagattctcacagcaaggcaaagaagaaccaggtgatccagacttccctttctcatttgtagtacaacctaactccacctctacttagaagaagtctctcttccataccacagtgatgcccttgttattgctgttgctatttctcaatggaacttgtcaatgaatacttttgagtgctgcttaggagtgactccttctctcactctgtttgctttactctttttcatccccatgctggtttttaccacaaacacccctggcctaaagaactctggaattcacacaatgggtcttggctttcgcatctctaagaggaaagaggctggagtaaaagatctcaaagtcctctactagcttcagtgttctagaaatgtacatttcaccatgaggtaggccaccatttaaggaagaaaatgggattttcattacatactgacattacttagctaaaattaagaggccttaagaattaataacatttaggtaaaagctgtgaaaggtgctaacaggaattgctgggataaaagcaaagatttgtgcaacagctgtaggtattttccatgacaatcttccttcatagcatgactttcagctgggacaccctcttctaatctctcaggaaacagaagagatgacgtcaacgtaggggaaaagaagtggcaattttctagtttatatatttaaaaaaatacaatcaagtaaaacatagctaaagagtatctgtcaataggtctgatgatctagatggattgtataaataaatataaaatctttatattcttaatatatactgattcactgtttcctaaaacatttcatataaattatcttgtttatttcctcaacactgtggaatagacagagcaaacattgtcagtacaaagggcggaggaagctgagcctctgagaagttacaggcatgtctaagttcacaaagctattatagtcagtggtagaatcagcaagtctttctattctagtttggctctttttttccgtggtactgttgtctccaatcagaaaataagttctcagcgggtcactctcttaggctgcacacacacgcctcaaagaaaagaattctgtggactttccctggttggagacagagtatgcgttccaaaatctgtcagccctagattgcatccaactaacctaggcccctcaagccccttattgaaaacataacccttctatcactggccattgtcagcaggataaagcctatctaactttgtcactaatttgccctttaagtctaatattatctgatgctccaaccaattctatttactcaacattcccagctagaatgtaatcttcacaaggcaggtttccttgcgttgttaattgatgaagcctactgtagccttcactcaataactatttgtagaattcaacaaatcagttcttgaattttcaggtatctgtgcattttctatgctgttctcttcaacagaatgcttcgaccctactctcacccatctcagtctacctcttcttgtctttcaaaaattacctcaaacattacttccacaacaaagcttccttgattatctaccactggaaagactttgtaggacttagatgtgtttctgttttcatgatagatatgaacgaacttggcttctcagtttgatcgtcttttttttctcttttttctatttctgaatgttaaaaagcattttaattgttgtgggggcagaacataagcaatgcctcccctatcctccaggggataggaaataggaagagaaaaggaaaaaatatatctatatctatatctatatctatatctatatctatatctatatatatgcacacacacacacacaccactatataatatatacctatataaaaccctcagctatggccaatgataccacctgtctttcaaaaatgccttttgaaaatcccaggttctccttgatacaagaatgctcattctttcttcaaaccccctaaaaatgtatcttttaaggcaagctacctgttctgattggtaaatgtgtacattttgagtggtctgtgcccattctctgtgggttgttaagtagtttgaatataacctatgcctctatggatgtcaccacaatctgcttcgtgttttctgttttgtagtcatatatctcttgacagttgatcctgcacagtttctagtccaacccctaaataaagttcttaagacataatgggtgtttaatgaaggttcgatggattattattttaaatgctaataaatactttgcaaacaggaaaaacaggtagtatgttttgctttttctgaatagctttcagtggaaggaagtagaaaggtaaaaagatactagattggtatattcaaaatttattttcacatatatatatatatatatatatatatatatatatatatatatatataaggttttctttcagtcacactagatctggcatcagctgtggttcagctcctgtctagaagggacagagctttgcagttggggtaactcctaattcttagctaggaaataaccccttgggatattagagatctcaaaatgttttactactttcatttgagagtctaaagctctattaaaacctactctagcttctccttgattgtcccttgatttactagtgacatgctttctactgaagaaagaggattttgtggtcaacaaaccacaccctaagctctatgatgtgaaatggaaattttctagtatacatagaatttatacttttaaaaatatagaaaagtttaaaaaataattgatacatgtccatatacctactgcccataaattatgattaacactttttcatatttaccacaaatctatgtatctctgtgcttttcatataagtcaaacttaacaaagtaaatatcccctttatcgcctacctataggtatacacatctttctctgttacttccctgcattacctggggccagaattaatctgcaccataagtttagagtatttcctccaatccattatcattttgctttctatagtacagataagtaaatttcatagaattggaataataatatacatatatatatatatatatattttttttttttttttttttgagatggagtctcgctctgtcacccaggctggagtgcagtggcatggtcttggctcactgcaacctccgcctcccaggttcacgccattctcctgcctcagcctcccaagtagctgggactacaggcgcccactaccatgcctggctaattttttgtatttttagtagagacggggtttcactgtgttaaccaggatggtctcgatcttctgacctcacgatccgcccgcctcagcctcccaaagtgctgggattactgtcgtgagccaccgtgcccggccagaataataatcttaaacataacagttgccacgttaaggtttttttttttttcatccagtttctctgccttcatacagtcaagctaagtgatggaattgccatgttatagattgtattaggcttctccaaagaaacagaaccaatagtgtgtatatgtatatatatatatatatatatatatatatatatatatatatatatatataatgagaattggcttgtgtgatgatggaggatgaggagtcacatgatctgctgtttgcaggctggaggaccttgaaagcaggcagtgtaattcagtctgtgtccaaagactcaagaacaaagcgagaggaggagggtgttggtggattgtgtgcatccttgcagggaactgggaataccgatgtctgaaagtcaagaagatgatggaggttgccgctcaaatagggggcaaatttgtccctcctccacctttggctctcttcaggccctcaagggatgccacccacattggtgaagatgatcttctttactcagtctactgattccaatgctaatctcttccagaaacatcctcacagacacaatcagaaatgtttttctagctatctgggcccattcaagtcgacatctaaaattgactgtcacacagatgaaaaaacagaaattcaaatatttgaaacaaattacctaaggccacgcggagattaagtagaagcagcaggattgaaatgcttttttttttttttttacttcaaagttttgctctcccctttgcatcgtgctgtaccttctaaaatgattgactaaaacatatgccaatactactgagcttggaagtccataatttttagtggtaccagtctccatgaattattctttggagcaagacagatgtaagacttatagcccttgcccttcctgtgagcatggtttagagtttgggagaaggtaaggattcataaacaaacatgtataaagtgtttccaagtgagactgcagtatgaactactcgggagaatcccagcggaagaaaagatgtttttgatcagagaaatctggagaaatatcagaggagaagaagcatttggactatcttgtagaatgaatatcacgggcaaagccataacgacatggcaatgtgacggttaagtcaacagacctcttcgctaagctaggaacatgaatggggactcacagctttacatatgaccaaaagtcaggaaagacaggccactgtacctactttggggatttttgccccagaaaggtttacagaattttagaaaaactctgttttcttggtactctacaactcagctaaacatctaaaactcgctagtgtggctagccacccctcataatatttagacaattgctcaattacccctggagtaacttgcaggttttacacgaagacatgtgggagaagcaggaaggctccaatgccttggggtgaacagcaaattgtgagtttcctagttttctgttttgtggctagctgatacagggaccattcattgtccacagaaacactggatactacgagagaagaatgaagttgcagaatgctgaccacagcgaactcatagtgactgcttttctctacagatggggatgccgttactcagataatttgagttattttcctaaggtcgcaaagtcagtgattgacctccagtgacttctcatgtctcttcttcacaccctgtgcaatactcttcctggcatgccatggtaataacactcctacatgtttctttaattttcttgcacaaagcatgctccagtttagcttcagccaagcaagagcacactaggaggatcctgagagtgaaatttgttccctagaatattaggtgaagagtgcaacctgccttcactgttcaggtcctataaaatacagaggccagtttgtttttctctaaacctcttagtgtttgggaattccactggggtctctagaaagagtgcctaccttggtcatctgatgccattagcttgtgccactgcacccatacactgctgtttctagattttcaacacaaagatacagagaagatacaggggaagatgacttctgtgggcccacttagagaggtagtcccatcaaagttagcaaatacctacgttcttcaaagacattaaatagtcatgctgctccaatatagccaaaagcacactttttggagcaaagttaatcctactgaaaccaccttcaattaaaccaagctctaggggctaagaaacagaaaagtgaattaagcttcagtgactcagctcctccttcctccctgcctttgtctatcagccaggaatgtggggaggaagtgagttagggcaaatctatgcttgagcttcacaatgcaaagaaccaggatctatacacacccaaggtgattttcctacaaatgctatttctttacagtaagtctcccctaacacttggctccagtgagtatgaatcttacatttgcaagtctggaaagaccatgaatggaaagaaaggcatggatgcccatgaagagtaaattaaggtgtggccatagtactctcattggcaatcacacgaactggatgatttactaattaaggttagattgcttaccatcggacccagaaaaaaattcataatgtacaagttatttaataacaaagttctccatgaaaaaatagtgacagaaacaggacaggagagaggaagaagccacacaagcaagaatacagtgtcaagcaaggtgggagacaatagcttcagcctgaacttgacactgaactatgaaatgtcaagtttcccaaaatagtctcaacctgaggctgggaaacttgggatgtcataatcctgtacctaccgatcattggtaaagggctctcccagggacatataaactcttaggaactctcagtttcctgtgtgttccaaaaatcccaggggcagtcatcgaaaaagaaccatcacaattgctggctgttggaagtgaaacataccaaagcccagggaacatacttaagaaaaaaaaattatctagaaaaacgtgatcttagaatatctaaaccttaatccaatagcatcctatcagctacatttcccaattttttaaattaatattaacaaggggatctatatttaagagagttaccttcatttactaatttacagacatcgcactaatgctttcatatatactctatgatacagcatggtggttttacatatactatatggcattgcaagatcactggagttcaacagttccaggtttaaattttgactcatccatattttagctgagtgactttgctgaagttgcttcacctgtctatgattcagcaggtaggtcagtgcctgtgagaaaggaatgaaataagcaagtacaacctttaataacccagtcatggcacatgacgaagtctcagtaaatacttgttatgaacaatgataagaataataataatgttctttatttattcatttatacaacaaatatttatcaagtaccttctatgtgtcagagcctcttcgaggacaggaattacagagggtttagttaaagaccctggcctcacagagtttatattctagcaagggacataaacaataagcacacaaataagtatttaatgcaatgtcagttactgattagcacaataaaaaaaactgaagcaaggcaaaaaacaaaacacagaatgagtggtagagtgaaagatggaggctatgattagagatgaaattcaggatatcaatgattagatatgaagtccaggaattcctctctgggtagaacagcagacacctgaataagtggaaaggataagatatcttaggagtagagttccaggaaaagataacaatgaaagcaaagaccctgggagggaatatatttgtaatgttaaagaaataggaagaagccctgtatgactgcagttttgtgaggcaggccaagagtattaggagacaaaatcagggacagtggcagagaacagactagatcacatagagtcaagtaacccaaggtaaacagtggaaatttcatttaacctgactgatagaaaattattggaggattctgagtagagaggaacaaacctggttatgtttgtaaagagtacacttttgactatatgctgccacagggcatgggagcatctgtgcaaaccactttcgaggaacagacctggagctcacagaatcctcaagctcagtgagcaaaccaaactaatagtaaacataggttactggttcattcatgtgtcagtattgtcattcagacaataagagtaattttctctaatagttgcaaatcaagtccacataagccatctctaatataaacataaggttacaaatgcaccatccgggtattctttacctttagtctgtaactttaatttttctgccttttagtggaatagtctctctcattggaataactgttttaggatgatcttatataactgtgcctgatttgctagaagcaaaattacatactgtagggaatttatttttttgaaatatcctgtttcacagaaactccctccattcacacacctctgatggctgccttttgatttcaaattaaagctcaactctgtagctgagtattccatacttcctcttccctgaaccaggtagagcactccaacgtgccttacctatgttctaacataagagaaccacctgccctgacacaaccaggcttcactgtccctcaaacataccatgcataattttgttaccacataactgctgatttattttatttgcttaagtgttatatttctctcaaggccaagttcaagtctcatctctatgtggggcctttcctcactagaaaacctcaatgatcattctctcatctgaactacttctgcagttgttagagttagcacatcatctctgggattatcactgatcttttaaagaatacatccttataggccatctgtaaacttctaaaagatagtgattatgtcttacaaatcttcgtattctagaaactctgcatgtagcacttgactttcccatagataattcaaactcatcatggttcattcatgctgagttcacactgctttccacccccagcacacctactctttttttccttttgtaacgtatatgaacctcactccacaaacgtaaatgtgcaagaaatttacctttctttatttttattaaatattgacttaatttctatttctttttttttttttgcaccaatgtggtaaaaaataaaaagcttctaggtcagttcaggctttcttcctttttaagttctggtagggcgcctcagatccactcagccaactgtccactcagccatctgtccactcagacatacactaaggattccataatcccatcagctccttagtcattaatccactagtgtcatttctggaaggcttctttctctcatcgccatggccattcaaggcctgcagctctatgtcaggcttagaagcatgggaatctctctggtgctgttttgggctcttcctgcctaggcttacatgcaatatggacatattaaagtcttgctactttcttaaggctctgttcttgaaaaaagaagcaagactcttctctccagtatctaccaatacctatcttctcatctctgcatcataactctgtctttccaagtgaggaaagcttttatggtctggaggaagccaactctcatctttcaaatatattttttctgcctaaattatttaagctaaccttaaatatttataaccaaactccagaaaaacactagcaagtaattttgtaactgtactttgaaaatttctcttttttaaagcaataattcctgatcaaccactactttgattaagcagtatgggaacatatagataaattactgggaaaaaatgattatgggtttaaaatgtcatccagcaacatttactttagtaattagcataaatgtatacttctcatctacctaatccattaacttggtagtcaacttagacttctttctcttgcccatctccattggtcttgttgtgcataaaccaacaccaaagagaaccctttggtgtaaaattaggtttaatggtaacattcatgcctagacaattctgcttgagaaaagcagaatgagtgaaataattcaaaagttcaaagtaggtagggttaaaccataagggaaaagaaataatatgtgctagaagttcagttgaaaaagaaataaattcttttgtcttgtcttccttaagtttcacctccctgcctacatgttctaggtattcagtagtcatttgttgttaatcatgataatgacaatggtgtccatttgcagctcaacttgtaagtttatttttccttttaaagcagcaatatggtgcttttttaataatagagagcatattatctttctgaaagacacttatagtcattcaggttgagaagtgcacacaggcacaacattccaaagttataacttttactgtagctatcatagccttgtatatttaggacaacaactgaagtaaagggtcttgagaaaaggacagcattttataattaacacatagctaccatatgagctagggactattctttcccctaagtatcaaagagctctctaatttggtcctcaaattttagttttaagaaccacctgagctctttatttcattcatacattctttaaaaatatatcacggtcattatttatttatttcattttttttttaatttttgtttttgacagggtcttgttctctgtggcccaggctggaatgcaatggcatgatcatggcttactgtaacctcaaactcttaagctcaagcaatcctcccgccttagcctactgagtagctgagattacaggtgtgtgccaccacactaggctctaaggctcattatttagttagaaataaaaaaggcatgacatcaaggacctgtaggcctcttctttccttaaacaaccatctactaatatatgcaaagcatggtattagttatcctcctgggaagaaccaaaataagattacagaagtatatcagacctagtcctcatcttctgaaagttgacattctaacttatgaaataacacaaaagcaaatggttacagtacaaagtgatgagagctgtaactgctgcccgtccaaaatgctgaggaaataccaagaaggaagcaactaactctaccttagggattaggagagagcctcagtgaggaagtcaggattgggacttcccaggcaaagaagagagggaatgcttcccagtaaaaggaaatagcacatgcctgtttcagggaagtaaataattctgctagaaaatattaactgagtctgtaacttggggtacagaagtgtggagaagacagataaagcaaatctggtaagactggctggagtctggtaagattatggaaacttgtttgtcaggacttagaacttgattttaaatgaatatgaaggagagaaatgcaactgtgtaaagaagaaacttcagatggtgttgggaagatctgaggcactctaccagctgagtattaaaactgtctatttggcagcactgtagaggtgggttggaggaaagcaaaggacaagtaaagggctagtccaggtaggactaggacaaacctaacttcaggaatctcttagccatctctgaccttgcaggacatcatggaaaagctctaatcatcttgacaagagtctaattctaagactcctgttgtctccacagcaccaagcagcttgtggtaacaagtttctgtatgccacccacttctgaggtcaaaaaagaatgaaggtgtttatttacagtcccttcattattgcctctgatagaattgtgcttctttctttcagagcatttttttaaatttggaattattctttcataagtatgattatttgataaatgtctgatgaatctcactagattataatcttcaaaatattaaaagtggtgcctgtattttgctcatcactgtctctggaggctagcagagaatctagcatatagtagacaatcaataaatgtttgctgaagaaacattgactaaaatattgagaaagttgatgtaatagcatgctacttagaaaaatgtttagtggttttttttgaaatgataaaatattcaattcgtgatttactgtttactagataatttttttgctgtttatagccaaattttctcttcaatattgcatgtgtacactaactttgtacctattaaatttcaggtctgaaatccacttaaattgggaagtaatttgaccaaattatttaattctctggactttatatcccaagataaagtaatatcttctgtaactaaaggggacttttttaacctaaggcaaacaatttttcaatcatgactttacatttgatgagattccattgagaaactctatgcatgtcctacttctaacaggcacacacaaaaagagacagtcaaagcacttcattaaaggcagaaagattaaggcaaaagattacacaacaaaaccacccacattcagttttgcaagtctaatagtcatcctgaatcatcaccctagagcggcctgtattccgttccaactcgtggcttcagcttaccattcaagatgcaagaaagtaaaatattaatattggtgaccaaaaaagcaggtgtgtttctctttccttagatactcttagccctggaggcagcaattgtttcttgatttttagttagtaaatatacatctcttgggaggtaattaaagaacaagcacatattttaaaatcaccaaagaataatacttgatcgtatttctggcaaccgaatcactgacttaagaatctcagaaactagtttttaaataagctttacccagccctggctcagccccataacaatatcctccagttattcatcctttcctttatcaggctcttctctcaaactaccctatttttctcttcttgtacatttgttcatgttgtgttcttaacctaaagcaatgtccttctcctggcaaagtgtgtgctccaatgctagcctaccatgttctccctcatcattctaggaaaactgtattattcctttgtctgcttccctacagcatgtggtttatgcacctgttacacagttttcttcctctctcctattccagttatttgactgcaagacctgttaagtcaggaaatatggaattcatccttataggcctacttagagactaatttgatgccccaaataatgtaggcaagtgataataaataatgagtgaatgaacttcataaaatactagagaatctttataataatagatacaggtacagtaagacaattaactaaaataaaaaagtttgttataaaactaagaaacaaggtcaaggtcaatataattgttgtacttaaatgttaaacgtaacatgaaacttccaggaaaccaatgaaaaaaatatataactaatggatcacatatttctcatgacttaatgaggagtaatgggaaactaaaaatagaacagttgttacaaggattgaaatttatcctcacattagcacaattccttatagaattgaaatgttataccctattctaactttgagcttaaaaaggaatacgaagatgttacaatcttttttacacaatgtcaagcattcaatccaaaatgattagctatttccgaataagacaaaatgaccaaaaggggaaaaaaaataacactagaaatcactgaacaggtgaaatgtatacagaaaaaaaaatttttttcagaactgactaaagaaattaattcacagatttaaaaagctctgtgtgtcttcaaaggaaatgatgaaatacagaatataacaaaatgctaatgctaaactattgaaaaacaaaattcacaaacaaaacctatatgccagttgaaaacatttttttaaatgaacataaaataattttcagaaaaataatgagaaaatttagtggcagcagacatgtactaaaagatatgctaaagaatattctttgggcagaaagaaagttatcctagacagaaatgcaagaagaaaatcaatccaggcaaaaatttaagtagaaataaaatcactggaaagaataaacatataaggaaacagaaatgaatactgatagtataaaatgacaatggtaatacaacatggggtttaaaatgcaggcataattaaaatacttgacaataatacaaaaaagcctgagtggggtaaatgaaattgaagagtcctcaagtgctcacatttcctaagaaatgaacaaaatattaattaagagtacatgctaataatttaaggctttctgttgtaatttttagtgtaatcactaaaataagagaaaaataatttctagctaataagctaatagatggcaaaatggaatgatagttgaaaaaataatcaattaaaaagaaggcaacaaaagagaaaaaaagaacagttgggataaacaaaaaaaatagcaatacggtagatttaaattaaaaaaaatcagtgataaattgcctgtaaatagactacaagtttcagatttgataaaaataaaaatgcacattcaatttatgagtcatttcttaaacaaatatgtagagaggttaaaaataaaagaatgggaaaaagaatatactatgcaaatattaaccaaaattcacttgggaaactaataaagtaggctttaaagtgaaaagcattattataaataataagaaaaattgtaacaattacaaatctgtcttcacctaataacatcgtctcaatatataaacatgtataatgtggcacaactgcagtcataatgagagatttacaactttcagtaactcataggacaagcagacaaaagtcagaacaaatatagtaggtttgaactaaataattaacaaatattgattcaaagtttaaaaaaatagagtgtgttcaacagaaaaatatactacatggtatttttataaaaataatggccgggcatggtggctcacacctctaatctcagaactttgggaggtcaaggcgggtggatcacttgaggtcaggagtttgagaccagcctggccaacagagcaaaaccccacctctactaaaaatacaaaaattagccaggcatggtggccggtgcctgtagtcccagctacttgggagcaattcaagcatgagaattgcttgaacctgggagactgcatgagctgagattacgccatggcactccagcctgggcaaaagagcaagactctgtctcaaataaataaataaataaataaaataaaataaaataaaatttgaatgtttggatgttaactataaactcctaaaataattgatgaaccaatgaataaattgcagtgtaaattgcaaaatattttggatgacttctaaagaaaaatatggaggtcaagctcatgcttgtaatcccagcactttgggaggtacaggtaggaggatcacttgagcccaggagtttgagaccagtctgggcaacataaagagtccctgtctctccaaaaaagttttaagaaataagccagacatggtggtgtgcagctgcattcccagctacacaagaggctgaggcaggaggatcacttgagtccaggagttcaaggctgtgattgtgccactgcactcctgcctagctgacagagcaagaccctgtctcacacacacacacacacacacacacacacacacacgcatactgaagctttacatacacagctaaatttgacaaaaaacaaacgaacaaacaaatactgatttgcaatcatctaccagaaaagaaaggctgtatttcatgatttaatgttttatcttaaaacattatgaaaataacatcaaagtaaacataaagaactcagaagaaaagaaataagagtagaaatgaatgaattaaaaccaaaagtacaacagagaatattaaaaaggcaagaattggtttctgaaaaataattagtgaagtggataagacttttttaaaagattgatcaagaaaagaaaaaagtacaaattaccatattaggaagtaaaagcgggcattatatgtattacaaaatataaattacagaatatttttgtaaattacaaaatgtattatttaaaatattataactttcatatattctttcatatacatatgaaagataagagaatactaagaataatttttgatcaatatatttgtaaatcagaggaaatgaagaaagtcattgtgaaacacaattgaccaaaacaaacacaaggaaaaagcgaaaaatctaacaaatccaacatatgatttttgaaaatgaatttgtaattttaaaacattttacaaataaatttcccagcacagatgactttacagaaaacctttccaatcatatataaaagaaagaacaccacaaaatcataaataaaggtaaaataaagcattttgtttttcttattcttaatctaaaatgtaactttaaattattaatagttacaatgtattggataattatagtacatagataagtaaaagggatgacaactttgtcataagaaatgggatggaggaattggaaattctctattgcaagatatctgtactacatctgaaatggcacagtattatttgaaaatgggctttgattaatttgaattgaataatgcaaatactacagtaatcactaaataatttagaataagtaataaattaaatttaaaactttaaaatgcatataatacatgggctaagagtggagataaatgcaattatgcaaaatgaataattaaccaaagaagaattttttataagtagagaaaaaaacagcaaatgcaacaaatagaaagcagtttaaaacacgttaaatttgaatccaactgtgtaaacaaccactgtaaatgtgaatgctctaaatacactaaataagacaatatcagagtgaataaaaacaagtaagatacaactacatgttgtctaccaagaacccactttaaaatgtaaagtctcaggttaaatgtaaagatataccatgctaattctaaccatgagaaagctagagtagttatgctaattttaaacaaagcacacttcagaacaaggacaattttgaaagataaagaggagtaatacataatgataaaagggttaactttctaaaaggagacaccaacccttaatgtgcatgtacccaacaacacagcataaaaatatgcaaagcaaatctgatagaactgcaaagaaaaattgacaagtccacgactatagtgggagacttcaccactcgtttaataattgatagatccaacaggtagaaatctgataaggatatagatgaccttaaaaacatcatcaatcacagtgacctaattgacatttgcagaaaactctatttaatatgatgaaatatactttttctcaagcccccatggaatattcgccaagaagaaccacattttaaatcataaaacacaccttaacaaatttaaaggaacagaagttatacaaagtatgctctcaatacacacaataaacaagaaatcagtaatataaagttgggtgaaaaatatcaaagtatctggtgattaaatagcatacatctaaataacacatgatcaaaaaagaagcctcaagataaatttaaacatgttttaactaaatgaaagtaaaacatcaatatttgtgagtggcaacaaaagcaatgattacaggaaaatttacaattacatgcttatgttagaaaaaaagggaaatatcttaaatcaatgacttaatcttctaccttaggaaactagagaaaaaaagagtcatgtaagcctaaagcaagcagaagaaaaaaaaacataaaaaatagagcagaaattgatgaaattaaaaacaaaaataaaatagaaaaaggtaaactaatatctatttctttggaaagatcaataaaattaatatgtaagaaagtaagaaaaagagaagacttaaattgccaatatcagaaatgaaaaagaggttattattttttattttatgaaaattaaaaggataatcatggaatactatgacaactctttgcctacaaatttgataacttagataaaattgaccaattccatgaagacagaatctactaaaactcatataaagagaaacagataatctgaataggcctacatctatcaaataagttgaattaataattgataacctacaataaaagaaataataggtccaaatggtttcatgacggattctaccaaatgttgaaggatgaattatacttattctccacaatctttcgagaaaatagaagcaaaacaaacacctcagctcattctatgaggacaatgtgactctagtaacaaaactagataaataaattacaaggaaaaaagaaacagttggttcagcatttaaaaaccaacataattcaccacatcaaaaggctaaaaaagaaaaatcatatgactaaatgcataaagtatttgataaaatctaacaccctttcatgattaaaaatgagcaaaagccaaccaaacaaaaactcagcaaactaggtataggaggaatatcctcaacttgataaaaatctatactcatggccgggcatggtggctcacgcctgtaatcccagcactttgggaggctgaggcgggtgagtcacgaggtcaggagatcgagaccatcctggctaacatggtgaaaccccatcggtactaaaaatacaaaaaaaattagccgggcgctaatttttggagcacctgtggaggtgggcgcctgtagtcccagctactcggaaggctcaggtaggagaatggcatgatcccgggaggcagagcttgcagtgagcagagatcgtgccagtgcaccccaacctgggtgacagagtgagactctgtctcaaaaaaaaaaaaaaaaaactactcatatttaatggtaagaaactggatactttcccctaggatcagaaacaaggtaaagataccctctcttaccatttctattcaacctggtactgtaagttctatctatctagtgtgataagacaagaaaacaaaataaaatgtatatacatgaggaagaaagaacactgtttttgcagacaaagcgattgtctatgtagaaagtccctcctggaactaagaagcaagtatagaaaggtgacaagttacaagttactatacaaaagcccattacttttctatatactagcaatgaacagttggaatttaaaattataaaaacaataccatttgcagtaatactataaaacaaaatatacatgtataaatctaacaaaatatgtgtacaatctaattgtggaaaacaacaaagctgtgatgaaataaatcaaatgcctaaataaatggaggtgttccatgttcatggattggaagacccaatattattaagatgtcaattcttttcaacttgatctatagattcaatgcaattgcaatcaaatctcagtaagctctttagcaaactgattctcaagtttatatttaaaggctaaagacctagcacagttaacacaatactcaagaacacagctggagggataagaacaatggaacagattagagaggtcacagataagcccattgcaaatacaatcaactgagaaaggtgcaaagtcaattcaatagtgaaagcacagattttcaacaaattgtgctgaatcctcacatgcaaaaaaagggtaaactagacagaccttataccattcacaaaaattaaatcaaaatgcaccttagtttaaaatatgaattgctacactacaaaacttctgaaagaaaaacaggagaaaatttatgtgatgagtttttagatataacatcaaatgcacaacctgtgaaataaaataaattatttgaactctattaaaattacaaacttctctgctgaaaacaatgttaagaaaataaaaatacaagccatagacttggataaaatatttgcaagacgtatatctgatagaaggcctgcatccaaaatgtagagtaatctcttaaaaatccacaataagaaaacaacccaataaaaatgggtagaagatctgaaaagacatctcatcaaaaaagatatactgatggtaaataaacatataaaatgcccagtaacatttgccattaggaagttaaaattaaaacaatatggaggtactactacacatctattagaatgggtaaaacaaaacaaataagaaaaaaaatcactaacaataccaattgctagtgcaaatgcagaggaacaagaatttttatttgctgctagtggtaatgaaaaatgatatgaccactttggaagacagcatgtgaggttcttacaaagctgaacttagtcttatgatctagcagtcacacttttaggtacatacctaattgatttgaagagttataacgaaacaaaaatctacacacaggtgtttagaacagctttatgtacatttgccaatcctagaaagaaccaaattcttcaataggtgaagagatacacaaattgtgatacattcatgcaatggaacattattcaatgataaaagtaaaataacaagtcataaaaatacacagaagagccttagtgttgactactatgtgaaagaagccagtctgaaaagactacaaacctgtatgattttcattatgaggatattctggaaaaggtaaaactgtagataaagttaaaaagtaagtgatatccaggactcagtgagagagggagatggttaaataggtaaaattcccgtgatttttagggtattaaaactatcctgtatgatgctataatggtatgtactataatgacattatgcatttatcaaaacccattgaacttcacaatacatagtgtgaaccttaatgcatgctcattttaaaaaatcatctctgaaactggaggattttaggaagaagtgtggactgtggcaagagaatcttactgtattacagatgtatgaaaaacttcaatgaaaggtttggagggaaaagatactggctaagtaaactttggaaattacttgagtgtgtaaaacggaaagtaaaataagctacttacaagcgctatactctagttagtaaagctgtttctagcaggtgctaatactgatggtgctatactgtatactgaaattgaacaattaagtaaatggatagtggatggtggacacaagtttctcagtttaggtgtgggttaacagataagcaaaaaaagtatgccgcgggtattcacatggtaatggattatatttggagacataagtatgaactcttgtttgcttaaattgtagatatagattattacaattaaaacatttataaatatgtgtatatacaccaattagtatctttccttggtttggttagctgatgacatggacaaaattcttgaatgacactatgatcaaaatgtacacaaaagaggccgggcacggtggctcacgcctgtaatcccagcactttgagaggccgaggcgggtggatcatgaggtcaggagatcgagaccatcctggctaacacggtgaaaccccatctctactaaaaatacaaaaattagctgggcgtggtggtgggcgcctataatcccagctactcgggaggctgaggcaggagaatggcgtgaacccggaaggcagagcttgcagtgagctgagatcgcgccactgcactccagcctgggcaacagagcaaaactctgtctcaaaaaaataaaaaataaaaataaaaataaaaagtacacaaaagaaatagaaaacaagaatagccctatgtctattaggaaaaatgaacttgtaatttacaaccttcatccacataaaactctagtcccagatggcttcatgagtgaattccatcaaatataagtttaaaaatataaagcaaattctacataaacttttacataaaataggagagacttgccaagtcattttatgaaaacaacgtcacattgttataaaaaccagactaaaacattagaaaaaaatactctgaaagaccatttttttcaggaatatattatcttgttaatatattagcaacttgaacctagcagtatacaaaatgataatacactttatcatatagagtttaatctcatgagtgcaaaattgggtgaacattagatattcaatcaatggaatttataataaaacaatagatgtggccaggcgcggtggctcacacctgtaatcccagcactttgggaggccgaggcgggtggatcacgaggtcaggagatagacaccatcctggctaacacggtgaaaccccatctctactaaaaaatacaaaaaaattagccgggcgtggtggcgggtgactgcagtcccagctactcgggaggctgaggcaggagaatggcgggaacccaggaggcagagtttgcagtgagcagagatcgcgccactgcactccagcctgggagacaaagcgaggctccgtctcaaaaaaaaaaaaaaaaaaaaaaaaaaaaaaaaaaaaaaaaaaaaaaaccaataaatgtgagaagattatcatttaaatggatggagagatagcatttgacaaaattaaattcattcatgatcaaaaaaacctctcatcaaattaggaataggaggtaattttttcaacttgatgaagtgcaccccataaaagtagaagctaatgttacacataatagtaaaagaaaatctgacaaaattgtcctgatgataaaagaaggtttgaaggaaactatttctttgattcctgcctattaatagacaattaatctctattgcaggcaaagtcaaataagcactttattcattttcatgggaatgtaaattggtataatctttcaggaaataagctgggcaacatgtgcaaggaactttaaaacgatcatatcctttgacctggagtttttaccttttagaatctactagaggatcttatcaaagatataggtaaatatttatatataagaaagttcactggataatttgtttttatttgtagaaccacgtattgaactttaaaactcagataagattacttaactttccttttagagaacacaatttgccttcaaaattatttaggaactaaattgttccctccttctcccacaaagagaaaggaaaggcatacaactatcctgttacctctccttaagccagcaaagaaaggataaccctgttggtgacaggactagtctaaggaacctctcagctaataggcaatgaacagaaaaataaataaataaataaataaataaaagctatgttcctcaaagtgtttgaaggcagacgacatcctgtgaaactgccccgtcttggaaaagtttgaccaaagtttttatctatccaattcagtagagcattgatttgacaagtgctaaccagaagctgcgctctaggttctagaagttggacttcccctattaaaattaggatgcttttttatccatatgcttttggcatcattccacccatcttccttagattctaagaagttctctctattttgtgacttacctttttaaataatgtttttattcccatcaccttaggttgtttacagagatgtatagccctttaattaaaaacaagactataattttataatccagagctatacacacacacatatgtatatatacacatctgtatgccagtaagtaatctgtatctgtacacataattttttttcacacacacacaatttttacagcattgcttaatgtgagattattggctcaaaggaccaaaaagggatcctctgatcagtgaggttttttggctgagacctcatactacaatagcagcgacaacagtgtcaaccatcaaaatagccattaggaagagatgggttaaaaaattgtgttattaatatataatggaatatttttcattcaactgttatgttttcgagcaaagagcatgagaacatttcacattttatacaataggtgctcaacatttcttgaattaatttaattttatgtgttttacaatactggcataataatgtatgcaagttaaatgtttttccccatttatcagtatcatcatgtctataaaccctctttgaaaatgcattttacacactacacaatattttactacacgatattttactacacaatgttttattatatgttagtaaacaaattaagaataactatgataccacacatcatactggccccaggataaaatatagatagttttttccagggttaattcattgtaccctgttgataaatatttgttaattttctcttaagatttacacatctgtctttaccctcccccgccaccgaaagtatagccccacatggaagcaattctctaattatctaaggctattaaagaagattccactgtaactaccatgaaataattatacttgtataaaattaattagtttataagtatgccctttcatactgagtataccatttgcaacttaaaatagctctattatattggcatgagaaggcataatctcatttagcaatgaaaatactgaaatcagagaagttaagtgaattacccaaagtcacctgaataattagcagtgaaatcatggcttaattttctgacactaaattcaatcagtgtcctttcactacatctttggtctcctgcgatgtttttccgtaataaatagaaaacatcaatcatgattttgcataagaagcacatggcatttcttgactgtacataaccaataaccatgtaccaatataaaaacttatttttgtttcactgtagttctgtttgtttgtttgcttgtttgtttgtttttaccaggtttatttagctgaacctcaggtatattgaaattctaggagaaatatttctattgcctcagcaaccagattttaaggagtgttctaggttctcattttgcaacagaattgatagagcttaacagatggctaaatcttaattctaaatttcagtgcaaagagtcaccggaatctaggtcgacactgggacaataggcaacactgtctgagagcttcacggtggtatggtcctagagctttcagtaggcataagaatcactttggggagttttaaaatgcagactccaaagatctatttctgattaagcataacaaatttggggggtttggaattagaatctgcattttaacaagatctccatgtaattttaatgcaggcacttattagaaacatttctgtgaagccctgcagacttgttttgtcatgtgggatgtttggcaaacagtgaaaaggcttcagaatccaaatatcccttcacaaagattatctgccatataataatgtccttttaagcataagcacttctgtctcttgtccttcattcaccccaaagatgacattggctattaaatgaaagaaagtgtcaaattagagtcaggacatttgattgctgtttcaccttagtatctccaacctaacatagagaacaatctctgcctagactggtgcatacagtggttatggaaatgaaatgagaaatgagaaagcttcaaaaaccaaatttcaaaataaaagagtatttctcagattaattgttctcaaactgttttttatattagatcttggggagctttcaaaaattctgatgcccaggcattagtatatatatttttaaactttccaagagatttcagtgtacaaccaagtttaagaaccagtatattagaccaatccttacattttgccttagtgcattcacttttgccttagtgcattcaatctcttaaaaaatgaataagcactgataaactcttgctgtagaattgattcattggattctttagaatcagctccactctcaccataaatcagcagtgttttatgttgggcatgagcagagctgccaaacaggccattttctttctgtccttcaacattgtttcctctaggtaactcactggccttggattaaggaaattggcaactaaatcgagtctcaggaaaagctgccatgctgtatcttgctgcaggggaaaagatcagcccttcatttctgggaattcttagtcataagccacatgtcatcagccataccaaccatttagttccatttcccttcaggtgagactgagtcacttctccagaatgaggacagcaatgcaagatggtagagagaagagatttgttaccaacgtgggaataaggataacttatctcacatgatttattcaacatacatgcctctgggtaccattagttcagaaaaatcattaaattattcaggtaccagtctagataaggtcctttacctcatataacattcatttcaggtttctgatacagatcagcacctaatccccaaacttattttgaaattaagaaacagtaccacgtgatggagagagtctgttgcaccaaaatgacatggtaggagactttatttgtccacctcggccttctgatagctgcaaaagtattcagaccttcaacattgctccaggccccttcgtatttacctgcactagagcatcggtctcaccaattcacacccctaactattccggcagaatatatgtcagtttacaaaatctctaaccccacgctactactaccaccaccattaattctagctgcaacattacaagaggacagcttgcatttccataccctcttaaagtttatttccatactagtaatgtaaagagactgtatttacatgtggctttgatccggctgtatgagtgtgagtcattaacatattgtttctgaatctcatttccctcatctgtaaaatggagctagtaattctgatttaataaggtgttgggagtttgtaatgtcatgttaaaattaagaaagcactctctaaaccagaaaactgttacaaattagattgtattatcactcttcagaaacccaggatgcccaagacacttcccccttcatttgaaatattatcctgtcattctgtcttatgaagatgtcccagtgtttcaggatctgcaacaggatgcctactgtgttaggaactctatggcccccttttgtgcaaggtgatgttagtgtgacacagtagcaagtgcattggataatgtcagaaaatctgtgttttattctcatgactgttcttagtaatttaagttccatgaatttcatttttctcacctgtgaaaatgagaattagatctatctcaaagatttattttaggacttacatttaatatttgtgacagtgctcataaatcattaaaaatatatataaaattatcagattatgatgaagggtaattaaatgttatgatgctagttaactattcttagtcaagtctctggtataataatttcatctatctagctatgtgagattactgtaatgttgaagttttaggatccagctatggggtggtttagggtcaaaatcaatggcctttgagtcattccaagcccacctttacatagattcatgcatatacacgcatcttttactgaatcaagagtctgggtttctcgttcctcccatttccaaaacccactcctcatcatccatcttccttgttaatcttgtcttctctgatgataaatctttttgctccctaagataatttttagcacaaactttttgtctttgaccccctttacttctcctttcacaatattaaagaagctcatacagttttatagttttgactaacatgcatttattaagagtgtaatatcagccagctcttgtgctttatgcaggtgacttctgaatttataattttagtgtttaaagttctcttcatttctagtcccacaatttcaactccctgccagacttctgctagaatttccatcagcactgataataataataaataacattataaaataacccatatggtgcctaaacccaaaacctctcctacctttttcacctcgcgttcttgttactactcctacaacttttcaggctataaatgcatctttaatcgagtgactgccaaataccgacaatgtttctttcatgatatttctcagacatatttccctgacaaccactgatctatttactgtctccatagttttaccttttccagaaagttgtaaagctataatcatacaggttgtagcctttcagactggcttctttcacttggcaaaatgcatttaaggcatttccgtgtcttgatggatcctttgtttttaatgctgaataatatttcattttatagaagtagcacagtttatttatccactcacctattgaaaggcatcttggttgcttccaagtttggcagttatgaataaagctgctatcaacattcatgggcaggtttttgtgtggacatgagttttcaattggtttggataaatactaaccagcatgattgctggatcatacggtaagagtcatccctcagtatcatagaggattggtttcagtaccccctgcagaaatcaaaatccatggatgctcaagttccttatataaaatggcacagtatttgcatataacctatgcacatcctcctatatactttaaatcatctccagattacttataatacctattatagtgtaaatgctatgtaagtagttgttataccgtaatgtttttatttgtatttttttattgttgtattgttatttttattgtagtggttttaccttttcaaatatttccagtctgtgtttggttgaatatgctgatgcaaaacccttggatattgagggctgactgtatttaattttttaatattaatttaattaatatttgtaagaaactaccaaactatcaaccagagtggctgtactattgacatccccatcaataatgaatgagagttcttgttgctccacattcttaccagcacttaatgttgttagtgtttgggatttaaaccattttaacaggtatgtagtggtatctcattttaatttcctaatggcatatgatatttagcatcttttcatattattacctgccacttatatacctagtttggtgagatgactgttcatatattatgacaattttatatttgagttgtttgttttcttattgttgagtttgaagatatctttctatatgttgtataccagtcctttggagataaatcttatgcaaatattttctctaagtctatggattatagtttcattctctggacatgtttttcacagagcagctttaaattttcatgaagtccaacttatcaatcttttcttttatagcttatgtttctggtgctgtacaaaaaagattatcaccaaaccgaatgtaaactggattttctacattgtttcccaggagttttccagttttgcattttagatttggctttatgatccattctgagcgaatttttgtgaaaagtataaggtctgtgtctagattcattttgttttgtatgtgggagtccagttgtttcagtgtcgcttattgaaaagactatttttacacatcctattgcctttgcgcttttgtcaaatgtgagttggctatatttgtgcagatttatttctgcgttctttattgtgttccgttgacttacttatttttcttttttctttttttttcccaatactcaccgtcttgaatactgtaacattatattaagccttatagttaggtagtgccagtccttcaactttgttcttctctttcaatattgtgttgaatttgctgaaagattcttttatcatgaatgggtatttgattttatcaaaggctttttctgcatttattgataggataatatgatctttcccttctttagtttttttttttattatactttaagttctgggatacatgtgcagaatatgcaggtttgttacataggtatacacatgccatggtggtttgcttcacccatcaacccgtcatctacattaggtatttctcctaatgctatccttcccctggcccccctcactccccaacaagtcccagtgtgtgatgttcccctccctgtgtccatgcattctcattgttcaactcccacttatgagtgagaacatgcagtgtttggttttctgttcctgtgttagtttgctgagaatgagagtttccagcttcatccatgtccctgtaaaagacatgaactcattctttttaacagctgcatggtattccatggtgtatatgtgccacattttcttaatccagtctatcatcgatgggcatttgggttggttccaagtctttgctattgtgaatagtgctgccataaacatatgtgcgcatctgcctttatagtagaataatttataaacccttgggtatatacccagtaatgggatcgttgggtcaaatggtatttctggttgtagatccttgaggaattgccacaccgtcttccacaatggttgaactaatttacactcccaccaacagtgtaaaagcattcctatgtctccacatcctctccagcatctgttgtttcctgactttttaatgatcgccattctaactggcgtgagatggtatctcactgtggttttgatttgcatttctctaatgacgagtgatgagctttttttcaaatgtttgttggccgcacaaatgtcttcttttgagaaatgtctgtgcatatcctttgcccactttttgatgggttttttttttttcttgtaaatttgtttaagttccttgtagattctggatattagccctttgtcagatagatacattgcaaaaattttctcccgttctgtaggttgcctgttccctctgatgatagtttcttttgttgtgcagaagctctttagtttaattacatcccatttgtcaatttcggcttctttgctgttgcttttggtgttttagtcataaactctttgcctatgcctatgttccgaatttttttttttttttgacaaggtctcattctgtcacccaggctagagtccaggtgtgatcacagttcactgcggcctagaactcctgggcttaagaaacacccccatctcggcctcccaaataactgggactacagggacatgccacagtatccagctacttttttttttaactttttttgtagagacagggtcttcctatgttgcccaggcttttagcctgttgatgtggtggattaaattaattaatttagtgaatgctgaaatagccgtgcatccctaaaataaatcccacttggtcatgatgtataattctttttgtaggttgttgcatttcatttgctaatagtatcttgaggatttttgcatctatgttcatgagtgtcaactaccttgtaatttatggattttaaaatgtagctctaacagtatcataaaattgcttcatataacttgcagtttattgtggatttattcattttaacatgaagaactcaggatgttatatttaatttgtattatcccagaatgttgcaagctgtgtggcatataggaaaacattaaataaatgttcattcactcaactgatcaactgaaaaagtaaccatgtgataagtaagtgcatatttagatcccactctgtgtatgaaactgggctaattaatggcaacacacgggtgaatcaaactaaactctgtctttatggatatcaccagacactacttgagacatggttaaattagaattgttataattatgagatctactctaaagttggaaccacagtcaatgtaaaaattggccaaacaacaagatcctccgtttgaaagctaattctctatactgcaaggcaaattttcagtagatattcaaggtattcaaatagcacaaattatagttggaaatatatctattatgcaaacaaaggggttataaataaagtttgtgaggctggtggatatggcatttttattttttcttgacttctttaatcaatttaattctttcttccctgatcactccccttcaccactaacacttattgaaccccatagtaagcaatgaaatggatactcagagtatgagtaaagcactggtagaaaatgttggcagtaacaaaggcaggacaaatgtgtattactgatttttctattcaagtgcaatgcagaaccttgtcataggaatctgaagaatagaggcagaaagaaagtaattctttacaaaataaaaggatattgggtgaggtttgttatctataaagatatgtcttcattcattatgtcatttatttaacaaatgtttaccaacttcttagcatgtgccaggcaggccttgtggtaagtacattcgagatgaataaaccatgttctctatgcctttgagaaacacacagtcaattaggaaagataagatatgcacataagtaactctaacacaatatctagcatagtataggtgatcaataggtatttaatgaaatttaatttaattgattcaataattatttatagagcatgtagtaccttccaaaccctattctgggtgcttgggatacaccattgaacaaaacagacagacaaaatccttgctctcaagttgcttttgtactcacaagggaaaaatttaatggtattgacagagataagttgctagaactcaaatgagaaatgcagataaaatgttactagcattatattgaaaggtggaatggttacagtggatgggggagagccaaggaatgtcatccatacattacagccccattttgttttgttttctgttcttctttggagattcagaagaaatgcaatccacgcagaaagggacagatgagtcagtctccagtgagcagctgggtgtgcatgtgtcctggatccttaggcacttcattgaatctatatgcattggacatgtggctgggactactaagacataaagttggagagttagatgaacgcctacaaactttaagtcggatggctctatagcatgcagagggaacctaacgaagatttttaaagagaaggatgatgttattcaagtgatgttgccagaaggatggatgtagtagtgtatccagaattggttccttccggtgggttcttggtctcactgacctcaagagtgaagctgtggaccctcgcggtgagtgatacaggtcttaaagatggtgtgtccggagtttgttccttcagatgttcagatgtgtccagagtttctccgttctggtgggttcatggtcttgctgacatcaggagtgaagccacagaccttcacagtgagtgttacagctcttaaaggtggtccagccggagttgttcattgctcccagtgggttcatggtcttgctgacttcacgagtgacaccgcagaccttcacattgaatgttacagctcataaaggtagtgcagacccaaagagaaaccagcagcaagatttactgtgaagagcgaaagaacacagcttccacagcgcagaatgggaccccagtgggttgccactgctggctctggtggccagcttttattcccttatttggccccacccacatcctgctgattggtccattttacagagtgctgattggtccattttgacagagtgctgattggtgcatttacaaacctttagctagacacagagcgctgattggtgcatttacaatcctttaggtagacagaaatgttctccaagtcccctacccaattagctagacacagagccctgattggtacatttacaaacctttagctagacagaaaagttctccaagtccccaccccacccagaagcccagctggcttcatgtctcagtagtgtcattgagaataaaagaaaggaattgcaggcaaaggcatatttatgttaatttaaaatagtaaaatagtgtgcatgtggatcgtttctagtctcaggacagaagctgaagtttgtgcccatggactatgcatcaaaatctgtgctcctgaagaaaggcaccaaaatactagagaagaatgttctgcaaaggcattgctggtctttctatgtctaccttgatatttataagtctcctgaggagattctactttctctgtggaatgatattacataatcatttaggagtttgacttcaaggcccgcacaatctatctattgcattatacatgacacaagatccgggcacagaacaatgtgttaagatgatgactctaccactgtgctcacatttgcctccagggaaaagaagggcactcttgtctccaaaatatgtaactccttttcatctcagccgttgtggctgcactttatttccttgtcatgttaagctgaagaaataagagcaaagcaaagggaatgagcaaagatatcacagctttgcttgtagtggttcgtctcaggaggtgagagaaatggtgaaaattattggggaaaaagcaagactgacaaggtagtgagagacacgtgagtgtctcccacagaggccaaacaggaacacacccaaggctaggggaagttgggaaataagaaaaataaaaacagaagataaaacatgagggatagcaaccttaaaagacacacatacacacagatggctattgggtggttcccagccatcctagtgaaaaagtgtatcaaataattaagttggcagatggagtagaagaacaacccagagccagcttgggcagggcctctgactgtttctaagataaagcatcctggaaccttgggagaaaagacacggcacctttttggtttgtttgttttttgtttgttttgttgttttgttttgttttgttgttttgttttgttaaggaggccttagattattcaactcttttttcccgttctctctcttgtaatgtgctctactacttgggaaataagaattttactgtgaacagaggtaaaatttttattacaattttagagaatgacatggctgagatctatggaatgtttagagcacgaaatgaactttaaacacttggattgtttattctatcttttctgttcctatttacttacttatatacatgtatccccttagctgtcatgcttttcactgcctggcaaactcctattcatttttctacacttaacaattaatttaattctcatgtaaaccttcactttccctctcaagttaagctacacctgactttgtcattccatgttattccattccatgtatatgccttcattcgcagtgattacaaggtgtttaatattttaaggtttatctttccctctaagtagacctgagggccagagctatctgattcatctttgttttccccatgcaaataatgcaatccttggtacatgaggagggaataatatatttaggggcagacgataggtactaataaaataaacaaatgagcaaatgagttggctgacctcttcccttctcttcctgagactatgggtgtaattagtgctttctagggcactgtgaggacctgccactcccgtgccctatccttgtgactggccccgtgggaatttgcctggcttttgctggataggtcaccctccttttggctggagaatgatcaggcctgggtgttggaagtcagatacaccactgagtcacagtacctcaaggcactgtggttccactccatgtgtgaaaggtctctctgcagcattcttgcaagatgagttttctaattagctttcccttataaggtgccagaggggatggttggtcagcattagagcctacagatgggaaaacaatggccaagagactttgcctaaggctactgggtgggccgaagccttccagttaacatctggtgagaatttaatgactcagtgctctattctccacccaaggttctctcacctgccagttcctttcattctgctgggccttgacctcaagagcacgaaattgagtgtgatgtgattctacgactgtgctactggctacatttcgcattggtgacaggaaagcacgggctgaggtgactttgtctttaaatggctagaagagggcagactgcatctctgagtatccttgaacctaacacctttgtttagggttgctctccagaaaaagcttgagagaaagcctaacagtttgcagagtggtcaagagattgaataataactgagtagcaataatactcagatgggttatgtcttttgactatttaaaggacttttgtataccactgtatgcagccttatgaaataaattgtgtatacacacacacagacacacacacacacatacatatatctaggataaaattaataaatggttcagtgttaaagattggaaaatatgggaagaaaaacaaaaaaattgacatttaatgccatcatctagatataactagtattaatatttttgtgtttctcttttctatttttattagataacatagttcctaatatagtaaaaaagtgacaattaaaatgtttatttatggaggaggatctgagatagcaagacggctggaagagtagttcaggaatggagagagaatcaggagttctgatgacttcatgtgtgatcttgagtagatcttaaaatttctcttagtccttaatttttgcccctacagaagggcaggaaatttgaaggcaatgtcaatagaagctcaatgccctaagttgaaggttaaaccttaaataccaagaattaatgtgataagaagttagtctacatgcagtagacaaagacaccaaaatacaccctttaacttcagagctactgccaggatttctatgtatcaagggactgtcttacaaattaatatatattttttttttgagatggagtttcgctcttgttgcccaggctggagtgcaatggcacaatctcggctcaccacaacttctgtctcccaggttcaagtgattctcctgcctcagcctccaagtagctgggattataggcatgcaccaccacctccggctaattttttatttttagtagagacggggtttctccatgtttgtcaggttggtctcgaactcccaacctcaggtgatccacccacctcagcctcccaaagtgctaggattacaggcatgagccactgtgcccagccgcaggaattaattatttacctgatccccatccttttttttatgcccatattttcaggttgccaaggtctaagaaaggaaagggatgaggtagcttaaatgactgtaagatgagcagctcttctatcatttaaatccacaaaactaggcaacaagggaatcatcacatcaaccatcctacactagtagttagctgtagtataaaaggtagttggcccggcgtggtggctcacacctgtaatcccagcattttgggaggccaaggcaggtggatcacctgaggtcaggagttcaagaccagcctggccaatatcatgaaaccctgtctctactaaaaatacaaaaattagctgggcatggtggtgtatgcctgtaattccagctacttgggaggtggagacaagagaattgcttgaacccgggaggcggaagttgcagtgagctaagatcacacctctgcactccagcctgtgcaacagagtaagactctgtctcaaaaaaaaaaaaaaaaaaaaaaaaaagaggtagttttctgcgttcaaattatctcccatttaatgatttgaacacacctgttctgtgggcctcataccctctcctctgatgcccagacatcacaacaagagaactctacctaaaacaggcaaagattggaggagatgaaaaaatgtccaccctgagtcaaaggaatggctccacctttagggcatacctctccagactgggaaagctagttggagaagaacaagaataaaggatatctgggttttcatttttgccttgctggcctgtggtttcttgagattttcttaccataaattgcccgctatttaggagagaattttgtttctgtgggttcaaagagaccaggtaggctgtggaagaaaaaggcaagggctagtggcattcacttactcctgggctctgagctctgggctctctctgggagagagaggacccttctatatacttcttagtgtgtaacccagtctaaggtagaaacgaagaagctctcagggcaaatgttctatccaggacattggctggagcattccttgtcttgtatcatattgtggattggcagcaaaaggcagagaacaacttaggtatataatttgcagaaatatccaaacattgttgtctgaacaaaactctgttgagtaaatggtgatggtgatgatgggatgatcataattactattattgatgtgttctgtatgccgctttacatgcattattctttctacaacccagtaaaaataggtattatttcttccattgcacagaggaggaagatgatgttcagaacacaaacaactcacccaaaatcccacagctagttattaaccaggacagtattgaggtttccatagcctgtgaaatgtctttcactatttgctgttcctagttttgtgttagagtcttgggtttggtctattacagagaatgcagaatgcaaattctggagactcccccacctaacatgtctgggctactgatccgagaaaccctcacaggtagatctaacttaactgggtacagctagatggcattttgtagcaaaggctgtgagcctaaaggctggggatggggagcgattcagccttcatgaattgcaaataatttttcaaggtatcttcttagggaagaaagagagttggggtctcaggatggtggggagaaggagctgtggtggggcccagtgtagaaagaggaggcctatagctattttctccctctgttagggcagatctcagctcacttattttccttcagcctcaaacaagttgttttgtttttttttaatttaggaaagagaaagattatctttaaacaaaagtggcaccaatatcctgccatcctgattaaaagcaggatgaagataaaatgatttctctatccctcatggcctacaaagccaacgcctcccagggaaatggccttctcccagcggcagcccagctcttcacctttgaagctgcctctaaacctgtttgcttttggagctccaataagatttgtgcttgctgccacaggctggcccagataggctgctcgtgctgagactatggctgtcttcccgggtgaacaacaatgacagttaattacatcaacccaattagcagcagcatccactttgtgcaagcaaggcccacctgtggtcttacctatgtctagcaaagggcccccgggccttagtggggaagctggtgaattgggaggctagatgtttagcagtgacctcctttcagagccctcttttcttggagataagtgagggaactggaaaaagaccaatgccttacaccatagtcagctaaacaggctgcagatcataatggttttcagattattctgctaggtaggaacagagtcttccctcagcacttagtggatgatcagaggtggaactttatcagattttgcctttggtaatgaggccgtgtttgccttgcctctaatagaacactaagaacaaaggcctctctatggaaggtgggaggtagacaagagaatgtgacagctgttttccttggctccacactcatgttttccagcctttacagggaatcagaagtgctgggttcaaaggctaaatctaatcacctattagctgcttaccttgggcaagttacttaaactttctgagccttgcttttttatttgttaaaaatgtgtctagacaaactgtaggggtggagagcaaaccagtgattgcccaggaatgagtcaggagggaatgacacaaagggcagcggggtgggtgggaggaggaaagttctgtgtcatgattacagtggtggtctcacagctccacgcatttgtgacccatagaagtgaacctgaaaataatgaattgtactgtatacacattttaaaagcaaataaattctaaagagtgattaaaataagggtgaggataataataaaagatttgaaaacatctaggacaataaatggctcacatgtgtgctaaatacatatcaattgctttcttttctgagtctgtttcttcattcataaaatgaaaattaaaatattacttcttataatcataaaacatgttatctttgtcataagtatggtatgattataggattttgaatgtaaacactttatcggtagtcaagatatagcaattcattcattcattcattcattcattcattcattcatgttttttgaatgctcttgttgggttaggccccgtggtgggagctgctgttaaaattttaacagaaccaatccctcccatcccaatgtagctcatagtccaggaaagagagagtgatgaaaacatgattgtaataaatgctgcatgtgtgaagttccaacagctatagttgtatatctaagaggcacctaatgcagactcggaggaccagagaaaactgcttgaaaaaaaaatattatctaaaaagagacatggaaggcgagtagaagataggccccaaaggaagcacggaaagagggagggtttgcaggcagacaagactatatagagaaagatctagaagggaggaagaacaaggctcattctggaacaaagcatctttcagtctggttggggccggggatgatggttcatcctgaaaattcaagccttagacggggtgttggaagaagtgaccactagagagatttccactcccataggcacataatttcataatgactcttggctccctccttgtattcatttctaggcaagtggggtctcagccctttgcttttcctcgggtatttggattggaacttaggggaaagtctctctattgagcagtgtgatcttctaggttccctgtgtctgcttttgcataatcactcaggttccagcctgcagtcttcaccgccagatagaaggtgacaggagtcatggaaaggattcacatcctctaaggctgctagatgatgtcaacccaattaccagagacagcactatctgaacggcaatgcccctcaggtgtctgcacctgtgtggatctgggtggaattctttaatgtgacatttgactggcagaagatctgctcctccctgcctcttttgtccccttctgaaaaggaagcagcatcagcagtagggcaaaattcagacaattcataaatagccactttccccagccccagcccctgaaccacagcaacctctgcctctagcctcctcagttcctcctttaaaaccttgagacctcctcacaatccaggttgtggccctggcccctgccctatgctttatttgttctggttgttgagaatgtactgcttgttatatattataaatgtcacctctttggaggacagaggacaactgactggatgactgccccgggaaaatcattttctttttctgggttactatgactcagtgaaaataaaaaaggattatcatatagttaaaatgtagagttaccagctttattaatttctacggtctcttccactcttagtagtctatgattctgtaaactgtgaataaattcagattcccatatcggaggtctgaatactgagtgctcatccaaagaaaggagtaaatagtctacatccggatatgagcaggaacttctgcgaatcttagattaatagaactttagagctgggagtgaaatcaagttcacccagcaagccttttcagatgtgttccacagagctctaggaagaccatggggaaagatgagaaggggttagcggagagagctccaaattcctgactcttgcttcagcccaagaagctctgatttttcctgctgtttacagtggtagtctgcctaagattttatttgacgaaatatttctgtgtctggcagacaaatcctaaaatccttatatcctgacttgacaacttgctctcagtagagccttgagtatttacctcccaacttctctgagcctcagttttcttgtctataaatggaaccaatcattctggctctcgaggttttagatcagcacaaaacgtccaagaaaatgactagcatgaactaggcgtttggtatctgttagttggatcttagtcccacttatgattacttccttaaggtggaaaccaggttcccagagctgctctgtgaagattgaagaggttggtaaacacatagagatacacatgagcacacacacacacacacacacacacacacacaccaggaactgccaagaggatattttaatagggacaattacatctgcaacacttggctatctctgcattaccaaacaaggtgacaacagcaaaatcctactcaaccacaacggcagaagcagatcgctctgataatcagtgcaacaccatgggtttggttactgcaggataactctctgaccttactgggcatcgtttccatacagtagctatacaggggaagttgaataaccggtcttgttcctcgcactcagggtgtggcagctgtagtgcttacagtgtctgtttcaaatttaccagtccagtcccttccaccctccccctttgccctaaaatggtggccagcttgttgctcccaccggagacagtgttgcagtaggcagaaagttgtgcgcacggtgacactgctttccatttccgaagacgtgatactcttctaagtggaacagcctagaacttagatgtgtgttctcttaagccttctgttctcatatgactaattcgagaatccacccactctgggggccattaggcgggtacgaggaaagatttccaggcagtgttctcgtcacaaattgtatcagtgcagggctttgtaatcagatggggactgactctctcttccaggctgccagggcaacatatccctgggctgaggttcaatcctagccctgtaacaaattgactgtgtcagcttgccccagtcacctgccttttgtgtgtttcctcacatatggaattctcatgtttcttgtatatatttttttctcagttggaatttgcacaatgcttctctttcccttgttattgtttattgctttcggaccctgtttagcaacaaatttggattctagtttatacttattattggctcctagactttgacagtaccattacttatagtattctcaaagagtattgagactaatgccttcattttttagttggagaaattaagaatttgagttagagggtaattttctcagagcctcccaaatcctgagtgattaagctaaaatttgtgactttcagtcagttgtcctttccaatgtgtttgttcactctttcatttaattgtgtgtacattaattaattactcatttaattaatagttatgcatttcttatctgtgtcatgtaatattttaaactgtggaaatatagcaatgaatggaactcttgtcttagaaactttattctgggcagaaggaggtagtcaataaatcaagttagattacacataagtaggaagttatgatattaagggttatgaagaaaataagcagaaggctgaggtgtgcagatcaactgaggtcgagagttcaagaccagcctgactaacatggagaaacctcgtctgtactaaaaatacaaaattaaccaggcgtggtggcacgtgtctgtaatcccagctactcaggaggctgaggcaggagaattgcttgaacccaggagatggaggttgtggtgagccgagattgcaccatcgcattccagcctaggcaacaagagtgaaactccgtctcaaaaaaaataataaaataaagaaaaaaaaaagaaaaaattaagcagagagagggagtgagaatgctgactgcaggagttcagttgtagagagaggtcatggcggacctccccaggaaggtgatctttgatctcaactagatagacatggggaatgagccacgtagacacctgagggaagaatggctcaggcataaacaacagtaaatgcaaagccctgaggctggagcatgctaggcaacaggcatgttctaggaagagtaaggaggccagtgccactgcagccaactaagcaaggggagagtatataggatataagaacagagaggtagctgcctcaggtcttggtggctgttctaatgacttagccaatgagatgcaaaaccaatgaaagatgtttgagcagaggaatgacataagctaatttatgtttaaaaggataccgctggctttttggttgagaatacaccgaaagaggacaagagtggaagcaaaaagaaaagttaggaggacactgcagtaattcagcagagagatagatgatggtggcttagacgaagatggtgagagtggtgatggcacaaagtccactaccatggacagcagaagacatgaatataaactcaataatgaggggtgtgagaagggtaaggaagggtgggtctggactactctgcagagagaccgaacttagcctaggagtacatggcatcagagaaggcttcctagaggaagaaatattccagacctaagccacaacatgatgtagagtgaccaactgtccaagtttggttttcagacccctcggtcccagggaaagtgggatgggtgaccacccacggtcggagtttcctggtgaaaccagagtggtggtgggaggacagtgttttcagcagagagaattgaatgtaaacatctgagagacaagagagggccatgctacagggtagggaataaagaggaggaaagtggcagcatgcagggttgaggaggtaggcaagggcaccagcgtacggcgaggaagccagaagatcgctgggatttgtttctacatcattggaagggttttaagcaaggagtgttagcactaacaactcagtgttacctgtcacctctgttaatttacctgtagaagagctattacaagtaatttataagttttcaggtaaagaaaaagatgacaagcaagaaatcactaaacagagtaggaaatacatttatacgaaaacattccagctaagtgttcagttgctacttgggagtccttctgtgtaataaggtcaaattccctatgaaactagttgatgttttccctcgtttctctgcttgcatcactgacatcattattttcttaggctcaacagctcagagacataatttttatcttttcttcttggtgactctgacagcctccaagttctgatttgtttttccttcaggatgtctctgcttagtcctttacccgcaagttccacggctgccagcctgcctcaggcttcctcacatgtgccttggcagtggtggactaagctaggcaatcagtaggattggatacccctgggtgcaggcaatgacgaggtgttgtctttagaggtctaaaaccaatacaaaaccaattcgaaattagcctgccttgtattatttctatgtgccagcaactatagacaatgacaaagataaaatctccttcccactggggcagactactcccaaattccctcccactcaactcttgattagtgtaacatttgctttattggtatccctgacaattgaccttttttcctccaatccttctcaggctgactttgttaatctaacaactacaatctataaccacctgtgtcgagctctcccaaacttcagaatcctccctgaggtttacaggatcaagccccaatgcctcagtttagctttcgatcaagccctcggtaacctgtaggtagcctaacagctgtgacttttccttttaatttcacatcccaaaccctcttttccagaggagtctatgttacaaatttatggtttcctgctgcagtagcagggaggaaaaccagttttggatcccagcagtactagtgactgtgtgagcttaagctagtcactttccctctaaaagccttgattttctcatagtaaaattaggataataatacctatagagctgaagtcacaggattacagcaagggcaaaatgacatcatggttgtgaaagcagctacccgtgtgagccacgatgactattataagcagtagaggtaagtaatacttgtcagggtagtggttgtcaacaattagtgttgatattcatggcaatgcatgacttactgatttttttaatgttgtctattttatcttttatatattttatctttaccactccatatcatactcgtcatttaagaaacaactctagactaacttcctgccaaatacttcctgggtcacactagcctaccgaactgaactctgctgtcctcaaaaacctccaaaacatataccctttgcattcaggcatgtaccacatgtatcatttcatatcatgttcccacctggactataaatgcaacatctcatgcttgtttagcatttctctaaatgctgagaacatagtagatgttaaatcaagacttgctaaaatgaacgaatgaatgaatgcatagaggcatctttcttttttatttttaacccctcacagcaaaatatgctaatagtgccctctgctgctgactaacgattctgttttttacacagttgaaaagcaagtgacagaaagctcagtaaaaggaaataggtaaaaattgtgtctttcaaggtgcttggcacaaacaaagtagctgtaatgtcacagtgcagacagaaagctaggctgtcagaaggaccaaatgaggtggctgcagacaagacaagcctctcttttctggactgctgattgacaaccagcttggagttcaaacactctcaattttgtaattttgtaacaagtttctcttctcctgtgcccctgccctagcaataagtgtgttatatatgatcagactatttctaaatgtaactgtatgaaaattaaccttgaaggttctgaggttgatcaagggataatgcactaaaaaaaaaaaagtcatgagagcaggtgaaatattgccagagaattcacagtcctattcattttccttcattttaggtgttctgatatagtcacaagtcattgtcttttaatgattacctgctatgtgttcggctctctttttataatctcaattaatctttcttcttttctttcttactctttttctttctttctttttctttcttctttctctttcttctttgtttctcctttctttctttcctttctccttccttccttccttccttccttctttccctcttcccttctcttccccttccctcccctcccctcccccactccctgcccctctcctcccgtcccctccccttcccttcctgacagtatctccatatgttgccgagcctggtctcaaactcctggcctcaagtgatcctccagccttggcctcctaaagtgctcagattacaggcatgagccatcatgcctggccaatgaaatctctagataacttatgaaatagatattaccagccttttctttatagataaagacactgaacagatgtgcgttaaaggccagtaaaaggcagaaagggctgcaagtccatggtaacacagtctgcactcttctctgcaattccatagcttctcagtgtggtctgccgctctaggagtcagaggagctggttttgagtccatatttctaccactaacatttcagtaaagttgagtaactcacatttctccacctctaaaataaagggcataaggtcaggtgctttctaaggtcccttccaacttggagtcagttgtttgcagtgtcgttcacttgctaagctataaatacaaagcacaattgtggctatgattcttccttggggtccctaccatggacttgatcttccctgtttagcctgctctgatgagttaaacgttgacctcagatcaataatgaaaagccctctgtgctgacgttgatggtcattcaaatatcattctatttaatttgtaaatccttctacacataaatgacatgttttgaccacagagttcaaggcatgatagtttatttcatgctgattctgaaggtagggcagttttcagttttggcacgccaatttgtcacatttgacatttttgacaatcagcctttagctggtaagtggcttggtgggcaacgtgtccctagttaataggcacaccccgatagctcacagtctccggggacagccatctctgcagcatgcctagcctttgaagcggctcacgcgttcttcaaagcaaactttctggtacagggagcaacatctgatgtacttttattacccttagagacaaaaaaaacatcactcagaagccaaacgtctgcaaggaaaaatctgtaccctgtgcttagccctgcggcctggaagttcagagaaggctgaaagtgctccctgtgaggaaaccagggcatgtttagacagagctttaatcatgtctaaccaactcagagcagtggcagcagccatcttgctgaagatgtcgcctccttctggatttgcactggagtgtgggggagtgggtgagatctagtgtcagataagctcataataattcctactgcttcctagttattcaaaacatcaaaactgcaaaatcttcttagagagttcagcaacttttttttaactcgaggaactgacatctactgactttgacttctgtcaaattatttatctattctggtgttttattttttccactatatgagaataatcatcacatctattgggatgtcgtgaaaaacttagtgaacccaatgctaaacgatgaagtggactactttcccagaaatctttactctgtctaggcaagggagcttcaacaataccgcccatgcctgttctttacttaatactaagtagctaaagttagaataaattgcagattaagaccactgagaatccagctgtcctaaaagatgtttacacctttatctgggatgctgaagggttgaaaatctccaagtagtctttatttaattaaccaatgttaacttcaaagaaatataattctggttcatattaccttgtgaaaaaaaagtcctgaaattctgaacttgaaatataataaaaattctagcatctattcagtaaatgttgctaaacactggactatcgtgatagaccagaatattactccagtgaccggaagccttgaagtcagcttctgagcctcagggactctataatttattctaaatctaagtagtctgattgcctaagaccttcaaaggcatttgcaaagtttaacacaccaaaaaacaggtacagagaacaaaaagggtgattggatgcttgttgctttatgtcctatttgaccctttggttttaaatgaggtaaattgctccaattaactcctgttggagatggttttctttcaatgattccttctgttccttgtttgacattggaaaagatgtcattagtaaagcattacatggctttcgggttttattaaaagactgactgagtttaccaaatgctaggcttttcagacaacatacctgtgtgctgcaatattgtcattattcccatttctgatacgaaagtctaataatttgagtgaagtagattagttccaggtcatgtagggtagaaagtttcatggacttatgggcagaggaatatgctgctggtagagcccattgactttagtctcaacgatgaattcacgtcagctatttctgatagtagctactcctctgcttttcagaaggaagctcagtccttcagggctttcacactacccacttgaataatacctcccttgcaagtgaatacaataaaacttactctttaactggtatttattaagattttcacagcattcattaaactgctttccgaaggctatctcattccactccaccccaaaacaccaactttctgaactctcattttctccgaaggctcagtcctttagaacccaagaaatcattcagttcaaatgcaacacttttcagctgaggacactgtgatgcaatgagaggttaagtggctcattcccacgtattggtggcagatcagtcaggccagatccagctcacctgtcaccttctctccaacttttccctaccactaaccactcctttcccacttgcatgacctttcctcctacccctatcacaatcctctgccgtaacattcaccttactttaagttgatcatttgtttacatctgtttttttccactttgagttccttaaaagaagggataacattttattcatctttaaatgcctccctaagcctacaacggcacggccactcaaaaactctctgtgaaatgaattcatacatgaccagggctttacgacatcaccttctctgtgcctgtttgttttttcccatttacacaatgaagatggcattgacgtgtctctcaagataattatgtagttacatatgattgctacgtgcttcaagaccataaaaaagctttataaagccaaccacgattacaacctttgaaacaatggaatatacgattaacaagtggaaagagatgagacataccaggtaatgcttattgtccattccctatttctacctccacccaccaccaggtagtaactcagaggtaaaaacataaacttggaataagagaggagggtttacctcgggtctggataagattattaggaccgatgtcatcacgctgccgtcatgcgggaaggcacctaggtctgatgagagaggcccagggcaagattcccatatatataggtaacactcaattcactgttgaaatgaggctcaaatcaagaaggcacgtggccttatctattcgaaattgattcgtccttcaaaagcagaagatatcagataattataaaagggactgttaaggtagaaagactgtcaaactctaggctaccctggctctcctctgtggccattctaccaaaataaagagagggggtacagcatgtcttattcaccttttgttctcccttctcctaaatgtaccatataaaatgactagaaaacatgtgtcatatgttgactaaacgaagcattcatttagattttcctccccagcaacatgtctggaaaaaaaatgataggaacatcttgtttggttatttttattgcgggcccttaataagatagttgaagccagaggtgcctcattaacccaactttttacagctgatgccatccattgtagagtgaaacagaatcaatagtcagattgttcccaacttggctttgcccagcctcggaggtcagcagtgggcacagaaaacactgtgaattacaaggaggaggaggtaaaggggaagctctgacagaatgagtctgcagctccaacccagcagtgagctcaatgagtggctgatgttacttgttttgatgtgtttgaagaaatcaaccaggttcaggagaaaaaaaaaaaaaaaaaaagtctatatgcctcctgttggaaacatcaaattatcagggagtagaggatttgtgggtgggcagttggaactatgtgagacgtcatttcacatcacagccttcttggcaggaggcagagagggcttgtaacacggacttgtggtctggagtttttgccaatggctcctgtttccctaaagtgctctggatgtgggaacaggtcaacattttcccacttctcagccataccactgtcagtcagctcagaatgttcaatgctttggcataaggaactggtgaggcggcccccattacctcctttcttgacgtcagtgagggagtcattccctgtggttctcttgtgtgaaataattcagcatttgtacttcaattaaaattcacaggtatatattattaaatagcactgactcattccatggcttctcctcagctgaggaagtgccaaaaatgttatgatgtcaatgatgaagagtgcattaaaagtcttaagagtgcgttaaaactgagatttccttgtcaaacaatgtaggttcggggctgtgaagaaagcacttcgcccatccacagtatcactctgtcctgtgtcagatttttctaaagaagaaatttaagttcaagattctttttgcctttgagggagagaaaaatgatacctgactagaatcctcttcccttttgtgagcttcataaaagcagttcaaggctcaaggtatgtctttaaaaatagggctgaatcaaattgaataatacaactcgtattcaatatatcacagaggagagaagaggaagtatgccaacatacatccctcagcaggaaaataaaagaggagaaaaaggttttcttgatttggtaaaatcagacttttttattcacatttattattttatatttaatatgcctagaagcacggccaggggagcaacaaccctggtgctcttttctcccttctgacccaaaagactgtgcttctagctcagacctggacactctgagccacgtgaccccaggtaatcacctctctccactgctctccatgtcctcatctgcaaaatggacatcacgatccatgcccagaatacctcctggggttcccagtgtgtgaacatgctctgtgtcataagcattcaaaacaataatataggtgaaaataaaagctttgtgagctgcagacttttcacacaagggaaggatcatcaccaagaccaacggtaattcctcagataacacagatgagatgtactattcatacacattggactatttaacctaggctgactcaagcacattcagcacttcgtgagaattagaaaagacaccctttctggaaagatatagccctgcaggtccatagattggtaagcaagtaccatctctgggcaaatagaataagacatcccttccaccagctggacaaagccctgtgccagggttccagcccccatgtacactcccacaggcattcttgcataaccaagttcaacagtcctggctttagctctggctctgttgctcaaattctgtaaccatgggatcatctttaccacctctaagcctcactttctacccaaaaagaaagatgaaattgaactgtgatttctacgccctccttcagctgtaaaagcaacaaatgtcctaatgggcttttccatccttctacacaagggattatgtgccatacgaactgcaatttctaagcttcacttttttttaagttgtctgatgacactccctaaatatttgaatgaaattatgtatggtttgatgtggagcagattaagaagtaaatgctttagtgtatacatgatgtttcaggtgtaaagcaacatggttaaccggcaaaacaagaataactaccctgtgttgtcagcaaatggcacaagatgtggctctaacacaacatctattcggccttcacccactttctgtgggactgaatataagaatcagtgatgaagtccctgcccctcccctagtgcccatcgcacccaggaagtgggaactttaaggagccaaacaaagactgtttctatgttgatgccttgttgccagaaaacagaaatcagatcctcacccagatcctccaatcagcaaatctgagtgccaacaagctcttcacagtgggaggacaaatagcaatctacctttgcaaagctcaagaacttttaggtctaaatttgggtttacgtttttaaaaatcaggtacacacataaaaaacccattcacggccgggcacagtagctcacacctgtaatctcagcactttgggaggccaaggcgagtggatcacctgaggtcaggagttcgagactagcctggccaacatagtgaaaccccatctctactaaaaatacaaaaattagctgggcattatggcaggcgtctgtaatcccagctactcaggaggccgaggcaggagaattgcttgaacccgggaggtggtggaggctgcagtgagctgagatcacaccacttcactccagcctgggcgacagtgagactccgtctcaaaaacaaaacaaaacaaaacaaaaccattcacaactctctaaatgttagcagaaataaatttggttgtgactatttctaaaatggttttggtttcttttttttctttttgctttgtttgtattttgagtaggagggagggccttgttacaatcaattatgtcattttcagagaagggaactaaagtctggaaagggaaagagagtagttccatatcacacagcagggcatatttgatagaactgggaggataatccaatgctctttccactctcccatgattaaaatacaaccagaaggttcagtaaaaaacagatggttcatacatcactaactcaaattttcaatttcaatggatgaatgttcttaccactcaccagattgtaaacggcagaaagtaaatcttccctccaccactgcccgtcataaaagcatgtgatctcccacgggcgaatgataaagtctcccctaggttagaagaaccccatcccccatgaactatgcctttgtagtgtctttcttagaagtgggacctggaaccaagcattctctggatcaatgcccagtttggtctccatctctgttcttcaagagcagtgtccccacctttcctgaggccactgcaccagcaacactagtcatgtcccaatgttgcccctaaagtggtcccaaaagctcctatgtaattacaaaaccattctgtgaagataaagcaaacatttgggagaacaaaaataaatgccagatattaaaataagcctccctttattaaacaaatcagacacaagcacaaatctgtgtgctggatagcagcaatgaggaggggcccccaaaatataagcagatgacggttaaaatcaatcagaaatttatttttcttatgtaagtacaaaacacctcttttcatcagtggaccccactcctaggcaacctggccatggtgcccggatgcaggcagtattcaagagtttcttccaaagtcaccagggtgaaaagccattctactacaacctctacatgaccttttaaagtgtacaacttataggacagtcctttctggagtactgtggagggtgaatcaaagcttccagtgtaagtttattgtctggcgaaaacaccagagccaaaaattccaccaaggccctggaaagactgaagtcccctctgtctcatacagtaatcatccatgagatctcccggagcctgggtgatcattacgcccatgatacctgaggcagcgtggactctgccaggggctcctcagacccaaagtggagctcactttggagagtcggagctcatggccgttagcacctaaggatgtggctgaaaggcacagagcaagaaagggcttcagagaccaggcacaggttaattttagaactggagcacacgtgagagttgtagtttcaccaccaagaagcttattctctctaagcttttgaatttgcagcaacttaattgtgtgtgtgtgtgtgtgtgtgtgtgtgtgtgtgtgtgtttgggggaggaggtgaactgggctttgatagatacatgaaatgatgaaaaaccaagaaagagagctgggtgtggtggcacgtgcctgtggtcccagctccccgggaggcttgaaggatcgcctgagcctaagagtttgaggctccagtgtgctatcatcacatctgtgaatagccactgccctatatcctagacaacatggttagaccatgtctctaagaagagagagagagagagagagagagaaaacaacccaaaacaagaaagatagattgatagattgaggcaacacaagcatccttaggaagagaaggaagatctgatgactgacattaacctgttaatttataccatggctctgtacaaaaataaaaaggttctcataagattaacaatttaaataaatatttgatagaacattctttctcatttttatagctcatctttagggttgatattcagttcatgcttcccttgctgttcttgatccagaattgcaatcacttcatcagcctgtattcgctcctgcaaaaaagacaatttacacatttctgttacaagcataataatcagtctcatcagcagacgcttcccaaacacttcctggcatccatctgtacatgggttgatgctatatccagttaagcattgaacacctcctcttcatccccagacagatgccttctcctaacattcctgggtcctaagcaattactttctaagattttctctcctgcaaagaacgactgtatgttcaactgcttcctactctttctaattcatggccccatagccatctccaactcagttcagtggtcaagctgaattcaccattttctccccaagaacctgctctgttcttcacgtaggctctctctcggtaaagaccaacatcgctcacctatttgccttaaaagcccgaccatggtttcctgcatctgataagtcacctattctgttctatctgctgtctctgcccctttctcctcaatgcactattgttgccggagtttaggtcagggtttctcaattttggtctgattgacatttttagaccagataattatttgttggggggtgggagggtggtgctgccctgtgccctgcaggatgctcagcgacaccccgtcctctctacctaccacaggtgtcagtaacaacctcccccaccaacccccagatattgccaaatgtccactagagggcaacattgctccctctttttttttttttttttttttgagatggaattttgctcttgttgcccaggctggagtccagtgacgcgatctcggctcactgcaacctctgcctcccgggttcaagcgattctcctgcctcagcctcccaagtacctgggtttacaggcgctcaccaccatgcccggctaattttttatatttttaccatgttgaccaggcttggtctcaaactcccgacctcaggtgatccacccgcctcgccctcccaaagtgctgggattacaggagtgagccactgcacccggccaatcgctccctcttgagagccaccattgtaagccctcatcatctcttaattactgccgcagcctccaaattgctctcgctgtttcactactcaccctcacatgcttctaatgaatcctactggcttatatctgattatatctctttcctactcacagctcttcaatgggtccctactgcttgaagtttcaggtctacttttcttggtgtgcttataagaagttgctttacacctgtctcttcttttttttttcttttatttatttatttattttttattattatactttaagttttagggtacatgtgcacaatgtgcaggttagttacatatgtatacatgtgccatgctggtgcgctgcacccactaactcgtcatctagcattaggtatatctcccaatgctatccctcccccctccccccaccccacaacagtccccagagtgtgatattccccttcctgtgtccatgtgttctcattgttcaattcccacctatgagtgagaatatgtggtgtttgttctctcttcttattcatacctttctctcttctttaggtttattttgcctcccttctcctcaccttatattccaacttcttaacttacctcaagctccctgaatgtaacactttcaaaagaattccctaatccgattctgaattaagtgtttctctggatgctcccattatattctacgctttcttttattcctcacattaatccatcagcaggtgcagctggtctacctccaaattgtatctcaaatcttccatctctttttaccttctctattactactccagtccaagaccacatgttagctaaagtaatcttctgtatcctgtaactctccaggactggaggagatatgtgtgtgtgtgtgtgtgtgtgtgtgtgtgtgtgtgtgtgtgtgtgtgtgtgttggggtgtggggactacatgactttccactagttagaatacaattcgcatttcccaatcagcctgtggaatccgactcctgccgcctccacgtcctctactgaagccactctccctcttgtctgccatattccagatgctctggctttctttgtttcttggtgactccaaacatcttattgtctctgggcctttgcagcttgtcctctgcctggaatactctcctccagacatttatcctagtggacttctaatttattaggtttcagctcacatgccacttgtttggcgaggcctcacaatttgccgctcctaccctttcactccagaatcagttgtcttgttccattttccacacatcattggtcaccgtgtgaaaaatctctcgattatttatttattttttacttttttgggatctgtttccttccactagaatgtaagctccagaagagcagggcatcttatcttcttggtcactactgtattttcagtgctcagaaaagccactgacatgtggcaagagttctattttctttgatgagtaaatgtgtaaatggatgaagcctttctctgtctaaaatgcctctccctaaagaaattgatcttttaaagctttttttcaaatgccatctcctcaaagaagcctttttggattgctttttgatatccctcaaaccacgcatttgctgctaaggattcttaccaaaacttgccttgtggtttaattagttgggtccttgtattatttctccaatttgacttcaagctccttgaaaataggaattatatcctactagtgtgcattccccataggcggtctttaacacagtgcctgcgcacagtaagttaaataaataactattttatgaattgaaaacaaagcccctttcatgcaagagcttgtaataaaaaatgtttacaaaaaggcaactaaaaggactagatatactaaataacagaaaattaagttgctgccacatctaaataaaaagcccaagtactactttacaaatataaggaaaacaatcttttttttttttttcaagacagagtctcactctgttgcccaggttggagtgcagtggtgcgatctcgggttacagcaacctccacctcctggattcaagtgattctcctgcctcagcctccccagtagctgggattacaggcatatgccaccatgcctggctaatttttgtaattttagtagagacggggtttcgccatgttggccaggctggtttcgaacccctgacctcaggtgatctgcccgcctcggcctccccaagtgctaggattacaggactgagccaccgtgcccggccaaggaaaacaatcctgatacttaacaaatcgacacacatacaaagctttcacatctgttgaccacaagaaatgattgtaatcttgtttctcattaagggaaagaaattcaagagctggaaccaatatatatgaagcttcgaacatccttgcatccttaaaaggatgttgctaaatactccttgagaactgagaccctttaaagtaattcataataagcatgagaatcggccagctctaatgacttgttcataggtaggcattttttttttctgagattcaatctgcaaaaagacgtctaggccaggaactcccaaactctgctgctgcacgttagaatcacctggggagctcttaaaactcctgaaacacagatgttagtagtttgtaacgatcaatagatgatctccacgtgtagcaaagaatgattcacagcctgggctaatttagctgaggaaaaagttctggtcatttcccactgtggccactagatgccgctacggcctgaagactggagaggttttctagtacttttccatcagaggaagggacgctggcgttgatgtttcctattacaaaggagcaaagagaacttactttgtgttagcttattttgagagaacaataccttggaaaagggagtttggatactgaatgatgaaatagattaaatgtctgctgaattgaatactagaatagtgaggtaatatttcaataattttatcttcactgtctacccattgacataggttccttagtttaaaaaaaaatgctttgatggaatgctaacattttggagatggaaaataccaggatataaagtaatgataggggaaggcaattgtttgctgctcccttgaggttttcaggactctgtgctctggtccttttgtcagggcctttctttctttttcttcttcttcttttttttttttttttttttttttaagacagagtctcactcttgtcgcccaggttacagtgtggtggagtgatctcagctcactgcaactgcagcctccacctcctgggttcaagcgattcccctgcctcaggctcccgagcagctgggattacaggtgcgtgccaccacgccaggctaatttttgtatttttagtagagatggggtttcaccatgttggccaggctggtctccaactcctgacctcaggtgatccacccacctaggcctccaaaagtgcagggattataggcgtgagccaccacgcccagccagggccattttttctatgcctggctatcgtctttgcttctaggtctgagcttatagaagtcttccttgattccctgctacaaaacatatactgttctcttcttacagcgtatttactagctgttctgtccaagctcctcatagtccaggaccttgttctgttcacccttctacccctagggcctatctagcagagtttccaaggcagataataatagctcttctgtacaataaaaggtcacaataggtcttttgtaaaatatgtgaaagaatggcttagggaatgtggggaaacactttgtaagttagatttgatggtgaaaagtatatgagaaaaacgcattatttttaaatagtttctgatgaaattggattcaacttaggaacctaacaaggaaaactggatagaatgccagttttaagaatagaatttcatagcttcatcaatacttagggctggagaagatttggaggtcatccagttaagcctctgctacgatgttttagattccatttcagcgttccatgctaagcagttgcctgggctttgattggagccatccaatcagtgcttccaaagcagacaattctgccaaactgtggtgactatttgaaagttattccttgtgtggagcccaaattttccctccagggcttctacccattggtcttaattcataccgctgcagctccacaggtggagactatgacatctgcccacgggcttccaaaacgatgagatcatggtctcctcctagctaagtgtcctcagctagtttgcatataataatattgaaatacttaatgtaataacagtaataagtaataataatcttttaggtttccttgccctctttcaaatacgtttcataaatgattcaatactatttcttatgatttgcgagaaaacaaatccgtcttctgggtgaccaataaaaagtgattcctccctgatgccacagagttgttttggtttttgtttttgttttgagacagagtcttgccctgttgcccaggctgaagtgcagtggtgcgatctcggcttactgcaatttacgcctcccaggttcaagcgattctcctgcctcagcttcccgagtaactggaactacaggcgcatgccaccacatccagctaattttgtatttttagtagagatggggtttcagcatgttgatcaggctggtctcgaactcccgacctcaagtgatctgcccacctcggcctcccaaagttctggggttataggagtgagccaccacgcccagccttacaatttgttaataagctcaagacagattagtgactatcaaatatcattgacacagctatttctacaaacatcttcacttactagaacatatctgattaagaactgcctttaccctcttcttttcctcagacgaagcaatttaaaggacttctcagataaagcagttcatgaagcaagcctaattggctttctgggcgggtgagaaactcaccaattctctataaagtgggtccaaggtgaaccacagagccacagcacacctctttcccttggtgactgccttcaccccatgagggttctctcctccagatgagaagctgatcatgcgcccacattttggttttatagaggcctgagaaagaaagcaaaaattagactttcaattgttgtatcataaacatttccacaactcctgcttctctggctatgagattatttgagttaaggcctgaggcgaagaataaaggaatcctgggattgaagatgatgtaaattatctcaattttctcacccgtaaaacaaaggtaaggtcaaaataatatatacctcagaaaataaaaagatgttcaagtaaaacgtgtagcaaagtccacatacatagtaatcattcaatacatgctatatatatgatagcctgcaaattcaaagaaggcaaagttttcctctcataattcatttctgtagcttttgcactagcaaagcatccggatacaaggtaggtgtccattccatacttgtgtaattaacaaacaaacaaacaggacatctgcagtgctggaaaagttgttattgatctaggtagtggttacatgagcgtatacgctttctaatttttaaattgtacattcatggtttatgtgcttttcacaatacaaacatttttaaaagtcataattctgtatgatatttcatttcctttacactattgtttcccagtggtcgtttaggcattgtttgaacatctctagtggtgagaacacgacaccccttggagtggcctattcgccatcacccttaagtgttttaagactgacacagtccttcgcagcttatgcactgctcaggtggagctgtccacagcgtttaggtagaagagtgtagtgagagaaaaagaaatactgctgggctcactgagccagggtagaagatcaaacaagctgcctcttcaaattgaatgagcttaagcaactgaatattagaggccttgcttctgtgtttaaagaagtgtctcatgtgagtagaatccattttgtctttcttgggaaatacagaaactttattcattcctctgtatagttcagaaaagtaaagtaaaaccaaaaaaaagtggcactagccattttactaggtgagaacatggactggttctcactagccattttctgaattgacttgagttgacatatgcccgtaaatctttggtttccttctgcataaaatgagctggctggtttttcactgctagcttcaggaagaatatttgaaaaatttcctaaagtccttggttgctacataacataggtactcccggactacgtgactacatatggaaggtaagaggtaggtggaggagtcggaaagccagagagacaggtgggtgcatatgtgtttatgtgggcatgtgtgtgtgtttatgtctgtgtgagtctgcatgtgtgtgtatatgtgacttgtgtgtgagtgttaaagggaagagcagaaaaaaagcagtcagtttctttttactcttctactcacaataactgaagagtgatttgtctttgctggaattgcttttcctaaacagctttaccaaggcaactgttctgatgtaaatctagagagttcacgagttagtcgtgtcctcagtatcaatgtgattctgtttttctggagaaggaatttgtacatttccacatagaagcagcaaaataagtgaaaggtaaaacctgtgaagtaggatagaaaagacttctgaggccgggcgtggtggctcacgcctataatcccagcactttaggaggccaaggcaggtggatcacctgaggtcaggagtttgagaccagcctggccaacatggtgaaaccccgtctctactaaaaatacaaaaattagccagatgtgttggtgggtgcctgtgatcccagctactcaggaggctgaggcaggagaatcgcttgaacctgggagacggaagttgcagtgagctgagatcgtgctactgcactccagtctgggtgacagaaaaaaaaaaaaagaaagaaagaaaagaaagaagagagggagagaaagaaagaaaaagagagagagaaagaaagaaaagaaagaaagaaagaaagagaaagaaggaaagaaaggaagaaagaaagaaagaaagaaaaaagaaagaaagaaagaaaaagaaagaaagaaagaaagaaagaaagaaagaaagaaagaaagaaagaaagaaagaaagaaagaaagaaaagccttctgagtaaatggccttgggatgttggaatttcaggttttctgggctgcccactccctggtgctggtcaggactcactcaaacacgcatttgccaatgtttctatgtgattaccaagtcttggtaacatcttataagactaaagaaaggatgtggggacaaattagtattttctttcttctggaattgaaagtaattttaaacatgaataactttgggaaagtttaggaaaagaagctgtgaactgtatgaggcggttggaactgattatagccgtggattcacaaggatctctaagacccatctcatcacacagcgaacatcatctgttacatgtcaggtatttggaggaataaacaagctttagtgaacctgcttccatgtgtgacattgtttctacagcaaaaatatgttatgaagtttggataaatgattataaacatacttttgtaacacagccccgtttatactgaatttaatttttcatgaaacaaatggaactcattaaattttatcttctttgaacctgcacagagaaaccaacatccttgaattcccaagtctcacaaacaaatgccgctattgaaaagaatccacatatcttccaagtatactaagcataatttaaaataatccttttgctttccgtctaactgatgattgatttagaaaagcgtttgtgctgtaattgtctttaataaaagtaaaatttcagccacctttcagataattacacaacttccagatttttccaaagtaaaggactcagaagacgttacttccttatgtggcctcaagttatgccatttctctgctccatttgtccctccacagtcaaagggaaactaaaacagaaaaatacccatttctgttgctgttgttgttgttgctggtactagaataaacttcctgtggggaaagataagaactgccttacgtaagaattttagttatctacgttattccctaaaatgccactgtatgcattaatatgaagtatttgaggaaataaatgttctcaaacaggagcttttggattaaaaatttgtttaaatcagttttagtttttttaggggaggttagattctgggtttttaaaaatagttatagattttaattaaaattgtagcactctgaaaggagaatattatctgctttcacatctttaaaaagtagaattccagttaacttgacattcaaaaattaatgaaacaggcatcccttgctttaagcatcaagcatttccaaatacggccactagatggaggagaagcaaagaaatcttttcaaccacctgtctgactttctttgaaactgcgaaatgccagtatttttgtacctcagtttcagtgttagcagagatcagaaataaaatgcttcctacactcaagctttcccagaagtgcaaaaaaccttctggactttaaactccaatacagatcgtcaatcttgatttaatttatcagattacggagtattaaagtactcccatattaacatttaagctttttacccaaaaagtgaggcatcatgactttttaacatatattattgtatgaagtcacatgaaatattttttcttagttattacaatgtttctagacaattttaacgagaatcctcataggaatcaccttttaacccagtaatacatactttttgatattacctgcttatatcacagtactgtgtgatgaataacatatgtgtgtcataaatgacagatatgtttcactaatgagaaaagcatgtaagctatgagggacaagcataaaattcaagaaaagtttagaaatagcttctataatgccaaaactttgaagtttatcacatacatgtcccaactacttagattttgtatggtggtgagagatacctggtcatctataatctgcttactttctcaattggaaatttttttattattattatacttttaagttctagtgtacatgtgcagaatgtgcaggtttgttacataggtgtacatgtgccatggtggtttgctgcaccccatcaaaccattatctacattaagtatttctcctaatgttatccccctgacaggccctggtgtgtgatgttaccctccgtgtccatgtgttctcattgttcagctcccacttatgagtgagaacatgtggtgtttggttttctgttcctgtgtttgtttgttgagaatgatggtttccagcttcatccatgtccctgaaaaggacatgaactcatccttttttatggctgcatagtcttccatggtatatatctgctacattttctttatccagtctatcattgatgagcatttgggttggtaccaagtctttgctattgtgaacagtgccgcaataaacatacatgtgcatatgtctttatagtagaatgatttataatcctttgggtatatacctagtaatgggattgctgggtcaaatggtatttctggttctagatccttgaggaattgccacactgtcttccacaatggttgaactaatttacactcccaccaacagtataaaagcattcctatttctccacatcctctccagcatctgttgtttcctgactttttaatgatcgccattctaactggcgtgagatggtatctcattgtggttttgatttgcatgtctctaatgaccagtgatgatgagctttttttcatatgtttgttggcctcataaatgtcttcttttgagaagtgtctgttcatatccttctcccactttttgatggggttgtttgtttgtttttcttgtaaatttgttaaagttctctgtagattctagatattagccctttgtcaaaaggataaattgcaaaaattttaaaacagaaaactgggctatattcttcaggccatggagggtttttgaccattccatggatcctatctgaggtatggcagtgagcagtaaacttttggctttactagcattttctttcggggtctgtaagtcccggtccaccaaaactgcttcctcgtgctccttccagcagtcataatctgtcaccatggcaatactcgcataacaaattccagcctctttagtgagaaccacctctggaactgtggtcatgttgataacatccaccccccaggtgcagaacatgaagctttctgctcaagagataaaacgaggtccctcagttgtgaccattgtcccctttgaatgtcaccagagtcctagcttcttagcagtctctataagaacgtctctcgtttgggggcttggcccccattggaatatggcacactcctctagcacaacaatgacttccatcatagagggactgaggtctcatactggtcctgtcaatgaactgatcaataatgacaatatcgccaggttgaatctcctccctcaaggagccacaagctgtggtccctatgacatgtgtacagccctcttccttcaaagcccagatggtcgcctggtagttgaccttggaagccaggatggtgtgctgcctcccatgccttgcaaggaggacacaatcaacattttttatcttccccaaaattaagacatcaaatggcttgccaaatggagtatccacatatttttcagttcttccttctaaaatttctggatcatccaggcctgttccaccaattattccaatcttcatggcagtggtggtggtgcttgaggccatgtccgcacaggaaaggaagctgcagtgggcgagcactcgggactaagggaatgaagtgggccaaagagcggcagcagtcgaaaatatatttttctaccattgtgtatgctctttggcttgaagaccaaaggatgctaactatatggtctttaagtaggcttctttccaaaggcacgggtttacttaggaagatggtcgttgaattgaacttttgatttactctttgtttacatatttgtgtgtgtgtgtgtgtgtgtgtatcaattttcaggtatgagtttattacatacagtcacaatcatatatctctctatgcatatatatatgcaaatatatatatatatatatatatatatatatatgaggccatgtctgcatgggaaaggaagctgcagtgggcatatttatatgtgtgtatatatattatatatatattttttatatatataatatatatacacacatacctataggtatatatacacatacacatacatacacacatatatgataattttggtaactatatatttgaaggccaatgctcagtccaagtacgtagaacaccaggttacttgaaagacaccttgtagaatcatttgacttattctagacttgggtgacttaggaactgacgttcttacgtttccatcagattacttgacaaagaattagaagacctaggttttggttccaagtctaacactaactagctatatgatcatgataaaatgattaatatctcaattctttaatttactcatctttacaacgtactagatttttatggatacttctaacagtttatgatctatacatttactgaaactctcttaacctcttgaaatcttgagctcagaagtatttgaaatctaaccatctgtaagtacttagaaaaatggcaatactgagctaaaacatactaaataagtattcaagaataggtttacttacagtcacagtcttagcatccatctctgtgaatatgaattctcctccttcaaagtcatcattcatatataggagagcactataagaatgaagagaaaactatttgtattattatatgcttatgagagcatggtcatagaatactgaagactggtgatgtactgtccagcctccattagtaagtttcctttgaactagacattgatataattggtcctccaaactcttaaagagaagatcagatagagtttgagttatacctctgaagagtttacttattccacctttactaaaattgtaatcatatgtcaaagcttgtctcacatttcacttcctccacagaaaacctaactagttttaatttaatctagccctttgaacgtgcccaccctaattacaactctctttttcccttatataactctaaaggatttttgtttgtttgtttgctttgttcttgttgttgtttttctctttaacttgctgaagagttccttgtggcctagccacgccttgcagatagcgggtgcttaataaatgttttggaatcgagtacagtgtagtaaggatgaacacatggtgactataactcctgattccttacacactgaaatctctctcaataggtaaggacattcttttctcctgagtttctagtgtcttatttataaaatgcagacatagttcttcttgggtatgaataaagataattgatgtgataaagtccttatgtaggccaagactccatgcccttgtattctgactgaaatacatataccacaaggaatatgttccagggagaacaggaatttttttaaaagaacataaaatttacttttcatattaaaacaaaatggacattttgtgaaatgggatgcaaactgtgcacaatttttaagataaaagtgagatgataatgccacttattggttatggcagctacttcagaagattatttgagaatttctgccttggagatatgaacacgacgtcaagcaaaacagagcacctttccagttttcactgcttgaaaactgttaggtaaaagctttgttttgaagcaggttctagctatacctatagtctcgaaatgtgtaagcaggaggctccttccagcattcgttggcctctggatccaacaaacagttgtcagcatggatgggatgactgaggtcatttcttctatcctgctgacctgccagaaaagggaatagggaagaacgagaaatgaagtgcagcaacttccaccaaagtaactgacaatctctcttctcccagtcctgatgatctttgcaaatgggcagaggcagacaacaggaaagaacagataaaagaacagtggacaagtagttgagattctggtctggttctgatatgaactggatgtgaaaccttgagcatgtcacacctgtctcttcagctataaaagttatttctgatataaacatacatgaacaaattccatgcgtttcatgggttgtacttcacatagggcagcaaaacaatgggaaggaaatgaaaattttttaaaaaaacaagatttaattcagctaaaacttcccaagtgtaaaagtctactcctatgacatcattattccccccgccccattcagaggggcataagacatagaggagaaataatgctgagcagggaccgagaaggtcccggtctttttaagagtatttctttcaatcatcccttcatcttctatgcttccacttaatgctggtagacatcatggtgactatgatttccatgttcctgattcctcaaaatgggaaacctacagcagaaaagcatttttttttcccaagacttcagcaattggggaaaattgatttggaagggaaaatgttcagaacgagggttttctggaacttcagaagaacaaaaggaagaaaaagaaagaagcagaataagagttcataagctacttttgttttggaaatctagaattcgtctaaaatattaatctaccatgctgttgagctttgttttgccttgtttcatttcccaatgtattgtgggtaaaagggaaccatttcagactgcaatctcaccagacagggctgttcggcagaccatgtgtgtataggaaaaatacagagttgagttcagcataaaataagattctacaatccttcgagccttttcgctgatgtcataaaacagacgagcgctcttcagtgggactcgaccttcataaccagactgaaaaaaaaaaacaaaacatgagaaacaaatgggttcattggaggtcgtaagaaaaacaggcactagcttgcattgagccaatataggattgtcatgtctgtattgactaatggggaaggctactacatatttgtggatttcggttaggacattgtgccattggaataaactcttcccaataacagatggaaagctaaaagctacttggttcctcttctcttctattataatatgttctaccatcctctctaacaacccttcctgcagtagagaataattacttgtttgaatatgttttcactattagatcatgattttacaaagagggattgtatgtattttttgtcaggaaatgtaaagtttgataagttgatggctgaacggaggaatgattaactacaatgaaggtttcatttcactcttgcttctttcagctgtcctgataggtatatattcaaaacttttttctttctttctttctttctttttttttttttttttttttttttttaagacggagtctcgctctcgcccaggccaggctggagtgcagtggcgcgatctcggctcaccgcaacctctgcctcccaggttcacgccattctcctgcctcagcctcccgagtagctgggactacaggcacccactaccatgcctggctaatttttttgtatttttagtagagacggggtttcaccatgttagccagggtggtctcgatctcctgacctcgtgatctgcctgcctcagcctcccaaagtgctgggattacaggcgtgagccactgtgcctggccacttttttctttcttatatctgccatttacagaagccttaggctgacactaaggaactcaaacccaaaagaagttaagactttactttgagtgctttcaggacagttgcaccttcaaacttttcattgggtgtatggggtgaagtttttcctctgtatccatcaccaacaagcatgattccctggaagcaaatacaagaagatacgtcatcaatgataactatgaattcatcaggtctttttctgctttggctgccagaaagctgagaactctagtcaatgctaagaaccatccccattttattccaggatgctggtataactattcttttaattaaaagagatatctttatttaatcttcattctacatttttattatcaaatactgtcactttttatggaaataaaaagaatgtaaattattgatatattgcctaataattaattaaaataaaaggatttctccagcaactttatatgataggcagggaccttgctagatggctgttgggctaggaaatatgccagtagctattccagatcccacccgggaaaactaaaataatcccaacccaaaggctataaaagttcctcaaaaataatttatgtctttctcctcaagaaagcaatatttcagggcttgttgtctggctccagatgagaccaggaccaaagcagttccagcttcttggcaggccagcgcagtgagctcccctccttctccggatcctcacactggccacgctgtggagctcccggcactgttcttccgacaggacgttatccaggagaacccgctgagtcccgttcagctgctccgagttgtagacgaatgtgatgttctcatagagtagaggaccacctacaggaacagagcacattgtcttccctgttacctctgtcccccgaaaggaagcacagaataccaaacagctttcaatgtgcaattcgagtgagtccatttcttcatgaatatttagaaatgcaaagacaaattgcaaattaggaagcttgataaacacaatatagattcaggagaaagatgtctatcgggtttcctcataatgtcataataatgttacatgcgtacattattttgggagagatagaactacttttgcatttgtttctaatgctgatcccaaaaagtcgactgggcatctcataggactctcttctgagaaagtccacccttgttcacagcccctcattcatacctctgtcgcaacaccacagattgcatgtttgggtccccaggtatgacatgcacacctcagtttcactggtgtctagagtaacaccttcactaggtgattcaggtagtcacacccaagtcacttgactctgcctcagtcttcttcctaccacacaactgaccctaggttgtcttgcttttggagtgtttttctctgttctccaggcaaaatcatcacgtttctatttccacacaggagggtttcccttaggcacctactctcttcccagagagagaccacaagtttataaaattaactccagataggctcttcagagggtgatgctttggggtcacattttaggttgtgaaattacaaaatccagacctgacaaagactttttttcatagagtaggactgaaggacaaagcctctgatagagaggaaatcttctggtgtagcatagtgcaaacattttgaaccagaaaatggaacagaaaagaaaacactgcttctaaccatttataacactcccaaggggcctggtgcgtgcttgtccatagcaacagccgaagataagacagcagggatagggaaggcacaactccctgccaccccaggtctagcaaaggtcaccaacacgttggcctaaaacactccaacagaggaagaagagacaattgaaattgatgtctactttataaaatagattagtcagcctagggaagagcactgtctaaatgtttattagtctagctaggataaaagactcactacagctcatgattaatggaatattaaaagcatgaggcagtgtttctcaatctagagtaatgttcggttatctgttaaagcagaagttccctaaggagctccagtgctgacaaattattttcactctaattttacttctatatatgcaatcataaaactatgttcacattatttattttagtagtttcatacagtacccaaaccaatttacaagactaaataaaagcaagaccataaaatgaactaaattccaatcaacaaatggaatggttaacgttatttttctgaaaataaaaagctaccttccacatgaatattctactcactgatctggtctgggttcttttgagccaggattccttatagtactgttggtgtgtaattactattctaccacaacttttctctagttaaactactacacaatcttaatgtaccacgattaattttggcctttgtatcagtccattttcttgctgctaataaacacatacctgagactgggaaattataaagaaaaagagatttaacggactcacagttccacgtctggggaggcctcacaatcatggtggaaggcaaagaaggagaaagtcatgtcttacatggtggcaggcaagagagcttgtgcagggaaactcccgtttataaaaccatcagatcttgtgagacttattcactaccaggagaacaaaccacccccatgattcaactatctccacctggccctgcccttgacatatggggattattacaattcaaggtgagatgtgggtggggacacagctaaaccatatcagctttcatctaagcctaatacattatttataagtgcatcgtatttcatttatttctagtctttaatcattgaagtattattagttggcgttaatatgaccataactgtgcccattgaactgtggaatcccatgacaccacctggctagaagttgatcccttagataacccctaattgcttctcttaatttttgaaaattgtgattaaaatttataaatatggaatttttggatcccaagaatatatccataagcctcgtttcagtatcacatcttttagttgaatagaaaaatatgcttttctacaattcactatttctcaaattgtgttgcattgaaagttctgggaaagtactgggttaaataaaagataagaggacgtctcagaacttttaatatgtgaacaggccttattaatgtcctaagtagtaacataatatgcagcagtttttagccttaattgaatacaattctctccctccccgactcccccaacttccttcctgcagcagagactttacgatgggctgcaatgatcattgcctcccaacattcatacagctgtgcaatttgcttcccacaagtgcgagtggaacctggaacttacttctaaacagtagaatatggcaaaggtgataaggtgtcatttctgtgactatgttacatgaaattgcaacttctctcttgccttctcagtctcttgccttctccgctgccatgctgtaatgaagcaagcggctgtactggagaggatcacatggcaaggaatagagggcgtcctccagccaacaaccaactaggaactaaaaggccatagtccaacaactttttgggaactgaattctcagccctggtcaacaacttgactgaaaccggtgagagatcctgaagccaaagacacagttatgttctctgcagactcttaccccacagaaactatgagataataaatggatattttcataagctgctatattttcattatgttttcacagcaataaaaaaagtagtctttaaaaaatttatttgacagggtctcactgtcactcaggctggagtgcagtggcaccaccatcatggctcactgcagccttgacctcctatcctcaagcaatcctcccacctcagactcccaagtagctgggactataggcacgcaccaccatgcctagtttatttttttttttgtcagggtcttgttatttttttattttcagggtcttgcccaggctggtcttcaactcctggactcaagccatcctccctcctcagcctcccaaagtgcttggattcgaggcatgagccaccacgcccagccaagtagtcttttaaaaatcccccctaaacgtcttagcatagaacgcaatttggaaaaggctgttctaatctatgtagctgattaaagtttgtagtcaaattgagacaggcattccctcgaatatttggaaaagtgaactgtatattccattccaatttagaagtataaaaagaaatgttccttcaagaagaaagataagatacttgtagattcactgaaaagcatctgctaataaaaactcaaatagaaatgaaagacagatgagagatttagtgaattaagttggtttatactttgccaagtttttctctttttctacattgaagatgtattattttaaacataattagcttattttcaaaatatgattttgaaagttagcaaaaatgaaatatttaggaaaatgtctaccagtcttaagctccattataagtatgtattatcattactatcgttattgttgaattaataagctacacctggaatactgtgttaagttctgattgagacaccttaataagcatagagccttaagagaaggctactgggatattgacaattctcctagcattccacaagtaacatgcaaagatcaagtgaaggaagaatgtaacatggagtcttcaatatctgaatcttgggttccgggagaatctgcattagaaatatgcctttcatgtagctaacataatcattaatattactaagtgtagaattttacacagatattttccacacgtacatttaacaaaaacgacccatcacccatcaactaaccaaaggaaaaaaaaaaagacaaatgaaaccctatcattcaacaggcaagtcaaccagcctccatatgactaccagtcttaagctggtagcttaatgactagtagcacaccagacagaggaaaacattaaaataaaaataaaaatgtagtccaataaattgtggtttctggggcctgatttgatggcttcagccaattcttaaccacagcccaagaagatttagtcaaacaagttaattcttccctcgtttataaagtatgtgcctggctctgactctctggttgcaacaagaagggatcatctaagaaagtactcccttgtactcactgtctggctcatggcaagaagaatcactttatcagaaattcgatagtgtcatacaaagcatgatggtgtaggcaggctaacaatccctatatgagtacagccttttacagttaaaaaaacaaaaaaagggcaaggcacggtggctcacacctgtaatcctagcactttgggaggctgaggcgagcagactgcctgagctcaggagttcaagaccagcctgggcaacacagtgaaaccccatctctactaaaacacaaaaaaaaaattcgctgggcatggtgatgcatgcctgtaatcctagctacttgggaggctgaggcaggagaatcacttgaacccaggaggcagaggttgcagtgagccaagatcgtgccactgtactccagcttgggcaacagagtgaggctctggctctaaaataaaaaaaaagaagaactttttatgtttaccatcttaaaacagttactatccttcttttatagataaaaatcatagggcccagaaaaattgagtcactagatcttcatcactgtggagcatatcagaataagataatcccagagactaagcctcctgattccaagtttattccccaacattgtttctactatccccctactgactctggaatcatatccagcacaacctttaaaaatctggtgggtgggctgagcacagtggctcaggcctgtaatcccagcactttgagagcccaaggcgggtggatcaactgaggtcgggagttcaagaccagcctgaccgagatggtgaaaccccgtctctactaaaaaaaaaaatgaagaaattagcctggcatggtggcatgcacctgtagtcccagctactcgggaggctgagacaggagaatcactggaatccaggaggcagagattgcagtgagctgagatcgcaccactgtactcccgcctgggtgacagagtgagactcttgtctcaaaaaataaaaataaaaaaaaaatctggtgagtgaaaggagaatggacaagtgacagcaatgttttttagtgagtttccatatcacgcatgtttcatggactgaagccactgtgggatgtaagtagccctgggtcaccccatcttaatgcttaggagctgtccagtaggaataatacaaaatactaaacactgataggcatccagagtagatgacagtctctttggggaagatgagaaaagctgaagtatactctattctgcgacacatgcctccctgacatccccttttagtcagattaagatatgaaaatgttgctgggcgtggtggctcatgcctgtaatctcagcactttgggaggccgtggcgggaggatcacaaggtcaggagttcgaggccagcctggccaatatggtgaaatctcgtctctactaaaaatgcaaaaattagccgggtatggtggcatgtgcctgtagtcctagctactcaggaggctgaggcagaagaatcgcttgaacccaggaggcggacgttgcagtgagctgagatcacgccactgcactccagcctgggggacagagcgagactccatctcaaaaaaaaagaaaaagagatatgaaagtgttttctggttggaaactttgtatctatctacaactgttcttaatttagatcctaattaggtatctgacgactggacaacaggctttttagttatgatataataagaaatatactggtttttatcccttgtttctagcacagagctcccaaatcccttggaatttcccaagggataagagtgtccttttttacttataatgagatccttttgatcacacttaagtttatgctgatgagatgaattagggtagggcccctaaaaagattcagaatggggctggtcaccagaaagaccaagtgattaggaaagtgaaacttttagccccacccacagacctctgggaaaggagtttggggcctgcaaattaagctctataaaaactcttgaacaacaagatgtgatgagcttctaggttgctgagcacgtggagatgctgggagggtggtttgcccatggagggcatggaagctcctcctgccatccccccataccttgccctatgtacctcttcatctagctgttcatctgtatcctttataataaaccagtaaacacaagagtttcctgagttctgtgggatgctctagcaaattaacctaacccaaagtcaggatcatgggaacactggtttgtagctggtcaatgaggggttggtcagaagtataggtaacaacttatacttgcaattggcatctgaaatgccagttgtgggctaagcccttaacctgtgggatctgacactatttccagacagacagtgtcagaactgaattggattagttcaccctcagttgctatccactggagaattactggtgcatgtgggtaaaacctcacacacatctggccacagaagtattctgtattgaatgaatatgagagtacggtaggtaaaaaaaaattttttttttccttcagaccagtaggttcccagaaaacattccatcctacaacccattagccacttcgtcctagacaattctgtggaaatggtagatttgtgtagcccaattaatcatgatccttctctatcacacgtagtcttctgttctgctcttctattaaccttctagtcagttgctggacaccagattccctggtagccaggggaaaaaaatgctttgaaatttggcaactgtcctggagtttcccaaacatttacagcaatgtgttggcttattggcatgaacttcctgggtgatttctcagcctaaaaggcaacatgaactctggctgaccatgaagctctctggcatttgtaaaacagcttgggctttgtggctgcaaactgaggtccaggtcagattagatgagcatattatcctgtttactggttcaaaactgacttggaagctaaggaaggaaagggatgggaaatgtgagagctgtagagagccaaaagttttcaaatcagaggaatgtgctgacaagatctcctggggaaagaagtactgtacagcttcccaatgcagcactttgaaagcctcttgtcttgcaattctttcatcaataggtgatatatttttgatatctacacaattctatgtttcatgcagcaggtgaagaagagaaaatgggatttcgggtcaaatgtctgggttcaaatgcctgttcaacctgttctgagcttctgagacttcagagaagtcattaaatcttcccacatgagatgctgttagtattaaaatagtatgcaaataaattgtgtgcaaatttgctgggaaatcataaaacatatagaaataataattgttaagaattaaccactgctgttagtattactattgctaatagtttttattaatataacaatgcttggctctacttaaaaactgtttttggcaacactccacctgttgtctctactaattttcatcttacataagtaggcacataatgttccgctcgcttcgtctctgtgcctttgctccgtttcttttaaccactagaatggttccctctacatccatgcatcctactacattctgctttctctaaaggccctgctcagatctatgtgcttcaagcatctaaggttaactaatctaatatcccatgtggctggtctcctctccaagttcctctgatatacagtctgtcccacactacctagctcttactgactgtgtaacagaagtaaaccataggaaattgctaagcctccggtattctgagaatggtgatgaccatgcaagtttccttgggaaacagatgagggagtgaatttggaggctttttctttttttctttttttttgaaacccatggtaataatacacaaatctaagacttttttttttaagtcttgctttcagactattagggagtgtagtagcttagtcttattctttattttctagagaaagtgggttcttccttgatctggaaaagaactggtcaagccccatcttccccttcccctcctttatagggtaaaagtgcacagctacttaccttctcttaggtctcgatctatcttgggtgatagcttttttcccattgagaatccatgaacttctgctccctctacgttcactcctgaagggaccctgcccattcaaaaaatttaaaatggcaatttgtcattgaataacgatgttcaaaggcaaagaatactttaccaaggtagtctgtgactctatattgtgtattttattgtaaacaagttcacctgtcttttaaagtcaaattaaatgtcactgcctctagtaagttctctatttcctcggtgagtttgccttatttcactcctctaaatcttcttagcaacctttattacaattaattgtatgcataacataactctcttcagattagactctaatctcaagtaggaaaaaaattatcatgagcatctttataatgctcagagtgtccaatggggcctggaacatggagtgcatcattgaacaactgttgaattgaaataaattatgtcataatgtagtagttcttgactattgtgaaaagaagacacaggtttgtcatatgcaaacacacaaatgatagcaggctgttctttatatacgtaaaaatcaaacaaatccaaatgtgtatatacattcttggatctctcgaaataattttatgccacccaagggttgagtttttgaagcctcgtgaaagacttcaagattctaaataagaagatctggatctgtccttcctttgtgatgagactctaagcaagttgcttgccttgtcagggcctcaggttcccaatttgaaaatgaataagctggaccatgtgatctcaagcaggtgtttctaaccttctgagtttcagtcctttaaaaagatgtaaattgacttttgtcatttttaaataatgagctttcttgggggaaggggagagggatagcat

SEQ ID NO: 54 - DNA, 5869 bp, Homo sapiens
gagttcctacttcgattttcacgaggttacaataaaatgtccttcaggactgctgttatatgaaggcttgatgtgggctggaggacccactcaaaggtggattgttcacacagatggcaaagtgggtactggctttcgaccagttcttctctgcaatgtcatctccatggggttacttgagtgtcctggaggcatggtagctgacttcctcctgagtaagcaaatcaggaaaacaaagccaaaatgctacaacaccttttaggacctactctcagaggtaatgccttgttatttctgccgtattctattggtcacagaggtcagggctaaatttttatgggaggtcataaaaatcaagaggcaaggatcattggggaccttttagaggtctggctagcacaaatacggacgcaggctgggcacagtagctcacatctgtaatgccagcactttggaaggctgaggcaggaggatcacttgagcccagcagcttgggaccagcctgggcagcatagtgaggcccgcacttctaaaataataataataataactcatgcattgcatatgacttacagtgagaaatctagaaaaggctggcaaatgcagtagtgtcaacagagtgctgggcaaaggatgtggcatttcatgggccaaatgtgaagctctctgggagagacctgatgtggagttacctaattggaaacatgggtataaaggccagaggcagcgatagattcatgaatatgaagcctgtacagccacacagagtcctccactaagaaggaccccacgcttcagtgtaatgcactgctattgccttcttgaaattctgaatactttctgaacaaggatcctgacattttcattttgcactgggccccacaaattatgtagccattcctaccttagtagccaagggtgaaaataatgatccatggaaataaagaggtgattgctctgtcaagaaaccatggatgagaaatccccagggatagggttttctaatgaactcataaaagcatctagaagaaagtaagttctaaactgctgcaaaaactaaacagatatctaattgctagatttctccaatagcctgagttcctccaaaaagcagagcttaagacaagagtttgcatgcaggtagtttgttttgggaaatgaccgctaaaataaaaaatgggagttagaaagagtaaaaggggaggagaaagagctggtccaaggttgcctcattgaactcattgctctgtgagcaactggggctcaatctcatcagagggctcgctaaggagctttgtaggatgctctgcaagactgaccttctgagacaaagaaagaggaagcatttgtcctccaattgccaccctgatggtcaaattgctctatgggtttacaatgctctcatgcatgcatcagaaaggctgagtgggttcctttgggatgctgcagggtggaagcaaagaggccctagagcagaaagcaaagaatacactatgtttccaagcgaatcccattacagctgttcgctgaagcaagagatggagtgaaaaagtggatgcgaggggctcccgtggcactacaatctccatgccaaagtcagccaaggaataaatttcttgctcctctactcactccttccctgagctccatccctggggagatgggggagggaaaaactagcaaagatagaagaaaccatctctagcccttccccagttgaggcttccctcttgagcaaggagaaggcaaaagcctcaactgtaatcaatttggaattacatgagaaaggggaatttaattcctccatttgagacatttctttatgataaagtgaaaagagcctaggaaagtcctggggcctgcctgagttttcttctagggccatggggattaacaagcccccagagtggacttaaaaggggttggatggtgtaagctggagaaagctgctttgtgattacctcctccaagcttgttcctcctaatcgaagacattagaaatatccagaatgcacttgatagaacaagaagacaatgttaagggggtgagggggaatgtgctgttagttgaattcctttcatgacacaaaaatggaagccagaactcacgtccatttcacatgtctttgctttttgagatggtgttcattctgcctgaaatatcctgctccaccatccgattcacctcccctatcatttgtcccctcctgagacagggagggttttgttattatcattatatggtattatcatatatatagtctgccttatactagaaatatttatggaggcttttttttaaatagagcaaaaatgacacaaattaaaaggcatggaaaatgaaagataaaaaataaaaaccaagtgcaggaaattataacagagtacagagcgagaccctgtctgtggctctacacataccttaagttactgacaggtgataagggtacagttttggctctcaactctcaattttgtaccagcagctgatacaaaaaagggaacatgttccagtctcagttagcatccataaggcaaaacccgcaccacttctcaggaaaagcagagctacttctgattctgaagtcaggtggacaattgcctctggggatctcctaaagagacacacaatgtacaaaaattgaacaacatgctccacaatatcttttctatttatttgttttatttatttatttatttttttgagacagagtctcgctctgtcgcccagactggagtacagtggagcaatcgtggctcactgcaacctccgcctccagagttcaaacgattcttgtgcctaagcctctcaagtggctgggattaccatgtgctaccatgcccggctaattttggtatttttaatagagacagactcctggcctcaagtgatccacccgcctcagcgtcccaaagtgctgggattacaggcatgagccacctcgcctggctaacatcttttctacaggcaattttttttttttttttttgagatggagtctcactgtcacccaggttggagtgcagtggcacgatctcggctcactgcaacttccacctcctgggttcaagcaattctcctgtctcagcctcccaagtagctgggactacaggcacccgccaccacacccggctaatttttgtatttttagtcaagacggggtttcaccatattggccaggctggtctcaaactcctgaccttgtgatctgcccacctcagtctcccaaagtgctgggattgcaggagtgagccactgcacccagctcaggcacttcttataatgtccctcaatctgtgacacagacatgacaaacaggagagaatcaccaagacccaggctgactgcagacaccttcgggccaccatgcactatggctttaatccccttcattgcactcaccatttctctttgcaatcatttctacatgaatctttctccctttattttttattatttatttaatttttgagatagggtctcactctgttgccaattctggagtgcagggttgcaatcacggctcactgcaacctcaacttcccaggcttaaatgatcctcccacctcagcctcctgaatacctgggaacataggtgtgtgtcgcaatgcctcactaattttttaaaattattttttatagacacagtgtcttgagggctaaaagccatgtcttgtttatgtccgtgccaccactgccaaacatttgggttggtatgtataccacaacatattttctgcataaatgagagaattacagattaatgcttttaattaatgcgaggcaacatagaaagcaggagacaaatgatcagatgccatttcgtctggagctcttgagaaactaaatcatgcacaaatgagatcagccagcctctaagctgcaagaagcaaggtggcacggaatcttccagtcaacctctgagtgaaagaatcaaaatgtatagtacagagatcacagattacaaaatgtctcaaaacttgtcatcaacgcccatgttggttacttatttctcgtagccatatctgcacggcatgtgcaccaagttggccaggctggtctcgaactcctgacctcggcctcccaaagtgctgggattacaggcatgagccaccgcgcccggccccttttcttctaagaattcatcttacaatttcagccatctctgaatttcacttagatgggcctctttggttggaggaacagcgtgataagtcttgatgtaaattttgacttagaagtgtaatagatagtgttcttttttatttttgttttgtttttgagacagggtctcactccattgcctaggctggagtgcagtggtgtgatcacagctccctgcagccttgacctcctaggctcaagcaatcctgccacctcagcctcctgagtaaaaaattagcatgtgccactgtgcctggctaatttttttttttttttttttttttttggagacagtcttactccgcctccagaggtcaagcgattctcgtgcctaagcctctcaagtagctgggattaccatgtgccaccacacctgactaattctggtatttttagtagagacagactcctagcctcaagtgatccacctgcctcagcctcccgaagtgctgggattacaggcgtgagccacctcgcctggctaacatcttctctacgggcaattcctttttttttttttgcgacggagtcttcctctgtcgcccaggctggagtgcagtggcatgatctcggctcactgcaacctccacctcccaggttcaagcaattctcctgcctcagcctccctagtagctgggactacacgcgcccgccaccacgtccagctaatttttgtatttttagtcgagacggggtttcaccatattgggcaggctggtctcaaactcctgaccttgtgatctgcctgcctcggactcccaaagtgctaggattacaagtgtgagcaaccgtgcctggataatttttaaattttgtagacatggaatcttactctgttgccctggctggtctcaaactccaggttacccaaagtactgcgattacaggtgtgagccaccactctcagccttttctttttctttttttacaaaataaaatacactaagatttgaagaaatctcactcatctttaaaaagattcaatggcaatgaatcacttttctgcaaaggctggagttaggagaacccgtggttgatggctttgttaattattattattattttttttaagattcaggttcacacttgtaatcccagcacattgggaggccaaggtggacagatcacctgagttcaggaattcgagaccagcctagccaacatggcaaaactccatccctactaaaaatacaaaaattagccaggtgtggtggcgcatgcctgtagtcccagctactcaggaggctgaggcaggagaatcacttaaacctgggaggggaatgttgaagtgaaccaagattgtgccactgcactccagcctgggtgacagagcaagactctgtctcaaaaaagcaaacaaacaaaaaaacaaattgcactgtttggtcaagaattcaattcacgataatcaagcagctcctaacacagttctctagtcacattttcagatttagcttctgtgggggaaaaaaaagtctgtatttccctgtagctctgtgagataagccttccatacctctcttgggttactatatttgaaggattcctgagatatggaatgaaagaactagcaaaaagctataatgatcactcacatttgattttaattagagaaaaaaacaaatctcttgctctcctccctcagctgggatgggattcttctccacgatgcctctatggtttctttccttgccttgagtgcaa

(19) United States
(12) Patent Application Publication
(10) Pub. No.: US 2011/0269143 A1
(43) Pub. Date: Nov. 3, 2011

(54) Genetic Variants as Markers for Use in Urinary Bladder Cancer Risk Assessment, Diagnosis, Prognosis and Treatment

(75) Inventors: Steinunn Thorlacius, Reykjavik (IS); Patrick Sulem, Reykjavik (IS)

(73) Assignee: deCODE Genetics ehf., Reykjavik (IS)

(21) Appl. No.: 13/002,614

(22) PCT Filed: Jul. 3, 2009
(86) PCT No.: PCT/IS2009/000007

§ 371 Date: Jan. 4, 2011

(30) Foreign Application Priority Data
Jul. 9, 2008 (IS) 8749

(51) Int. Cl. C12Q 1/68; C12M 1/34

(52) U.S. Cl. 435/6.14; 435/287.2

(57) ABSTRACT

It has been discovered that certain genetic variants correlate with risk of urinary bladder cancer in humans. The invention relates to use of such variants in methods of disease management of urinary bladder cancer, including various diagnostic methods.

### INTRODUCTION

Cancer, the uncontrolled growth of malignant cells, is a major health problem of the modern medical era and is one of the leading causes of death in developed countries. In the United States, one in four deaths is caused by cancer.

Urinary bladder cancer is the 6th most common type of cancer in the United States with approximately 67,000 new cases and 14,000 deaths from the disease in 2007.

Urinary bladder cancer (UBC) tends to occur most commonly in individuals over 60 years of age. Exposure to certain industrially used chemicals (derivatives of compounds called arylamines) is strong risk factor for the development of bladder cancers. Tobacco use (specifically cigarette smoking) is thought to cause 50% of bladder cancers discovered in male patients and 30% of those found in female patients. Thirty percent of bladder tumors probably result from occupational exposure in the workplace to carcinogens such as benzidine. Occupations at risk are metal industry workers, rubber industry workers, workers in the textile industry and people who work in printing. Certain drugs such as cyclophosphamide and phenacetin are known to predispose to bladder cancer. Chronic bladder irritation (infection, bladder stones, catheters, and bilharzia) predisposes to squamous cell carcinoma of the bladder.

Familial clustering of UBC cases suggests that there is a genetic component to the risk of the disease (Aben, K. K. et al. “Familial aggregation of urothelial cell carcinoma”. Int J Cancer 98, 274-8 (2002); Amundadottir, L. T. et al. “Cancer as a Complex Phenotype: Pattern of Cancer Distribution within and beyond the Nuclear Family.” PLoS Med 1, e65 Epub 2004 Dec. 28 (2004); Murta-Nascimento, C. et al. “Risk of bladder cancer associated with family history of cancer: do low-penetrance polymorphisms account for the increase in risk?” Cancer Epidemiol Biomarkers Prev 16, 1595-600 (2007)). Genetic segregation analyses have suggested that this component is multifactoral with many genes conferring small risks (Aben, K. K. et al. “Segregation analysis of urothelial cell carcinoma.” Eur J Cancer 42, 1428-33 (2006)). Many epidemiological studies have evaluated potential associations between sequence variants in candidate genes and bladder cancer, but the most consistent risk association to the disease is found for variations in the NAT2 gene. (Sanderson, S. et al., “Joint effects of the N-acetyltransferase 1 and 2 (NAT1 and NAT2) genes and smoking on bladder carcinogenesis: a literature-based systematic HUGE review and evidence synthesis.” Am J Epidemiol 166, 741-51 (2007)). Majority (>90%) of bladder cancers are transitional cell carcinomas (TCC) and arise from the urothelium. Other bladder cancer types include squamous cell carcinoma, adenocarcinoma, sarcoma, small cell carcinoma and secondary deposits from cancers elsewhere in the body.

TCCs are often multifocal, with 30-40% of patients having a more than one tumor at diagnosis. The pattern of growth of TCCs can be papillary, sessile (flat) or carcinoma-in-situ (CIS). Superficial tumors are defined as tumors that either do not invade, or those that invade but stay superficial to the deep muscle wall of the bladder. At initial diagnosis, 70% of patients with bladder cancers have superficial disease. Tumors that are clinically superficial are composed of three distinctive pathologic types. The majority of superficial urothelial carcinomas present as noninvasive, papillary tumors (pathologic stage pTa). 70% of these superficial papillary tumors will recur over a prolonged clinical course, causing significant morbidity. In addition, 5-10% of these papillary lesions will eventually progress to invasive carcinomas. These tumors are pathologically graded as either low malignant potential, low grade or high grade. High grade tumors have a higher risk of progression. Flat urothelial carcinoma in situ (CIS) are highly aggressive lesions and progress more rapidly than the papillary tumors. A minority of tumors invade only superficially into the lamina propria. These tumors recur 80% of the time, and eventually invade the detrusor muscle in 30% of cases. Approximately 30% of urothelial carcinomas invade the detrusor muscle at presentation. These cancers are highly aggressive. Those invasive tumors may spread by way of the lymph and blood systems to invade bone, liver, and lungs and have high morbidity (Kaufman, D. S. Ann Oncol 17, v106-112 (2006)).

The treatment of transitional cell or urothelial carcinoma is different for superficial tumors and muscle invasive tumors. Superficial bladder cancers can be managed without cystectomy (removing the bladder). The standard initial treatment of superficial tumors includes cystoscopy with trans-urethral resection of the tumor (TUR). The cystoscope allows visualization and entire removal of a bladder tumor. Adjuvant intravesical drug therapy after TUR is commonly prescribed for patients with tumors that are large, multiple, high grade or superficially invasive. Intravesical therapy consists of drugs placed directly into the bladder through a urethral catheter, in an attempt to minimize the risk of tumor recurrence and progression. About 50-70% of patients with superficial bladder cancer have a very good response to intravesical therapy. The current standard of care consists of urethra-cystoscopy and urine cytology every 3-4 months for the first two years and at a longer interval in subsequent years.

Cystectomy is indicated when bladder cancer is invasive into the muscle wall of the bladder or when patients with superficial tumors have frequent recurrences that are not responsive to intravesical therapy. The benefits of surgically removing the bladder are disease control, eradication of symptoms associated with bladder cancer, and long-term survival. For advanced bladder cancer that has extended beyond the bladder wall, radiation and chemotherapy are treatment options. Local lymph nodes are frequently radiated as part of the therapy to treat the Microscopic cancer cells which may have spread to the nodes. Current treatment of advanced bladder cancer can involve a combination of radiation and chemotherapy.

Early detection can improve prognosis, treatment options as well as quality of life of the patient. If screening methods could detect bladder cancers destined to become muscle invading while they are still superficial it is likely that a significant reduction in morbidity and mortality would result.

Cystoscopic examination is costly and causes substantial discomfort for the patient. Urine Cytology has poor sensitivity in detecting low-grade disease and its accuracy can vary between Pathology labs. Many urine-based tumor markers have been developed for detection and surveillance of the disease and some of these are used in routine patient care (Lokeshwar, V. B. et al. Urology 66, 35-63 (2005); Friedrich, M. G. et al. BJU Int 92, 389-92 (2003); Ramakumar, S. et al. J Urol 161, 388-94 (1999); Sozen, S. et al. Eur Urol 36, 225-9 (1999); Heicappell, R. et al. Urol Int 65, 181-4 (2000)).

However, no biomarker reported to date has shown sufficient sensitivity and specificity for detecting all types of bladder cancers in the clinic. It should be remembered that efficiency of screening increases with the disease's prevalence in the screened population. Therefore, the efficiency of the test could be increased by limiting the screening program to people at high risk. For bladder cancer, this may mean restricting participation to people with occupational exposure to known bladder carcinogens or individuals with known cancer predisposing variants.

There is clearly a need for improved diagnostic procedures that would facilitate early-stage bladder cancer detection and prognosis, as well as aid in preventive and curative treatments of the disease. In addition, there is a need to develop tools to better identify those patients who are more likely to have aggressive forms of bladder cancer from those patients that are diagnosed with the superficial disease. This would help to avoid invasive and costly procedures for patients not at significant risk.

Genetic risk is conferred by subtle differences in the genome among individuals in a population. Variations in the human genome are most frequently due to single nucleotide polymorphisms (SNPs), although other variations are also important. SNPs are located on average every 1000 base pairs in the human genome. Accordingly, a typical human gene containing 250,000 base pairs may contain 250 different SNPs. Only a minor number of SNPs are located in exons and alter the amino acid sequence of the protein encoded by the gene. Most SNPs may have little or no effect on gene function, while others may alter transcription, splicing, translation, or stability of the mRNA encoded by the gene. Additional genetic polymorphisms in the human genome are caused by insertions, deletions, translocations or inversion of either short or long stretches of DNA. Genetic polymorphisms conferring disease risk may directly alter the amino acid sequence of proteins, may increase the amount of protein produced from the gene, or may decrease the amount of protein produced by the gene.

As genetic polymorphisms conferring risk of common diseases are uncovered, genetic testing for such risk factors is becoming increasingly important for clinical medicine. Examples are apolipoprotein E testing to identify genetic carriers of the apoE4 polymorphism in dementia patients for the differential diagnosis of Alzheimer's disease, and of Factor V Leiden testing for predisposition to deep venous thrombosis. More importantly, in the treatment of cancer, diagnosis of genetic variants in tumor cells is used for the selection of the most appropriate treatment regime for the individual patient. In breast cancer, genetic variation in estrogen receptor expression or heregulin type 2 (Her2) receptor tyrosine kinase expression determine if anti-estrogenic drugs (tamoxifen) or anti-Her2 antibody (Herceptin) will be incorporated into the treatment plan. In chronic myeloid leukemia (CML) diagnosis of the Philadelphia chromosome genetic translocation fusing the genes encoding the Bcr and Abl receptor tyrosine kinases indicates that Gleevec (STI571), a specific inhibitor of the Bcr-Abl kinase should be used for treatment of the cancer. For CML patients with such a genetic alteration, inhibition of the Bcr-Abl kinase leads to rapid elimination of the tumor cells and remission from leukemia. Furthermore, genetic testing services are now available, providing individuals with information about their disease risk based on the discovery that certain SNPs have been associated with risk of many of the common diseases.

Loci Associated with Bladder Cancer

The genetic polymorphisms in a number of metabolic enzymes and other genes have been found as the modulators of bladder cancer risk. The most studied polymorphisms in connection with bladder cancer risk are polymorphisms in genes for some important enzymes, especially N-acetyltransferases (NATs), glutathione S-transferases (GSTs), DNA repair enzymes, and many others. An improved understanding of the molecular biology of urothelial malignancies is helping to define more clearly the role of new prognostic indices and multidisciplinary treatment for this disease.

It has been suggested that some of the NAT variants modify individual susceptibility to cancer. Slow NAT2 acetylation capacity has been suggested as conferring an increased risk of bladder, breast, liver and lung cancers, and a decreased risk of colon cancer, whereas a prominent change in the NAT1 gene, putatively associated with increased NAT1 activity, has been suggested as increasing the risk of bladder and colon cancer, and decreasing that of lung cancer (A. Hirvonen, IARC Sci Publ 148 (1999), pp. 251-270). NAT1 polymorphisms may affect the individual bladder cancer risk by interacting with environmental factors and interacting with the NAT2 gene (Cascorbi I, et al. Cancer Res 61:5051-6).

Glutathione S-transferases (GST) comprise a major group of enzymes that play a key role in detoxification of carcinogenic compounds. At least five GST families have been identified, and the effects of polymorphisms in these genes have been studied in bladder cancer. The results from these studies are contradictory but association between GSTM1 null genotype and bladder cancer is fairly constant (Wu, X. et al. Front Biosci 12, 192-213 (2007)).

Polymorphisms in genes coding for other metabolic enzymes such as NQO1, MPO or the CYP enzyme superfamily have also in some studies been found to be associated with bladder cancer but the results are controversial (Wu, X. et al. supra). Since bladder cancer has strong environmental risk factors, polymorphisms in DNA repair genes have been studied in bladder Cancer patients. These include genes for Xeroderma pigmentosum (XP) and X-ray repair cross-complementing (XRCC) genes. Many different polymorphisms have been tested but larger sample size and better matching between cases and controls is needed to conclude the effects of these variants on bladder cancer risks.

In short, despite the effort of many groups around the world, the genes that account for a substantial fraction of bladder cancer risk have not been identified. Although studies have implied that genetic factors are likely to be prominent in bladder cancer, only few genes have been identified as being associated with an increased risk for the disease. Thus, it is clear that the majority of genetic risk factors for bladder cancer remain to be found. It is likely that these genetic risk factors will include a relatively high number of low-to-medium risk genetic variants. These low-to-medium risk genetic variants may, however, be responsible for a substantial fraction of bladder cancer, and their identification, therefore, a great benefit for public health.

Clearly, identification of markers and genes that are responsible for susceptibility to particular forms of cancer (e.g., prostate cancer, breast cancer, lung cancer, melanoma, colon cancer, testicular cancer) is one of the major challenges facing oncology today. Some of the pathways underlying cancer are shared in different forms of cancer. As a consequence, genetic risk factors identified for one particular form of cancer may also represent a risk factor for other cancer types. Diagnostic and therapeutic methods utilizing these risk factors may therefore have a common utility. Accordingly, therapeutic measures developed to target such risk factors may have implications for cancer in general, and not necessarily only the cancer for which the risk factor is originally identified. There is a need to identify means for the early detection of individuals that have a genetic susceptibility to cancer so that more aggressive screening and intervention regimens may be instituted for the early detection and treatment of cancer. Cancer genes may also reveal key molecular pathways that may be manipulated (e.g., using small or large molecule weight drugs) and may lead to more effective treatments regardless of the cancer stage when a particular cancer is first diagnosed.

### SUMMARY OF THE INVENTION

A genome wide SNP association study of urinary bladder cancer (UBC) was conducted. The inventors were able to identify common sequence variants that associate with UBC in populations of European ancestry. The present invention relates to methods of risk assessment of urinary bladder cancer (UCB). This includes methods of determining an increased susceptibility to UBC in an individual, as well as methods of determining a decreased susceptibility to UBC or diagnosing a protection against UBC, in an individual, by evaluating certain markers or haplotypes that have been found to be associated with UBC, as described further herein.

In a first general aspect, the invention provides a method for determining a susceptibility to urinary bladder cancer in a human individual, the method comprising determining the presence or absence of at least one allele of at least one polymorphic marker in a nucleic acid sample obtained from the individual or in a genotype dataset derived from the individual, wherein determination of the presence of the at least one allele is indicative of a susceptibility to urinary bladder cancer for the individual. This implies that determination of the absence of said at least one allele is indicative that the susceptibility due to the allele is not present in the individual. Said at least one polymorphic marker is suitably selected from the polymorphic markers set forth in Table 1, Table 4, and Table 5, and markers in linkage disequilibrium therewith, and the at least one polymorphic marker preferably is a polymorphic marker as shown in anyone of SEQ ID NO:1-10, or a marker in linkage disequilibrium with any of said markers. The at least one polymorphic marker may also be selected from the group consisting of rs9642880, rs710521, rs12982672, rs12584999, rs233716, rs233722, rs10240737, rs17418689 and rs4733677, and markers in linkage disequilibrium therewith. More preferably, the at least one polymorphic marker is selected from the group consisting of rs9642880 as set forth in SEQ ID NO:1, rs710521 as set forth in SEQ ID NO:2, and markers in linkage disequilibrium therewith. Further useful polymorphic markers include rs12547643 (SEQ ID NO: 11), and rs17186926 (SEQ ID NO: 12). In another embodiment, the at least one polymorphic marker is selected from rs710521 (SEQ ID NO: 2), and markers in linkage disequilibrium therewith. In one embodiment, markers in linkage disequilibrium with rs710521 are selected from the group consisting of the markers listed in Table 3 (SEQ ID NO: 13-52). In another embodiment, the at least one polymorphic marker is selected from the group consisting of rs4733677 and markers in linkage disequilibrium therewith.

In certain embodiments, markers associated with risk of urinary bladder cancer are located Within a linkage disequilibrium (LD) block. In certain embodiments, markers predictive of risk of urinary bladder cancer on chromosome 8q24 (e.g., rs9642880, and markers in linkage disequilibrium therewith) are located within LD block C08, as set forth in SEQ ID NO:54. In certain other embodiments, markers predictive of risk of urinary bladder cancer on chromosome 3q28 (e.g., rs710521, and markers in linkage disequilibrium therewith) are located within LD block C03, as set forth in SEQ ID NO:53 herein.

In another aspect, the invention provides a method of determining a susceptibility to urinary bladder cancer in a human individual, the method comprising obtaining nucleic acid sequence data about a human individual identifying at least one allele of at least one polymorphic marker selected from the group consisting of rs9642880, rs710521, rs12982672, rs12584999, rs233716, rs233722, rs10240737, rs17418689 and rs4733677, and markers in linkage disequilibrium therewith, wherein different alleles of the at least one polymorphic marker are associated with different susceptibilities to urinary bladder cancer in humans, and determining a susceptibility to urinary bladder cancer from the nucleic acid sequence data.

In a general sense, genetic markers lead to alternate sequences at the nucleic acid level. If the nucleic acid marker changes the codon of a polypeptide encoded by the nucleic acid, then the marker will also result in alternate sequence at the amino acid level of the encoded polypeptide (polypeptide markers).

Determination of the identity of particular alleles at polymorphic markers in a nucleic acid or particular alleles at polypeptide markers comprises whether particular alleles are present at a certain position in the sequence. Sequence data identifying a particular allele at a marker comprises sufficient sequence to detect the particular allele. For single nucleotide polymorphisms (SNPs) or amino acid polymorphisms described herein, sequence data can comprise sequence at a single position, i.e. the identity of a nucleotide or amino acid at a single position within a sequence.

In certain embodiments, it may be useful to determine the nucleic acid sequence for at least two polymorphic markers. In other embodiments, the nucleic acid sequence for at least three, at least four or at least five or more polymorphic markers is determined. Haplotype information an be derived from an analysis of two or more polymorphic markers. Thus, in certain embodiments, a further step is performed, whereby haplotype information is derived based on sequence data for at least two polymorphic markers. In some embodiments, the susceptibility determined by the method which is conferred by the presence of the at least one allele or haplotype is increased susceptibility.

The invention also provides a method of determining a susceptibility to urinary bladder cancer (UBC) in a human individual, the method comprising obtaining nucleic acid sequence data about a human individual identifying both alleles of at least two polymorphic markers associated with UBC, determine the identity of at least one haplotype based on the sequence data, and determining a susceptibility to UBC from the haplotype data.

In certain embodiments, determination of a susceptibility comprises comparing the nucleic acid sequence data to a database containing correlation data between polymorphic markers from those described herein (e.g., markers as shown in Tables 1, 4 and 5) and/or those in linkage disequilibrium therewith and susceptibility to UBC. The sequence database can for example be provided as a look-up table that contains data that indicates the susceptibility of UBC for any one, or a plurality of, particular polymorphisms. The database may also contain data that indicates the susceptibility for a particular haplotype that comprises at least two polymorphic Markers.

In some embodiments of the method, presence of allele T in rs9642880, allele A in rs710521, allele G in rs12982672, allele A in rs12584999, allele A in rs233716, allele T in rs233722, allele A in rs10240737, allele G in rs17418689 and allele T in rs4733677 is indicative of increased susceptibility to urinary bladder cancer.

As described in more detail herein, the presence of the at least one allele or haplotype is in some embodiments indicative of increased susceptibility to urinary bladder cancer with a relative risk (RR) or odds ratio (OR) of at least 1.20.

In some other embodiments, the susceptibility conferred by the presence of the at least one allele or haplotype is decreased susceptibility.

It will be appreciated that the method can in some embodiments further comprise analyzing non-genetic information to make risk assessment, diagnosis, or prognosis of the individual. Such non-genetic information may include but is not limited to one or more of age, gender, ethnicity, socioeconomic status, previous disease diagnosis, medical history of subject, family history of urinary bladder cancer, history of occupational exposure to chemicals, biochemical measurements, and clinical measurements. As discussed further herein, information concerning tobacco smoking habits and/or tobacco smoking history of said individual may be particularly useful in connection with the genetic assessment of the invention.

In a further aspect, a kit is provided for assessing susceptibility to urinary bladder cancer in a human individual. The kit may comprise reagents for selectively detecting at least one allele of at least one polymorphic marker in the genome of the individual, wherein the at least one polymorphic marker is selected from the group consisting of rs9642880, rs710521, rs12982672, rs12584999, rs233716, rs233722, rs10240737, rs17418689 and rs4733677, and markers in linkage disequilibrium therewith, and collection of data comprising correlation data between the at least one polymorphism and susceptibility to urinary bladder cancer.

Preferably, the at least one polymorphic marker is one or more of the above mentioned, including but not limited to rs9642880 (SEQ ID NO:1) or rs710521 (SEQ ID NO:2).

In some embodiments of the kit, the reagents comprise at least one contiguous oligonucleotide that hybridizes to a fragment of the genome of the individual comprising the at least one polymorphic marker, a buffer and a detectable label.

In certain embodiments, the reagents in said kit comprise at least one pair of oligonucleotides that hybridize to opposite strands of a genomic nucleic acid segment obtained from the subject, wherein each oligonucleotide primer pair is designed to selectively amplify a fragment of the genome of the individual that includes one polymorphic marker, wherein the fragment is preferably at least 30 base pairs in size.

In certain embodiments of the kit of the invention, the at least one oligonucleotide is completely complementary to the genome of the individual.

The kit may in some embodiments comprise: a detection oligonucleotide probe that is from 5-100 nucleotides in length; an enhancer oligonucleotide probe that is from 5-100 nucleotides in length; and an endonuclease enzyme; wherein the detection oligonucleotide probe specifically hybridizes to a first segment of the nucleic acid whose nucleotide sequence is set forth in Table 1 (SEQ ID NO: 1-10), Table 4 (SEQ ID NO: 11-12) or Table 5 (SEQ ID NO: 13-52), and wherein the detection oligonucleotide probe comprises a detectable label at its 3′ terminus and a quenching moiety at its 5′ terminus; wherein the enhancer oligonucleotide is from 5-100 nucleotides in length and is complementary to a second segment of the nucleotide sequence that is 5′ relative to the oligonucleotide probe, such that the enhancer oligonucleotide is located 3′ relative to the detection oligonucleotide probe when both oligonucleotides are hybridized to the nucleic acid; wherein a single base gap exists between the first segment and the second segment, such that when the oligonucleotide probe and the enhancer oligonucleotide probe are both hybridized to the nucleic acid, a single base gap exists between the oligonucleotides; and wherein treating the nucleic acid with the endonuclease will cleave the detectable label from the 3′ terminus of the detection probe to release free detectable label when the detection probe is hybridized to the nucleic acid.

Obtaining nucleic acid sequence data can in certain embodiments comprise obtaining a biological sample from the human individual and analyzing sequence of the at least one polymorphic marker in nucleic acid in the sample. Analyzing sequence can comprise determining the presence or absence of at least one allele of the at least one polymorphic marker. Determination of the presence of a particular susceptibility allele (e.g., an at-risk allele) is indicative of susceptibility to the condition in the human individual. Determination of the absence of a particular susceptibility allele is indicative that the particular susceptibility is not present in the individual.

In some embodiments, obtaining nucleic acid sequence data comprises obtaining nucleic acid sequence information from a pre-existing record. The pre-existing record can for example be a computer file or database containing sequence data, such as genotype data, for the human individual, for at least one polymorphic marker.

Susceptibility determined by the diagnostic methods of the invention can be reported to a particular entity. In some embodiments, the at least one entity is selected from the group consisting of the individual, a guardian of the individual, a genetic service provider, a physician, a medical organization, and a medical insurer. In another aspect, the invention relates to a method of diagnosing a susceptibility to urinary bladder cancer in a human individual, the method comprising determining the presence or absence of at least one allele of at least one polymorphic marker in a nucleic acid sample obtained from the individual, wherein the at least one polymorphic marker is associated UBC, and wherein the presence of the at least one allele is indicative of a susceptibility to UBC. In particular, said at least one polymorphic marker is from the group of markers in Tables 1, 4 and 5 and those in linkage disequilibrium therewith. The method may also include determination of the presence or absence of the at least one allele of at least one polymorphic marker in a genotype dataset from the individual.

In yet a further aspect, a method is provided for genotyping a nucleic acid sample obtained from a human individual at risk for, or diagnosed with, urinary bladder cancer, comprising determining the presence or absence of at least one allele of at least one polymorphic marker in the sample, wherein the at least one marker is selected from the group consisting of rs9642880, rs710521, rs12982672, rs12584999, rs233716, rs233722, rs10240737, rs17418689 and rs4733677, and markers in linkage disequilibrium therewith, and wherein the presence or absence of the at least one allele of the at least one polymorphic marker is indicative of a susceptibility of urinary bladder cancer. The at least one marker is preferably selected from rs9642880 (SEQ ID NO:1) and rs710521 (SEQ ID NO:2).

The genotyping method comprises in some embodiments amplifying a segment of a nucleic acid that comprises the at least one polymorphic marker by Polymerase Chain Reaction (PCR), using a nucleotide primer pair flanking the at least one polymorphic marker.

The genotyping can be performed but is not limited to using a process such as allele-specific probe hybridization, allele-specific primer extension, allele-specific amplification, nucleic acid sequencing, 5′-exonuclease digestion, molecular beacon assay, oligonucleotide ligation assay, size analysis, and single-stranded conformation analysis.

In some embodiments the genotyping method comprises:
contacting copies of the nucleic acid with a detection oligonucleotide probe and an enhancer oligonucleotide probe under conditions for specific hybridization of the oligonucleotide probe with the nucleic acid; wherein
   the detection oligonucleotide probe is from 5-100 nucleotides in length and specifically hybridizes to a first segment of a nucleic acid whose nucleotide sequence is comprised in any of SEQ ID NO:1, SEQ ID NO:2, or SEQ ID:11-52;
   the detection oligonucleotide probe comprises a detectable label at its 3′ terminus and a quenching moiety at its 5′ terminus;
   the enhancer oligonucleotide is from 5-100 nucleotides in length and is complementary to a second segment of the nucleotide sequence that is 5′ relative to the oligonucleotide probe, such that the enhancer oligonucleotide is located 3′ relative to the detection oligonucleotide probe when both oligonucleotides are hybridized to the nucleic acid; and
   a single base gap exists between the first segment and the second segment, such that when the oligonucleotide probe and the enhancer oligonucleotide probe are both hybridized to the nucleic acid, a single base gap exists between the oligonucleotides;
treating the nucleic acid with an endonuclease that will cleave the detectable label from the 3′ terminus of the detection probe to release free detectable label when the detection probe is hybridized to the nucleic acid; and
measuring free detectable label, wherein the presence of the free detectable label indicates that the detection probe specifically hybridizes to the first segment of the nucleic acid, and indicates the sequence of the polymorphic site as the complement of the detection probe.

In another aspect of the invention a method is provided for selecting candidates for screening programs for urinary bladder cancer, comprising: assessing with the methods described herein susceptibility to urinary bladder cancer in a group of individuals, wherein individuals who are determined to have increased susceptibility to urinary bladder cancer are selected as candidates for a screening program for urinary bladder cancer. Said screening program can preferably be selected from a urine dipstick test for hematuria, cystoscopy and urine cytology.

A further aspect of the invention provides a method of assessing an individual for probability of response to a urinary bladder cancer therapeutic agent, comprising: determining the presence or absence of at least one allele of at least one polymorphic marker in a nucleic acid sample obtained from the individual, wherein the at least one polymorphic marker is selected from the group consisting of rs9642880, rs710521, rs12982672, rs12584999, rs233716, rs233722, rs10240737, rs17418689 and rs4733677, and markers in linkage disequilibrium therewith, wherein the presence of the at least one allele of the at least one marker is indicative of a probability of a positive response to the therapeutic agent.

The invention provides in yet another aspect a method of predicting prognosis of an individual diagnosed with urinary bladder cancer, the method comprising determining the presence or absence of at least one allele of at least one polymorphic marker in a nucleic acid sample obtained from the individual, wherein the at least one polymorphic marker is selected from the group consisting of rs9642880, rs710521, rs12982672, rs12584999, rs233716, rs233722, rs10240737, rs17418689 and rs4733677, and markers in linkage disequilibrium therewith, Wherein the presence of the at least one allele is indicative of a worse prognosis of the urinary bladder cancer in the individual.

Another aspect of the invention provides a method of monitoring progress of treatment of an individual undergoing treatment for urinary bladder cancer, the method comprising determining the presence or absence of at least one allele of at least one polymorphic marker in a nucleic acid sample obtained from the individual, wherein the at least one polymorphic marker is suitably selected from the group consisting of rs9642880, rs710521, rs12982672, rs12584999, rs233716, rs233722, rs10240737, rs17418689 and rs4733677, and markers in linkage disequilibrium therewith, wherein the presence of the at least one allele is indicative of the treatment outcome of the individual. The at least one polymorphic marker can be selected in certain embodiments from rs9642880 (SEQ ID NO:1), rs710521 (SEQ ID NO:2) and markers in linkage disequilibrium therewith.

Still another aspect of the invention provides for the use of an oligonucleotide probe in the manufacture of a reagent for diagnosing and/or assessing susceptibility to urinary bladder cancer in a human individual, wherein the probe hybridizes to a segment of a nucleic acid whose nucleotide sequence is set forth in SEQ ID NO:1, SEQ ID NO:2 or SEQ ID NO:11-52, wherein said probe can be 15-500 nucleotides in length.

The invention provides in yet a further aspect a computer-readable medium having computer executable instructions, for determining susceptibility to urinary bladder cancer in an individual, the computer readable medium comprising:
data indicative of at least one polymorphic marker; and
a routine stored on the computer readable medium and adapted to be executed by a processor to determine risk of developing urinary bladder cancer for the least one polymorphic marker, wherein the at least one polymorphic marker is selected from the group consisting of rs9642880, rs710521, rs12982672, rs12584999, rs233716, rs233722, rs10240737, rs17418689 and rs4733677, and markers in linkage disequilibrium therewith.

Said data representing at least one polymorphic marker can in some embodiments comprise parameters indicative of susceptibility to urinary bladder linked to said at least one polymorphic marker. In certain embodiments said data may comprise data indicative of allelic status of said at least one allelic marker in said individual.

Said routine can in some embodiments be adapted to receive input data indicative of allelic status of said at least one allelic marker in said individual.

As will be appreciated, said at least one polymorphic marker is preferably selected from the markers described herein, including those listed in Table 1 (SEQ ID NO: 1-10), Table 4 (SEQ ID NO: 11-12) and Table 5 (SEQ ID NO: 13-52), and markers in linkage disequilibrium therewith.

In a related aspect, an apparatus is provided for determining a genetic indicator for urinary bladder cancer in a human individual, comprising:
a processor,
a computer readable memory having computer executable instructions adapted to be executed on the processor to analyze marker and/or haplotype information for at least one human individual with respect to at least one polymorphic marker selected from the group consisting of rs9642880, rs710521, rs12982672, rs12584999, rs233716, rs233722, rs10240737, rs17418689 and rs4733677, and markers in linkage disequilibrium therewith, and generate an output based on the marker or haplotype information, wherein the output comprises a risk measure of the at least one marker or haplotype as a genetic indicator of urinary bladder cancer for the human individual.

The computer readable memory of said apparatus comprises in some embodiments data indicative of the frequency of at least one allele of at least one polymorphic marker or at least one haplotype in a plurality of individuals diagnosed with, or presenting symptoms associated with, urinary bladder cancer, and data indicative of the frequency of at the least one allele of at least one polymorphic marker or at least one haplotype in a plurality of reference individuals, and wherein a risk measure is based on a comparison of the at least one marker and/or haplotype status for the human individual to the data indicative of the frequency of the at least one marker and/or haplotype information for the plurality of individuals diagnosed with urinary bladder cancer.

In one embodiment, the computer readable memory further comprises data indicative of the frequency of at least one allele of at least one polymorphic marker or at least one haplotype in a plurality of individuals diagnosed with the condition, and data indicative of the frequency of at the least one allele of at least one polymorphic marker or at least one haplotype in a plurality of reference individuals, and wherein the risk measure of developing the condition is based on a comparison of the frequency of the at least one allele or haplotype in individuals diagnosed with the condition and reference individuals.

Determination of the presence or absence of an allele implies the determination of the presence or absence of a particular allele, or alternatively multiple alleles. Determination of the presence or absence of one particular allele of a biallelic marker (for which there are only two alleles possible) indirectly provides information about the presence or absence of the alternate allele. For example, for a C/T SNP polymorphism, determination of the absence of a C at the SNP in a particular genome implies that the genome contains two copies of the alternate allele (the T allele). Determination of the presence of one copy of the C allele likewise indicates the presence of one copy of the alternate T allele. For polymorphisms that have more than two possible alleles, such as microsatellites, the determination of the presence or absence of an allele does hot provide by itself provide information about the presence or absence of other alleles of the marker. In certain embodiments, the identity of particular alleles is performed, i.e. the nucleotide sequence at the particular allelic site is determined. Such embodiments provide a direct indication of the presence or absence of particular alleles.

In certain embodiments of the invention, linkage disequilibrium is characterized by particular numerical values of the linkage disequilibrium measures r² and |D′|. In certain embodiments, linkage disequilibrium between genetic elements (e.g., markers) is defined as r²>0.1 (r² greater than 0.1). In other words, for genetic markers with a correlation coefficient r² of greater than 0.1 are considered bo be in linkage disequilibrium. In some embodiments, linkage disequilibrium is defined as r²>0.2. Other embodiments can include other definitions of linkage disequilibrium, such as r²>0.25, r²>0.3, r²>0.35, r²>0.4, r²>0.45, r²>0.5, r²>0.55, r²>0.6, r²>0.65, r²>0.7, r²>0.75, r²>0.8, r²>0.85, r²>0.9, r²>0.95, r²>0.96, r²>0.97, r²>0.98, or r²>0.99. Linkage disequilibrium can in certain embodiments also be defined as |D′|>0.2, or as |D′|>0.3, |D′|>0.4, |D′|>0.5, |D′|>0.6, |D′|>0.7, |D′|>0.8, |D′|>0.9, |D′|>0.95, |D′|>0.98 or |D′|>0.99. In certain embodiments, linkage disequilibrium is defined as fulfilling two criteria of r² and |D′|, such as r²>0.2 and |D′|>0.8. Other combinations of values for r² and |D′| are also possible and within scope of the present invention, including but not limited to the values for these parameters set forth in the above.

### BRIEF DESCRIPTION OF THE DRAWINGS

The foregoing and other objects, features and advantages of the invention will be apparent from the following more particular description of preferred embodiments of the invention.

FIG. 1: A schematic view of the structure and association results in the cancer-associated region on chromosome 8q24.21

A) The pair-wise correlation structure in an 800 kb interval (128.1-128.9 Mb, NCBI B35) on chromosome 8q24. The upper plot shows pair-wise D′ for 959 common SNPs (with MAF>5%) from the HapMap (v21) CEU dataset. The lower plot shows the corresponding r² values.
B) Estimated recombination rates (saRR) in cM/Mb from the HapMap (v21) Phase II data,
C) Location of known genes in the region.
D) Schematic view of the association with bladder cancer for all SNPs tested in the region for the initial scan (Iceland and the Netherlands). Also indicated (red arrows) are the locations of previously identified associations to prostate cancer (PrCa), breast cancer (BrCa) and colorectal cancer (CoCa).

FIG. 2: Schematic view of an exemplary computer system for implementing the invention.

### DETAILED DESCRIPTION

### Definitions

Unless otherwise indicated, nucleic acid sequences are written left to right in a 5′ to 3′ orientation. Numeric ranges recited within the specification are inclusive of the numbers defining the range and include each integer or any non-integer fraction within the defined range. Unless defined otherwise, all technical and scientific terms used herein have the same meaning as commonly understood by the ordinary person skilled in the art to which the invention pertains.

The following terms shall, in the present context, have the meaning as indicated:

A “polymorphic marker”, sometime referred to as a “marker”, as described herein, refers to a genomic polymorphic site. Each polymorphic marker has at least two sequence variations characteristic of particular alleles at the polymorphic site. Thus, genetic association to a polymorphic marker implies that there is association to at least one specific allele of that particular polymorphic marker. The marker can comprise any allele of any variant type found in the genome, including SNPs, mini- or microsatellites, translocations and copy number variations (insertions, deletions, duplications). Polymorphic markers can be of any measurable frequency in the population. For mapping of disease genes, polymorphic markers with population frequency higher than 5-10% are in general most useful. However, polymorphic markers may also have lower population frequencies, such as 1-5% frequency, or even lower frequency, in particular copy number variations (CNVs). The term shall, in the present context, be taken to include polymorphic markers with any population frequency.

An “allele” refers to the nucleotide sequence of a given locus (position) on a chromosome. A polymorphic marker allele thus refers to the composition (i.e., sequence) of the marker on a chromosome. Genomic DNA from an individual contains two alleles (e.g., allele-specific sequences) for any given polymorphic marker, representative of each copy of the marker on each chromosome. Sequence codes for nucleotides used herein are: A=1, C=2, G=3, T=4. For microsatellite alleles, the CEPH sample (Centre d'Etudes du Polymorphisme Humain, genomics repository, CEPH sample 1347-02) is used as a reference, the shorter allele of each microsatellite in this sample is set as 0 and all other alleles in other samples are numbered in relation to this reference. Thus, e.g., allele 1 is 1 by longer than the shorter allele in the CEPH sample, allele 2 is 2 by longer than the shorter allele in the CEPH sample, allele 3 is 3 by longer than the lower allele in the CEPH sample, etc., and allele −1 is 1 by shorter than the shorter allele in the CEPH sample, allele −2 is 2 by shorter than the shorter allele in the CEPH sample, etc.

Sequence conucleotide ambiguity as described herein is as proposed by IUPAC-IUB. These codes are compatible with the codes used by the EMBL, GenBank, and PIR databases.

| IUB code | Meaning |
|---|---|
| A | Adenosine |
| C | Cytidine |
| G | Guanine |
| T | Thymidine |
| R | G or A |
| Y | T or C |
| K | G or T |
| M | A or C |
| S | G or C |
| W | A or T |
| B | C, G or T |
| D | A, G or T |
| H | A, C or T |
| V | A, C or G |
| N | A, C, G or T (any base) |

A nucleotide position at which more than one sequence is possible in a population (either a natural population or a synthetic population, e.g., a library of synthetic molecules) is referred to herein as a “polymorphic site”.

A “Single Nucleotide Polymorphism” or “SNP” is a DNA sequence variation occurring when a single nucleotide at a specific location in the genome differs between members of a species or between paired chromosomes in an individual. Most SNP polymorphisms have two alleles. Each individual is in this instance either homozygous for one allele of the polymorphism (i.e. both chromosomal copies of the individual have the same nucleotide at the SNP location), or the individual is heterozygous (i.e. the two sister chromosomes of the individual contain different nucleotides). The SNP nomenclature as reported herein refers to the official Reference SNP (rs) ID identification tag as assigned to each unique SNP by the National Center for Biotechnological Information (NCBI).

A “variant”, as described herein, refers to a segment of DNA that differs from the reference DNA. Consequently, a “marker” or a “polymorphic marker”, as defined herein, is the location of a variant. Alleles that differ from the reference are referred to as “variant” alleles.

A “microsatellite” is a polymorphic marker that has multiple small repeats of bases that are 2-8 nucleotides in length (such as CA repeats) at a particular site, in which the number of repeat lengths varies in the general population. An “indel” is a common form of polymorphism comprising a small insertion or deletion that is typically only a few nucleotides long.

A “haplotype,” as described herein, refers to a segment of genomic DNA that is characterized by a specific combination of alleles arranged along the segment. For diploid organisms such as humans, a haplotype comprises one member of the pair of alleles for each polymorphic marker or locus along the segment. In a certain embodiment, the haplotype can comprise two or more alleles, three or more alleles, four or more alleles, or five or more alleles. Haplotypes are described herein in the context of the marker name and the allele of the marker in that haplotype, e.g., “4 rs9642880” refers to the 4 allele of marker rs9642880 being in the haplotype, and is equivalent to “rs9642880 allele 4”. Furthermore, allelic codes in haplotypes are as for individual markers, i.e. 1=A, 2=C, 3=G and 4=T.

The term “susceptibility”, as described herein, refers to the proneness of an individual towards the development of a certain state (e.g., a certain trait, phenotype or disease), or towards being less able to resist a particular state than the average individual. The term encompasses both increased susceptibility and decreased susceptibility. Thus, particular alleles at polymorphic markers and/or haplotypes of the invention as described herein may be characteristic of increased susceptibility (i.e., increased risk) of urinary bladder cancer (UBC), as characterized by a relative risk (RR) or odds ratio (OR) of greater than one for the particular allele or haplotype. Alternatively, the markers and/or haplotypes of the invention are characteristic of decreased susceptibility (i.e., decreased risk) of UBC, as characterized by a relative risk of less than one.

The terms Urinary bladder cancer, UBC and bladder cancer are synonymously used in this text.

The term “and/or” shall in the present context be understood to indicate that either or both of the items connected by it are involved. In other words, the term herein shall be taken to mean “one or the other or both”.

The term “look-up table”, as described herein, is a table that correlates one form of data to another form, or one or more forms of data to a predicted outcome to which the data is relevant, such as phenotype or trait. For example, a look-up table can comprise a correlation between allelic data for at least one polymorphic marker and a particular trait or phenotype, such as a particular disease diagnosis, that an individual who comprises the particular allelic data is likely to display, or is more likely to display than individuals who do not comprise the particular allelic data. Look-up tables can be multidimensional, i.e. they can contain information about multiple alleles for single markers simultaneously, or they can contain information about multiple markers, and they may also comprise other factors, such as particulars about diseases diagnoses, racial information, biomarkers, biochemical measurements, therapeutic methods or drugs, etc.

A “computer-readable medium”, is an information storage medium that can be accessed by a Computer using a commercially available or custom-made interface. Exemplary computer-readable media include memory (e.g., RAM, ROM, flash memory, etc.), optical storage media (e.g., CD-ROM), magnetic storage media (e.g., computer hard drives, floppy disks, etc.), punch cards, or other commercially available media. Information may be transferred between a system of interest and a medium, between computers, or between computers and the computer-readable medium for storage or access of stored information. Such transmission can be electrical, or by other available methods, such as IR links, wireless connections, etc.

A “nucleic acid sample” as described herein, refers to a sample obtained from an individual that contains nucleic acid (DNA or RNA). In certain embodiments, i.e. the detection of specific polymorphic markers and/or haplotypes, the nucleic acid sample comprises genomic DNA. Such a nucleic acid sample can be obtained from any source that contains genomic DNA, including a blood sample, sample of amniotic fluid, sample of cerebrospinal fluid, or tissue sample from skin, muscle, buccal or conjunctival mucosa, placenta, gastrointestinal tract or other organs.

The term “UBC therapeutic agent” refers to an agent that can be used to ameliorate or prevent symptoms associated with urinary bladder cancer.

The term “UBC-associated nucleic acid”, as described herein, refers to a nucleic acid that has been found to be associated to urinary bladder cancer. This includes, but is not limited to, the markers and haplotypes described herein and markers and haplotypes in strong linkage disequilibrium (LD) therewith

The term “nucleic acid sequence data” in the context of this application refers to generally any data indicating the sequence of a nucleic acid containing one or more nucleotide. The data may also comprise information about the location of said sequence within the genome and/or within a gene, gene segment or otherwise defined location of a sequence. Consequently, such data can refer to only one nucleotide, e.g. the allelic status of a known polymorphic site, in which case the data would generally also indicate a position reference or other definition of the nucleotide site.

Nucleic acid sequence data can be represented in any format, such as for example as text Strings, in digital or paper format and as representations of gels and chips, etc.

Genome wide association studies have repeatedly reported cancer associated variants on 8q24, 200-700 kb proximal to rs9642880 and c-Myc. c-Myc is the only known gene close to rs9642880, but a predicted gene, BC042052, is also in the same region. C-Myc is a known oncogene, however, as described in the accompanying Results section, genotyping for known miss-sense mutations in the c-Myc gene (G175C/rs4645960 and N26S/rs4645959) found no association with UBC.

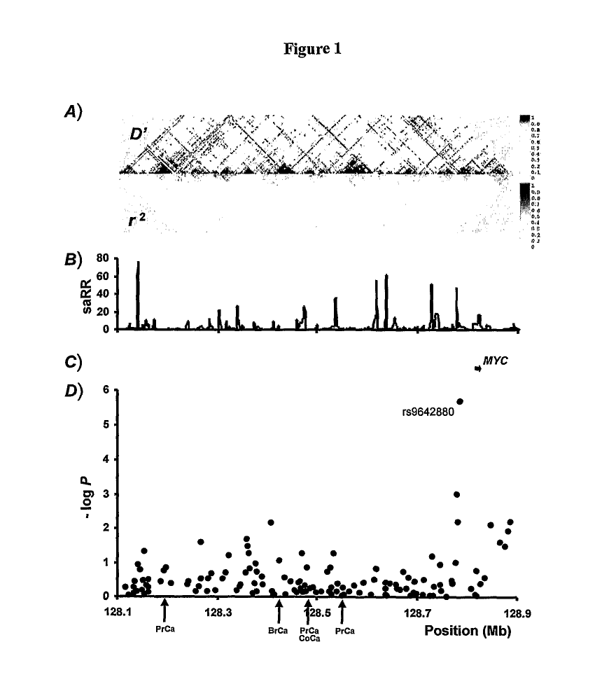

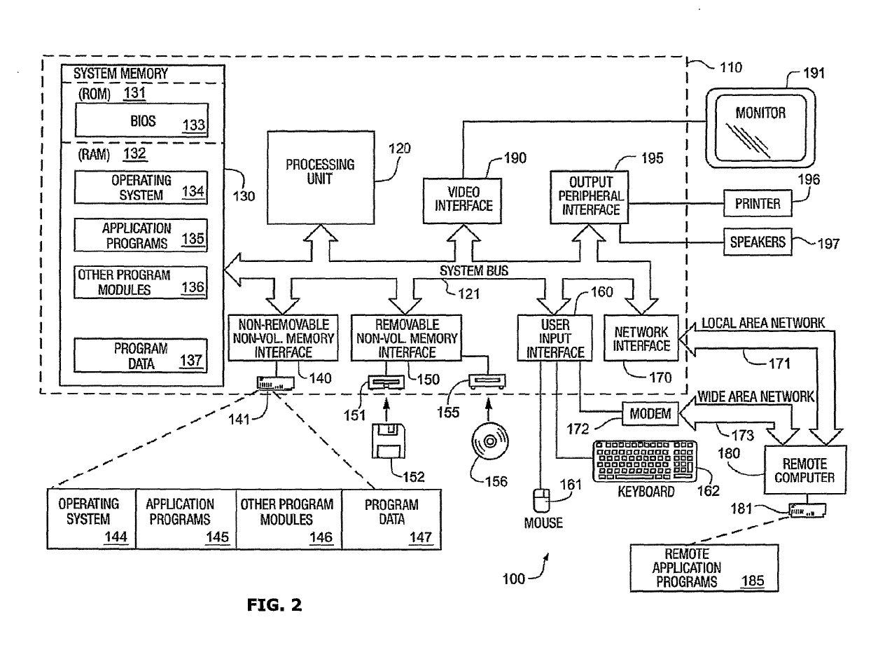

We and others have previously found SNPs at 8q24.21 to be strongly associated with cancer of the prostate (rs1447295, rs6983267 and rs16901979) (Gudmundsson, J., et al. Nat Genet. 39(5):631-7 (2007), Eeles, R. A., et al. Nat Genet. 2008; 40(3):316-21 (2008); Amundadottir L. T., et al. Nat Genet. 7:7 (2006); Thomas, G., Nat Genet. 2008; 40(3):310-5 (2008)). Subsequently, rs6983267 was also shown to associate with colorectal cancer (Tomlinson, I. et al. Nat Genet. 39, 984-8 (2007); Haiman, C. A. et al. Nat Genet. 39, 954-6 (2007); Zanke, B. W. et al. Nat Genet. 39, 989-94 (2007)) and most recently rs13281615 with breast cancer (Easton, D. F. et al. Nature 447, 1087-93 (2007)). These 4 variants are dispersed over a 500 kb region (FIG. 1) and are in weak LD with each other and with rs9642880 (Table 3). We found no association between these 4 SNPs and UBC in the combined study groups (Table 7). Moreover, we found no association between rs9642880 and prostate, breast or colorectal cancer in Icelandic case control samples (Table 8). Association of markers on chromosome 3 (rs710521 and surrogate markers thereof) to UBC was also identified.

### Methods of Determining Susceptibility to Disease

The present invention provides methods of determining a susceptibility to disease in a human individual.

### Assessment for Markers and Haplotypes

The genomic sequence within populations is not identical when individuals are compared. Rather, the genome exhibits sequence variability between individuals at many locations in the genome. Such variations in sequence are commonly referred to as polymorphisms, and there are many such sites within each genome For example, the human genome exhibits sequence variations which occur on average every 500 base pairs. The most common sequence variant consists of base variations at a single base position in the genome, and such sequence variants, or polymorphisms, are commonly called Single Nucleotide Polymorphisms (“SNPs”). These SNPs are believed to have occurred in a single mutational event, and therefore there are usually two possible alleles possible at each SNP site; the original allele and the mutated allele. Due to natural genetic drift and possibly also selective pressure, the original mutation has resulted in a polymorphism characterized by a particular frequency of its alleles in any given population. Many other types of sequence variants are found in the human genome, including microsatellites, insertions, deletions, inversions and copy number variations. A polymorphic Microsatellite has multiple small repeats of bases (such as CA repeats, TG on the complimentary strand) at a particular site in which the number of repeat lengths varies in the general population. In general, polymorphisms can comprise any number of specific alleles within the (population, although each human individual has two alleles at each polymorphic site—one maternal and one paternal allele. Thus in one embodiment of the invention, the polymorphism is characterized by the presence of two or more alleles in any given population. In another embodiment, the polymorphism is characterized by the presence of three or more alleles in a population. In another embodiment, the polymorphism is characterized by the presence of three or more alleles. In other embodiments, the polymorphism is characterized by four or more alleles, five or more alleles, six or more alleles, seven or more alleles, nine or more alleles, or ten or more alleles. All such polymorphisms can be utilized in the methods and kits of the present invention, and are thus within the scope of the invention.

Due to their abundance, SNPs account for a majority of sequence variation in the human Genome. Over 6 million human SNPs have been validated to date (www.ncbi.nlm.nih.gov/projects/SNP/snp_summary.cgi). However, CNVs (copy number variants or copy number polymorphisms) are receiving increased attention. These large-scale Polymorphisms (typically 1 kb or larger) account for polymorphic variation affecting a substantial proportion of the assembled human genome; known CNVs covery over 15% of the human genome sequence (Estivill, X., Armengol, L., PloS Genetics 3:1787-99 (2007); http://projects.tcag.ca/variation/). Most of these polymorphisms are however very rare, and on average affect only a fraction of the genomic sequence of each individual. CNVs are known to affect gene expression, phenotypic variation and adaptation by disrupting gene dosage, and are also known to cause disease (microdeletion and microduplication disorders) and confer risk of common complex diseases, including HIV-1 infection and glomerulonephritis (Redon, R., et al. Nature 23:444-454 (2006)). It is thus possible that either previously described or unknown CNVs represent causative variants in linkage disequilibrium with the markers described herein to be associated with UBC. Methods for detecting CNVs include comparative genomic hybridization (CGH) and genotyping, including use of genotyping arrays, as described by Carter (Nature Genetics 39:S16-S21 (2007)). The Database of Genomic Variants (http://projects.tcag.ca/variation/) contains updated information about the location, type and size of described CNVs. The database currently contains data for over 21,000 CNVs.

In some instances, reference is made to different alleles at a polymorphic site without choosing a reference allele. Alternatively, a reference sequence can be referred to for a particular polymorphic site. The reference allele is sometimes referred to as the “wild-type” allele and it Usually is chosen as either the first sequenced allele or as the allele from a “non-affected” individual (e.g., an individual that does not display a trait or disease phenotype).

Alleles for SNP markers as referred to herein refer to the bases A, C, G or T as they occur at the polymorphic site. The allele codes for SNPs used herein are as follows: 1=A, 2=C, 3=G, 4=T. Since human DNA is double-stranded, the person skilled in the art will realise that by assaying or reading the opposite DNA strand, the complementary allele can in each case be measured. Thus, for a polymorphic site (polymorphic marker) characterized by an A/G polymorphism, the methodology employed to detect the marker may be designed to specifically detect the presence of one or both of the two bases possible, i.e. A and G. Alternatively, by designing an assay that designed to detect the complimentary strand on the DNA template, the presence of the complementary bases T and C can be measured. Quantitatively (for example, in terms of relative risk), identical results would be obtained from measurement of either DNA strand (+strand or −strand).

Typically, a reference sequence is referred to for a particular sequence. Alleles that differ from the reference are sometimes referred to as “variant” alleles. A variant sequence, as used herein, refers to a sequence that differs from the reference sequence but is otherwise substantially similar. Alleles at the polymorphic genetic markers described herein are variants. Variants can include changes that affect a polypeptide. Sequence differences, when compared to a reference nucleotide sequence, can include the insertion or deletion of a single nucleotide, or of more than one nucleotide, resulting in a frame shift; the change of at least one nucleotide, resulting in a change in the encoded amino acid; the change of at least one nucleotide, resulting in the generation of a premature stop codon; the deletion of several nucleotides, resulting in a deletion of one or more amino acids encoded by the nucleotides; the insertion of one or several nucleotides, such as by unequal recombination or gene conversion, resulting in an interruption of the coding sequence of a reading frame; duplication of all or a part of a sequence; transposition; or a rearrangement of a nucleotide sequence. Such sequence changes can alter the polypeptide encoded by the nucleic acid. For example, if the change in the nucleic acid sequence causes a frame shift, the frame shift can result in a change in the encoded amino acids, and/or can result in the generation of a premature stop codon, causing generation of a truncated polypeptide. Alternatively, a polymorphism can be a synonymous change in one or more nucleotides (i.e., a change that does not result in a change in the amino acid sequence). Such a polymorphism can, for example, alter splice sites, affect the stability or transport of mRNA, or otherwise affect the transcription or translation of an encoded polypeptide. It can also alter DNA to increase the possibility that structural changes, such as amplifications or deletions, occur at the somatic level. The polypeptide encoded by the reference nucleotide sequence is the “reference” polypeptide with a particular reference amino acid sequence, and polypeptides encoded by variant alleles are referred to as “variant” polypeptides with variant amino acid sequences.

A haplotype refers to a single stranded segment of DNA that is characterized by a specific combination of alleles arranged along the segment. For diploid organisms such as humans, a haplotype comprises one member of the pair of alleles for each polymorphic marker or locus. In a certain embodiment, the haplotype can comprise two or more alleles, three or more alleles, four or more alleles, or five or more alleles, each allele corresponding to a specific polymorphic marker along the segment. Haplotypes can comprise a combination of various polymorphic markers, e.g., SNPs and microsatellites, having particular alleles at the polymorphic sites. The haplotypes thus comprise a combination of alleles at various genetic markers.

Detecting specific polymorphic markers and/or haplotypes can be accomplished by methods known in the art for detecting sequences at polymorphic sites. For example, standard techniques for genotyping for the presence of SNPs and/or microsatellite markers can be used, such as fluorescence-based techniques (e.g., Chen, X. et al., Genome Res. 9(5): 492-98 (1999); Kutyavin et al., Nucleic Acid Res. 34:e128 (2006)), utilizing PCR, LCR, Nested PCR and other techniques for nucleic acid amplification. Specific commercial methodologies available for SNP genotyping include, but are not limited to, TaqMan genotyping assays and SNPIex platforms Applied Biosystems), gel electrophoresis (Applied Biosystems), mass spectrometry (e.g., MassARRAY system from Sequenom), minisequencing methods, real-time PCR, Bio-Plex system (BioRad), CEQ and SNPstream systems (Beckman), array hybridization technology (e.g., Affymetrix GeneChip; Perlegen), BeadArray Technologies (e.g., Illumina GoldenGate and Infinium assays), array tag technology (e.g., Parallele), and endonuclease-based fluorescence hybridization technology (Invader; Third Wave). Some of the available array platforms, including Affymetrix SNP Array 6.0 and Illumina CNV370-Duo and 1M BeadChips, include SNPs that tag certain CNVs. This allows detection of CNVs via surrogate SNPs included in these platforms. Thus, by use of these or other methods available to the person skilled in the art, one or more alleles at polymorphic markers, including microsatellites, SNPs or other types of polymorphic markers, can be identified.

In certain embodiments, polymorphic markers are detected by sequencing technologies. Obtaining sequence information about an individual identifies particular nucleotides in the context of a sequence. For SNPs, sequence information about a single unique sequence site is sufficient to identify alleles at that particular SNP. For markers comprising more than one nucleotide, sequence information about the nucleotides of the individual that contain the polymorphic site identifies the alleles of the individual for the particular site. The sequence information can be obtained from a sample from the individual. In certain embodiments, the sample is a nucleic acid sample. In certain other embodiments, the sample is a protein sample.

Various methods for obtaining nucleic acid sequence are known to the skilled person, and all such methods are useful for practicing the invention. Sanger sequencing is a well-known Method for generating nucleic acid sequence information. Recent methods for obtaining large amounts of sequence data have been developed, and such methods are also contemplated to be useful for obtaining sequence information. These include pyrosequencing technology (Ronaghi, M. et al. Anal Biochem 267:65-71 (1999); Ronaghi, et al. Biotechniques 25:876-878 (1998)), e.g. 454 pyrosequencing (Nyren, P., et al. Anal Biochem 208:171-175 (1993)), Illumina/Solexa sequencing technology (http://www.illumina.com; see also Strausberg, R L, et al Drug Disc Today 13:569-577 (2008)), and Supported Oligonucleotide Ligation and Detection Platform (SOLiD) technology (Applied Biosystems, http://www.appliedbiosystems.com); Strausberg, R L, et al Drug Disc Today 13:569-577 (2008).

It is possible to impute or predict genotypes for un-genotyped relatives of genotyped individuals. For every un-genotyped case, it is possible to calculate the probability of the genotypes of its relatives given its four possible phased genotypes. In practice it may be preferable to include only the genotypes of the case's parents, children, siblings, half-siblings (and the half-sibling's parents), grand-parents, grand-children (and the grand-children's parents) and spouses. It will be assumed that the individuals in the small sub-pedigrees created around each case are not related through any path not included in the pedigree. It is also assumed that alleles that are not transmitted to the case have the same frequency—the population allele frequency. Let us consider a SNP marker with the alleles A and G. The probability of the genotypes of the case's relatives can then be computed by:

$$Pr ( genotypes of relatives ; θ ) = \begin{matrix}{\sum }_{h ∈ \{ AA , AG , GA , GG \}} Pr ( h ; θ ) Pr \\ ( genotypes of relatives \vert h ) ,\end{matrix}$$

where θ denotes the A allele's frequency in the cases. Assuming the genotypes of each set of relatives are independent, this allows us to write down a likelihood function for θ:

$$L ( θ ) = {\prod }_{i} Pr ( genotypes of relatives of case i ; θ ) . \qquad (* )$$

This assumption of independence is usually not correct. Accounting for the dependence between individuals is a difficult and potentially prohibitively expensive computational task. The likelihood function in (*) may be thought of as a pseudolikelihood approximation of the full likelihood function for θ which properly accounts for all dependencies. In general, the genotyped cases and controls in a case-control association study are not independent and applying the case-control method to related cases and controls is an analogous approximation. The method of genomic control (Devlin, B. et al., Nat Genet. 36, 1129-30; author reply 1131 (2004)) has proven to be successful at adjusting case-control test statistics for relatedness. We therefore apply the method of genomic control to account for the dependence between the terms in our pseudolikelihood and produce a valid test statistic.

Fisher's information can be used to estimate the effective sample size of the part of the pseudolikelihood due to un-genotyped cases. Breaking the total Fisher information, I, into the part due to genotyped cases, I_{g}, and the part due to ungenotyped cases, Iᵤ, I=I_{g}+Iᵤ, and denoting the number of genotyped cases with N, the effective sample size due to the un-genotyped cases is estimated by

$$\frac{{I}_{u}}{{I}_{g}} N .$$

In the present context, an individual who is at an increased susceptibility (i.e., increased risk) for a disease (e.g., urinary bladder cancer), is an individual in whom at least one specific allele at one or more polymorphic marker or haplotype conferring increased susceptibility (increased risk) for UBC is identified (i.e., at-risk marker alleles or haplotypes). The at-risk marker or haplotype is one that confers an increased risk (increased susceptibility) of the disease. In one embodiment, significance associated with a marker or haplotype is measured by a relative risk (RR). In another embodiment, significance associated with a marker or haplotype is measured by an odds ratio (OR). In a further embodiment, the significance is measured by a percentage. In one embodiment, a significant increased risk is measured as a risk (relative risk and/or odds ratio) of at least 1.1, including but not limited to: at least 1.12, at least 1.13, at least 1.14, at least 1.15, at least 1.16, at least 1.17, at least 1.18, at least 1.19, at least 1.20, at least 1.21, at least 1.22, at least 1.23, at least 1.25, at least 1.30, at least 1.40, and at least 1.50. In a particular embodiment, a risk (relative risk and/or odds ratio) of at least 1.10 is significant. In another particular embodiment, a risk of at least 1.20 is significant. In yet another embodiment, a risk of at least 1.21 is significant. In a further embodiment, a relative risk of at least 1.40 is significant. In another further embodiment, a significant increase in risk is at least 1.45 is significant. However, other cutoffs are also contemplated, including any numerical values bridging the numbers above, and such cutoffs are also within scope of the present invention. In other embodiments, a significant increase in risk is at least about 10%, including but not limited to about 11%, about 12%, about 13%, about 14%, about 15%, about 16%, about 17%, about 18%, about 19%, about 20%, about 21%, about 25%, about 30%, about 35%, about 40%, and about 45%. In one particular embodiment, a significant increase in risk is at least 20%. Other cutoffs or ranges as deemed suitable by the person skilled in the art to characterize the invention are however also contemplated, and those are also within scope of the present invention. In certain embodiments, a significant increase in risk is characterized by a p-value, such as a p-value of less than 0.05, less than 0.01, less than 0.001, less than 0.0001, less than 0.00001, less than 0.000001, less than 0.0000001, less than 0.00000001, or less than 0.000000001.

An at-risk polymorphic marker or haplotype of the present invention is one where at least one allele of at least one marker or haplotype is more frequently present in an individual at risk for the disease or trait (affected), or diagnosed with the disease or trait, compared to the frequency of its presence in a comparison group (control), such that the presence of the marker or haplotype is indicative of susceptibility to the disease or trait, which in this case is urinary bladder cancer (UBC). The control group may in one embodiment be a population sample, i.e. a random sample from the general population. In another embodiment, the control group is represented by a group of individuals who are disease-free. Such disease-free control may in one embodiment be characterized by the absence of one or more specific disease-associated symptoms. In another embodiment, the disease-free control group is characterized by the absence of one or more disease-specific risk factors. Such risk factors are in one embodiment at least one environmental risk factor. Representative environmental factors are natural products, minerals or other chemicals which are known to affect, or contemplated to affect, the risk of developing the specific disease or trait. Other environmental risk factors are risk factors related to lifestyle, including but not limited to food and drink habits, geographical location of main habitat, and occupational risk factors. In another embodiment, the risk factors comprise at least one additional genetic risk factor.

As an example of a simple test for correlation would be a Fisher-exact test on a two-by-two table. Given a cohort of chromosomes, the two-by-two table is constructed out of the number of chromosomes that include both of the markers or haplotypes, one of the markers or haplotypes but not the other and neither of the markers or haplotypes. Other statistical tests of association known to the skilled person are also contemplated and are also within scope of the invention. The person skilled in the art will appreciate that for markers with two alleles present in the population being studied (such as SNPs), and wherein one allele is found in increased frequency in a group of individuals with a trait or disease in the population, compared with controls, the other allele of the marker will be found in decreased frequency in the group of individuals with the trait or disease, compared with controls. In such a case, one allele of the marker (the one found in increased frequency in individuals with the trait or disease) will be the at-risk allele, while the other allele will be a protective allele.

Thus in other embodiments of the invention, an individual who is at a decreased susceptibility (i.e., at a decreased risk) for a disease or trait is an individual in whom at least one specific allele at one or more polymorphic marker or haplotype conferring decreased susceptibility for the disease or trait is identified. The marker alleles and/or haplotypes conferring decreased risk are also said to be protective. In one aspect, the protective marker or haplotype is one that confers a significant decreased risk (or susceptibility) of the disease or trait. In one embodiment, significant decreased risk is measured as a relative risk (or odds ratio) of less than 0.9, including but not limited to less than 0.9, less than 0.8, less than 0.7, less than 0.6, less than 0.5, less than 0.4, less than 0.3, less than 0.2 and less than 0.1. In one particular embodiment, significant decreased risk is less than 0.7. In another embodiment, significant decreased risk is less than 0.5. In yet another embodiment, significant decreased risk is less than 0.3. In another embodiment, the decrease in risk (or susceptibility) is at least 20%, including but not limited to at least 25%, at least 30%, at least 35%, at least 40%, at least 45%, at least 50%, at feast 55%, at least 60%, at least 65%, at least 70%, at least 75%, at least 80%, at least 85%, at least 90%, at least 95% and at least 98%. In one particular embodiment, a significant decrease in risk is at least about 30%. In another embodiment, a significant decrease in risk is at least about 50%. In another embodiment, the decrease in risk is at least about 70%. Other cutoffs or ranges as deemed suitable by the person skilled in the art to characterize the invention are however also contemplated, and those are also within scope of the present invention.

A genetic variant associated with a disease or trait (e.g. urinary bladder cancer) can be used alone to predict the risk of the disease for a given genotype. For a biallelic marker, such as a SNP, there are 3 possible genotypes: homozygote for the at-risk variant, heterozygote, and non carrier of the at-risk variant. Risk associated with variants at multiple loci can be used to estimate overall risk. For multiple SNP variants, there are k possible genotypes k=3ⁿ×2^{p}; where n is the number autosomal loci and p the number of gonosomal (sex chromosomal) loci. Overall risk assessment calculations usually assume that the relative risks of different genetic variants multiply, i.e. the overall risk (e.g., RR or OR) associated with a particular genotype combination is the product of the risk values for the genotype at each locus. If the risk presented is the relative risk for a person, or a specific genotype for a person, compared to a reference population with matched gender and ethnicity, then the combined risk—is the product of the locus specific risk values—and which also corresponds to an overall risk estimate compared with the population. If the risk for a person is based on a comparison to non-carriers of the at risk allele, then the combined risk corresponds to an estimate that compares the person with a given combination of genotypes at all loci to a group of individuals who do not carry risk variants at any of those loci. The group of non-carriers of any at risk variant has the lowest estimated risk and has a combined risk, compared with itself (i.e., non-carriers) of 1.0, but has an overall risk, compare with the population, of less than 1.0. It should be noted that the group of non-carriers can potentially be very small, especially for large number of loci, and in that case, its relevance is correspondingly small.

The multiplicative model is a parsimonious model that usually fits the data of complex traits reasonably well. Deviations from multiplicity have been rarely described in the context of common variants for common diseases, and if reported are usually only suggestive since very large sample sizes are usually required to be able to demonstrate statistical interactions between loci.

By way of an example, let us consider the case where a total of eight variants that have been associated with a disease. One such example is provided by eight loci associated with prostate cancer (Gudmundsson, J., et al., Nat Genet. 39:631-7 (2007), Gudmundsson, J., et al., Nat Genet 39:977-83 (2007); Yeager, M., et al, Nat Genet. 39:645-49 (2007), Amundadottir, L., et al., Nat Genet. 38:652-8 (2006); Haiman, C. A., et al., Nat Genet 39:638-44 (2007)). Seven of these loci are on autosomes, and the remaining locus is on chromosome X. The total number of theoretical genotypic combinations is then 3⁷×2¹=4374. Some of those genotypic classes are very rare, but are still possible, and should be considered for overall risk assessment.

It is likely that the multiplicative model applied in the case of multiple genetic variant will also be valid in conjugation with non-genetic risk variants assuming that the genetic variant does not clearly correlate with the “environmental” factor. In other words, genetic and non-genetic at-risk variants can be assessed under the multiplicative model to estimate combined risk, assuming that the non-genetic and genetic risk factors do not interact.

Using the same quantitative approach, the combined or overall risk associated with any plurality of variants associated with urinary bladder cancer may be assessed.

### Linkage Disequilibrium

The natural phenomenon of recombination, which occurs on average once for each chromosomal pair during each meiotic event, represents one way in which nature provides variations in sequence (and biological function by consequence). It has been discovered that recombination does not occur randomly in the genome; rather, there are large variations in the frequency of recombination rates, resulting in small regions of high recombination frequency (also called recombination hotspots) and larger regions of low recombination frequency, which are commonly referred to as Linkage Disequilibrium (LD) blocks (Myers, S. et al., Biochem Soc Trans 34:526-530 (2006); Jeffreys, A. J., et al., Nature Genet. 29:217-222 (2001); May, C. A., et al., Nature Genet 31:272-275 (2002)).

Linkage Disequilibrium (LD) refers to a non-random assortment of two genetic elements. For example, if a particular genetic element (e.g., an allele of a polymorphic marker, or a haplotype) occurs in a population at a frequency of 0.50 (50%) and another element occurs at a frequency of 0.50 (50%), then the predicted occurrence of a person's having both elements is 0.25 (25%), assuming a random distribution of the elements. However, if it is discovered that the two elements occur together at a frequency higher than 0.25, then the elements are said to be in linkage disequilibrium, since they tend to be inherited together at a higher rate than what their independent frequencies of occurrence (e.g., allele or haplotype frequencies) would predict. Roughly speaking, LD is generally correlated with the frequency of recombination events between the two elements. Allele or haplotype frequencies can be determined in a population by genotyping individuals in a population and determining the frequency of the occurrence of each allele or haplotype in the population. For populations of diploids, e.g., human populations, individuals will typically have two alleles or allelic combinations for each genetic element (e.g., a marker, haplotype or gene).

Many different measures have been proposed for assessing the strength of linkage disequilibrium (LD; reviewed in Devlin, B. & Risch, N., Genomics 29:311-22 (1995))). Most capture the strength of association between pairs of biallelic sites. Two important pairwise measures of LD are r² (sometimes denoted Δ²) and |D′| (Lewontin, R., Genetics 49:49-67 (1964); Hill, W. G. & Robertson, A. Theor. Appl. Genet. 22:226-231 (1968)). Both measures range from 0 (no disequilibrium) to 1 (‘complete’ disequilibrium), but their interpretation is slightly different. |D′| is defined in such a way that it is equal to 1 if just two or three of the possible haplotypes for two markers are present, and it is <1 if all four possible haplotypes are present. Therefore, a value of |D′| that is <1 indicates that historical recombination may have occurred between two sites (recurrent mutation can also cause |D′| to be <1, but for single nucleotide polymorphisms (SNPs) this is usually regarded as being less likely than recombination). The measure r² represents the statistical correlation between two sites, and takes the value of 1 if only two haplotypes are present.

The r² measure is arguably the most relevant measure for association mapping, because there is a simple inverse relationship between r² and the sample size required to detect association between susceptibility loci and SNPs. These measures are defined for pairs of sites, but for some applications a determination of how strong LD is across an entire region that contains many polymorphic sites might be desirable (e.g., testing whether the strength of LD differs significantly among loci or across populations, or whether there is more or less LD in a region than predicted under a particular model). Roughly speaking, r measures how much recombination would be required under a particular population model to generate the LD that is seen in the data. This type of method can potentially also provide a statistically rigorous approach to the problem of determining whether LD data provide evidence for the presence of recombination hotspots. For the methods described herein, a significant r² value between markers indicative of the markers being in linkage disequilibrium can be at least 0.1, such as at least 0.15, 0.20, 0.25, 0.30, 0.35, 0.40, 0.45, 0.50, 0.55, 0.60, 0.65, 0.70, 0.75, 0.80, 0.85, 0.90, 0.91, 0.92, 0.93, 0.94, 0.95, 0.96, 0.97, 0.98, or at least 0.99. In one preferred embodiment, the significant r² value can be at least 0.2. Alternatively, markers in linkage disequilibrium are characterized by values of |D′| of at least 0.2, such as 0.3, 0.4, 0.5, 0.6, 0.7, 0.8, 0.85, 0.9, 0.95, 0.96, 0.97, 0.98, or at least 0.99. Thus, linkage disequilibrium represents a correlation between alleles of distinct markers. In certain embodiments, linkage disequilibrium is defined in terms of values for both the r² and |D′| measures. In one such embodiment, a significant linkage disequilibrium is defined as r²>0.1 and |D′|>0.8, and markers fulfilling these criteria are said to be in linkage disequilibrium. In another embodiment, a significant linkage disequilibrium is defined as r²>0.2 and |D′|>0.9.

Other combinations and permutations of values of r² and |D′| for determining linkage disequilibrium are also contemplated, and are also within the scope of the invention. Linkage disequilibrium can be determined in a single human population, as defined herein, or it can be determined in a collection of samples comprising individuals from more than one human population. In one embodiment of the invention, LD is determined in a sample from one or more of the HapMap populations (Caucasian, African (Yuroban), Japanese, Chinese), as defined (http://www.hapmap.org). In one such embodiment, LD is determined in the CEU population of the HapMap samples (Utah residents with ancestry from northern and western Europe). In another embodiment, LD is determined in the YRI population of the HapMap samples (Yuroba in Ibadan, Nigeria). In another embodiment, LD is determined in the CHB population of the HapMap samples (Han Chinese from Beijing, China). In another embodiment, LD is determined in the JPT population of the HapMap samples (Japanese from Tokyo, Japan). In yet another embodiment, LD is determined in samples from the Icelandic population.

If all polymorphisms in the genome were independent at the population level (i.e., no LD), then every single one of them would need to be investigated in association studies, to assess all the different polymorphic states. However, due to linkage disequilibrium between polymorphisms, tightly linked polymorphisms are strongly correlated, which reduces the number of polymorphisms that need to be investigated in an association study to observe a significant association. Another consequence of LD is that many polymorphisms may give an association signal due to the fact that these polymorphisms are strongly correlated.

Genomic LD maps have been generated across the genome, and such LD maps have been proposed to serve as framework for mapping disease-genes (Risch, N. & Merkiangas, K, Science 273:1516-1517 (1996); Maniatis, N., et al., Proc Natl Acad Sci USA 99:2228-2233 (2002); Reich, D E et al, Nature 411:199-204 (2001)).

It is now established that many portions of the human genome can be broken into series of discrete haplotype blocks containing a few common haplotypes; for these blocks, linkage disequilibrium data provides little evidence indicating recombination (see, e.g., Wall., J. D. and Pritchard, J. K., Nature Reviews Genetics 4:587-597 (2003); Daly, M. et al., Nature Genet. 29:229-232 (2001); Gabriel, S. B. et al., Science 296:2225-2229 (2002); Patil, N. et al., Science 294:1719-1723 (2001); Dawson, E. et al., Nature 418:544-548 (2002); Phillips, M. S. et al., Nature Genet. 33: 382-387 (2003)).

There are two main methods for defining these haplotype blocks: blocks can be defined as regions of DNA that have limited haplotype diversity (see, e.g., Daly, M. et al., Nature Genet. 29:229-232 (2001); Patil, N. et al., Science 294:1719-1723 (2001); Dawson, E. et al., Nature 418:544-548 (2002); Zhang, K. et al., Proc. Natl. Acad. Sci. USA 99:7335-7339 (2002)), or as regions between transition zones having extensive historical recombination, identified using linkage disequilibrium (see, e.g., Gabriel, S. B. et al., Science 296:2225-2229 (2002); Phillips, M. S. et al., Nature Genet. 33:382-387 (2003); Wang, N. et al., Am. J. Hum. Genet. 71:1227-1234 (2002); Stumpf, M. P., and Goldstein, D. B., Curr. Biol. 13:1-8 (2003)). More recently, a fine-scale map of recombination rates and corresponding hotspots across the human genome has been generated (Myers, S., et al., Science 310:321-32324 (2005); Myers, S. et al., Biochem Soc Trans 34:526530 (2006)). The map reveals the enormous variation in recombination across the genome, with recombination rates as high as 10-60 cM/Mb in hotspots, while closer to 0 in intervening regions, which thus represent regions of limited haplotype diversity and high LD. The map can therefore be used to define haplotype blocks/LD blocks as regions flanked by recombination hotspots. As used herein, the terms “haplotype block” or “LD block” includes blocks defined by any of the above described characteristics, or other alternative methods used by the person skilled in the art to define such regions.

Haplotype blocks (LD blocks) can be used to map associations between phenotype and haplotype status, using single markers or haplotypes comprising a plurality of markers. The main haplotypes can be identified in each haplotype block, and then a set of “tagging” SNPs or markers (the smallest set of SNPs or markers needed to distinguish among the haplotypes) can then be identified. These tagging SNPs or markers can then be used in assessment of samples from groups of individuals, in order to identify association between phenotype and haplotype. If desired, neighboring haplotype blocks can be assessed concurrently, as there may also exist linkage disequilibrium among the haplotype blocks.

It has thus become apparent that for any given observed association to a polymorphic marker in the genome, it is likely that additional markers in the genome also show association. This is a natural consequence of the uneven distribution of LD across the genome, as observed by the large variation in recombination rates. The markers used to detect association thus in a sense represent “tags” for a genomic region (i.e., a haplotype block or LD block) that is associating with a given disease or trait, and as such are useful for use in the methods and kits of the present invention. One or more causative (functional) variants or mutations may reside within the region found to be associating to the disease or trait. The functional variant may be another SNP, a tandem repeat polymorphism (such as a minisatellite or a microsatellite), a transposable element, or a copy number variation, such as an inversion, deletion or insertion. Such variants in LD with the variants described herein may confer a higher relative risk (RR) or odds ratio (OR) than observed for the tagging markers used to detect the association. The present invention thus refers to the markers used for detecting association to the disease, as described herein, as well as markers in linkage disequilibrium with the markers. Thus, in certain embodiments of the invention, markers that are in LD with the markers originally used to detect an association may be used as surrogate markers. The surrogate markers have in one embodiment relative risk (RR) and/or odds ratio (OR) values smaller than originally detected. In other embodiments, the surrogate markers have RR or OR values greater than those initially determined for the markers initially found to be associating with the disease. An example of such an embodiment would be a rare, or relatively rare (such as <10% allelic population frequency) variant in LD with a more common variant (>10% population frequency) initially found to be associating with the disease. Identifying and using such surrogate markers for detecting the association can be performed by routine methods well known to the person skilled in the art, and are therefore within the scope of the present invention.

### Haplotype Analysis

One general approach to haplotype analysis involves using likelihood-based inference applied to NEsted MOdels (Gretarsdottir S., et al., Nat. Genet. 35:131-38 (2003)). The method is implemented in the program NEMO, which allows for many polymorphic markers, SNPs and microsatellites. The method and software are specifically designed for case-control studies where the purpose is to identify haplotype groups that confer different risks. It is also a tool for studying LD structures. In NEMO, maximum likelihood estimates, likelihood ratios and p-values are calculated directly, with the aid of the EM algorithm, for the observed data treating it as a missing-data problem.

Even though likelihood ratio tests based on likelihoods computed directly for the observed data, which have captured the information loss due to uncertainty in phase and missing genotypes, can be relied on to give valid p-values, it would still be of interest to know how much information had been lost due to the information being incomplete. The information measure for haplotype analysis is described in Nicolae and Kong (Technical Report 537, Department of Statistics, University of Statistics, University of Chicago; Biometrics, 60(2):368-75 (2004)) as a natural extension of information measures defined for linkage analysis, and is implemented in NEMO.

### Association Analysis

For single marker association to a disease, the Fisher exact test can be used to calculate two-sided p-values for each individual allele. Correcting for relatedness among patients can be done by extending a variance adjustment procedure previously described (Risch, N. & Teng, J. Genome Res., 8:1273-1288 (1998)) for sibships so that it can be applied to general familial relationships. The method of genomic controls (Devlin, B. & Roeder, K. Biometrics 55:997 (1999)) can also be used to adjust for the relatedness of the individuals and possible stratification. For both single-marker and haplotype analyses, relative risk (RR) and the population attributable risk (PAR) can be calculated assuming a multiplicative model (haplotype relative risk model) (Terwilliger, J. D. & Ott, J., Hum, Hered. 42:337-46 (1992) and Falk, C. T. & Rubinstein, P, Ann. Hum. Genet. 51 (Pt 3):227-33 (1987)), i.e., that the risks of the two alleles/haplotypes a person carries multiply. For example, if RR is the risk of A relative to a, then the risk of a person homozygote AA will be RR times that of a heterozygote ‘Aa’ and RR² times that of a homozygote ‘aa’. The multiplicative model has a nice property that simplifies analysis and computations—haplotypes are independent, i.e., in Hardy-Weinberg equilibrium, within the affected population as well as within the control population. As a consequence, haplotype counts of the affecteds and controls each have multinomial distributions, but with different haplotype frequencies under the alternative hypothesis. Specifically, for two haplotypes, hᵢ and hⱼ, risk(hᵢ)/risk(hⱼ)=(fᵢ/pᵢ)/(fⱼ/pⱼ), where f and p denote, respectively, frequencies in the affected population and in the control population. While there is some power loss if the true model is not multiplicative, the loss tends to be mild except for extreme cases. Most importantly, p-values are always valid since they are computed with respect to null hypothesis.

An association signal detected in one association study may be replicated in a second cohort, ideally from a different population (e.g., different region of same country, or a different country) of the same or different ethnicity. The advantage of replication studies is that the number of tests performed in the replication study is usually quite small, and hence the less stringent the statistical measure that needs to be applied. For example, for a genome-wide search for susceptibility variants for a particular disease or trait using 300,000 SNPs, a correction for the 300,000 tests performed (one for each SNP) can be performed. Since many SNPs on the arrays typically used are correlated (i.e., in LD), they are not independent. Thus, the correction is conservative. Nevertheless, applying this correction factor requires an observed P-value of less than 0.05/300,000=1.7×10⁻⁷ for the signal to be considered significant applying this conservative test on results from a single study cohort. Obviously, signals found in a genome-wide association study with P-values less than this conservative threshold (i.e., more significant) are a measure of a true genetic effect, and replication in additional cohorts is not necessarily from a statistical point of view. Importantly, however, signals with P-values that are greater than this threshold may also be due to a true genetic effect. The sample size in the first study may not have been sufficiently large to provide an observed P-value that meets the conservative threshold for genome-wide significance, or the first study may not have reached genome-wide significance due to inherent fluctuations due to sampling. Since the correction factor depends on the number of statistical tests performed, if one signal (one SNP) from an initial study is replicated in a second case-control cohort, the appropriate statistical test for significance is that for a single statistical test, i.e., P-value less than 0.05. Replication studies in one or even several additional case-control cohorts have the added advantage of providing assessment of the association signal in additional populations, thus simultaneously confirming the initial finding and providing an assessment of the overall significance of the genetic variant(s) being tested in human populations in general.

The results from several case-control cohorts can also be combined to provide an overall assessment of the underlying effect. The methodology commonly used to combine results from multiple genetic association studies is the Mantel-Haenszel model (Mantel and Haenszel, J Natl Cancer Inst 22:719-48 (1959)). The model is designed to deal with the situation where association results from different populations, with each possibly having a different population frequency of the genetic variant, are combined. The model combines the results assuming that the effect of the variant on the risk of the disease, a measured by the OR or RR, is the same in all populations, while the frequency of the variant may differ between the populations. Combining the results from several populations has the added advantage that the overall power to detect a real underlying association signal is increased, due to the increased statistical power provided by the combined cohorts. Furthermore, any deficiencies in individual studies, for example due to unequal matching of cases and controls or population stratification will tend to balance out when results from multiple cohorts are combined, again providing a better estimate of the true underlying genetic effect.

### Risk Assessment and Diagnostics

### Risk Calculations

The creation of a model to calculate the overall genetic risk involves two steps: i) conversion of odds-ratios for a single genetic variant into relative risk and ii) combination of risk from multiple variants in different genetic loci into a single relative risk value.

Deriving Risk from Odds-Ratios

Most gene discovery studies for complex diseases that have been published to date in authoritative journals have employed a case-control design because of their retrospective setup. These studies sample and genotype a selected set of cases (people who have the specified disease condition) and control individuals. The interest is in genetic variants (alleles) which frequency in cases and controls differ significantly.

The results are typically reported in odds ratios, that is the ratio between the fraction (probability) with the risk variant (carriers) versus the non-risk variant (non-carriers) in the groups of affected versus the controls, i.e. expressed in terms of probabilities conditional on the affection status:

OR=(Pr(c|A)/Pr(nc|A))/(Pr(c|C)/Pr(nc|C))

Sometimes it is however the absolute risk for the disease that we are interested in, i.e. the fraction of those individuals carrying the risk variant who get the disease or in other words the probability of getting the disease. This number cannot be directly measured in case-control studies, in part, because the ratio of cases versus controls is typically not the same as that in the general population. However, under certain assumption, we can estimate the risk from the odds ratio.

It is well known that under the rare disease assumption, the relative risk of a disease can be approximated by the odds ratio. This assumption may however not hold for many common diseases. Still, it turns out that the risk of one genotype variant relative to another can be estimated from the odds ratio expressed above. The calculation is particularly simple under the assumption of random population controls where the controls are random samples from the same population as the cases, including affected people rather than being strictly unaffected individuals. To increase sample size and power, many of the large genome-wide association and replication studies use controls that were neither age-matched with the cases, nor were they carefully scrutinized to ensure that they did not have the disease at the time of the study. Hence, while not exactly, they often approximate a random sample from the general population. It is noted that this assumption is rarely expected to be satisfied exactly, but the risk estimates are usually robust to moderate deviations from this assumption.

Calculations show that for the dominant and the recessive models, where we have a risk variant carrier, “c”, and a non-carrier, “nc”, the odds ratio of individuals is the same as the risk ratio between these variants:

OR=Pr(A|c)/Pr(A|nc)=r

And likewise for the multiplicative model, where the risk is the product of the risk associated with the two allele copies, the allelic odds ratio equals the risk factor:

OR=Pr(A|aa)/Pr(A|ab)=Pr(A|ab)/Pr(A|bb)=r

Here “a” denotes the risk allele and “b” the non-risk allele. The factor “r” is therefore the relative risk between the allele types.

For many of the studies published in the last few years, reporting common variants associated with complex diseases, the multiplicative model has been found to summarize the effect adequately and most often provide a fit to the data superior to alternative models such as the dominant and recessive models.

### The Risk Relative to the Average Population Risk

It is most convenient to represent the risk of a genetic variant relative to the average population since it makes it easier to communicate the lifetime risk for developing the disease compared with the baseline population risk. For example, in the multiplicative model we can calculate the relative population risk for variant “aa” as:

RR(aa)=Pr(A|aa)/Pr(A)=(Pr(A|aa)/Pr(A|bb))/(Pr(A)/Pr(A|bb))=r²/(Pr(aa)r²+Pr(ab)r+Pr(bb))=r²/(p²r²+2pqr+q²)=r²/R

Here “p” and “q” are the allele frequencies of “a” and “b” respectively. Likewise, we get that RR(ab)=r/R and RR(bb)=1/R. The allele frequency estimates may be obtained from the publications that report the odds-ratios and from the HapMap database. Note that in the case where we do not know the genotypes of an individual, the relative genetic risk for that test or marker is simply equal to one.

By way of an example, for UBC risk, allele T of the disease associated marker rs9642880 in has an allelic OR of 1.21 and a frequency (p) around 0.48 in white populations. The genotype relative risk compared to genotype GG are estimated based on the multiplicative model.

For TT it is 1.21×1.21=1.46; for TG it is simply the OR 1.21, and for GG it is 1.0 by definition.

The frequency of allele G is q=1−p=1−0.48=0.52. Population frequency of each of the three possible genotypes at this marker is:

Pr(TT)=p²=0.23,Pr(TG)=2pq=0.50,and Pr(GG)=q²=0.27

The average population risk relative to genotype GG (which is defined to have a risk of one) is:

R=0.23'1.46+0.50×1.21+0.27×1=1.21

Therefore, the risk relative to the general population (RR) for individuals who have one of the following genotypes at this marker is:

RR(TT)=1.46/1.21=1.21,RR(TG)=1.21/1.21=1.0,RR(GG)=1/1.21=0.83.

Combining the Risk from Multiple Markers

When genotypes of many SNP variants are used to estimate the risk for an individual a multiplicative model for risk can generally be assumed. This means that the combined genetic risk relative to the population is calculated as the product of the corresponding estimates for individual markers, e.g. for two markers g1 and g2:

RR(g1,g2)=RR(g1)RR(g2)

The underlying assumption is that the risk factors occur and behave independently, i.e. that the joint conditional probabilities can be represented as products:

Pr(A|g1,g2)=Pr(A|g1)Pr(A|g2)/Pr(A)and Pr(g1,g2)=Pr(g1)Pr(g2)

Obvious violations to this assumption are markers that are closely spaced on the genome, i.e. in linkage disequilibrium, such that the concurrence of two or more risk alleles is correlated. In such cases, we can use so called haplotype modeling where the odds-ratios are defined for all allele combinations of the correlated SNPs.

As is in most situations where a statistical model is utilized, the model applied is not expected to be exactly true since it is not based on an underlying bio-physical model. However, the multiplicative model has so far been found to fit the data adequately, i.e. no significant deviations are detected for many common diseases for which many risk variants have been discovered.

As an example, an individual who has the following genotypes at 4 hypothetical markers associated with a particular disease along with the risk relative to the population at each marker:

| Marker | Genotype | Calculated risk |
|---|---|---|
| M1 | CC | 1.03 |
| M2 | GG | 1.30 |
| M3 | AG | 0.88 |
| M4 | TT | 1.54 |

Combined, the overall risk relative to the population for this individual is: 1.03×1.30×0.88×1.54=1.81.

### Adjusted Life-Time Risk

The lifetime risk of an individual is derived by multiplying the overall genetic risk relative to the population with the average life-time risk of the disease in the general population of the same ethnicity and gender and in the region of the individual's geographical origin. As there are usually several epidemiologic studies to choose from when defining the general population risk, we will pick studies that are well-powered for the disease definition that has been used for the genetic variants.

For example, for a particular disease, if the overall genetic risk relative to the population is 1.8, and if the average life-time risk of the disease for individuals of the same demographic is 20%, then the adjusted lifetime risk for the individual is 20%×1.8=36%.

Note that since the average RR for a population is one, this multiplication model provides the Same average adjusted life-time risk of the disease. Furthermore, since the actual life-time risk cannot exceed 100%, there must be an upper limit to the genetic RR.

### Risk Assessment for Urinary Bladder Cancer

As described herein, certain polymorphic markers and haplotypes comprising such markers are found to be useful for risk assessment of urinary bladder cancer (UBC). Risk assessment can involve the use of the markers for diagnosing a susceptibility to UBC. Particular alleles of certain polymorphic markers are found more frequently in individuals with UBC, than in individuals without diagnosis of UBC. Therefore, these marker alleles have predictive value for detecting UBC, or a susceptibility to UBC, in an individual.

Tagging markers in linkage disequilibrium with at-risk variants (or protective variants) described herein can be used as surrogates for these markers (and/or haplotypes). Such surrogate markers can be located within a particular haplotype block or LD block. Such surrogate markers can also sometimes be located outside the physical boundaries of such a haplotype block or LD block, either in close vicinity of the LD block/haplotype block, but possibly also located in a more distant genomic location.

Long-distance LD can for example arise if particular genomic regions (e.g., genes) are in a functional relationship. For example, if two genes encode proteins that play a role in a shared metabolic pathway, then particular variants in one gene may have a direct impact on observed variants for the other gene. Let us consider the case where a variant in one gene leads to increased expression of the gene product. To counteract this effect and preserve overall flux of the particular pathway, this variant may have led to selection of one (or more) variants at a second gene that confers decreased expression levels of that gene. These two genes may be located in different genomic locations, possibly on different chromosomes, but variants within the genes are in apparent LD, not because of their shared physical location within a region of high LD, but rather due to evolutionary forces. Such LD is also contemplated and within scope of the present invention. The skilled person will appreciate that many other scenarios of functional gene-gene interaction are possible, and the particular example discussed here represents only one such possible scenario.

Markers with values of r² equal to 1 are perfect surrogates for the at-risk variants (anchor variants), i.e. genotypes for one marker perfectly predicts genotypes for the other. Markers with smaller values of r² than 1 can also be surrogates for the at-risk variant, or alternatively represent variants with relative risk values as high as or possibly even higher than the at-risk variant. In certain preferred embodiments, markers with values of r² to the at-risk anchor variant are useful surrogate markers. The at-risk variant identified may not be the functional variant itself, but is in this instance in linkage disequilibrium with the true functional variant. The functional variant may be a SNP, but may also for example be a tandem repeat, such as a minisatellite or a microsatellite, a transposable element (e.g., an Alu element), or a structural alteration, such as a deletion, insertion or inversion (sometimes also called copy number variations, or CNVs). The present invention encompasses the assessment of such surrogate markers for the markers as disclosed herein. Such markers are annotated, mapped and listed in public databases, as well known to the skilled person, or can alternatively be readily identified by sequencing the region or a part of the region identified by the markers of the present invention in a group of individuals, and identify polymorphisms in the resulting group of sequences. As a consequence, the person skilled in the art can readily and without undue experimentation identify and genotype surrogate markers in linkage disequilibrium with the markers and/or haplotypes as described herein. The tagging or surrogate markers in LD with the at-risk variants detected also have predictive value since they capture the effect observed by the at-risk variants (e.g., rs9642880, rs710521).

The present invention can in certain embodiments be practiced by assessing a sample comprising genomic DNA from an individual for the presence of certain variants described herein to be associated with UBC. Such assessment includes steps of detecting the presence or absence of at least one allele of at least one polymorphic marker, using methods well known to the skilled person and further described herein, and based on the outcome of such assessment, determine whether the individual from whom the sample is derived is at increased or decreased risk (i.e. increased or decreased susceptibility) of UBC. Detecting particular alleles of polymorphic markers can in certain embodiments be done by obtaining nucleic acid sequence data about a particular human individual that identifies at least one allele of at least one polymorphic marker. Different alleles of the at least one marker are associated with different susceptibility to the disease in humans. Obtaining nucleic acid sequence data can comprise nucleic acid sequence at a single nucleotide position, which is sufficient to identify alleles at SNPs. The nucleic acid sequence data can also comprise sequence at any other number of nucleotide positions, in particular for genetic markers that comprise multiple nucleotide positions, and can be anywhere from two to hundreds of thousands, possibly even millions, of nucleotides (in particular, in the case of copy number variations (CNVs)). In certain embodiments, the invention can be practiced utilizing a dataset comprising information about the genotype status of at least one polymorphic marker associated with a disease (or markers in linkage disequilibrium with at least one marker associated with the disease). In other words, a dataset containing information about such genetic status, for example in the form of genotype counts at a certain polymorphic marker, or a plurality of markers (e.g., an indication of the presence or absence of certain at-risk alleles), or actual genotypes for one or more markers, can be queried for the presence or absence of certain at-risk alleles at certain polymorphic markers shown by the present inventors to be associated with the disease. A positive result for a variant (e.g., marker allele) associated with the disease, is indicative of the individual from which the dataset is derived is at increased susceptibility (increased risk) of UBC.

In certain embodiments of the invention, a polymorphic marker is correlated to UBC by referencing genotype data for the polymorphic marker to a database, such as a look-up table that comprises correlations datas between at least one allele of the polymorphism and UBC. In some embodiments, the table comprises a correlation for one polymorphism. In other embodiments, the table comprises a correlation for a plurality of polymorphisms. In both scenarios, by referencing to a look-up table that gives an indication of a correlation between a marker and UBC, a risk for UBC, or a susceptibility to UBC, can be identified in the individual from whom the sample is derived. In some embodiments, the correlation is reported as a statistical measure. The statistical measure may be reported as a risk measure, such as a relative risk (RR), an absolute risk (AR) or an odds ratio (OR).

Risk markers may be useful for risk assessment and diagnostic purposes, either alone or in combination. Results of disease risk assessment based on the markers described herein can also be combined with data for other genetic markers or risk factors for the disease, to establish overall risk. Thus, even in cases where the increase in risk by individual markers is relatively modest, e.g. on the order of 10-30%, the association may have significant implications when combined with other risk markers. Thus, relatively common variants may have significant contribution to the overall risk (Population Attributable Risk is high), or combination of markers can be used to define groups of individual who, based on the combined risk of the markers, is at significant combined risk of developing the disease.

Thus, in one embodiment of the invention, a plurality of variants (genetic markers, biomarkers and/or haplotypes) is used for overall risk assessment. These variants are in one embodiment selected from the variants as disclosed herein. Other embodiments include the use of the variants of the present invention in combination with other variants known to be useful for diagnosing a susceptibility to UBC. In such embodiments, the genotype status of a plurality of markers and/or haplotypes is determined in an individual, and the status of the individual compared with the population frequency of the associated variants, or the frequency of the variants in clinically healthy subjects, such as age-matched and sex-matched subjects. Methods known in the art, such as multivariate analyses or joint risk analyses, such as those described herein, or other methods known to the skilled person, may subsequently be used to determine the overall risk conferred based on the genotype status at the multiple loci. Assessment of risk based on such analysis may subsequently be used in the methods, uses and kits of the invention, as described herein.

### Study Population

In a general sense, the methods and kits described herein can be utilized from samples containing nucleic acid material (DNA or RNA) from any source and from any individual, or from genotype or sequence data derived from such samples. In preferred embodiments, the individual is a human individual. The individual can be an adult, child, or fetus. The nucleic acid source may be any sample comprising nucleic acid material, including biological samples, or a sample comprising nucleic acid material derived therefrom. The present invention also provides for assessing markers and/or haplotypes in individuals who are members of a target population. Such a target population is in one embodiment a population or group of individuals at risk of developing the disease, based on factors, e.g., other genetic factors, biomarkers, biophysical parameters, history of UBC, previous diagnosis of UBC, family history of UBC.

The invention provides for embodiments that include individuals from specific age subgroups, such as those over the age of 40, over age of 45, or over age of 50, 55, 60, 65, 70, 75, 80, or 85. Other embodiments of the invention pertain to other age groups, such as individuals aged less than 85, such as less than age 80, less than age 75, or less than age 70, 65, 60, 55, 50, 45, 40, 35, or age 30. Other embodiments relate to individuals with age at onset of UBCin any of the age ranges described in the above. It is also contemplated that a range of ages may be relevant in certain embodiments, such as age at onset at more than age 45 but less than age 60. Other age ranges are however also contemplated, including all age ranges bracketed by the age values listed in the above. The invention furthermore relates to individuals of either gender, males or females.

The Icelandic population is a Caucasian population of Northern European ancestry. A large number of studies reporting results of genetic linkage and association in the Icelandic population have been published in the last few years. Many of those studies show replication of variants, originally identified in the Icelandic population as being associating with a particular disease, in other populations (Sulem, P., et al. Nat Genet May 17, 2009 (Epub ahead of print); Rafnar, T., et al. Nat Genet. 41:221-7 (2009); Gretarsdottir, S., et al. Ann Neurol 64:402-9 (2008); Stacey, S. N., et al. Nat Genet. 40:1313-18 (2008); Gudbjartsson, D. F., et al. Nat Genet. 40:886-91 (2008); Styrkarsdottir, U., et al. N Engl J Med 358:2355-65 (2008); Thorgeirsson, T., et al. Nature 452:638-42 (2008); Gudmundsson, 3., et al. Nat. Genet. 40:281-3 (2008); Stacey, S. N., et al., Nat. Genet. 39:865-69 (2007); Helgadottir, A., et al., Science 316:1491-93 (2007); Steinthorsdottir, V., et al., Nat. Genet. 39:770-75 (2007); Gudmundsson, J., et al., Nat. Genet. 39:631-37 (2007); Frayling, T M, Nature Reviews Genet. 8:657-662 (2007); Amundadottir, L. T., et al., Nat. Genet. 38:652-58 (2006); Grant, S. F., et al., Nat. Genet. 38:320-23 (2006)). Thus, genetic findings in the Icelandic population have in general been replicated in other populations, including populations from Africa and Asia.

It is thus believed that the markers described herein to be associated with risk of UBC will show similar association in other human populations. Particular embodiments comprising individual human populations are thus also contemplated and within the scope of the invention. Such embodiments relate to human subjects that are from one or more human population including, but not limited to, Caucasian populations, European populations, American populations, Eurasian populations, Asian populations, Central/South Asian populations, East Asian populations, Middle Eastern populations, African populations, Hispanic populations, and Oceanic populations. European populations include, but are not limited to, Swedish, Norwegian, Finnish, Russian, Danish, Icelandic, Irish, Celt, English, Scottish, Dutch, Belgian, French, German, Spanish, Portuguese, Italian, Polish, Bulgarian, Slavic, Serbian, Bosnian, Czech, Greek and Turkish populations. In certain embodiments, the invention relates to individuals of Caucasian origin.

The racial contribution in individual subjects may also be determined by genetic analysis. Genetic analysis of ancestry may be carried out using unlinked microsatellite markers such as those set out in Smith et al. (Am J Hum Genet. 74, 1001-13 (2004)).

In certain embodiments, the invention relates to markers and/or haplotypes identified in specific populations, as described in the above. The person skilled in the art will appreciate that measures of linkage disequilibrium (LD) may give different results when applied to different populations. This is due to different population history of different human populations as well as differential selective pressures that may have led to differences in LD in specific genomic regions. It is also well known to the person skilled in the art that certain markers, e.g. SNP markers, have different population frequency in different populations, or are polymorphic in one population but not in another. The person skilled in the art will however apply the methods available and as thought herein to practice the present invention in any given human population. This may include assessment of polymorphic markers in the LD region of the present invention, so as to identify those markers that give strongest association within the specific population. Thus, the at-risk variants of the present invention may reside on different haplotype background and in different frequencies in various human populations. However, utilizing methods known in the art and the markers of the present invention, the invention can be practiced in any given human population.

### Utility of Genetic Testing

The person skilled in the art will appreciate and understand that the variants described herein in general do not, by themselves, provide an absolute identification of individuals who will develop urinary bladder cancer. The variants described herein do however indicate increased and/or decreased likelihood that individuals carrying the at-risk or protective variants of the invention will develop UBC, or symptoms associated with UBC. This information is however extremely valuable in itself, as outlined in more detail in the below, as it can be used to, for example, initiate preventive measures at an early stage, perform regular physical exams to monitor the progress and/or appearance of symptoms, or to schedule exams at a regular interval to identify early symptoms, so as to be able to apply treatment at an early stage.

Bladder cancer is a disease with a high prevalence and potential for improved survival with early detection. Understanding of the genetic factors contributing to the residual genetic risk for bladder cancer is very limited. No universally successful method for the prevention or treatment of bladder cancer is currently available. Management of the disease currently relies on a combination of early diagnosis, appropriate treatments and secondary prevention. There are clear clinical imperatives for integrating genetic testing into all aspects of these management areas. Identification of cancer susceptibility genes may also reveal key molecular pathways that may be manipulated (e.g., using small or large molecular weight drugs) and may lead to more effective treatments.

A screening program that would result in detection of bladder cancer at an earlier stage, prior to muscle invasion or metastasis, could render a significant improvement in patient morbidity and overall survival. In order for bladder cancer screening to become a reality, first a high incidence population has to be identified and secondly a cost-effective marker with good performance characteristics has to be available. Individuals with many environmental risk factors, such as older male smokers and who have the high-risk genetic profile may benefit from periodic screening. Clinical screening for bladder cancer is mainly performed by urine cytology, cystoscopy or Hematuria tests.

Home urine dipstick to assess for hematuria is convenient, inexpensive, and noninvasive. However, utility of widespread screening with hematuria testing is limited due to the low positive predictive value (PPV) of the test. The PPV for hematuria dipstick for screening ranges between 5 and 8.3% resulting in many unnecessary workouts with their attendant patient anxiety and cost. Due to the relatively low sensitivity and low PPV of the reagent strip for hemoglobin as well as cytology, multiple urine-based bladder markers have been developed to try to assist in detecting bladder cancer non-invasively (Lotan Y, Roehrborn C G (2003) Urology 61(1):109-118). These include the NMP22 BladderChek Test (Matritech Inc., Newton, Mass., USA) and UroVysion (Vysis Downer's Grove, Ill., USA) (Grossman, H B et al. JAMA 293:810-816, 2005).

In accordance with the present invention, individuals that are homozygous for both the 3q and 8q bladder cancer variants may in particular benefit from screening for the disease, especially if they are also smokers. A simple urine test such as the NMP-22 assay may be useful for early diagnosis in this group.

Genetic variants described herein can be used alone or in combination, as well as in combination with other factors, including other genetic risk factors or biomarkers, for risk assessment of an individual for UBC. Many factors known to affect the predisposition of an individual towards developing risk of developing UBC are known to the person skilled in the art and can be utilized in such assessment. These include, but are not limited to, age, gender, smoking status and/or smoking history, family history of cancer, and of UBC in particular. Methods known in the art can be used for such assessment, including multivariate analyses or logistic regression.

### Methods

Methods for disease risk assessment and risk management are described herein and are encompassed by the invention. The invention also encompasses methods of assessing an individual for probability of response to a therapeutic agents, methods for predicting the effectiveness of a therapeutic agents, nucleic acids, polypeptides and antibodies and computer-implemented functions. Kits for use in the various methods presented herein are also encompassed by the invention.

### Diagnostic and Screening Methods

In certain embodiments, the present invention pertains to methods of diagnosing, or aiding in the diagnosis of UBC or a susceptibility to UBC, by detecting particular alleles at genetic markers that appear more frequently in UBC subjects or subjects who are susceptible to UBC. In particular embodiments, the invention is a method of determining a susceptibility to UBC by detecting at least one allele of at least one polymorphic marker (e.g., the markers described herein). In other embodiments, the invention relates to a method of diagnosing a susceptibility to UBC by detecting at least one allele of at least one polymorphic marker. The present invention describes methods whereby detection of particular alleles of particular markers or haplotypes is indicative of a susceptibility to UBC. Such prognostic or predictive assays can also be used to determine prophylactic treatment of a subject prior to the onset of symptoms of UBC.

The present invention pertains in some embodiments to methods of clinical applications of diagnosis, e.g., diagnosis performed by a medical professional. In other embodiments, the invention pertains to methods of diagnosis or determination of a susceptibility performed by a layman. The layman can be the customer of a genotyping service. The layman may also be a genotype service provider, who performs genotype analysis on a DNA sample from an individual, in order to provide service related to genetic risk factors for particular traits or diseases, based on the genotype status of the individual (i.e., the customer). Recent technological advances in genotyping technologies, including high-throughput genotyping of SNP markers, such as Molecular Inversion Probe array technology (e.g., Affymetrix GeneChip), and BeadArray Technologies (e.g., Illumina GoldenGate and Infinium assays) have made it possible for individuals to have their own genome assessed for up to one million SNPs simultaneously, at relatively little cost. The resulting genotype information, which can be made available to the individual, can be compared to information about disease or trait risk associated with various SNPs, including information from public literature and scientific publications. The diagnostic application of disease-associated alleles as described herein, can thus for example be performed by the individual, through analysis as described herein of his/her genotype data, by a health professional based on results of a clinical test, or by a third party, including the genotype service provider. The third party may also be service provider who interprets genotype information from the customer to provide service related to specific genetic risk factors, including the genetic markers described herein. In other words, the diagnosis or determination of a susceptibility of genetic risk can be made by health professionals, genetic counselors, third parties providing genotyping service or by the layman (e.g., the individual), based on information about the genotype status of an individual and knowledge about the risk conferred by particular genetic risk factors (e.g., particular SNPs). In the present context, the term “diagnosing”, “diagnose a susceptibility” and “determine a susceptibility” is meant to refer to any available diagnostic method, including those mentioned above.

In certain embodiments, a sample containing genomic DNA from an individual is collected. Such sample can for example be a buccal swab, a saliva sample, a blood sample, or other suitable samples containing genomic DNA, as described further herein. The genomic DNA is then analyzed using any common technique available to the skilled person, such as high-throughput array technologies. Results from such genotyping are stored in a convenient data storage unit, such as a data carrier, including computer databases, data storage disks, or by other convenient data storage means. In certain embodiments, the computer database is an object database, a relational database or a post-relational database. The genotype data is subsequently analyzed for the presence of certain variants known to be susceptibility variants for particular human conditions, such as the genetic variants described herein. Genotype data can be retrieved from the data storage unit using any convenient data query method. Calculating risk conferred by a particular genotype for the individual can be based on comparing the genotype of the individual to previously determined risk (expressed as a relative risk (RR) or and odds ratio (OR), for example) for the genotype, for example for an heterozygous carrier of an at-risk variant for a particular disease or trait. The calculated risk for the individual can be the relative risk for a person, or for a specific genotype of a person, compared to the average population with matched gender and ethnicity. The average population risk can be expressed as a weighted average of the risks of different genotypes, using results from a reference population, and the appropriate calculations to calculate the risk of a genotype group relative to the population can then be performed. Alternatively, the risk for an individual is based on a comparison of particular genotypes, for example heterozygous carriers of an at-risk allele of a marker compared with non-carriers of the at-risk allele. Using the population average may in certain embodiments be more convenient, since it provides a measure which is easy to interpret for the user, i.e. a measure that gives the risk for the individual, based on his/her genotype, compared with the average in the population. The calculated risk estimated can be made available to the customer via a website, preferably a secure website.

In certain embodiments, a service provider will include in the provided service all of the steps of isolating genomic DNA from a sample provided by the customer, performing genotyping of the isolated DNA, calculating genetic risk based on the genotype data, and report the risk to the customer. In some other embodiments, the service provider will include in the service the interpretation of genotype data for the individual, i.e., risk estimates for particular genetic variants based on the genotype data for the individual. In some other embodiments, the service provider may include service that includes genotyping service and interpretation of the genotype data, starting from a sample of isolated DNA from the individual (the customer).

Overall risk for multiple risk variants can be performed using standard methodology. For example, assuming a multiplicative model, i.e. assuming that the risk of individual risk variants multiply to establish the overall effect, allows for a straight-forward calculation of the overall risk for multiple markers.

In addition, in certain other embodiments, the present invention pertains to methods of diagnosing, or aiding in the diagnosis of, a decreased susceptibility to UBC, by detecting particular genetic marker alleles or haplotypes that appear less frequently in UBC patients than in individual not diagnosed with UBC or in the general population.

As described and exemplified herein, particular marker alleles or haplotypes (e.g. the markers as listed in Table 1 and markers in linkage disequilibrium therewith, including those listed in Tables 4 and 5) are associated with UBC. In one embodiment, the marker allele or haplotype is one that confers a significant risk or susceptibility to UBC. In another embodiment, the invention relates to a method of diagnosing a susceptibility to UBC in a human individual, the method comprising determining the presence or absence of at least one allele of at least one polymorphic marker in a nucleic acid sample obtained from the individual, wherein the at least one polymorphic marker is selected from the group consisting of the polymorphic markers listed in Table 1, and markers in linkage disequilibrium (defined as r²>0.2) therewith, such as those listed in Tables 4 and 5.

In another embodiment, the invention pertains to methods of diagnosing a susceptibility to UBC in a human individual, by screening for at least one marker allele as listed in Table 1 or markers in linkage disequilibrium therewith, such as those listed in Tables 4 and 5. In another embodiment, the marker allele or haplotype is more frequently present in a subject having, or who is susceptible to, UBC (affected), as compared to the frequency of its presence in a healthy subject (control, such as population controls). In certain embodiments, the significance of association of the at least one marker allele or haplotype is characterized by a p value <0.05. In other embodiments, the significance of association is characterized by smaller p-values, such as <0.01, <0.001, <0.0001, <0.00001, <0.000001, <0.0000001, <0.00000001 or <0.000000001.

In these embodiments, determination of the presence of the at least one marker allele or haplotype is indicative of a susceptibility to UBC. These diagnostic methods involve detecting the presence or absence of at least one marker allele or haplotype that is associated with UBC. The detection of the particular genetic marker alleles that make up particular haplotypes can be performed by a variety of methods described herein and/or known in the art. For example, genetic markers can be detected at the nucleic acid level (e.g., by direct nucleotide sequencing or by other means known to the skilled in the art) or at the amino acid level if the genetic marker affects the coding sequence of a protein encoded by a UBC-associated nucleic acid (e.g., by protein sequencing or by immunoassays using antibodies that recognize such a protein). The marker alleles or haplotypes correspond to fragments of a genomic DNA sequence associated with UBC. Such fragments encompass the DNA sequence of the polymorphic marker or haplotype in question, but may also include DNA segments in strong LD (linkage disequilibrium) with the marker or haplotype. In one embodiment, such segments comprises segments in LD with the marker or haplotype as determined by a value of r² greater than 0.1 and/or |D′|>0.8).

In one embodiment, diagnosis of a susceptibility to UBC can be accomplished using hybridization methods. (see Current Protocols in Molecular Biology, Ausubel, F. et al., eds., John Wiley & Sons, including all supplements). The presence of a specific marker allele can be indicated by sequence-specific hybridization of a nucleic acid probe specific for the particular allele. The presence of more than one specific marker allele or a specific haplotype can be indicated by using several sequence-specific nucleic acid probes, each being specific for a particular allele. In one embodiment, a haplotype can be indicated by a single nucleic acid probe that is specific for the specific haplotype (i.e., hybridizes specifically to a DNA strand comprising the specific marker alleles characteristic of the haplotype). A sequence-specific probe can be directed to hybridize to genomic DNA, RNA, or cDNA. A “nucleic acid probe”, as used herein, can be a DNA probe or an RNA probe that hybridizes to a complementary sequence. One of skill in the art would know how to design such a probe so that sequence specific hybridization will occur only if a particular allele is present in a genomic sequence from a test sample. The invention can also be reduced to practice using any convenient genotyping method, including commercially available technologies and methods for genotyping particular polymorphic markers.

To determine a susceptibility to UBC, a hybridization sample can be formed by contacting the test sample, such as a genomic DNA sample, with at least one nucleic acid probe. A non-limiting example of a probe for detecting mRNA or genomic DNA is a labeled nucleic acid probe that is capable of hybridizing to mRNA or genomic DNA sequences described herein. The nucleic acid probe can be, for example, a full-length nucleic acid molecule, or a portion thereof, such as an oligonucleotide of at least 15, 30, 50, 100, 250 or 500 nucleotides in length that is sufficient to specifically hybridize under stringent conditions to appropriate mRNA or genomic DNA. In certain embodiments, the oligonucleotide is from about 15 to about 100 nucleotides in length. In certain other embodiments, the oligonucleotide is from about 20 to about 50 nucleotides in length. For example, the nucleic acid probe can comprise all or a portion of the nucleotide sequence of any the sequences of SEQ ID NO:1-52, in particular all or portion of SEQ ID NO:1 or SEQ ID NO:2, as described herein, optionally comprising at least one allele of a marker described herein, or at least one haplotype described herein, or the probe can be the complementary sequence of such a sequence. Other suitable probes for use in the diagnostic assays of the invention are described herein. Hybridization can be performed by methods well known to the person skilled in the art (see, e.g., Current Protocols in Molecular Biology, Ausubel, F. et al., eds., John Wiley & Sons, including all supplements). In one embodiment, hybridization refers to specific hybridization, i.e., hybridization with no mismatches (exact hybridization). In one embodiment, the hybridization conditions for specific hybridization are high stringency.

Specific hybridization, if present, is detected using standard methods. If specific hybridization occurs between the nucleic acid probe and the nucleic acid in the test sample, then the sample contains the allele that is complementary to the nucleotide that is present in the nucleic acid probe. The process can be repeated for any markers of the present invention, or markers that make up a haplotype of the present invention, or multiple probes can be used concurrently to detect more than one marker alleles at a time. It is also possible to design a single probe containing more than one marker alleles of a particular haplotype (e.g., a probe containing alleles complementary to 2, 3, 4, 5 or all of the markers that make up a particular haplotype). Detection of the particular markers of the haplotype in the sample is indicative that the source of the sample has the particular haplotype (e.g., a haplotype) and therefore is susceptible to UBC.

In one preferred embodiment, a method utilizing a detection oligonucleotide probe comprising a fluorescent moiety or group at its 3′ terminus and a quencher at its 5′ terminus, and an enhancer oligonucleotide, is employed, as described by Kutyavin et al. (Nucleic Acid Res. 34:e128 (2006)). The fluorescent moiety can be Gig Harbor Green or Yakima Yellow, or other suitable fluorescent moieties. The detection probe is designed to hybridize to a short nucleotide sequence that includes the SNP polymorphism to be detected. Preferably, the SNP is anywhere from the terminal residue to −6 residues from the 3′ end of the detection probe. The enhancer is a short oligonucleotide probe which hybridizes to the DNA template 3′ relative to the detection probe. The probes are designed such that a single nucleotide gap exists between the detection probe and the enhancer nucleotide probe when both are bound to the template. The gap creates a synthetic a basic site that is recognized by an endonuclease, such as Endonuclease IV. The enzyme cleaves the dye off the fully complementary detection probe, but cannot cleave a detection probe containing a mismatch. Thus, by measuring the fluorescence of the released fluorescent moiety, assessment of the presence of a particular allele defined by nucleotide sequence of the detection probe can be performed.

The detection probe can be of any suitable size, although preferably the probe is relatively short. In one embodiment, the probe is from 5-100 nucleotides in length. In another embodiment, the probe is from 10-50 nucleotides in length, and in another embodiment, the probe is from 12-30 nucleotides in length. Other lengths of the probe are possible and within scope of the skill of the average person skilled in the art.

In a preferred embodiment, the DNA template containing the SNP polymorphism is amplified by Polymerase Chain Reaction (PCR) prior to detection. In such an embodiment, the amplified DNA serves as the template for the detection probe and the enhancer probe.

Certain embodiments of the detection probe, the enhancer probe, and/or the primers used for amplification of the template by PCR include the use of modified bases, including modified A and modified G. The use of modified bases can be useful for adjusting the melting temperature of the nucleotide molecule (probe and/or primer) to the template DNA, for example for increasing the melting temperature in regions containing a low percentage of G or C bases, in which modified A with the capability of forming three hydrogen bonds to its complementary T can be used, or for decreasing the melting temperature in regions containing a high percentage of G or C bases, for example by using modified G bases that form only two hydrogen bonds to their complementary C base in a double stranded DNA molecule. In a preferred embodiment, modified bases are used in the design of the detection nucleotide probe. Any modified base known to the skilled person can be selected in these methods, and the selection of suitable bases is well within the scope of the skilled person based on the teachings herein and known bases available from commercial sources as known to the skilled person.

Alternatively, a peptide nucleic acid (PNA) probe can be used in addition to, or instead of, a nucleic acid probe in the hybridization methods described herein. A PNA is a DNA mimic having a peptide-like, inorganic backbone, such as N-(2-aminoethyl)glycine units, with an organic base (A, G, C, T or U) attached to the glycine nitrogen via a methylene carbonyl linker (see, for example, Nielsen, P., et al., Bioconjug. Chem. 5:3-7 (1994)). The PNA probe can be designed to specifically hybridize to a molecule in a sample suspected of containing one or more of the marker alleles or haplotypes that are associated with UBC. In one embodiment of the invention, a test sample containing genomic DNA obtained from the subject is collected and the polymerase chain reaction (PCR) is used to amplify a fragment comprising one or more markers or haplotypes of the present invention. As described herein, identification of a particular marker allele or haplotype associated with UBC, can be accomplished using a variety of methods (e.g., sequence analysis, analysis by restriction digestion, specific hybridization, single stranded conformation polymorphism assays (SSCP), electrophoretic analysis, etc.). In another embodiment, diagnosis is accomplished by expression analysis, for example by using quantitative PCR (kinetic thermal cycling). This technique can, for example, utilize commercially available technologies, such as TaqMan® (Applied Biosystems, Foster City, Calif.). The technique can assess the presence of an alteration in the expression or composition of a polypeptide or splicing variant(s) that is encoded by a nucleic acid associated with UBC. Further, the expression of the variant(s) can be quantified as physically or functionally different.

In another embodiment of the methods of the invention, analysis by restriction digestion can be used to detect a particular allele if the allele results in the creation or elimination of a restriction site relative to a reference sequence. Restriction fragment length polymorphism (RFLP) analysis can be conducted, e.g., as described in Current Protocols in Molecular Biology, supra. The digestion pattern of the relevant DNA fragment indicates the presence or absence of the particular allele in the sample.

Sequence analysis can also be used to detect specific alleles or haplotypes associated with UBC (e.g. a combination of any of the polymorphic markers of Table 1 and/or markers in linkage disequilibrium therewith). Therefore, in one embodiment, determination of the presence or absence of a particular marker alleles or haplotypes comprises sequence analysis of a test sample of DNA or RNA obtained from a subject or individual. PCR or other appropriate methods can be used to amplify a portion of a nucleic acid associated with UBC, and the presence of a specific allele can then be detected directly by sequencing the polymorphic site (or multiple polymorphic sites in a haplotype) of the genomic DNA in the sample.

In another embodiment, arrays of oligonucleotide probes that are complementary to target nucleic acid sequence segments from a subject, can be used to identify polymorphisms in a nucleic acid associated with UBC (e.g. the polymorphic markers of Table 1 and markers in linkage disequilibrium therewith). For example, an oligonucleotide array can be used. Oligonucleotide arrays typically comprise a plurality of different oligonucleotide probes that are coupled to a surface of a substrate in different known locations. These arrays can generally be produced using mechanical synthesis methods or light directed synthesis methods that incorporate a combination of photolithographic methods and solid phase oligonucleotide synthesis methods, or by other methods known to the person skilled in the art (see, e.g., Bier, F. F., et al. Adv Biochem Eng Biotechnol 109:433-53 (2008); Hoheisel, J. D., Nat Rev Genet. 7:200-10 (2006); Fan, J. B., et al. Methods Enzymol 410:57-73 (2006); Raqoussis, J. & Elvidge, G., Expert Rev Mol Diagn 6:145-52 (2006); Mockler, T. C., et al Genomics 85:1-15 (2005), and references cited therein, the entire teachings of each of which are incorporated by reference herein). Many additional descriptions of the preparation and use of oligonucleotide arrays for detection of polymorphisms can be found, for example, in U.S. Pat. No. 6,858,394, U.S. Pat. No. 6,429,027, U.S. Pat. No. 5,445,934, U.S. Pat. No. 5,700,637, U.S. Pat. No. 5,744,305, U.S. Pat. No. 5,945,334, U.S. Pat. No. 6,054,270, U.S. Pat. No. 6,300,063, U.S. Pat. No. 6,733,977, U.S. Pat. No. 7,364,858, EP 619 321, and EP 373 203, the entire teachings of which are incorporated by reference herein.

Other methods of nucleic acid analysis that are available to those skilled in the art can be used to detect a particular allele at a polymorphic site. Representative methods include, for example, direct manual sequencing (Church and Gilbert, Proc. Natl. Acad. Sci. USA, 81: 19914995 (1988); Sanger, F., et al., Proc. Natl. Acad. Sci. USA, 74:5463-5467 (1977); Beavis, et al., U.S. Pat. No. 5,288,644); automated fluorescent sequencing; single-stranded conformation polymorphism assays (SSCP); clamped denaturing gel electrophoresis (CDGE); denaturing gradient gel electrophoresis (DGGE) (Sheffield, V., et al., Proc. Natl. Acad. Sci. USA, 86:232-236 (1989)), mobility shift analysis (Orita, M., et al., Proc. Natl. Acad. Sci. USA, 86:2766-2770 (1989)), restriction enzyme analysis (Flavell, R., et al., Cell, 15:25-41 (1978); Geever, R., et al., Proc. Natl. Acad. Sci. USA, 78:5081-5085 (1981)); heteroduplex analysis; chemical mismatch cleavage (CMC) (Cotton, R., et al., Proc. Natl. Acad. Sci. USA, 85:4397-4401 (1985)); RNase protection assays (Myers, R., et al., Science, 230:1242-1246 (1985); use of polypeptides that recognize nucleotide mismatches, such as E. coli mutS protein; and allele-specific PCR.

In another embodiment of the invention, diagnosis of UBC or a susceptibility to UBC can be made by examining expression and/or composition of a polypeptide encoded by a nucleic acid associated with UBC in those instances where the genetic marker(s) or haplotype(s) of the present invention result in a change in the composition or expression of the polypeptide. Thus, diagnosis of a susceptibility to UBC can be made by examining expression and/or composition of one of these polypeptides, or another polypeptide encoded by a nucleic acid associated with UBC, in those instances where the genetic marker or haplotype of the present invention results in a change in the composition or expression of the polypeptide. The markers described herein that show association to UBC may also affect expression of nearby genes, e.g. other genes on chromosomes 8 or 3, in the vicinity of the marker rs9642880 (SEQ ID NO: 1) or rs710521 (SEQ ID NO: 2), respectively. Surprisingly, though, as mentioned above, the first mentioned marker (rs9642880) appears not to be associated with known miss-sense mutations in the oncogene c-Myc. It is well known that regulatory element affecting gene expression may be located far away, even as far as tenths or hundreds of kilobases away, from the promoter region of a gene. By assaying for the presence or absence of at least one allele of at least one polymorphic marker of the present invention, it is thus possible to assess the expression level of such nearby genes. Possible mechanisms affecting these genes include, e.g., effects on transcription, effects on RNA splicing, alterations in relative amounts of alternative splice forms of mRNA, effects on RNA stability, effects on transport from the nucleus to cytoplasm, and effects on the efficiency and accuracy of translation.

A variety of methods can be used for detecting protein expression levels, including enzyme linked immunosorbent assays (ELISA), Western blots, immunoprecipitations and immunofluorescence. A test sample from a subject is assessed for the presence of an alteration in the expression and/or an alteration in composition of the polypeptide encoded by a nucleic acid associated with UBC. An alteration in expression of a polypeptide encoded by a nucleic acid associated with UBC can be, for example, an alteration in the quantitative polypeptide expression (i.e., the amount of polypeptide produced). An alteration in the composition of a polypeptide encoded by a nucleic acid associated with UBC is an alteration in the qualitative polypeptide expression (e.g., expression of a mutant polypeptide or of a different splicing variant). In one embodiment, diagnosis of a susceptibility to UBC is made by detecting a particular splicing variant encoded by a nucleic acid associated with UBC, or a particular pattern of splicing variants.

Both such alterations (quantitative and qualitative) can also be present. An “alteration” in the polypeptide expression or composition, as used herein, refers to an alteration in expression or composition in a test sample, as compared to the expression or composition of the polypeptide in a control sample. A control sample is a sample that corresponds to the test sample (e.g., is from the same type of cells), and is from a subject who is not affected by, and/or who does not have a susceptibility to, UBC. In one embodiment, the control sample is from a subject that does not possess a marker allele or haplotype associated with UBC, as described herein. Similarly, the presence of one or more different splicing variants in the test sample, or the presence of significantly different amounts of different splicing variants in the test sample, as compared with the control sample, can be indicative of a susceptibility to UBC. An alteration in the expression or composition of the polypeptide in the test sample, as compared with the control sample, can be indicative of a specific allele in the instance where the allele alters a splice site relative to the reference in the control sample. Various means of examining expression or composition of a polypeptide encoded by a nucleic acid are known to the person skilled in the art and can be used, including spectroscopy, colorimetry, electrophoresis, isoelectric focusing, and immunoassays (e.g., David et al., U.S. Pat. No. 4,376,110) such as immunoblotting (see, e.g., Current Protocols in Molecular Biology, particularly chapter 10, supra).

For example, in one embodiment, an antibody (e.g., an antibody with a detectable label) that is capable of binding to a polypeptide encoded by a nucleic acid associated with UBC can be used. Antibodies can be polyclonal or monoclonal. An intact antibody, or a fragment thereof (e.g., Fv, Fab, Fab′, F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a labeled secondary antibody (e.g., a fluorescently-labeled secondary antibody) and end-labeling of a DNA probe with biotin such that it can be detected with fluorescently-labeled streptavidin.

In one embodiment of this method, the level or amount of polypeptide encoded by a nucleic acid associated with UBC in a test sample is compared with the level or amount of the polypeptide in a control sample. A level or amount of the polypeptide in the test sample that is higher or lower than the level or amount of the polypeptide in the control sample, such that the difference is statistically significant, is indicative of an alteration in the expression of the polypeptide encoded by the nucleic acid, and is diagnostic for a particular allele or haplotype responsible for causing the difference in expression. Alternatively, the composition of the polypeptide in a test sample is compared with the composition of the polypeptide in a control sample. In another embodiment, both the level or amount and the composition of the polypeptide can be assessed in the test sample and in the control sample.

In another embodiment, the diagnosis of a susceptibility to UBC is made by detecting at least one marker or haplotypes of the present invention (e.g., associated alleles of the markers listed in Table 1, and markers in linkage disequilibrium therewith), in combination with an additional protein-based, RNA-based or DNA-based assay.

### Kits

Kits useful in the methods of the invention comprise components useful in any of the methods described herein, including for example, primers for nucleic acid amplification, hybridization probes, restriction enzymes (e.g., for RFLP analysis), allele-specific oligonucleotides, antibodies that bind to an altered polypeptide encoded by a nucleic acid of the invention as described herein (e.g., a genomic segment comprising at least one polymorphic marker and/or haplotype of the present invention) or to a non-altered (native) polypeptide encoded by a nucleic acid of the invention as described herein, means for amplification of a nucleic acid associated with UBC, means for analyzing the nucleic acid sequence of a nucleic acid associated with UBC, means for analyzing the amino acid sequence of a polypeptide encoded by a nucleic acid associated with UBC, etc. The kits can for example include necessary buffers, nucleic acid primers for amplifying nucleic acids of the invention (e.g., a nucleic acid segment comprising one or more of the polymorphic markers as described herein), and reagents for allele-specific detection of the fragments amplified using such primers and necessary enzymes (e.g., DNA polymerase). Additionally, kits can provide reagents for assays to be used in combination with the methods of the present invention, e.g., reagents for use with other UBC diagnostic assays.

In one embodiment, the invention pertains to a kit for assaying a sample from a subject to detect the presence of UBC, symptoms associated with UBC, or a susceptibility to UBC in a subject, wherein the kit comprises reagents necessary for selectively detecting at least one allele Of at least one polymorphism of the present invention in the genome of the individual. In one embodiment, the kit further correlation data between the at least one polymorphism and risk of UBC. In a particular embodiment, the reagents comprise at least one contiguous oligonucleotide that hybridizes to a fragment of the genome of the individual comprising at least one polymorphism of the present invention. In another embodiment, the reagents comprise at least one pair of oligonucleotides that hybridize to opposite strands of a genomic segment obtained from a subject, wherein each oligonucleotide primer pair is designed to selectively amplify a fragment of the genome of the individual that includes at least one polymorphism, wherein the polymorphism is selected from from the group consisting of the polymorphisms as listed in Table 1, and polymorphic markers in linkage disequilibrium therewith, including those in Tables 4 and 5. In yet another embodiment the fragment is at least 20 base pairs in size. Such oligonucleotides or nucleic acids (e.g., oligonucleotide primers) can be designed using portions of the nucleic acid sequence flanking polymorphisms (e.g., SNPs or microsatellites) that are indicative of UBC. In another embodiment, the kit comprises one or more labeled nucleic acids capable of allele-specific detection of one or more specific polymorphic markers or haplotypes associated with UBC, and reagents for detection of the label. Suitable labels include, e.g., a radioisotope, a fluorescent label, an enzyme label, an enzyme co-factor label, a magnetic label, a spin label, an epitope label.

In particular embodiments, the polymorphic marker or haplotype to be detected by the reagents of the kit comprises one or more markers, two or more markers, three or more markers, four or more markers or five or more markers selected from the group consisting of the markers set forth in Table 1, 4 and 5. In another embodiment, the marker or haplotype to be detected comprises at least one marker from the group of markers in strong linkage disequilibrium, as defined by values of r² greater than 0.2, to at least one of the group of markers listed in Table 1, including those listed in Tables 4 and 5. In another embodiment, the marker or haplotype to be detected is selected from rs9642880 and rs710521.

In a preferred embodiment, the DNA template containing the SNP polymorphism is amplified by Polymerase Chain Reaction (PCR) prior to detection, and primers for such amplification are included in the reagent kit. In such an embodiment, the amplified DNA serves as the template for the detection probe and the enhancer probe.

In one embodiment, the DNA template is amplified by means of Whole Genome Amplification (WGA) methods, prior to assessment for the presence of specific polymorphic markers as described herein. Standard methods well known to the skilled person for performing WGA may be utilized, and are within scope of the invention. In one such embodiment, reagents for performing WGA are included in the reagent kit.

In certain embodiments, the presence of a particular marker allele or haplotype is indicative of a susceptibility (increased susceptibility or decreased susceptibility) to urinary bladder cancer (UBC). In another embodiment, the presence of the marker allele or haplotype is indicative of response to a UBC therapeutic agent. In another embodiment, the presence of the marker allele or haplotype is indicative of UBC prognosis. In yet another embodiment, the presence of the marker or haplotype is indicative of progress of UBC treatment. Such treatment may include intervention by surgery, medication or by other means (e.g., lifestyle changes).

In a further aspect of the present invention, a pharmaceutical pack (kit) is provided, the pack comprising a therapeutic agent and a set of instructions for administration of the therapeutic agent to humans diagnostically tested for one or more variants of the present invention, as disclosed herein. The therapeutic agent can be a small molecule drug, an antibody, a peptide, an antisense or RNAi molecule, or other therapeutic molecules. In one embodiment, an individual identified as a carrier of at least one variant of the present invention is instructed to take a prescribed dose of the therapeutic agent. In one such embodiment, an individual identified as a homozygous carrier of at least one variant of the present invention is instructed to take a prescribed dose of the therapeutic agent. In another embodiment, an individual identified as a non-carrier of at least one variant of the present invention is instructed to take a prescribed dose of the therapeutic agent.

In certain embodiments, the kit further comprises a set of instructions for using the reagents comprising the kit.

### Therapeutic Agents

The variants (markers and/or haplotypes) disclosed herein to confer increased risk of UBC can be useful in the identification of novel therapeutic targets for UBC. For example, genes containing, or in linkage disequilibrium with, variants (markers and/or haplotypes) associated with UBC, or their products, as well as genes or their products that are directly or indirectly regulated by or interact with these variant genes or their products, can be targeted for the development of therapeutic agents to treat UBC, or prevent or delay onset of symptoms associated with UBC. Therapeutic agents may comprise one or more of, for example, small non-protein and non-nucleic acid molecules, proteins, peptides, protein fragments, nucleic acids (DNA, RNA), PNA (peptide nucleic acids), or their derivatives or mimetics which can modulate the function and/or levels of the target genes or their gene products.

The nucleic acids and/or variants of the invention, or nucleic acids comprising their complementary sequence, may be used as antisense constructs to control gene expression in cells, tissues or organs. The methodology associated with antisense techniques is well known to the skilled artisan, and is described and reviewed in AntisenseDrug Technology: Principles, Strategies, and Applications, Crooke, ed., Marcel Dekker Inc., New York (2001).

In general, antisense agents (antisense oligonucleotides) are comprised of single stranded oligonucleotides (RNA or DNA) that are capable of binding to a complimentary nucleotide segment. By binding the appropriate target sequence, an RNA-RNA, DNA-DNA or RNA-DNA duplex is formed. The antisense oligonucleotides are complementary to the sense or coding strand of a gene. It is also possible to form a triple helix, where the antisense oligonucleotide hinds to duplex DNA.

Several classes of antisense oligonucleotide are known to those skilled in the art, including cleavers and blockers. The former bind to target RNA sites, activate intracellular nucleases (e.g., RnaseH or Rnase L), that cleave the target RNA. Blockers bind to target RNA, inhibit protein translation by steric hindrance of the ribosomes. Examples of blockers include nucleic acids, morpholino compounds, locked nucleic acids and methylphosphonates (Thompson, Drug Discovery Today, 7:912-917 (2002)). Antisense oligonucleotides are useful directly as therapeutic agents, and are also useful for determining and validating gene function, for example by gene knock-out or gene knock-down experiments. Antisense technology is further described in Layery et al., Curr. Opin. Drug Discov. Devel. 6:561-569 (2003), Stephens at al., Curr. Opin. Mol. Ther. 5:118-122 (2003), Kurreck, Eur. J. Biochem. 270:1628-44 (2003), Dias et al., Mol. Cancer. Ter. 1:347-55 (2002), Chen, Methods Mol. Med. 75:621-636 (2003), Wang et al., Curr. Cancer Drug Targets 1:177-96 (2001), and Bennett, Antisense Nucleic Acid Drug. Dev. 12:215-24 (2002).

The variants described herein can also be used for the selection and design of antisense reagents that are specific for particular variants. Using information about the variants described herein, antisense oligonucleotides or other antisense molecules that specifically target mRNA molecules that contain one or more variants of the invention can be designed. In this manner, expression of mRNA molecules that contain one or more variant of the present invention (i.e. certain marker alleles and/or haplotypes) can be inhibited or blocked. In one embodiment, the antisense molecules are designed to specifically bind a particular allelic form (i.e., one or several variants (alleles and/or haplotypes)) of the target nucleic acid, thereby inhibiting translation of a product originating from this specific allele or haplotype, but which do not bind other or alternate variants at the specific polymorphic sites of the target nucleic acid molecule. As antisense molecules can be used to inactivate mRNA so as to inhibit gene expression, and thus protein expression, the molecules can be used for disease treatment. The methodology can involve cleavage by means of ribozymes containing nucleotide sequences complementary to one or more regions in the mRNA that attenuate the ability of the mRNA to be translated. Such mRNA regions include, for example, protein-coding regions, in particular protein-coding regions corresponding to catalytic activity, substrate and/or ligand binding sites, or other functional domains of a protein.

The phenomenon of RNA interference (RNAI) has been actively studied for the last decade, since its original discovery in C. elegans (Fire et al., Nature 391:806-11 (1998)), and in recent years its potential use in treatment of human disease has been actively pursued (reviewed in Kim & Rossi, Nature Rev. Genet. 8:173-204 (2007)). RNA interference (RNAi), also called gene silencing, is based on using double-stranded RNA molecules (dsRNA) to turn off specific genes. In the cell, cytoplasmic double-stranded RNA molecules (dsRNA) are processed by cellular complexes into small interfering RNA (siRNA). The siRNA guide the targeting of a protein-RNA complex to specific sites on a target mRNA, leading to cleavage of the mRNA (Thompson, Drug Discovery Today, 7:912-917 (2002)). The siRNA molecules are typically about 20, 21, 22 or 23 nucleotides in length. Thus, one aspect of the invention relates to isolated nucleic acid molecules, and the use of those molecules for RNA interference, i.e. as small interfering RNA molecules (siRNA). In one embodiment, the isolated nucleic acid molecules are 18-26 nucleotides in length, preferably 19-25 nucleotides in length, more preferably 20-24 nucleotides in length, and more preferably 22 or 23 nucleotides in length.

Another pathway for RNAi-mediated gene silencing originates in endogenously encoded primary microRNA (pri-miRNA) transcripts, which are processed in the cell to generate precursor miRNA (pre-miRNA). These miRNA molecules are exported from the nucleus to the cytoplasm, where they undergo processing to generate mature miRNA molecules (miRNA), which direct translational inhibition by recognizing target sites in the 3′ untranslated regions of mRNAs, and subsequent mRNA degradation by processing P-bodies (reviewed in Kim & Rossi, Nature Rev. Genet. 8:173-204 (2007)).

Clinical applications of RNAi include the incorporation of synthetic siRNA duplexes, which preferably are approximately 20-23 nucleotides in size, and preferably have 3′ overlaps of 2 nucleotides. Knockdown of gene expression is established by sequence-specific design for the target mRNA. Several commercial sites for optimal design and synthesis of such molecules are known to those skilled in the art.

Other applications provide longer siRNA molecules (typically 25-30 nucleotides in length, preferably about 27 nucleotides), as well as small hairpin RNAs (shRNAs; typically about 29 nucleotides in length). The latter are naturally expressed, as described in Amarzguioui et al. (FEBS Lett. 579:5974-81 (2005)). Chemically synthetic siRNAs and shRNAs are substrates for in vivo processing, and in some cases provide more potent gene-silencing than shorter designs (Kim et al., Nature Biotechnol. 23:222-226 (2005); Siolas et al., Nature Biotechnol. 23:227-231 (2005)). In general siRNAs provide for transient silencing of gene expression, because their intracellular concentration is diluted by subsequent cell divisions. By contrast, expressed shRNAs mediate long-term, stable knockdown of target transcripts, for as long as transcription of the shRNA takes place (Marques et al., Nature Biotechnol. 23:559-565 (2006); Brummelkamp et al., Science 296: 550-553 (2002)).

Since RNAi molecules, including siRNA, miRNA and shRNA, act in a sequence-dependent manner, the variants presented herein can be used to design RNAi reagents that recognize specific nucleic acid molecules comprising specific alleles and/or haplotypes (e.g., the alleles and/or haplotypes of the present invention), while not recognizing nucleic acid molecules comprising other alleles or haplotypes. These RNAi reagents can thus recognize and destroy the target nucleic acid molecules. As with antisense reagents, RNAi reagents can be useful as therapeutic agents (i.e., for turning off disease-associated genes or disease-associated gene variants), but may also be useful for characterizing and validating gene function (e.g., by gene knock-out or gene knock-down experiments).

Delivery of RNAi may be performed by a range of methodologies known to those skilled in the art. Methods utilizing non-viral delivery include cholesterol, stable nucleic acid-lipid particle (SNALP), heavy-chain antibody fragment (Fab), aptamers and nanoparticles. Viral delivery methods include use of lentivirus, adenovirus and adeno-associated virus. The siRNA molecules are in some embodiments chemically modified to increase their stability. This can include modifications at the 2′ position of the ribose, including 2′-O-methylpurines and 2′-fluoropyrimidines, which provide resistance to Rnase activity. Other chemical modifications are possible and known to those skilled in the art.

The following references provide a further summary of RNAi, and possibilities for targeting specific genes using RNAi: Kim & Rossi, Nat. Rev. Genet. 8:173-184 (2007), Chen & Rajevvsky, Nat. Rev. Genet. 8: 93-103 (2007), Reynolds, et al., Nat. Biotechnol. 22:326-330 (2004), Chi et al., Proc. Natl. Acad. Sci. USA 100:6343-6346 (2003), Vickers et al., J. Biol. Chem. 278:7108-7118 (2003), Agami, Curr. Opin. Chem. Biol. 6:829-834 (2002), Layery, et al., Curr. Opin. Drug Discov. Devel. 6:561-569 (2003), Shi, Trends Genet. 19:9-12 (2003), Shuey et al., Drug Discov. Today 7:1040-46 (2002), McManus et al., Nat. Rev. Genet. 3:737-747 (2002), Xia et al., Nat. Biotechnol. 20:1006-10 (2002), Plasterk et al., Curr Opin Genet Dev 10:562-7 (2000), Bosher et al., Nat. Cell Biol. 2:E31-6 (2000), and Hunter, Curr. Biol. 9:R440-442 (1999).

A genetic defect leading to increased predisposition or risk for development of a disease, including UBC, or a defect causing the disease, may be corrected permanently by administering to a subject carrying the defect a nucleic acid fragment that incorporates a repair sequence that supplies the normal/wild-type nucleotide(s) at the site of the genetic defect. Such site-specific repair sequence may encompass an RNA/DNA oligonucleotide that operates to promote endogenous repair of a subject's genomic DNA. The administration of the repair sequence may be performed by an appropriate vehicle, such as a complex with polyethylenimine, encapsulated in anionic liposomes, a viral vector such as an adenovirus vector, or other pharmaceutical compositions suitable for promoting intracellular uptake of the adminstered nucleic acid. The genetic defect may then be overcome, since the chimeric oligonucleotides induce the incorporation of the normal sequence into the genome of the subject, leading to expression of the normal/wild-type gene product. The replacement is propagated, thus rendering a permanent repair and alleviation of the symptoms associated with the disease or condition.

The present invention provides methods for identifying compounds or agents that can be used to treat UBC. Thus, the variants of the invention are useful as targets for the identification and/or development of therapeutic agents. In certain embodiments, such methods include assaying the ability of an agent or compound to modulate the activity and/or expression of a nucleic acid that includes at least one of the variants (markers and/or haplotypes) of the present invention, or the encoded product of the nucleic acid sequences comprising the variants or located in the vicinity of the variants. This in turn can be used to identify agents or compounds that inhibit or alter the undesired activity or expression of the encoded nucleic acid product. Assays for performing such experiments can be performed in cell-based systems or in cell-free systems, as known to the skilled person. Cell-based systems include cells naturally expressing the nucleic acid molecules of interest, or recombinant cells that have been genetically modified so as to express a certain desired nucleic acid molecule.

Variant gene expression in a patient can be assessed by expression of a variant-containing nucleic acid sequence (for example, a gene containing at least one variant of the present invention, which can be transcribed into RNA containing the at least one variant, and in turn translated into protein), or by altered expression of a normal/wild-type nucleic acid sequence due to variants affecting the level or pattern of expression of the normal transcripts, for example variants in the regulatory or control region of the gene. Assays for gene expression include direct nucleic acid assays (mRNA), assays for expressed protein levels, or assays of collateral compounds involved in a pathway, for example a signal pathway. Furthermore, the expression of genes that are up- or down-regulated in response to the signal pathway can also be assayed. One embodiment includes operably linking a reporter gene, such as luciferase, to the regulatory region of the gene(s) of interest.

Modulators of gene expression can in one embodiment be identified when a cell is contacted with a candidate compound or agent, and the expression of mRNA is determined. The expression level of mRNA in the presence of the candidate compound or agent is compared to the expression level in the absence of the compound or agent. Based on this comparison, candidate compounds or agents for treating UBC can be identified as those modulating the gene expression of the variant gene. When expression of mRNA or the encoded protein is statistically significantly greater in the presence of the candidate compound or agent than in its absence, then the candidate compound or agent is identified as a stimulator or up-regulator of expression of the nucleic acid. When nucleic acid expression or protein level is statistically significantly less in the presence of the candidate compound or agent than in its absence, then the candidate compound is identified as an inhibitor or down-regulator of the nucleic acid expression.

The invention further provides methods of treatment using a compound identified through drug (compound and/or agent) screening as a gene modulator (i.e. stimulator and/or inhibitor of gene expression).

### Methods of Assessing Probability of Response to Therapeutic Agents, Methods of Monitoring Progress of Treatment and Methods of Treatment

As is known in the art, individuals can have differential responses to a particular therapy (e.g., a therapeutic agent or therapeutic method). Pharmacogenomics addresses the issue of how genetic variations (e.g., the variants (markers and/or haplotypes) of the present invention) affect drug response, due to altered drug disposition and/or abnormal or altered action of the drug. Thus, the basis of the differential response may be genetically determined in part. Clinical outcomes due to genetic variations affecting drug response may result in toxicity of the drug in certain individuals (e.g., carriers or non-carriers of the genetic variants of the present invention), or therapeutic failure of the drug. Therefore, the variants of the present invention may determine the manner in which a therapeutic agent and/or method acts on the body, or the way in which the body metabolizes the therapeutic agent.

Accordingly, in one embodiment, the presence of a particular allele at a polymorphic site or haplotype is indicative of a different response, e.g. a different response rate, to a particular treatment modality. This means that a patient diagnosed with UBC, and carrying a certain allele at a polymorphic or haplotype of the present invention (e.g., the at-risk and protective alleles and/or haplotypes of the invention) would respond better to, or worse to, a specific therapeutic, drug and/or other therapy used to treat the disease. Therefore, the presence or absence of the marker allele or haplotype could aid in deciding what treatment should be used for a patient. For example, for a newly diagnosed patient, the presence of a marker or haplotype of the present invention may be assessed (e.g., through testing DNA derived from a blood sample, as described herein). If the patient is positive for a marker allele or haplotype (that is, at least one specific allele of the marker, or haplotype, is present), then the physician recommends one particular therapy, while if the patient is negative for the at least one allele of a marker, or a haplotype, then a different course of therapy may be recommended (which may include recommending that no immediate therapy, other than serial monitoring for progression of the disease, be performed). Thus, the patient's carrier status could be used to help determine whether a particular treatment modality should be administered. The value lies within the possibilities of being able to diagnose the disease at an early stage, to select the most appropriate treatment, and provide information to the clinician about prognosis/aggressiveness of the disease in order to be able to apply the most appropriate treatment. To strengthen these applications of the inventions, further genetic sampling is useful, of defined groups, e.g., patients who have responded favourably or not favourably to specific treatment, and to analyse the statistical distribution/allelic status of the markers of the invention within these groups. Thereby, individuals can be classified with greater certainty to such pre-defined groups.

As described above, current clinical treatment options for UBC include different surgical Procedures, depending on the severity of the cases, e.g. whether the cancer is invasive into the muscle wall of the bladder. Treatment options also include radiation therapy, for which a proportion of patients experience adverse symptoms. The markers of the invention, as described herein, may be used to assess response to these therapeutic options, or to predict the progress of therapy using any one of these treatment options. Thus, genetic profiling can be used to select the appropriate treatment strategy based on the genetic status of the individual, or it may be used to predict the outcome of the particular treatment option, and thus be useful in the strategic selection of treatment options or a combination of available treatment options. Again, such profiling and classification of individuals is supported further by first analysing known groups of patients for marker and/or haplotype status, as described further herein.

The present invention also relates to methods of monitoring progress or effectiveness of a treatment for a UBC, which is extremely valuable in general for all types of cancer treatment. This can be done based on the genotype and/or haplotype status of the markers and haplotypes of the present invention, i.e., by assessing the absence or presence of at least one allele of at least one polymorphic marker as disclosed herein, or by monitoring expression of genes that are associated with the variants (markers and haplotypes) of the present invention. The risk gene mRNA or the encoded polypeptide can be measured in a tissue sample (e.g., a peripheral blood sample, or a biopsy sample). Expression levels and/or mRNA levels can thus be determined before and during treatment to monitor its effectiveness. Alternatively, or concomitantly, the genotype and/or haplotype status of at least one risk variant for UBC as presented herein is determined before and during treatment to monitor its effectiveness.

Alternatively, biological networks or metabolic pathways related to the markers and haplotypes of the present invention can be monitored by determining mRNA and/or polypeptide levels. This can be done for example, by monitoring expression levels or polypeptides for several genes belonging to the network and/or pathway, in samples taken before and during treatment. Alternatively, metabolites belonging to the biological network or metabolic pathway can be determined before and during treatment. Effectiveness of the treatment is determined by comparing observed changes in expression levels/metabolite levels during treatment to corresponding data from healthy subjects.

In a further aspect, the markers of the present invention can be used to increase power and effectiveness of clinical trials. Thus, individuals who are carriers of at least one at-risk variant of the present invention, i.e. individuals who are carriers of at least one allele of at least one polymorphic marker conferring increased risk of developing UBC may be more likely to respond to a particular treatment modality. In one embodiment, individuals who carry at-risk variants for gene(s) in a pathway and/or metabolic network for which a particular treatment (e.g., small molecule drug) is targeting, are more likely to be responders to the treatment. In another embodiment, individuals who carry at-risk variants for a gene, which expression and/or function is altered by the at-risk variant, are more likely to be responders to a treatment modality targeting that gene, its expression or its gene product. This application can improve the safety of clinical trials, but can also enhance the chance that a clinical trial will demonstrate statistically significant efficacy, which may be limited to a certain sub-group of the population. Thus, one possible outcome of such a trial is that carriers of certain genetic variants, e.g., the markers and haplotypes of the present invention, are statistically significantly likely to show positive response to the therapeutic agent, i.e. experience alleviation of symptoms associated with UBC when taking the therapeutic agent or drug as prescribed.

In a further aspect, the markers and haplotypes of the present invention can be used for targeting the selection of pharmaceutical agents for specific individuals. Personalized selection of treatment modalities, lifestyle changes or combination of the two, can be realized by the utilization of the at-risk variants of the present invention. Thus, the knowledge of an individual's status for particular markers of the present invention, can be useful for selection of treatment options that target genes or gene products affected by the at-risk variants of the invention. Certain combinations of variants may be suitable for one selection of treatment options, while other gene variant combinations may target other treatment options. This becomes readily apparent upon analysis of known groups, who have undergone said treatments and been classified according to the results. Such combination of variant may include one variant, two variants, three variants, or four or more variants, as needed to determine with clinically reliable accuracy the selection of treatment module.

### Computer-Implemented Aspects

As understood by those of ordinary skill in the art, the methods and information described herein may be implemented, in all or in part, as computer executable instructions on known computer readable media. For example, the methods described herein may be implemented in hardware. Alternatively, the method may be implemented in software stored in, for example, one or more memories or other computer readable medium and implemented on one or more processors. As is known, the processors may be associated with one or more controllers, calculation units and/or other units of a computer system, or implanted in firmware as desired. If implemented in software, the routines may be stored in any computer readable memory such as in RAM, ROM, flash memory, a magnetic disk, a laser disk, or other storage medium, as is also known. Likewise, this software may be delivered to a computing device via any known delivery method including, for example, over a communication channel such as a telephone line, the Internet, a wireless connection, etc., or via a transportable medium, such as a computer readable disk, flash drive, etc.

More generally, and as understood by those of ordinary skill in the art, the various steps described above may be implemented as various blocks, operations, tools, modules and techniques which, in turn, may be implemented in hardware, firmware, software, or any combination of hardware, firmware, and/or software. When implemented in hardware, some or all of the blocks, operations, techniques, etc. may be implemented in, for example, a custom integrated circuit (IC), an application specific integrated circuit (ASIC), a field programmable logic array (FPGA), a programmable logic array (PLA), etc.

When implemented in software, the software may be stored in any known computer readable medium such as on a magnetic disk, an optical disk, or other storage medium, in a RAM or ROM or flash memory of a computer, processor, hard disk drive, optical disk drive, tape drive, etc. Likewise, the software may be delivered to a user or a computing system via any known delivery method including, for example, on a computer readable disk or other transportable computer storage mechanism.

FIG. 2 illustrates an example of a suitable computing system environment 100 on which a system for the steps of the claimed method and apparatus may be implemented. The computing system environment 100 is only one example of a suitable computing environment and is not intended to suggest any limitation as to the scope of use or functionality of the method or apparatus of the claims. Neither should the computing environment 100 be interpreted as having any dependency or requirement relating to any one or combination of components illustrated in the exemplary operating environment 100.

The steps of the claimed method and system are operational with numerous other general purpose or special purpose computing system environments or configurations. Examples of well known computing systems, environments, and/or configurations that may be suitable for use with the methods or system of the claims include, but are not limited to, personal computers, server computers, hand-held or laptop devices, multiprocessor systems, microprocessor-based systems, set top boxes, programmable consumer electronics, network PCs, minicomputers, mainframe computers, distributed computing environments that include any of the above systems or devices, and the like.

The steps of the claimed method and system may be described in the general context of computer-executable instructions, such as program modules, being executed by a computer. Generally, program modules include routines, programs, objects, components, data structures, etc. that perform particular tasks or implement particular abstract data types. The methods and apparatus may also be practiced in distributed computing environments where tasks are performed by remote processing devices that are linked through a communications network. In both integrated and distributed computing environments, program modules may be located in both local and remote computer storage media including memory storage devices.

With reference to FIG. 2, an exemplary system for implementing the steps of the claimed method and system includes a general purpose computing device in the form of a computer 110.

Components of computer 110 may include, but are not limited to, a processing unit 120, a system memory 130, and a system bus 121 that couples various system components including the system memory to the processing unit 120. The system bus 121 may be any of several types of bus structures including a memory bus or memory controller, a peripheral bus, and a local bus using any of a variety of bus architectures. By way of example, and not limitation, such architectures include Industry Standard Architecture (USA) bus, Micro Channel Architecture (MCA) bus, Enhanced ISA (EISA) bus, Video Electronics Standards Association (VESA) local bus, and Peripheral Component Interconnect (PCI) bus also known as Mezzanine bus.

Computer 110 typically includes a variety of computer readable media. Computer readable media can be any available media that can be accessed by computer 110 and includes both volatile and nonvolatile media, removable and non-removable media. By way of example, and pot limitation, computer readable media may comprise computer storage media and communication media. Computer storage media includes both volatile and nonvolatile, removable and non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can accessed by computer 110. Communication media typically embodies computer readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of the any of the above should also be included within the scope of computer readable media.

The system memory 130 includes computer storage media in the form of volatile and/or nonvolatile memory such as read only memory (ROM) 131 and random access memory (RAM) 132. A basic input/output system 133 (BIOS), containing the basic routines that help to transfer information between elements within computer 110, such as during start-up, is typically stored in ROM 131. RAM 132 typically contains data and/or program modules that are immediately accessible to and/or presently being operated on by processing unit 120. By way of example, and not limitation, FIG. 2 illustrates operating system 134, application programs 135, other program modules 136, and program data 137.

The computer 110 may also include other removable/non-removable, volatile/nonvolatile computer storage media. By way of example only, FIG. 2 illustrates a hard disk drive 140 that reads from or writes to non-removable, nonvolatile magnetic media, a magnetic disk drive 151 that reads from or writes to a removable, nonvolatile magnetic disk 152, and an optical disk drive 155 that reads from or writes to a removable, nonvolatile optical disk 156 such as a CD ROM or other optical media. Other removable/non-removable, volatile/nonvolatile computer storage media that can be used in the exemplary operating environment include, but are not limited to, magnetic tape cassettes, flash memory cards, digital versatile disks, digital video tape, solid state RAM, solid state ROM, and the like. The hard disk drive 141 is typically connected to the system bus 121 through a non-removable memory interface such as interface 140, and magnetic disk drive 151 and optical disk drive 155 are typically connected to the system bus 121 by a removable memory interface, such as interface 150.

The drives and their associated computer storage media discussed above and illustrated in FIG. 2, provide storage of computer readable instructions, data structures, program modules and other data for the computer 110. In FIG. 2, for example, hard disk drive 141 is illustrated as storing operating system 144, application programs 145, other program modules 146, and program data 147. Note that these components can either be the same as or different from operating system 134, application programs 135, other program modules 136, and program data 137. Operating system 144, application programs 145, other program modules 146, and program data 147 are given different numbers here to illustrate that, at a minimum, they are different copies. A user may enter commands and information into the computer 20 through input devices such as a keyboard 162 and pointing device 161, commonly referred to as a mouse, trackball or touch pad. Other input devices (not shown) may include a microphone, joystick, game pad, satellite dish, scanner, or the like. These and other input devices are often connected to the processing unit 120 through a user input interface 160 that is coupled to the system bus, but may be connected by other interface and bus structures, such as a parallel port, game port or a universal serial bus (USB). A monitor 191 or other type of display device is also connected to the system bus 121 via an interface, such as a video interface 190. In addition to the monitor, computers may also include other peripheral output devices such as speakers 197 and printer 196, which may be connected through an output peripheral interface 190.

The computer 110 may operate in a networked environment using logical connections to one or more remote computers, such as a remote computer 180. The remote computer 180 may be a personal computer, a server, a router, a network PC, a peer device or other common network node, and typically includes many or all of the elements described above relative to the computer 110, although only a memory storage device 181 has been illustrated in FIG. 2. The logical connections depicted in FIG. 2 include a local area network (LAN) 171 and a wide area network (WAN) 173, but may also include other networks. Such networking environments are commonplace in offices, enterprise-wide computer networks, intranets and the Internet.

When used in a LAN networking environment, the computer 110 is connected to the LAN 171 through a network interface or adapter 170. When used in a WAN networking environment, the computer 110 typically includes a modem 172 or other means for establishing communications over the WAN 173, such as the Internet. The modem 172, which may be internal or external, may be connected to the system bus 121 via the user input interface 160, or other appropriate mechanism. In a networked environment, program modules depicted relative to the computer 110, or portions thereof, may be stored in the remote memory storage device. By way of example, and not limitation, FIG. 2 illustrates remote application programs 185 as residing on memory device 181. It will be appreciated that the network connections shown are exemplary and other means of establishing a communications link between the computers may be used.

Although the forgoing text sets forth a detailed description of numerous different embodiments of the invention, it should be understood that the scope of the invention is defined by the words of the claims set forth at the end of this patent. The detailed description is to be construed as exemplary only and does not describe every possibly embodiment of the invention because describing every possible embodiment would be impractical, if not impossible. Numerous alternative embodiments could be implemented, using either current technology or technology developed after the filing date of this patent, which would still fall within the scope of the claims defining the invention.

While the risk evaluation system and method, and other elements, have been described as preferably being implemented in software, they may be implemented in hardware, firmware, etc., and may be implemented by any other processor. Thus, the elements described herein may be implemented in a standard multi-purpose CPU or on specifically designed hardware or firmware such as an application-specific integrated circuit (ASIC) or other hard-wired device as desired, including, but not limited to, the computer 110 of FIG. 2. When implemented in software, the software routine may be stored in any computer readable memory such as on a magnetic disk, a laser disk, or other storage medium, in a RAM or ROM of a computer or processor, in any database, etc. Likewise, this software may be delivered to a user or a diagnostic system via any known or desired delivery method including, for example, on a computer readable disk or other transportable computer storage mechanism or over a communication channel such as a telephone line, the Internet, wireless communication, etc. (which are viewed as being the same as or interchangeable with providing such software via a transportable storage medium).

Thus, many modifications and variations may be made in the techniques and structures described and illustrated herein without departing from the spirit and scope of the present invention. Thus, it should be understood that the methods and apparatus described herein are illustrative only and are not limiting upon the scope of the invention.

Accordingly, the invention relates to computer-implemented applications of the polymorphic markers and haplotypes described herein to be associated with urinary bladder cancer (UBC). Such applications can be useful for storing, manipulating or otherwise analyzing genotype data that is useful in the methods of the invention. One example pertains to storing genotype information derived from an individual on readable media, so as to be able to provide the genotype information to a third party (e.g., the individual, a health care provider or genetic analysis service provider), or for deriving information from the genotype data, e.g., by comparing the genotype data to information about genetic risk factors contributing to increased susceptibility to UBC, and reporting results based on such comparison.

In general terms, computer-readable media has capabilities of storing (i) identifier information for at least one polymorphic marker or a haplotype, preferably one or more of those listed in Tables 1, 4 or 5; (ii) an indicator of the frequency of at least one allele of said at least one marker, or the frequency of a haplotype, in individuals with UBC; and an indicator of the frequency of at least one allele of said at least one marker, or the frequency of a haplotype, in a reference population. The reference population can be a disease-free population of individuals. Alternatively, the reference population is a random sample from the general population, and is thus representative of the population at large. The frequency indicator may be a calculated frequency, a count of alleles and/or haplotype copies, or normalized or otherwise manipulated values of the actual frequencies that are suitable for the particular medium. The media may further include genotype data for one or more individuals, in a suitable format, such as genotype identity, genotype counts of particular alleles at particular markers, sequence data that include particular polymorphic positions, etc. Data stored on computer-readable media may thus be used to determine risk of UBC for particular markers and particular individuals.

The markers and haplotypes described herein to be associated with increased susceptibility (increased risk) of urinary bladder cancer (UBC), are in certain embodiments useful for interpretation and/or analysis of genotype data. Thus in certain embodiments, determination of the presence of an at-risk allele for UBC, as shown herein, or determination of the presence of an allele at a polymorphic marker in LD with any such risk allele, is indicative of the individual from whom the genotype data originates is at increased risk of urinary bladder cancer. In one such embodiment, genotype data is generated for at least one polymorphic marker shown herein to be associated with UBC, or a marker in linkage disequilibrium therewith. The genotype data is subsequently made available to a third party, such as the individual from whom the data originates, his/her guardian or representative, a physician or health care worker, genetic counselor, or insurance agent, for example via a user interface accessible over the internet, together with an interpretation of the genotype data, e.g., in the form of a risk measure (such as an absolute risk (AR), risk ratio (RR) or odds ratio (OR)) for the disease. In another embodiment, at-risk markers identified in a genotype dataset derived from an individual are assessed and results from the assessment of the risk conferred by the presence of such at-risk variants in the dataset are made available to the third party, for example via a secure web interface, or by other communication means. The results of such risk assessment can be reported in numeric form (e.g., by risk values, such as absolute risk, relative risk, and/or an odds ratio, or by a percentage increase in risk compared with a reference), by graphical means, or by other means suitable to illustrate the risk to the individual from whom the genotype data is derived.

### Nucleic Acids and Polypeptides

The nucleic acids and polypeptides described herein can be used in methods and kits of the present invention. An “isolated” nucleic acid molecule, as used herein, is one that is separated from nucleic acids that normally flank the gene or nucleotide sequence (as in genomic sequences) and/or has been completely or partially purified from other transcribed sequences (e.g., as in an RNA library). For example, an isolated nucleic acid of the invention can be substantially isolated with respect to the complex cellular milieu in which it naturally occurs, or culture medium when produced by recombinant techniques, or chemical precursors or other chemicals when chemically synthesized. In some instances, the isolated material will form part of a composition (for example, a crude extract containing other substances), buffer system or reagent mix. In other circumstances, the material can be purified to essential homogeneity, for example as determined by polyacrylamide gel electrophoresis (PAGE) or column chromatography (e.g., HPLC). An isolated nucleic acid molecule of the invention can comprise at least about 50%, at least about 80% or at least about 90% (on a molar basis) of all macromolecular species Present. With regard to genomic DNA, the term “isolated” also can refer to nucleic acid molecules that are separated from the chromosome with which the genomic DNA is naturally associated. For example, the isolated nucleic acid molecule can contain less than about 250 kb, 200 kb, 150 kb, 100 kb, 75 kb, 50 kb, 25 kb, 10 kb, 5 kb, 4 kb, 3 kb, 2 kb, 1 kb, 0.5 kb or 0.1 kb of the nucleotides that flank the nucleic acid molecule in the genomic DNA of the cell from which the nucleic acid molecule is derived.

The nucleic acid molecule can be fused to other coding or regulatory sequences and still be considered isolated. Thus, recombinant DNA contained in a vector is included in the definition of “isolated” as used herein. Also, isolated nucleic acid molecules include recombinant DNA molecules in heterologous host cells or heterologous organisms, as well as partially or substantially purified DNA molecules in solution. “Isolated” nucleic acid molecules also encompass in vivo and in vitro RNA transcripts of the DNA molecules of the present invention. An isolated nucleic acid molecule or nucleotide sequence can include a nucleic acid molecule or nucleotide sequence that is synthesized chemically or by recombinant means. Such isolated nucleotide sequences are useful, for example, in the manufacture of the encoded polypeptide, as probes for isolating homologous sequences (e.g., from other mammalian species), for gene mapping (e.g., by in situ hybridization with chromosomes), or for detecting expression of the gene in tissue (e.g., human tissue), such as by Northern blot analysis or other hybridization techniques.

The invention also pertains to nucleic acid molecules that hybridize under high stringency hybridization conditions, such as for selective hybridization, to a nucleotide sequence described herein (e.g., nucleic acid molecules that specifically hybridize to a nucleotide sequence containing a polymorphic site associated with a marker or haplotype described herein). Such nucleic acid molecules can be detected and/or isolated by allele- or sequence-specific hybridization (e.g., under high stringency conditions). Stringency conditions and methods for nucleic acid hybridizations are well known to the skilled person (see, e.g., Current Protocols in Molecular Biology, Ausubel, F. et al, John Wiley & Sons, (1998), and Kraus, M. and Aaronson, S., Methods Enzymol., 200:546-556 (1991), the entire teachings of which are incorporated by reference herein.

The percent identity of two nucleotide or amino acid sequences can be determined by aligning the sequences for optimal comparison purposes (e.g., gaps can be introduced in the sequence of a first sequence). The nucleotides or amino acids at corresponding positions are then compared, and the percent identity between the two sequences is a function of the number of identical positions shared by the sequences (i.e., % identity=# of identical positions/total # of positions×100). In certain embodiments, the length of a sequence aligned for comparison purposes is at least 30%, at least 40%, at least 50%, at least 60%, at least 70%, at least 80%, at least 90%, Or at least 95%, of the length of the reference sequence. The actual comparison of the two sequences can be accomplished by well-known methods, for example, using a mathematical algorithm. A non-limiting example of such a mathematical algorithm is described in Karlin, S. and Altschul, S., Proc. Natl. Acad. Sci. USA, 90:5873-5877 (1993). Such an algorithm is incorporated into the NBLAST and XBLAST programs (version 2.0), as described in Altschul, S. et al., Nucleic Acids Res., 25:3389-3402 (1997). When utilizing BLAST and Gapped BLAST programs, the default parameters of the respective programs (e.g., NBLAST) can be used. See the website on the world wide web at ncbi.nlm.nih.gov. In one embodiment, parameters for sequence comparison can be set at score=100, wordlength=12, or can be varied (e.g., W=5 or W=20). Another example of an algorithm is BLAT (Kent, W. J. Genome Res. 12:656-64 (2002)).

Other examples include the algorithm of Myers and Miller, CABIOS (1989), ADVANCE and ADAM as described in Torellis, A. and Robotti, C., Comput. Appl. Biosci. 10:3-5 (1994); and FASTA described in Pearson, W. and Lipman, D., Proc. Natl. Acad. Sci. USA, 85:2444-48 (1988).

In another embodiment, the percent identity between two amino acid sequences can be accomplished using the GAP program in the GCG software package (Accelrys, Cambridge, UK). The nucleic acid fragments of the invention are used as probes or primers in assays such as those described herein. “Probes” or “primers” are oligonucleotides that hybridize in a base-specific manner to a complementary strand of a nucleic acid molecule. In addition to DNA and RNA, such probes and primers include polypeptide nucleic acids (PNA), as described in Nielsen, P. et al., Science 254:1497-1500 (1991). A probe or primer comprises a region of nucleotide sequence that hybridizes to at least about 15, typically about 20-25, and in certain embodiments about 40, 50 or 75, consecutive nucleotides of a nucleic acid molecule. In one embodiment, the probe or primer comprises at least one allele of at least one polymorphic marker or at least one haplotype described herein, or the complement thereof. In particular embodiments, a probe or primer can comprise 100 or fewer nucleotides; for example, in certain embodiments from 6 to 50 nucleotides, or, for example, from 12 to 30 nucleotides. In other embodiments, the probe or primer is at least 70% identical, at least 80% identical, at least 85% identical, at least 90% identical, or at least 95% identical, to the contiguous nucleotide sequence or to the complement of the contiguous nucleotide sequence. In another embodiment, the probe or primer is capable of selectively hybridizing to the contiguous nucleotide sequence or to the complement of the contiguous nucleotide sequence. Often, the probe or primer further comprises a label, e.g., a radioisotope, a fluorescent label, an enzyme label, an enzyme co-factor label, a magnetic label, a spin label, an epitope label.

The nucleic acid molecules of the invention, such as those described above, can be identified and isolated using standard molecular biology techniques well known to the skilled person. The amplified DNA can be labeled (e.g., radiolabeled, fluorescently labeled) and used as a probe for screening a cDNA library derived from human cells. The cDNA can be derived from mRNA and contained in a suitable vector. Corresponding clones can be isolated, DNA obtained following in vivo excision, and the cloned insert can be sequenced in either or both orientations by art-recognized methods to identify the correct reading frame encoding a polypeptide of the appropriate molecular weight. Using these or similar methods, the polypeptide and the DNA encoding the polypeptide can be isolated, sequenced and further characterized.

### Antibodies

Polyclonal antibodies and/or monoclonal antibodies that specifically bind one form of the gene product but not to the other form of the gene product are also provided. Antibodies are also provided which bind a portion of either the variant or the reference gene product that contains the polymorphic site or sites. The term “antibody” as used herein refers to immunoglobulin molecules and immunologically active portions of immunoglobulin molecules, i.e., molecules that contain antigen-binding sites that specifically bind an antigen. A molecule that specifically binds to a polypeptide of the invention is a molecule that binds to that polypeptide or a fragment thereof, but does not substantially bind other molecules in a sample, e.g., a biological sample, which naturally contains the polypeptide. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind to a polypeptide of the invention. The term “monoclonal antibody” or “monoclonal antibody composition”, as used herein, refers to a population of antibody molecules that contain only one species of an antigen binding site capable of immunoreacting with a particular epitope of a polypeptide of the invention. A monoclonal antibody composition thus typically displays a single binding affinity for a particular polypeptide of the invention with which it immunoreacts.

Polyclonal antibodies can be prepared as described above by immunizing a suitable subject with a desired immunogen, e.g., polypeptide of the invention or a fragment thereof. The antibody titer in the immunized subject can be monitored over time by standard techniques, such as with an enzyme linked immunosorbent assay (ELISA) using immobilized polypeptide. If desired, the antibody molecules directed against the polypeptide can be isolated from the mammal (e.g., from the blood) and further purified by well-known techniques, such as protein A chromatography to obtain the IgG fraction. At an appropriate time after immunization, e.g., when the antibody titers are highest, antibody-producing cells can be obtained from the subject and used to prepare monoclonal antibodies by standard techniques, such as the hybridoma technique originally described by Kohler and Milstein, Nature 256:495-497 (1975), the human B cell hybridoma technique (Kozbor et al., Immunol. Today 4: 72 (1983)), the EBV-hybridoma technique (Cole et al., Monoclonal Antibodies and Cancer Therapy, Alan R. Liss, 1985, Inc., pp. 77-96) or trioma techniques. The technology for producing hybridomas is well known (see generally Current Protocols in Immunology (1994) Coligan et al., (eds.) John Wiley & Sons, Inc., New York, N.Y.). Briefly, an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds a polypeptide of the invention.

Any of the many well known protocols used for fusing lymphocytes and immortalized cell lines can be applied for the purpose of generating a monoclonal antibody to a polypeptide of the invention (see, e.g., Current Protocols in Immunology, supra; Galfre et al., Nature 266:55052 (1977); R. H. Kenneth, in Monoclonal Antibodies: A New Dimension In Biological Analyses, Plenum Publishing Corp., New York, N.Y. (1980); and Lerner, Yale J. Biol. Med. 54:387-402 (1981)). Moreover, the ordinarily skilled worker will appreciate that there are many variations of such methods that also would be useful.

Alternative to preparing monoclonal antibody-secreting hybridomas, a monoclonal antibody to a polypeptide of the invention can be identified and isolated by screening a recombinant combinatorial immunoglobulin library (e.g., an antibody phage display library) with the polypeptide to thereby isolate immunoglobulin library members that bind the polypeptide. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al., Bio/Technology 9: 1370-1372 (1991); Hay et al., Hum. Antibod. Hybridomas 3:81-85 (1992); Huse et al., Science 246: 1275-1281 (1989); and Griffiths et al., EMBO J. 12:725-734 (1993).

Additionally, recombinant antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art.

In general, antibodies of the invention (e.g., a monoclonal antibody) can be used to isolate a polypeptide of the invention by standard techniques, such as affinity chromatography or immunoprecipitation. A polypeptide-specific antibody can facilitate the purification of natural polypeptide from cells and of recombinantly produced polypeptide expressed in host cells. Moreover, an antibody specific for a polypeptide of the invention can be used to detect the polypeptide (e.g., in a cellular lysate, cell supernatant, or tissue sample) in order to evaluate the abundance and pattern of expression of the polypeptide. Antibodies can be used diagnostically to monitor protein levels in tissue as part of a clinical testing procedure, e.g., to, for example, determine the efficacy of a given treatment regimen. The antibody can be coupled to a detectable substance to facilitate its detection. Examples of detectable substances include various enzymes, prosthetic groups, fluorescent materials, luminescent materials, bioluminescent materials, and radioactive materials. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and acquorin, and examples of suitable radioactive material include ¹²⁵I, ¹³¹I, ^{±}S or ³H.

Antibodies may also be useful in pharmacogenomic analysis. In such embodiments, antibodies against variant proteins encoded by nucleic acids according to the invention, such as variant proteins that are encoded by nucleic acids that contain at least one polymorphic marker of the invention, can be used to identify individuals that require modified treatment modalities.

Antibodies can furthermore be useful for assessing expression of variant proteins in disease states, such as in active stages of a disease, or in an individual with a predisposition to a disease related to the function of the protein, in particular urinary bladder cancer (UBC). Antibodies specific for a variant protein of the present invention that is encoded by a nucleic acid that comprises at least one polymorphic marker or haplotype as described herein can be used to screen for the presence of the variant protein, for example to screen for a predisposition to UBC as indicated by the presence of the variant protein.

Antibodies can be used in other methods. Thus, antibodies are useful as diagnostic tools for evaluating proteins, such as variant proteins of the invention, in conjunction with analysis by electrophoretic mobility, isoelectric point, tryptic or other protease digest, or for use in other physical assays known to those skilled in the art. Antibodies may also be used in tissue typing. In one such embodiment, a specific variant protein has been correlated with expression in a specific tissue type, and antibodies specific for the variant protein can then be used to identify the specific tissue type.

Subcellular localization of proteins, including variant proteins, can also be determined using antibodies, and can be applied to assess aberrant subcellular localization of the protein in cells in various tissues. Such use can be applied in genetic testing, but also in monitoring a particular treatment modality. In the case where treatment is aimed at correcting the expression level or presence of the variant protein or aberrant tissue distribution or developmental expression of the variant protein, antibodies specific for the variant protein or fragments thereof can be used to monitor therapeutic efficacy.

Antibodies are further useful for inhibiting variant protein function, for example by blocking the binding of a variant protein to a binding molecule or partner. Such uses can also be applied in a therapeutic context in which treatment involves inhibiting a variant protein's function. An antibody can be for example be used to block or competitively inhibit binding, thereby modulating (i.e., agonizing or antagonizing) the activity of the protein. Antibodies can be prepared against specific protein fragments containing sites required for specific function or against an intact protein that is associated with a cell or cell membrane. For administration in vivo, an antibody may be linked with an additional therapeutic payload, such as radionuclide, an enzyme, an immunogenic epitope, or a cytotoxic agent, including bacterial toxins (diphtheria or plant toxins, such as ricin). The in vivo half-life of an antibody or a fragment thereof may be increased by pegylation through conjugation to polyethylene glycol.

The present invention further relates to kits for using antibodies in the methods described herein. This includes, but is not limited to, kits for detecting the presence of a variant protein in a test sample. One preferred embodiment comprises antibodies such as a labelled or labelable antibody and a compound or agent for detecting variant proteins in a biological sample, means for determining the amount or the presence and/or absence of variant protein in the sample, and means for comparing the amount of variant protein in the sample with a standard, as well as instructions for use of the kit.

The present invention will now be exemplified by the following non-limiting example.

### Example

Patient and Control Selection: Collection of blood samples and medical information was conducted with informed consent and ethical review board approval in accordance with the Declaration of Helsinki.

Collection of blood samples and medical information was conducted with informed consent and ethical review board approval in accordance with the Declaration of Helsinki.

Study populations: Seven study populations were used in this work, two discovery populations (Iceland and the Netherlands) and 5 follow up sample sets. The Icelandic sample set consists of 525 patients and 32,504 controls and the Dutch sample set consists of 1,278 cases and 1,832 controls. Follow up genotyping was performed in sample sets from Leeds, UK (724 cases and 530 controls), Torino, Italy (329 cases and 389 controls), Brescia, Italy (182 cases and 192 controls), Leuven, Belgium (195 cases and 382 controls) and an Eastern European sample set obtained from the German Cancer Research Center (DKFZ) in Heidelberg (213 cases and 521 controls).

1. Icelandic study population. Records of all urinary bladder cancer diagnoses were obtained from the Icelandic Cancer Registry (ICR) (http://www.krabbameinsskra.is). The ICR contains all cancer diagnoses in Iceland from Jan. 1, 1955. The ICR contained records of 1,642 Icelandic UBC patients diagnosed until Dec. 31, 2006, and all prevalent cases were eligible to participate. The mean participation rate for newly diagnosed cases was (55%. Patients Were recruited by trained nurses on behalf of the patients' treating physicians, through special recruitment clinics. Participants in the study donated a blood sample and answered a lifestyle questionnaire. A total of 545 patients (76% males; diagnosed from December 1974 to June 2006) were included in a genome-wide SNP genotyping effort, using the Infinium II assay method and either the Sentrix HumanHap 300 or HumanCNV370-duo BeadChip (Illumina). A total of 525 individuals (96%) were successfully genotyped. The median age at diagnosis for all consenting cases was 67 years (range 22-94 years) as compared to 68.5 years for all UBC patients in the ICR. The 32,504 controls (41% males; mean age 61 years; SD=21) used in this study consisted of individuals from other ongoing genome-wide association studies at deCODE. The controls were absent from the nationwide list of UBC patients according to the ICR. Samples from prostate, breast and colorectal cancer patients as well as individuals used for the analysis of smoking variables come from other ongoing project at deCODE Genetics (1-3). The study was approved by the Data Protection Authority of Iceland and the National Bioethics Committee. Written informed consent was obtained from all patients, relatives and controls. Personal identifiers associated with medical information and blood samples were encrypted with a third-party encryption system in which the Data Protection Authority maintains the code.

Genealogical Database: deCODE Genetics maintains a computerized database of the genealogy of Icelanders. The records include almost all individuals born in Iceland in the last two centuries and for that period around 95% of the parental connections are known (Sigurdardottir, et al., Am J Hum Genet, 66, 1599-609 (2000)). In addition, a county of residence identifier is recorded for most individuals, based on census and parish records. The information is stored in a relational database with encrypted personal identifiers that match those used on the biological samples and ICR records, allowing cross-referencing of the genotypes and phenotypes of the study participants with their genealogies.

2. Nijmegen Bladder Cancer Study, the Netherlands (PI: Dr. Lambertus Kiemeney). The Dutch patients were recruited for the Nijmegen Bladder Cancer Study (see http://dceg.cancer.gov/icbc/membership.html). The Nijmegen Bladder Cancer Study identified patients through the population-based regional cancer registry held by the Comprehensive Cancer Centre East, Nijmegen that serves a region of 1.3 million inhabitants in the eastern part of the Netherlands (www.ikcnet.nl). Patients diagnosed between 1995 and 2006 under the age of 75 years were selected and their vital status and current addresses updated through the hospital information systems of the 7 community hospitals and 1 university hospital (Radboud University Nijmegen Medical Center, RUNMC) that are covered by the cancer registry. All patients still alive on Aug. 1, 2007 were invited to the study by the Comprehensive Cancer Center on behalf of the patients' treating physicians. In case of consent, patients were sent a lifestyle questionnaire to fill out and blood samples were collected by Thrombosis Service centers which hold offices in all the communities in the region. 1,651 patients were invited to participate. Of all the invitees, 1,082 gave informed consent (66%): 992 filled out the questionnaire (60%) and 1016 (62%) provided a blood sample. The number of participating patients was increased with a non-overlapping series of 376 bladder cancer patients who were recruited previously for a study on gene-environment interactions in three hospitals (RUNMC, Canisius Wilhelmina Hospital, Nijmegen, and Streekziekenhuis Midden-Twente, Hengelo, the Netherlands). Ultimately, completed questionnaires and blood samples were available for 1,276 and 1,392 patients, respectively. All the patients that were selected for the analyses (N=1,278) were of self-reported European descent. The median age at diagnosis was 62 (range 25-93) years. 82% of the participants were males. Data on tumor stage and grade were obtained through the cancer registry.

The 1,832 control individuals (46% males) were cancer free and frequency-matched for age with the cases. They were recruited within a project entitled “Nijmegen Biomedical Study”. The details of this study were reported previously (Wetzels, J. F., et al. Kidney Int 72(5):632-7.(2007)). Briefly, this is a population-based survey conducted by the Department of Epidemiology and Biostatistics and the Department of Clinical Chemistry of the Radboud University Nijmegen Medical Center (RUNMC), in which 9,371 individuals participated from a total of 22,500 age and sex stratified, randomly selected inhabitants of Nijmegen. Control individuals from the Nijmegen Biomedical Study were invited to participate in a study on gene-environment interactions in multifactoral diseases, such as cancer. All the 1,832 participants in the present study are of self-reported European descent and were fully informed about the goals and the procedures of the study. The study protocols of the Nijmegen Bladder Cancer Study and the Nijmegen Biomedical Study were approved by the Institutional Review Board of the RUNMC and all study subjects gave written informed consent.

3. Leeds Bladder Cancer Study, United Kingdom (PIs: Dr. Anne Kiltie and Dr. Timothy Bishop). Details of the Leeds Bladder Cancer Study have been reported previously (Sak, S. C., et al. Br J Cancer 92(12):2262-5 (2005)). In brief, patients from the urology department of St James's University Hospital, Leeds were recruited from August 2002 to March 2006. All those patients attending for cystoscopy or transurethral resection of a bladder tumor (TURBT) who had previously been found, or were subsequently shown, to have urothelial cell carcinoma of the bladder were included. Exclusion criteria were significant mental impairment, or a blood transfusion in the past month. All non-Caucasians were excluded from the study leaving 764 patients. Genotyping was successful in 724 patients or 95%. The median age at diagnosis of the patients was 73 years (range 30-101). 71% of the patients were male and 61% of all the patients had a low risk tumor (pTaG1/2). The controls were recruited from the otolaryngology outpatients and ophthalmology inpatient and outpatient departments at St James's Hospital, Leeds, from August 2002 to March 2006. All controls of appropriate age for frequency matching with the cases were approached and recruited if they gave their informed consent. As for the cases, exclusion criteria for the controls were significant mental impairment, or a blood transfusion in the past month. Also, controls were excluded if they had symptoms suggestive of bladder cancer, such as hematuria. 2.8% of the controls were non-Caucasian leaving 545 Caucasian controls for the study. 71% of the controls were male. Data were collected by a health questionnaire on smoking habits and smoking history (non-ex- or current smoker, smoking dose in pack-years), occupational exposure history (to plastics, rubber, laboratories, printing, dyes and paints, diesel fumes), family history of bladder cancer, ethnicity and place of birth, and places of birth of parents. The response rate of cases was approximately 99%, that among the controls approximately 80%. Ethical approval for the study was obtained from Leeds (East) Local Research Ethics Committee, project number 02/192.

4. Torino Bladder Cancer Case Control Study, Italy (PIs: Dr. Giuseppe Matullo and Dr. Paolo Vineis). The source of cases for the Torino bladder cancer study are two urology departments of the main hospital in Torino, the San Giovanni Battista Hospital (Matullo G, et al. Cancer Epidemiol Biomarkers Prev 14(11 Pt 1):2569-78 (2005)). Cases are all Caucasian men, aged 40 to 75 years (median 63 years) and living in the Torino metropolitan area. They were newly diagnosed between 1994 and 2006 with a histologically confirmed, invasive or in situ, bladder cancer. Of all the patients with information on stage and grade, 56% were low risk (pTaG1/2). The source of controls are urology, medical and surgical departments of the same hospital in Torino. All controls are Caucasian men resident in the Torino metropolitan area. They were diagnosed and treated between 1994 and 2006 for benign diseases (such as prostatic hyperplasia, cystitis, hernias, heart failure, asthma, and benign ear diseases). Controls with cancer, liver or renal diseases and smoking related conditions were excluded. The median age of the controls was 57 years (range 40 to 74). Data were collected by a professional interviewer who used a structured questionnaire to interview both cases and controls face-to-face. Data were collected on demographics (age, sex, ethnicity, region and education), active and passive smoking (including brand and tobacco type), occupational exposures, drug use, family history, intake of fruit and vegetables, tea and coffee intake, and fluid intake. For cases, additional data were collected on tumor histology, tumor site, size, stage, grade, and treatment of the primary tumor. The response rates were 90% for cases and 75% for controls resulting in 333 cases and 392 controls. Ethical approval for the study was obtained from Comitato Etico Interaziendale, A.O.U. San Giovanni Batista—A.). C.T.O./Maria Adelaide.

5. The Brescia bladder cancer study, Italy (PI: Dr. Stefano Porru). The Brescia bladder cancer study is a hospital-based case-control study. The study was reported in detail previously (Shen, M, et al. Cancer Epidemiol Biomarkers Prev 12(11 Pt 1):1234-40.(2003)). In short, the catchment area of the cases and controls was the Province of Brescia, a highly Industrialized area in Northern Italy (mainly metal and mechanical industry, construction, transport, textiles) but also with relevant agricultural areas. Cases and controls were enrolled in 1997 to 2000 from the two main city hospitals. The total number of eligible subjects was 216 cases and 220 controls. The response rate (enrolled/eligible) was 93% (N=201) for cases and 97% (N=214) for controls. Only males were included. All cases and controls had Italian nationality and were of Caucasian ethnicity. All cases had to be residents of the Province of Brescia, aged between 20 and 80, and newly diagnosed with histologically confirmed bladder cancer. The median age of the patients was 63 years (range 22-80). 29% of all the patients with known stage and grade had a low risk tumor (pTaG1/2). Controls were patients admitted for various urological non-neoplastic diseases and were frequency matched to cases on age, hospital and period of admission. The study was formally approved by the ethical committee of the hospital where the majority of subjects were recruited. A written informed consent was obtained from all participants. Data were collected from clinical charts (tumor histology, site, grade, stage, treatments, etc.) and by means of face-to-face interviews during hospital admission, using a standardized semi-structured questionnaire. The questionnaire included data on demographics (age, ethnicity, region, education, residence, etc.), occupation (lifetime; industrial activity, job title and individual activities, specific evaluation for PAH and aromatic amines), smoking (lifetime, active and passive for non and ex-smokers), diet (food frequency, with emphasis on fruit, vegetables and PAH containing foods), liquid consumption (alcohol, water, soft drinks, coffee, tea), diuresis, certain environmental exposures (lifetime residential history, PAH, water chlorination byproducts), leisure time activities. ISCO and ISIC codes and expert assessments were used for occupational coding. Blood samples were collected from cases and controls for genotyping and DNA adducts analyses.

6. The Belgian Case Control Study of Bladder Cancer (PIs: Dr. Frank Buntinx and Dr. Maurice Zeegers). The Belgian study has been reported in detail (Kellen, E, et al. Int J Cancer 118(10):2572-8.(2006)). In brief, cases were selected from the Limburg Cancer Registry (LIKAR) and were approached through urologists and general practitioners. All cases were diagnosed with histologically confirmed urothelial cell carcinoma of the bladder between 1999 and 2004, and were Caucasian inhabitants of the Belgian province of Limburg. Exclusion criteria were mental sickness or incapacity to comprehend the questions or unable to understand or speak Dutch. The median age of the patients was 68 years. 86% of all the patients were males. For the recruitment of controls, a request was made to the “Kruispuntbank” of the social security for simple random sampling, stratified by municipality and socio-economic status, among all citizens above 50 years of age of the province. Subsequently, an invitation letter was sent to the selected subjects through the “Kruispuntbank”. Exclusion criteria for the controls were similar as those for the cases. The median age of the controls was 64 years; 59% of the controls were males. Three trained interviewers visited cases and controls at home. Information was collected through a structured interview and a standardized food frequency questionnaire. In addition, biological samples were collected. Preference was given to blood samples. However, if this was refused by the participant, buccal swabs were collected (less than <5% of all participants). Genomic DNA was extracted from peripheral blood lymphocytes or buccal swabs using standard methods. Data were collected on medical history, lifetime smoking history, family history of bladder cancer, 20-year residential history, and a lifetime occupational history. Based on the lifetime occupational history, occupational exposure to PAHs and aromatic amines were blindly coded by two experienced occupational physicians. A standardized food frequency questionnaire, derived from the IMMEDIATE study (Iacoviello, L., et al. Nutr Metab Cardiovasc Dis 11 (4 Suppl):122-6 (2001)), was used to register nutritional characteristics. Finally, blood levels of selenium and cadmium were measured. An exact response rate of the cases is not known because the participating clinicians did not register the non-responders. The response rate of the controls was ˜30%. Informed consent was obtained from all participants and the study was approved by the ethical review board of the Medical School of the Catholic University of Leuven, Belgium.

7. The Eastern Europe study population (PIs: Dr. Rajiv Kumar and Dr. Tony Fletcher). The details of this study have been described previously (Thirumaran, R. K., et al. Carcinogenesis 27(8):1676-81 (2006)). Cases and controls were recruited as part of a study designed to evaluate the risk of various cancers due to environmental arsenic exposure in Hungary, Romania and Slovakia between 2002 and 2004. The recruitment was carried out in the counties of Bacs, Bekes, Csongrad and Jasz-Nagykun-Szolnok in Hungary; Bihor and Arad in Romania; and Banska Bytrica and Nitra in Slovakia. The cases (N=212) and controls (N=532) selected were of Hungarian, Romanian and Slovak nationalities. Bladder cancer patients were invited on the basis of histopathological examinations by pathologists. Hospital-based controls were included in the study, subject to fulfillment of a set of criteria. All general hospitals in the study areas were involved in the process of control recruitment. A rotation scheme was used in order to achieve appropriate geographical distribution. The controls were frequency matched with cases for age, gender, country of residence and ethnicity. Controls included general surgery, orthopedic and trauma patients aged 30-79 years with conditions like appendicitis, abdominal hernias, duodenal ulcers, cholelithiasis and fractures. Patients with malignant tumors, diabetes and cardiovascular diseases were excluded as controls. The median age for the bladder cancer patients was 65 years (range 36-90). 83% of the patients were males. The median age for the controls was 61 years (range 28-83). 51% of the controls were males. The response rates among cases and controls were ˜70%. Of all the patients with known stage and grade information, 28% had a low risk tumor (pTaG1/2). Clinicians took venous blood and other biological samples from cases and controls after consent forms had been signed. Cases and controls recruited to the study were interviewed by trained personnel and completed a general lifestyle questionnaire. Ethnic background for cases and controls was recorded along with other characteristics of the study population. Local ethical boards approved the study plan and design.

Classification of “low risk” and “high risk” patients. Based on stage and grade information, all patients were classified with regards to risk of progression. Patients with low risk of progression were defined as having TNM stage pTa in combination with WHO 1973 differentiation grade 1 or 2 or WHO/ISUP 2004 low grade (Epstein, J. I. et al. The World Health Organization/International Society of Urological Pathology consensus classification of urothelial (transitional cell) neoplasms of the urinary bladder. Bladder Consensus Conference Committee. Am J Surg Pathol 22(12):1435-48 (1998)). All other tumors were classified as high risk of progression (stage pTis or ≧pT1 or WHO 1973 grade 3 or WHO/ISUP 2004 high grade).

### Genotyping—Overview

Samples from Iceland and the Netherlands were used for the genome-wide association study (GWA) and were assayed with either the Infinium humanHap300 or humanCNV370 SNP chips (IIlumina). The analysis was restricted to 302,140 SNPs that passed quality filters and were deemed usable due to yield, faithfulness to Hardy-Weinberg expectations and consistency in genotype frequencies between the two arrays. All samples had call rates above 98%. All follow up genotyping was carried out by deCODE Genetics in Reykjavik, Iceland applying single track Centaurus assays (Nanogen) (Kutyavin, I. V., et al. Nucleic Acids Res 34(19):e128 (2006)). The quality of the Centaurus SNP assays was evaluated by genotyping each assay in the CEU HapMap samples and comparing the results with the publicly released HapMap data. Assays with >1.5% mismatch rate were not used and a linkage disequilibrium (LD) test was used for markers known to be in LD. The concordance rate of genotypes derived from the two genotyping platforms (Illumine and Centaurus) was >99.5%.

### Statistical Analysis

Association analysis. A likelihood procedure described in a previous publication (Gretarsdottir S, et al. Nat Genet. 35(2):131-8 (2003)) and implemented in the NEMO software was used for the association analyses. An attempt was made to genotype all individuals and all SNPs reported had a yield that was higher than 95% in every study group. The SNPs rs4645960 and rs16901979 are not a part of the Human Hap300/HumanCNV370-duo chips. For these SNPs, a subset of the large Icelandic control set as well as all Icelandic cases and all individuals from the other study groups were genotyped by single track assays. We tested the association of an allele to UBC using a standard likelihood ratio statistic that, if the subjects were unrelated, would have asymptotically a χ² distribution with one degree of freedom under the null hypothesis. Allelic frequencies rather than carrier frequencies are presented for the markers in the main text. Allele-specific ORs and associated P values were calculated assuming a multiplicative model for the two chromosomes of an individual (Falk, C. T., Rubinstein, P. Ann Hum Genet. 51 (Pt 3):227-33 (1987)), Results from multiple case-control groups were combined using a Mantel-Haenszel model (Mantel N, Haenszel W. J Nat/Cancer Inst 22(4):719-48 (1959)) in which the groups were allowed to have different population frequencies for alleles and genotypes but were assumed to have common relative risks.

Correction of the GWA studies by genomic control. To adjust for possible population stratification and the relatedness amongst individuals, we divided the χ² test statistics from the individual scans using the method of genomic control (Devlin, B., Roeder, K. Biometrics 55(4):997-1004 (1999)), i.e. the 302,140 χ² test statistics were divided by their means, which were 1.04 and 1.075 for Iceland and the Netherlands, respectively. Appendix FIG. 1 is a quantile-quantile (Q-Q) plot of the chi-square statistics, before and after adjustment, against the chi-square distribution,

Correlation between genotype and expression of c-Myc in whole blood and adipose tissue. c-Myc expression was analyzed in whole blood and adipose tissue from 744 and 602 individuals respectively and correlated with rs9642880 genotype status. Collection of whole blood and adipose tissue samples, mRNA isolation and expression profiling was described previously (Emilsson, V., et al. Nature 452(7186):423 (2008)). Expression changes between two samples were quantified as mean logarithm (log₁₀) expression ratio (MLR), i.e. expression ratios compared to background corrected intensity values for the two channels for each spot on the arrays (Schadt, E. E., et al. Nature 422(6929):297-302 (2003). The hybridizations went through the standard QC process, i.e. signal to noise ratio, reproducibility and accuracy at spike-in compounds. The correlation between MLR for c-Myc and the genotypes of the SNP rs9642880 was tested by regressing the MLR's on the number of copies of the at-risk T allele of rs9642880, adjusting for age, sex and, for whole blood, the differential blood cell count. All P-values were adjusted for relatedness of the individuals by simulating genotypes through the Icelandic genealogy as previously described (Stefansson, H., et al. Nat Genet. 37(2):129 (2005)). The probe used to test the expression of c-Myc was NM_{—}002467.

### Results

We genotyped 525 cases and 32,504 controls from Iceland as well as 1,278 cases and 1,832 controls from the Netherlands on the HumanHap300/HumanCNV370-duo BeadChips (Table 2). After removing SNPs failing quality control checks, 302,140 SNPs were tested for association with UBC. The results were adjusted for relatedness between individuals and for potential population stratification using the method of genomic control.

No single SNP reached our genome wide significance threshold (P<1.6×10⁻⁷; corresponding to 0.05/302,140) either in the combined or individual analysis of the Icelandic or Dutch GWA sample sets. The 10 most significant SNPs (all P<5×10⁻⁵, see Table 1) were genotyped using Centaurus single track assays in additional 1,643 UBC cases and 2,014 controls from five follow up groups all of European ancestry (Table 2).

The strongest association with UBC, reaching genome wide significance in the overall analysis of the discovery and follow up groups, was observed for the T allele of rs9642880 at 8q24.21 (combined odds ratio (OR)=1.23 (95% confidence interval 1.16-1.31), P=2.82×10⁻¹¹). This was followed by rs710521 (A) on 3q28 (combined OR=1.20 (95% CI 1.12-1.29), P=3.38×10⁻⁷) (Table 1). Both rs9642880 and rs710521 were nominally significant (P<0.05) in the combined analysis of the follow-up groups. The association of the 8 other SNPs to UBC did not significantly replicate in the follow up groups.

TABLE 1
Eight most significant markers in the GWA for Urinary Bladder Cancer

| | | | | | Avg. | | | | | | | | | |
|---|---|---|---|---|---|---|---|---|---|---|---|---|---|---|
| | Seq | Chro- | | | control | | | | | | | | | |
| | ID | mo- | Position | Al- | fre- | Genome wide association | | | Follow up groups | | | All combined | | |
| SNP | No | some | Build 36 | lele | quency | OR | 95% CI | P | OR | 95% CI | P | OR | 95% CI | P |
| rs9642880 | 1 | 8 | 128,787,250 | T | 45% | 1.21 | 1.12-1.31 | 2.72 × 10⁻⁶ | 1.27 | 1.15-1.39 | 2.72 × | 1.23 | 1.16-1.31 | 2.82 × |
| | | | | | | | | | | | 10⁻⁶ | | | 10⁻¹¹ |
| rs710521 | 2 | 3 | 191,128,627 | A | 73% | 1.23 | 1.13-1.35 | 7.10 × 10⁻⁶ | 1.16 | 1.03-1.29 | 7.10 × | 1.20 | 1.12-1.29 | 3.38 × |
| | | | | | | | | | | | 10⁻⁶ | | | 10⁻⁷ |
| rs12982672 | 3 | 19 | 40,960,311 | G | 91% | 1.33 | 1.17-1.52 | 2.35 × 10⁻⁵ | | | | | | |
| rs12584999 | 4 | 13 | 85,735,283 | A | 20% | 1.24 | 1.13-1.36 | 2.02 × 10⁻⁵ | | | | | | |
| rs233716 | 5 | 12 | 111,524,326 | A | 59% | 1.22 | 1.12-1.33 | 3.69 × 10⁻⁶ | | | | | | |
| rs233722 | 6 | 12 | 111,515,857 | T | 59% | 1.22 | 1.12-1.33 | 3.31 × 10⁻⁶ | | | | | | |
| rs10240737 | 7 | 7 | 131,715,919 | A | 89% | 1.32 | 1.16-1.51 | 1.81 × 10⁻⁵ | | | | | | |
| rs17418689 | 8 | 2 | 137,706,145 | G | 6% | 1.36 | 1.17-1.56 | 4.11 × 10⁻⁵ | | | | | | |

TABLE 2
Description of the case control groups used in the studies

| | | | Average age | | |
|---|---|---|---|---|---|
| | | | at diagnosis | % males | |
| Study Group | #cases | #controls | (range) | (cases) | Study type |
| Discovery Groups (GWA) | | | | | |
| Iceland | 525 | 32.504 | 67 (22-94) | 76 | Population based |
| the Netherlands | 1.278 | 1.832 | 62 (25-93) | 81 | Population based |
| Follow-up Groups | | | | | |
| UK (Leeds) | 724 | 530 | 73 (30-101) | 71 | Hospital-based |
| Italy (Torino) | 329 | 389 | 63 (40-75) | 100 | Hospital-based |
| Italy (Brescia) | 182 | 192 | 63 (22-80) | 100 | Hospital-based |
| Belgium (Leuven) | 195 | 382 | 68 (40-93) | 86 | Population based |
| Eastern Europe (Hungary, | 213 | 521 | 65 (36-90) | 83 | Hospital-based |
| Romania, Slovakia) | | | | | |
| Total | 3.446 | 36.350 | | | |

TABLE 3
Association results for rs9642880 (T) on 8q24.21 and rs710521 (A) on 3q28 and UBC in Iceland, The Netherlands, UK, Italy, Belgium,
Eastern Europe, Sweden and Spain.

| | rs9642880 | | | | | rs710521 | | | | |
|---|---|---|---|---|---|---|---|---|---|---|
| Study population | Frequency | | | | | Frequency | | | | |
| (N cases/N controls) | Cases | Controls | OR | 95% CI | P value | Cases | Controls | OR | 95% CI | P value |
| Discovery groups (GWA) | | | | | | | | | | |
| Iceland (523/32,458)^{a} | 0.53 | 0.48 | 1.21 | 1.07-1.37 | 3.10 × 10⁻³ | 0.76 | 0.72 | 1.23 | 1.07-1.42 | 4.32 × 10⁻³ |
| The Netherlands (1,269/1,824)^{a} | 0.53 | 0.48 | 1.22 | 1.10-1.36 | 2.53 × 10⁻⁴ | 0.76 | 0.72 | 1.24 | 1.10-1.40 | 4.87 × 10⁻⁴ |
| Follow up groups | | | | | | | | | | |
| Leeds, UK (695/507) | 0.49 | 0.45 | 1.21 | 1.03-1.42 | 0.021 | 0.75 | 0.72 | 1.14 | 0.95-1.36 | 0.147 |
| Torino, Italy (323/381) | 0.50 | 0.41 | 1.43 | 1.16-1.77 | 8.69 × 10⁻⁴ | 0.76 | 0.75 | 1.04 | 0.82-1.32 | 0.747 |
| Brescia, Italy (174/183) | 0.44 | 0.41 | 1.13 | 0.84-1.52 | 0.419 | 0.74 | 0.69 | 1.23 | 0.89-1.70 | 0.207 |
| Leuven, Belgium (195/372) | 0.49 | 0.45 | 1.15 | 0.90-1.46 | 0.254 | 0.79 | 0.73 | 1.34 | 1.00-1.79 | 0.048 |
| Eastern Europe (186/515) | 0.52 | 0.44 | 1.40 | 1.10-1.78 | 5.73 × 10⁻³ | 0.78 | 0.75 | 1.13 | 0.85-1.50 | 0.401 |
| Stockholm, Sweden (341/930) | 0.52 | 0.48 | 1.16 | 0.97-1.40 | 0.10 | 0.79 | 0.75 | 1.27 | 1.03-1.56 | 0.026 |
| Zaragoza, Spain (172/888) | 0.48 | 0.46 | 1.08 | 0.85-1.36 | 0.53 | 0.73 | 0.74 | 0.97 | 0.75, 1.25 | 0.80 |
| GWA (1,792/34,282)^{b} | | 0.48 | 1.21 | 1.12-1.31 | 2.72 × 10⁻⁶ | | 0.72 | 1.23 | 1.13-1.35 | 7.10 × 10⁻⁶ |
| Follow up groups (2,086/3,776)^{b} | | 0.44 | 1.22 | 1.13-1.33 | 1.1 × 10⁻⁶ | | 0.73 | 1.15 | 1.05-1.27 | 0.0028 |
| All combined (3,878/38,058)^{b} | | 0.46 | 1.21 | 1.15-1.28 | 1.2 × 10⁻¹¹ | | 0.73 | 1.19 | 1.12-1.27 | 1.10 × 10⁻⁷ |

All P values shown are two-sided. Shown are the corresponding numbers of cases and controls (N), allelic frequencies of variants in affected and control individuals, the allelic odds-ratio (OR) with P values based on the multiplicative model.
^{a}Results presented for Iceland and the Netherlands were individually adjusted by the method of genomic control (see Supplementary Methods).
^{b}For the combined study populations, the reported control frequency was the average, unweighted control frequency of the individual populations, while the OR and the P value were estimated using the Mantel-Haenszel model.

TABLE 4
C08
All markers with r2 > 0.2 with rs9642880 (1Mb each side)

| snp1 | snp2 | D′ | R2 | p value | chromosome | position | Seq ID No | pos SEQ ID NO: 54 |
|---|---|---|---|---|---|---|---|---|
| rs12547643 | rs9642880 | 0.838760 | 0.354058 | 1.74e−10 | C08 | 128,782,355 | 11 | 300 |
| rs17186926 | rs9642880 | 1.000000 | 0.289805 | 4.03e−11 | C08 | 128,787,625 | 12 | 5570 |

TABLE 5
C03
All markers with r2 > 0.2 with rs710521 (1Mb each side)

| snp1 | snp2 | D′ | R2 | p value | pos SEQ ID NO53 | position | Seq ID No. |
|---|---|---|---|---|---|---|---|
| rs6780540 | rs710521 | 0.696058 | 0.206370 | 3.28e−06 | 300 | 191,045,364 | 13 |
| rs9817981 | rs710521 | 0.696058 | 0.206370 | 3.28e−06 | 1,160 | 191,046,224 | 14 |
| rs9818301 | rs710521 | 0.700732 | 0.213540 | 2.26e−06 | 1,311 | 191,046,375 | 15 |
| rs2056124 | rs710521 | 0.904800 | 0.651655 | 6.78e−16 | 5,448 | 191,050,512 | 16 |
| rs2378526 | rs710521 | 0.871045 | 0.241186 | 3.40e−08 | 12,077 | 191,057,141 | 17 |
| rs1913720 | rs710521 | 0.902811 | 0.450778 | 1.46e−13 | 12,457 | 191,057,521 | 18 |
| rs17448036 | rs710521 | 0.869406 | 0.235635 | 4.74e−08 | 17,141 | 191,062,205 | 19 |
| rs1913721 | rs710521 | 0.869406 | 0.235635 | 4.74e−08 | 18,623 | 191,063,687 | 20 |
| rs11924151 | rs710521 | 0.887856 | 0.361974 | 1.19e−10 | 18,650 | 191,063,714 | 21 |
| rs3773928 | rs710521 | 0.904648 | 0.440671 | 7.53e−14 | 21,335 | 191,066,399 | 22 |
| rs6783043 | rs710521 | 1.000000 | 0.254032 | 2.24e−10 | 22,838 | 191,067,902 | 23 |
| rs1399773 | rs710521 | 1.000000 | 0.595142 | 2.79e−21 | 23,660 | 191,068,724 | 24 |
| rs1399774 | rs710521 | 0.809402 | 0.518237 | 6.14e−13 | 23,812 | 191,068,876 | 25 |
| rs6790167 | rs710521 | 1.000000 | 0.393258 | 3.17e−15 | 24,904 | 191,069,968 | 26 |
| rs9865857 | rs710521 | 1.000000 | 0.572708 | 9.43e−20 | 25,680 | 191,070,744 | 27 |
| rs9882348 | rs710521 | 1.000000 | 0.397993 | 2.05e−15 | 28,987 | 191,074,051 | 28 |
| rs9812089 | rs710521 | 1.000000 | 0.411765 | 7.80e−16 | 29,832 | 191,074,896 | 29 |
| rs7610966 | rs710521 | 1.000000 | 0.397993 | 2.05e−15 | 30,284 | 191,075,348 | 30 |
| rs1515490 | rs710521 | 0.781714 | 0.513753 | 1.21e−13 | 34,485 | 191,079,549 | 31 |
| rs7613791 | rs710521 | 1.000000 | 0.397993 | 2.05e−15 | 36,954 | 191,082,018 | 32 |
| rs1543969 | rs710521 | 1.000000 | 0.836334 | 1.07e−28 | 37,304 | 191,082,368 | 33 |
| rs12107036 | rs710521 | 1.000000 | 0.384615 | 5.25e−15 | 37,790 | 191,082,854 | 34 |
| rs1554132 | rs710521 | 1.000000 | 0.455621 | 3.73e−17 | 41,328 | 191,086,392 | 35 |
| rs6790068 | rs710521 | 0.572167 | 0.230853 | 4.88e−06 | 45,256 | 191,090,320 | 36 |
| rs4687100 | rs710521 | 1.000000 | 1.000000 | 1.15e−35 | 48,656 | 191,093,720 | 37 |
| rs9681004 | rs710521 | 0.582182 | 0.255506 | 3.21e−07 | 48,824 | 191,093,888 | 38 |
| rs4687102 | rs710521 | 1.000000 | 0.929204 | 4.84e−31 | 54,172 | 191,099,236 | 39 |
| rs17514925 | rs710521 | 1.000000 | 0.964222 | 1.37e−33 | 55,179 | 191,100,243 | 40 |
| rs7628595 | rs710521 | 1.000000 | 0.930070 | 4.16e−32 | 56,126 | 191,101,190 | 41 |
| rs7642848 | rs710521 | 1.000000 | 0.397993 | 2.05e−15 | 65,702 | 191,110,766 | 42 |
| rs12493699 | rs710521 | 1.000000 | 1.000000 | 1.15e−35 | 68,069 | 191,113,133 | 43 |
| rs12490406 | rs710521 | 0.960724 | 0.883996 | 2.21e−25 | 68,279 | 191,113,343 | 44 |
| rs1447931 | rs710521 | 1.000000 | 1.000000 | 4.23e−34 | 69,189 | 191,114,253 | 45 |
| rs4479569 | rs710521 | 1.000000 | 1.000000 | 2.74e−34 | 69,562 | 191,114,626 | 46 |
| rs4687103 | rs710521 | 1.000000 | 1.000000 | 1.78e−35 | 70,294 | 191,115,358 | 47 |
| rs4687104 | rs710521 | 1.000000 | 1.000000 | 5.11e−35 | 70,530 | 191,115,594 | 48 |
| rs12491886 | rs710521 | 1.000000 | 1.000000 | 1.15e−35 | 75,217 | 191,120,281 | 49 |
| rs11706540 | rs710521 | 1.000000 | 1.000000 | 1.15e−35 | 78,683 | 191,123,747 | 50 |
| rs837776 | rs710521 | 1.000000 | 0.358974 | 3.24e−14 | 81,230 | 191,126,294 | 51 |
| rs710555 | rs710521 | 0.580974 | 0.214793 | 1.45e−06 | 139,075 | 191,184,139 | 52 |

TABLE 6
Association of markers rs733677 and rs12547643 on 8q24 to UBC.

| | | | | | | | Average | | | | | | |
|---|---|---|---|---|---|---|---|---|---|---|---|---|---|
| | | | | | | | allele | | | | | | |
| | Al- | Position | | | LD to rs9642880 | | fre- | | | | OR | | P value |
| Marker | lele | (build 36) | #cases^{a} | #controls | D′ | r² | quency | OR | 95% CI | P value | adjusted | 95% CI | adjusted |
| rs4733677 | T | 128,781,003 | 3.409 | 36.224 | 0.6460 | 0.1855 | 0.22 | 1.20 | 1.12-1.29 | 8.00 × 10⁻⁷ | 1.10 | 1.01-1.19 | 0.021 |
| rs12547643 | A | 128,782,355 | 3.318 | 36.157 | 0.8388 | 0.3541 | 0.40 | 1.08 | 1.01-1.15 | 0.018 | 0.96 | 0.89-1.04 | 0.307 |

OR and P-value for association to UBC is shown before and after adjustment to rs9642880.
^{a}The cases and controls are from Iceland and the Netherlands.

TABLE 7
Markers at 8q24.21 previously reported for cancer association, their LD to rs9642880, average control frequency and
association to UBC in all 7 study groups combined

| | | Position | # UBC | | Allele | Association to UBC | | | LD to rs9642880 | | Published Cancer |
|---|---|---|---|---|---|---|---|---|---|---|---|
| Marker | Allele^{a} | (build 36) | cases | # controls | Freq | OR | 95% CI | P value | D′ | r² | Association |
| rs16901979 | A | 128,194,098 | 3,115 | 12,020^{b} | 0.03 | 0.98 | 0.81-1.19 | 0.84 | 0.259 | 0.001 | Prostate |
| rs672888^{c} | C | 128,424,800 | 3,383 | 36,212 | 0.40 | 1.02 | 0.96-1.08 | 0.51 | 0.156 | 0.017 | Breast |
| rs6983267 | G | 128,482,487 | 3,420 | 36,241 | 0.51 | 1.03 | 0.97-1.10 | 0.36 | 0.209 | 0.042 | Prostate, Colorectal |
| rs1447295 | A | 128,554,220 | 3,437 | 36,280 | 0.09 | 1.06 | 0.95-1.18 | 0.30 | 0.230 | 0.0031 | Prostate |

The SNPs rs16901979, rs672888, rs6983267 and rs1447295 were typed in UBC cases and controls from Iceland, The Netherlands, UK, Italy (Torino and Brescia), Belgium and Eastern Europe
^{a}Shown are the reported at risk alleles corresponding to published cancer association
^{b}The rs16901979 marker is not on the Illumina 317/370 chip and was typed by a single track assay for all UBC cases and all control samples, except in Iceland, where it was typed in 8,828 of the 32,504 control individuals.
^{c}Tagging G allele of rs13281615; r² = 0.97

TABLE 8
Association of rs9642880 (T) to other cancers in Iceland

| | | | Cases | Control | | | |
|---|---|---|---|---|---|---|---|
| Cancer type | #cases | #controls | frequency | frequency | OR | 95% CI | P-value |
| Prostate | 1597 | 31384 | 0.494 | 0.481 | 1.056 | 0.98-1.14 | 0.158 |
| Breast | 1781 | 31200 | 0.466 | 0.482 | 0.938 | 0.87-1.01 | 0.0766 |
| Colorectal | 961 | 32020 | 0.482 | 0.481 | 1.001 | 0.93-1.08 | 0.979 |

Association of UBC with 8q24

The variant rs9642880 on chromosome 8q24.21 reached genome wide significance in the combined analysis. The individual ORs for the SNP in the seven case control groups of the study were between 1.13-1.43 and no heterogeneity was observed between the estimates in the 7 groups (Pₕₑₜ=0.71). The association reached nominal significance in 5 out of 7 groups (Table 3). Two other SNPs at this locus, rs4733677 (P=8.0×10⁻⁷) and rs12547643 (P=0.018), showed nominal significance in the GWA analysis. rs12547643 was no longer significant after adjusting for the effect of rs9642880 (P=0.31). While the significance was greatly reduced, rs4733677 remained nominally significant (P=0.02) after adjusting for rs9642880 (Table 6). To investigate the mode of inheritance more carefully, we computed the genotype-specific OR for rs9642880. Results from all groups combined, demonstrated that the association of rs9642880 (T) to UBC did not deviate from the multiplicative model (P=0.37). Relative to the non-carriers, the ORs for heterozygous and homozygous carriers of the risk allele T were 1.23 and 1.51, respectively. Assuming that the frequency of the allele is 45%, i.e. the average of the frequency of all the populations studied (Table 3), then individuals homozygous for rs9642880 (TI) represent ˜20% of the population. The estimated population attributable risk (PAR) of rs9642880 (T) is 18%.

The SNP rs9642880 is located in the same LD block as the c-Myc oncogene and only 30 kb upstream of it (FIG. 1). c-Myc is the only known gene close to rs9642880, but a predicted gene, BC042052, is also in the same region. We genotyped samples from all the study groups for two known missense mutations in the c-Myc gene (G175C/rs4645960 and N26S/rs4645959) but found no association with UBC. rs4645960 (T) was very rare, with only two cases in the combined sample sets carrying the allele. rs4645959 (G) was weakly correlated with rs9642880 (T) (D′=1, r²=0.04) with a frequency of 3.3% in cases and 3.7% in controls (OR=0.91, P=0.29). To determine if the rs9642880 (T) would affect the expression of c-Myc we analyzed its expression in whole blood from 744 individuals and adipose tissue from 602 individuals and correlated the results with genotype data for rs9642880. No significant correlation was observed between c-Myc mRNA expression and the number of copies of the T allele of rs9642880 carried, either in whole blood or in adipose tissue (P=0.86 for whole blood and P=0.74 for adipose tissue).

Genome wide association studies have repeatedly reported cancer associated variants on 8q24, 200-700 kb proximal to rs9642880 and c-Myc. We and others found SNPs at 8q24.21 to be strongly associated with cancer of the prostate (rs1447295, rs6983267 and rs16901979) (Gudmundsson J., et al. Nat Genet. 39(5):631-7 (2007); Eeles, R. A., et al. Nat Genet. 40(3):316-21 (2008); Amundadottir L. T., et al. Nat Genet 7:7 (2006); Thomas, G., et al. Nat Genet; 40(3):310-5 (2008). Subsequently, rs6983267 was also shown to associate with colorectal cancer (Tomlinson, I., et al. Nat Genet. 39(8):984-8 (2007); Heiman, C. A., et al. Nat Genet. 39(8):954-6 (2007), Zanke, B. W., et al. Nat Genet. 39(8):989-94 (2007)) and most recently rs13281615 with breast cancer (Easton, D. F., et al. Nature 447(7148):1087-93 (2007)). These 4 variants are dispersed over a 500 kb region (FIG. 1) and are in weak LD with each other and with rs9642880 (Table 7). We found no association between these 0.4 SNPs and UBC in the combined study groups (Table 7). Moreover, we found no association between rs9642880 and prostate, breast or colorectal cancer in Icelandic case control samples (Table 8).

Information on stage and grade was available for 5 of the 7 groups. Based on this information the UBC cases were classified into patients with a good prognosis (‘low risk’: tumor confined to the bladder mucosa and not poorly differentiated) or patients with a considerable risk of tumor progression (‘high risk’: tumor invasion in or beyond the lamina propria or poorly differentiated). Comparing the frequency of rs9642880 (T) between patients with low and high risk tumors showed some heterogeneity across the study groups. Patients with low risk tumors from the Netherlands and Eastern Europe had a higher frequency of rs9642880 (T) than patients with high risk tumors, while no difference was detected in the other groups (combined OR=1.13, P=0.05). This potential association with tumor aggressiveness needs to be investigated further in a large number of cases and controls.

Association with 3q28

The second strongest signal in the combined analysis was observed for rs710521 (A) on chromosome 3q28, which nearly reached genome-wide significance (OR=1.20, P=3.38×10⁻⁷) (Table 1). No heterogeneity was observed between the ORs of the 7 study groups (Pₕₑₜ=0.83). The association of rs710521 (A) to UBC did not deviate from the multiplicative model (P=0.35). The estimated population attributable risk (PAR) of rs710521 (A) is 24%. No significant interaction was observed between the effects of rs9642880 and rs710521 (P=0.51). No difference was detected in frequency of rs710521 (A) between patients with low vs. high risk of progression. The rs710521 SNP is located in an LD block that overlaps the TP63 gene (encoding tumor protein p63), a homologue of the tumor suppressor gene TP53.

### Smoking-Related Effects

We and others have recently found an association between variation in the nicotinic acetylcholine receptor gene cluster on 15q24 and nicotine addiction, smoking behavior, lung cancer and peripheral arterial disease (Saccone, S. F., et al. Hum Mol Genet. 16(1):36-49 (2007); Thorgeirsson, T. E., et al. Nature 452(7187):638-42 (2008); Amos, C. I. et al. Nat Genet. 98(2):274-8 (2008); Hung, R. J., et al. Nature 452(7187):633-7 (2008)). Since smoking is a strong risk factor for UBC, we tested the reported smoking-associated variant, rs1051730, in all the 7 UBC case control groups but found no association between the risk allele and disease (combined OR=1,005, P=0.88). No difference in the frequencies of rs9642880 (T) on chromosome 8 and rs710521 (A) on chromosome 3 was observed between ever smoking and never smoking cases (P=0.38 and P=0.79 respectively). Similarly, analysis of the Icelandic and Dutch controls showed that the results observed for rs9642880 and rs710521 cannot be explained by an association of these SNPs to smoking initiation or smoking quantity (data not shown). rs9642880 (T) and rs710521 (A) did not correlate with age at diagnosis nor did we observe any gender effect (all P>0.05).

1. A method for determining a susceptibility to urinary bladder cancer in a human individual, comprising determining the presence or absence of at least one allele of at least one polymorphic marker in a nucleic acid sample obtained from the individuals, wherein the at least one polymorphic marker is selected from the group consisting of rs9642880, rs710521, rs12982672, rs12584999, rs233716, rs233722, rs10240737, rs17418689 and rs4733677, and markers in linkage disequilibrium therewith, wherein the linkage disequilibrium is characterized by a value for r² of at least 0.1,
and determining a susceptibility to urinary bladder cancer in the subject from the presence or absence of the at least one allele, wherein determination of the presence of the at least one allele is indicative of a susceptibility to urinary bladder cancer for the individual.

2. The method according to claim 1, wherein the at least one polymorphic marker is selected from the group consisting of rs9642880, rs12547643, rs17186926, and rs4733677.

3. The method according to claim 2, wherein the at least one polymorphic marker is rs9642880.

4. The method according to claim 1, wherein the at least one polymorphic marker is selected from the group consisting of rs710521, rs6780540, rs9817981, rs9818301, rs2056124, rs2378526, rs1913720, rs17448036, rs1913721, rs11924151, rs3773928, rs6783043, rs1399773, rs1399774, rs6790167, rs9865857, rs9882348, rs9812089, rs7610966, rs1515490, rs7613791, rs1543969, rs12107036, rs1554132, rs6790068, rs4687100, rs9681004, rs4687102, rs17514925, rs7628595, rs7642848, rs12493699, rs12490406, rs1447931, rs4479569, rs4687103, rs4687104, rs12491886, rs11706540, rs837776 and rs710555.

5. The method according to claim 4 wherein the at least one polymorphic marker is rs710521.

6. The method according to claim 1, further comprising assessing the frequency of at least one haplotype comprising at least two polymorphic markers in the individual.

7. The method of claim 1, wherein the susceptibility conferred by the presence of the at least one allele or haplotype is increased susceptibility.

8. The method according to claim 7, wherein the at least one allele is selected from the group consisting of allele T in rs9642880, allele A in rs710521, allele G in rs12982672, allele A in rs12584999, allele A in rs233716, allele T in rs233722, allele A in rs10240737, allele G in rs17418689 and allele T in rs4733677, and wherein the presence of the allele is indicative of increased susceptibility to urinary bladder cancer.

9. The method according to claim 7, wherein the presence of the at least one allele or haplotype is indicative of increased susceptibility to urinary bladder cancer with a relative risk (RR) or odds ratio (OR) of at least 1.20.

10. The method according to claim 1, wherein the susceptibility conferred by the presence of the at least one allele or haplotype is decreased susceptibility.

11. The method of claim 1, further comprising analyzing non-genetic information to make risk assessment, diagnosis, or prognosis of the individual.

12. The method of claim 11 wherein the non-genetic information is selected from age, gender, ethnicity, socioeconomic status, previous disease diagnosis, medical history of subject, family history of urinary bladder cancer, history of occupational exposure to chemicals, biochemical measurements, and clinical measurements.

13. The method of claim 11 wherein said non-genetic information comprises information relating to tobacco smoking habits and/or tobacco smoking history of said individual.

14. A method of determining a susceptibility to urinary bladder cancer in a human individual, the method comprising:
obtaining nucleic acid sequence data about a human individual from a biological sample comprising nucleic acid from the individual, identifying at least one allele of at least one polymorphic marker selected from the group consisting of rs9642880, rs710521, rs12982672, rs12584999, rs233716, rs233722, rs10240737, rs17418689 and rs4733677, and markers in linkage disequilibrium therewith, wherein the linkage disequilibrium is characterized by a value for r² of at least 0.1, and wherein different alleles of the at least one polymorphic marker are associated with different susceptibilities to urinary bladder cancer in humans, and
determining a susceptibility to urinary bladder cancer from the nucleic acid sequence data.

15. The method of claim 14, wherein said at least one polymorphic marker is selected from the group consisting of rs9642880 (SEQ ID NO: 1) and rs710521 (SEQ ID NO: 2), and markers in linkage disequilibrium therewith.

16. The method of claim 15, wherein markers in linkage disequilibrium with rs9642880 are selected from the group consisting of rs12547643 and rs17186926.

17. (canceled)

18. (canceled)

19. (canceled)

20. A method for selecting candidates for screening programs for urinary bladder cancer, comprising:
determining with the method of claim 1 or claim 14 a susceptibility to urinary bladder cancer in a group of individuals, wherein individuals who are determined to have increased susceptibility to urinary bladder cancer are selected as candidates fora screening program for urinary bladder cancer.

21. The method of claim 20, wherein said screening program is selected from a urine dipstick test for hematuria, cystoscopy and urine cytology.

22. (canceled)

23. (canceled)

24. (canceled)

25. (canceled)

26. (canceled)

27. A computer-readable medium having computer executable instructions for determining susceptibility to urinary bladder cancer in an individual, the computer readable medium comprising:
data indicative of at least one polymorphic marker;
a routine stored on the computer readable medium and adapted to be executed by a processor to determine risk of developing urinary bladder cancer for the at least one polymorphic marker,
wherein the at least one polymorphic marker is selected from the group consisting of rs9642880, rs710521, rs12982672, rs12584999, rs233716, rs233722, rs10240737, rs17418689 and rs4733677, and markers in linkage disequilibrium therewith, wherein the linkage disequilibrium is characterized by a value for r² of at least 0.1.

28. The computer-readable medium of claim 27, wherein said data indicative of at least one polymorphic marker comprises parameters indicative of risk of urinary bladder linked to said at least one polymorphic marker.

29. An apparatus for determining a genetic indicator for urinary bladder cancer in a human individual, comprising:
a processor,
a computer readable memory having computer executable instructions adapted to be executed on the processor to analyze marker and/or haplotype information for at least one human individual with respect to at least one polymorphic marker selected from the group consisting of rs9642880, rs710521, rs12982672, rs12584999, rs233716, rs233722, rs10240737, rs17418689 and rs4733677, and markers in linkage disequilibrium therewith, wherein the linkage disequilibrium is characterized by a value for r² of at least 0.1, and generate an output based on the marker or haplotype information, wherein the output comprises a risk measure of the at least one marker or haplotype as a genetic indicator of urinary bladder cancer for the human individual.

30. The apparatus according to claim 29, wherein the computer readable memory further comprises data indicative of the frequency of at least one allele of at least one polymorphic marker or at least one haplotype in a plurality of individuals diagnosed with the condition, and data indicative of the frequency of at the least one allele of at least one polymorphic marker or at least one haplotype in a plurality of reference individuals, and wherein the risk measure of developing the condition is based on a comparison of the frequency of the at least one allele or haplotype in individuals diagnosed with the condition and reference individuals.

31. A method of assessing a subject's risk or Urinary Bladder Cancer, the method comprising:
a) obtaining sequence information about a human subject identifying at least one allele of at least one polymorphic marker selected from the group consisting of rs9642880, rs710521, rs12982672, rs12584999, rs233716, rs233722, rs10240737, rs17418689 and rs4733677, and markers in linkage disequilibrium therewith, wherein the linkage disequilibrium is characterized by a value for r² of at least 0.1, in the genome of the subject;
b) representing the sequence information as digital genetic profile data;
c) electronically processing the digital genetic profile data to generate a risk assessment report for Urinary Bladder Cancer for the individual; and
d) displaying the risk assessment report on an output device.
